(19) United States
(12) Patent Application Publication
(10) Pub. No.: US 2020/0339548 A1
(43) Pub. Date: Oct. 29, 2020

(54) PYRIMIDINE DERIVATIVES AND THEIR USE FOR THE TREATMENT OF CANCER

(71) Applicant: Adorx Therapeutics Limited, Edinburgh (GB)

(72) Inventors: Clive McCarthy, Edinburgh (GB); Calum MacLeod, Hoddesdon (GB); Ben Moulton, Alderley Edge (GB); Gabriel Lenagh-Snow, Alderley Edge (GB)

(21) Appl. No.: 16/955,491

(22) PCT Filed: Dec. 21, 2018
(86) PCT No.: PCT/GB18/53761

§ 371 Date: Jun. 18, 2020

(30) Foreign Application Priority Data
Dec. 22, 2017 (GB) 1721751.4

(51) Int. Cl. C07D 405/04; C07D 405/14; C07D 239/42; C07D 401/12; C07D 413/14; C07D 401/14; C07D 417/14; C07D 239/47; C07D 403/12; A61K 39/395; A61K 35/26; A61K 31/53; A61K 31/506; A61K 31/505

(52) U.S. Cl. CPC C07D 405/04; C07D 405/14; C07D 239/42; C07D 401/12; C07D 413/14; C07D 401/14; A61K 31/505; C07D 239/47; C07D 403/12; A61K 39/3955; A61K 35/26; A61K 31/53; A61K 31/506; C07D 417/14

(57) ABSTRACT

Compounds of general formula (I): (Formula (I)) wherein R¹, R², X¹, and X² are defined herein as useful for the treatment of cancer, particularly solid tumors.

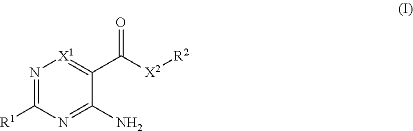

### INTRODUCTION

The present invention relates to certain compounds that function as antagonists of the adenosine A2a receptor. The present invention also relates to processes for the preparation of these compounds, to pharmaceutical compositions comprising them, and to their use in the treatment of diseases or conditions in which adenosine A2a receptor activity is implicated, such as, for example, cancer.

### BACKGROUND OF THE INVENTION

A number of immunosuppressive pathways are active in the tumour microenvironment which enable tumour cells to evade elimination by cytotoxic T cells and can diminish the clinical response of patients to immunotherapy with anti-checkpoint antibodies. The anti-PD-1 antibodies pembrolizumab and nivolumab and anti-PD-L1 antibodies durvalumab, avelumab and atezolizumab are approved for the treatment of number of solid tumours including non-small cell lung cancer, head and neck squamous cancer and urothelial cancer. However, only 20-30% of patients respond to checkpoint blockade and the side effects of such treatments are significant (Sukari et al, 2016). Consequently, other approaches to enhance the cytotoxic potential of the tumour microenvironment are actively being investigated. This includes agents that could be used as monotherapies or, more likely, used in combination with checkpoint inhibitors and cytotoxic agents to enhance their efficacy.

One approach that has attracted attention is to interfere with the production and/or action of adenosine in the tumour microenvironment (Vijayan et al, 2017). Adenosine has immunosuppressive properties and is present in the tumour microenvironment at high concentrations. Recent studies estimate the concentration of adenosine to be about 10 μM in human tumours compared to <1 μM in normal tissue (Houthuys et al 2017). Adenosine is formed at both intracellular and extracellular sites by two distinct pathways that involve two different substrates. Intracellular adenosine is derived from AMP and S-adenosyl homocysteine whilst the high extracellular adenosine concentrations observed during metabolic stress are associated with the release and degradation of precursor adenine nucleotides (ATP, ADP and AMP) by the concerted action of CD39 and CD73 (Vijayan et al, 2017).

CD39 and CD73 are upregulated in the tumour microenvironment in response to hypoxia. CD73 represents a putative patient stratification method for adenosine antagonists as its expression on tumour cells is also associated with poor overall prognosis in many different cancer types suggesting that adenosine production contributes to the undesirable immunosuppressive phenotype of the tumour microenvironment (Gao et al 2014; Loi et al, 2013). CD73 expression by tumour-infiltrating immune cells is also important in promoting tumour immune suppression as CD73 negative Treg cells fail to suppress effector T cell functions (Deaglio et al, 2007; Reinhardt et al, (2017). Furthermore, patients resistant to anti-PD1 treatment have elevated levels of CD73 (Reinhardt et al, 2017).

Adenosine regulates cell function via occupancy of specific GPCRs on the cell surface of the P1 purinoceptor subtypes. The P1 receptor family is further subdivided into A1, A2a, A2b and A3.

A2 receptors are subdivided into A2a and A2b, based on high and low affinity for adenosine, respectively. A2a is expressed by lymphocytes and activation of A2a leads to suppression of cytokine production and other effector functions. Tumour growth is inhibited by genetic ablation of A2a in syngeneic mouse models and this effect has been demonstrated to be due to enhanced lymphocyte activation and cytotoxic function (Ohta et al, 2006; Waickman et al 2012; Beavis et al, 2013; Mittal et al, 2014; Cekic et al, 2014). A2a^{-/-} mice show an increased response to inhibition of checkpoint pathways such as PD-1, with an improvement in both tumour free survival and overall survival. Adenosine-mediated A2a activation also limits the efficacy of ant-CTLA4 treatment (lannone et al, 2014).

The effects of genetic deficiency of A2a in mouse models is mimicked by pharmacological blockade of A2a. A2a antagonists have been shown to enhance the cytotoxic CD8⁺T cells and to enhance the ability of NK cells prevent metastasis of CD73-expressing tumours (Beavis et al, 2013). Importantly, A2a antagonists enhance the efficacy of anti-PD1 antibodies (Beavis et al, 2015).

These findings have prompted the development of selective A2a antagonists for use in cancer immunotherapy and clinical trials are ongoing with CPI-444, the first selective A2a antagonist to be evaluated in cancer, being used as both as a monotherapy and in combination with the anti-PDL1 antibody atezolizumab. The preliminary data indicated that the compound was well tolerated, and showed early indications of reducing tumour size and enhancing CD8⁺T infiltration into tumour tissue. The major challenge for this field is the identification of second generation compounds with improved potency and that retain activity in the presence of the high concentrations of adenosine that are present in the tumour microenvironment.

Here we describe a series of highly potent and selective A2a antagonists which retain activity in the presence of high concentrations of both plasma protein and adenosine receptor agonist ligands.

### SUMMARY OF THE INVENTION

According to a first aspect of the present invention, there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of antagonising adenosine A2a receptors in vitro or in vivo, said method comprising contacting a cell with an effective amount of a compound as defined herein.

According to a further aspect of the present invention, there is provided a method of selectively antagonising adenosine A2a receptors in vitro or in vivo, said method comprising contacting a cell with an effective amount of a compound as defined herein.

According to a further aspect of the present invention, there is provided a method of inhibiting cell proliferation, in vitro or in vivo, said method comprising contacting a cell with an effective amount of a compound as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating a disease or disorder associated with adenosine A2a receptor activity in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in therapy.

According to a further aspect of the present invention, there is provided a compound or a pharmaceutically acceptable salt, hydrate or solvate thereof as defined herein, or a pharmaceutical composition as defined herein, for use in the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, as defined herein for use as an adenosine A2a antagonist. In an embodiment, the compounds of the invention are selective adenosine A2a antagonists. In an alternative embodiment, certain compounds of the invention are selective adenosine A2a and adenosine A2b antagonists.

According to a further aspect of the present invention, there is provided a compound, or a pharmaceutically acceptable salt, hydrate or solvate thereof, as defined herein for use in the treatment of a disease or disorder in which adenosine A2a is implicated.

According to a further aspect of the present invention, there is provided the use of a compound, or a pharmaceutically acceptable salt, hydrate or solvate thereof, as defined herein in the manufacture of a medicament for the treatment of a proliferative condition.

Suitably, the compound or pharmaceutical composition is administered in combination with one or more additional antiproliferative agents (e.g. checkpoint inhibitors and/or cytotoxic agents).

According to a further aspect of the present invention, there is provide the use of a compound, or a pharmaceutically acceptable salt, hydrate or solvate thereof, as defined herein in the manufacture of a medicament for the treatment of cancer. Suitably, the cancer is a human cancer. Suitably, the compound or pharmaceutical composition is administered in combination with one or more additional anticancer agents (e.g. checkpoint inhibitors and/or cytotoxic agents).

According to a further aspect of the present invention, there is provided a use of a compound, or a pharmaceutically acceptable salt, hydrate or solvate thereof, as defined herein in the manufacture of a medicament for use as an adenosine A2a antagonist.

According to a further aspect of the present invention, there is provided a use of a compound, or a pharmaceutically acceptable salt, hydrate or solvate thereof, as defined herein in the manufacture of a medicament for the treatment of a disease or disorder in which adenosine A2a is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

Features, including optional, suitable, and preferred features in relation to one aspect of the invention may also be features, including optional, suitable and preferred features in relation to any other aspect of the invention.

### DETAILED DESCRIPTION OF THE INVENTION

In the present invention there is provided a compound of general formula (I) including all tautomeric forms, enantiomers, isotopic variants, salts and solvates thereof:

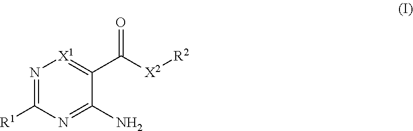

wherein
X¹ is CR³ or N;
R³ is
(i) H; or
(ii) halo; or
(iii) C₁₋₆ alkyl, —O(C₁₋₆ alkyl), —NH(C₁₋₆ alkyl) or —N(C₁₋₆ alkyl)₂, any of which may optionally be substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl;
   wherein R⁴ is H or methyl and each R⁹ and R¹⁰ is independently selected from H, C₁₋₆ alkyl and C₁₋₆ haloalkyl; or
(iv) carbocyclyl, heterocyclyl, aryl or heteroaryl;
wherein any carbocyclyl, heterocyclyl, aryl and heteroaryl groups are optionally substituted with one or more substituents selected from halo, C₁₋₆ alkyl, C₁₋₆ haloalkyl and —NR¹¹R¹²;
   wherein each R¹¹ and R¹² is independently selected from H, C₁₋₆ alkyl and C₁₋₆ haloalkyl;

### X² is O or NH;

R¹ is aryl or heteroaryl optionally substituted with one or more substituents selected from halo, OH, CN, R⁵, OR⁵ and NR⁵R⁶,
each R⁵ and R⁶ is independently H, C₁₋₆ alkyl or C₃₋₇ cycloalkyl, either of which is optionally substituted with one or more substituents selected from halo, OH, aryl and heteroaryl, wherein aryl and heteroaryl groups are optionally substituted with one or more substituents selected from halo, OH, C₁₋₆ alkyl and C₁₋₆ haloalkyl

### R² is:

(i) C₁₋₆ alkyl optionally substituted with one or more substituents selected from halo, OH, O—C₁₋₆ alkyl, NH(C₁₋₆ alkyl), N(C₁₋₆ alkyl)₂ and R⁸; or
   (ii) R⁸;
      wherein each R⁸ is independently aryl or heteroaryl, either or which may optionally be substituted with one or more substituents selected from halo, OH, NH₂, CN, NO₂, R⁷, OR⁷NHR⁷ or N(R⁷)₂;
         and each R⁷ is independently C₁₋₆ alkyl optionally substituted with one or more substituents selected from halo, OH, —O(C₁₋₈ alkyl) and —O(C₁₋₆ haloalkyl).

The compounds of formula (I) are selective antagonists of the A2a receptor and are useful for the treatment of cancer, especially solid tumours.

Some A_{2A} receptor antagonists are known, for example WO2016/081290 discloses compounds of formulae

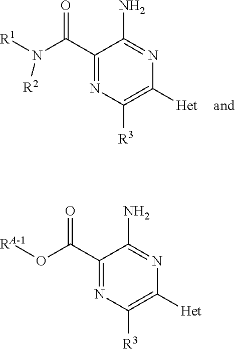

However, the present inventors have surprisingly discovered that the compounds of the present invention also have selective A2a antagonist activity. Additionally, certain compounds of the present invention show high cellular potency which is retained in the presence of whole blood.

In the present specification, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

In the present specification, references to “pharmaceutical use” refer to use for administration to a human or an animal, in particular a human or a mammal, for example a domesticated or livestock mammal, for the treatment or prophylaxis of a disease or medical condition. The term “pharmaceutical composition” refers to a composition which is suitable for pharmaceutical use and “pharmaceutically acceptable” refers to an agent which is suitable for use in a pharmaceutical composition. Other similar terms should be construed accordingly.

Appropriate pharmaceutically and veterinarily acceptable salts of the compounds of general formulae (I) and (II) include basic addition salts such as sodium, potassium, calcium, aluminium, zinc, magnesium and other metal salts as well as ammonium, choline, diethylamine, tromethamine (TRIS), diethanolamine, ethanolamine, ethyl diamine, megulmine piperazine and other well known basic addition salts as summarised in Paulekuhn et al., (2007) J. Med. Chem. 50: 6665-6672 and/or known to those skilled in the art.

Where appropriate, pharmaceutically or veterinarily acceptable salts may also include salts of organic acids, especially carboxylic acids, including but not limited to acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, pamoate, pectinate, 3-phenylpropionate, picrate, pivalate, proprionate, tartrate, lactobionate, pivolate, camphorate, undecanoate and succinate, organic sulfonic acids such as methanesulfonate, ethanesulfonate, 2-hydroxyethane sulfonate, camphorsulfonate, 2-naphthalenesulfonate, benzenesulfonate, p-chlorobenzenesulfonate and p-toluenesulfonate; and inorganic acids such as hydrochloride, hydrobromide, hydroiodide, sulfate, bisulfate, hemisulfate, thiocyanate, persulfate, phosphoric and sulfonic acids.

In the present specification, the term “C₁₋₆” alkyl refers to a straight or branched fully saturated hydrocarbon group having from 1 to 6 carbon atoms. The term encompasses methyl, ethyl, n-propyl, isopropyl, n-butyl, isobutyl and t-butyl. Other alkyl groups, for example C₁₋₁₂ alkyl and C₁₋₄ alkyl are as defined above but contain different numbers of carbon atoms.

The terms “carbocyclic” and “carbocyclyl” refer to a non-aromatic hydrocarbon ring system containing from 3 to 10 ring carbon atoms, unless otherwise indicated, and optionally one or more double bond. The carbocyclic group may be a single ring or may contain two or three rings which may be fused or bridged. Examples include cyclopropyl, cyclobutyl, cyclopentyl, cyclohexyl, cyclopentenyl and cyclohexenyl.

In the context of the present specification, the terms “heterocyclic” and “heterocyclyl” refer to a non-aromatic ring system containing 3 to 10 ring atoms including at least one heteroatom selected from N, O and S. The heterocyclic group may be a single ring or may contain two or three rings which may be fused or bridged. Examples include tetrahydrofuranyl, tetrahydroypranyl, pyrrolidine, piperidinyl, morpholinyl, piperazinyl and thiomorpholinyl.

The terms “aryl” and “aromatic” in the context of the present specification refer to a ring system with aromatic character having from 5 to 14 ring carbon atoms and containing up to three rings. Where an aryl group contains more than one ring, not all rings must be fully aromatic in character. For example, in some instances, an “aryl” or “aromatic” group may comprise two rings, one of which is an aromatic ring and a fused non-aromatic ring. In such cases, the aryl or aromatic group may be linked to the compound of formula I by either the aromatic or the non-aromatic ring. Examples of aromatic moieties are benzene, naphthalene, fluorene, indane, indene, benzodioxole and tetrahydroquinoline.

The terms “heteroaryl” and “heteroaromatic” in the context of the specification refer to a ring system with aromatic character having from 5 to 14 ring atoms, at least one of which is a heteroatom selected from N, O and S, and containing up to three rings. Where a heteroaryl group contains more than one ring, not all rings must be fully aromatic in character. For example, in some instances, a “heteroaryl” or “heteroaromatic” group may comprise two rings, one of which is a heteroaromatic ring and a fused non-aromatic ring. In such cases, the heteroaryl or heteroaromatic group may be linked to the compound of formula I by either the aromatic or the non-aromatic ring. Examples of heteroaryl groups include pyridine, pyrimidine, indole, benzofuran, benzimidazole and indolene.

The term “halogen” refers to fluorine, chlorine, bromine or iodine, the term “halo” to fluoro, chloro, bromo or iodo groups and “halide” to fluoride, chloride, bromide or iodide.

The term “C₁₋₆ haloalkyl” as used herein refers to a C₁₋₆ alkyl group as defined above in which one or more of the hydrogen atoms is replaced by a halo group. Any number of hydrogen atoms may be replaced, up to perhalo substitution. Examples include trifluoromethyl, chloroethyl and 1,1-difluoroethyl. Other haloalkyl groups, for example C₁₋₁₂ haloalkyl are as defined above except that they contain the specified number (e.g. 1 to 12) carbon atoms.

The term “isotopic variant” refers to isotopically-labelled compounds which are identical to those recited in formula (I) but for the fact that one or more atoms are replaced by an atom having an atomic mass or mass number different from the atomic mass or mass number most commonly found in nature, or in which the proportion of an atom having an atomic mass or mass number found less commonly in nature has been increased (the latter concept being referred to as “isotopic enrichment”). Examples of isotopes that can be incorporated into compounds of the invention include isotopes of hydrogen, carbon, nitrogen, oxygen, fluorine, iodine and chlorine such as 2H (deuterium), ³H, ¹¹C, ¹³C, ¹⁴C, ¹⁸F, ¹²³I or ¹²⁵I (e.g. ³H, ¹¹C, ¹⁴C, ¹⁸F, ¹²³I or ¹²⁵I), which may be naturally occurring or non-naturally occurring isotopes.

In some compounds of general formula (I), X¹ is N.

In other compounds of general formula (I), X¹ is CR³, wherein R³ is as defined above.

More suitably, R³ is H; halo; —O(C₁₋₆ alkyl), optionally substituted as described above; or aryl or heteroaryl, either or which is optionally substituted as described above.

In some compounds of general formula (I), X¹ is CR³ and R³ is H.

In other compounds of general formula (I), X¹ is CR³ and R³ is halo.

Suitably, R³ is
(i) H; or
(ii) halo; or
(iii) —O(C₁₋₆ alkyl) or —NH(C₁₋₆ alkyl), any of which may optionally be substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl; wherein R⁴ is H or methyl and each R⁹ and R¹⁰ is independently selected from H, C₁₋₆ alkyl and C₁₋₆ haloalkyl; or
(iv) heteroaryl;
   wherein any carbocyclyl, heterocyclyl, aryl and heteroaryl groups are optionally substituted with one or more substituents selected from halo, C₁₋₆ alkyl, C₁₋₆haloalkyl and —NR¹¹R¹²; wherein each R¹¹ and R¹² is independently selected from H, C₁₋₆ alkyl and C₁₋₆ haloalkyl.

Suitably, R³ is
(i) halo; or
(ii) —O(C₁₋₆ alkyl) or —NH(C₁₋₆ alkyl), any of which may optionally be substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl; wherein R⁴ is H or methyl and each R⁹ and R¹⁰ is independently selected from H, C₁₋₆ alkyl and C₁₋₆ haloalkyl; or
(iii) heteroaryl;
   wherein any carbocyclyl, heterocyclyl, aryl and heteroaryl groups are optionally substituted with one or more substituents selected from halo, C₁₋₆ alkyl, C₁₋₆haloalkyl and —NR¹¹R¹²; wherein each R¹¹ and R¹² is independently selected from H, C₁₋₆ alkyl and C₁₋₆ haloalkyl.

More suitably, R³ is:
—O(C₁₋₆ alkyl) or —NH(C₁₋₆ alkyl), each of which is optionally substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl; wherein R⁴ is H or methyl and each R⁹ and R¹⁰ is independently selected from H, C₁₋₆ alkyl and C₁₋₆ haloalkyl; or
heteroaryl which is optionally substituted with one or more substituents selected from halo, C₁₋₆ alkyl, C₁₋₆ haloalkyl and —NR¹¹R¹²; wherein each R¹¹ and R¹² is independently selected from H, C₁₋₆ alkyl and C₁₋₆ haloalkyl.

In other compounds of general formula (I), X¹ is CR³ and R³ is —O(C₁₋₆ alkyl) or —NH(C₁₋₆ alkyl), optionally substituted as described above.

In other compounds of general formula (I), X¹ is CR³ and R³ is —O(C₁₋₆ alkyl) optionally substituted as described above.

In an embodiment, R³ is an unsubstituted —O(C₁₋₆ alkyl).

In other compounds of general formula (I), X¹ is CR³ and R³ is —O(C₁₋₆ alkyl) optionally substituted with one or more substituents selected from heterocyclyl, NH₂, halo, OH and —O(C₁₋₆ alkyl).

In other compounds of general formula (I), X¹ is CR³ and R³ is —O(C₁₋₆ alkyl) optionally substituted with one or more substituents selected from halo, OH and —O(C₁₋₆ alkyl).

In other compounds of general formula (I), X¹ is CR³ and R³ is —O(C₁₋₆ alkyl) optionally substituted with one or more substituents selected from halo, OH and —O(C₁₋₃ alkyl).

In other compounds of general formula (I), X¹ is CR³ and R³ is —O(C₁₋₆ alkyl) substituted with one or more OH groups.

In other compounds of general formula (I), X¹ is CR³ and R³ is —O(C₁₋₆ alkyl) substituted with one or more —O(C₁₋₃ alkyl) groups.

In other compounds of general formula (I), X¹ is CR³ and R³ is —O(C₁₋₆ alkyl), substituted with one or more substituents selected from halo. R³ may be —O(C₁₋₄ alkyl) substituted with one or more substituents selected from halo. Suitably, the one or more halo groups are fluoro. In a particular embodiment, R³ is —O—CH₂CH₂F.

In other compounds of general formula (I), X¹ is CR³ and R³ is 6-membered aryl or 5- or 6-membered heteroaryl, either of which is optionally substituted with one or more substituents selected from halo, OH, C₁₋₆ alkyl, —O(C₁₋₆ alkyl), C₁₋₆ haloalkyl and —O(C₁₋₆ haloalkyl).

More suitably in these compounds, R³ is an unsubstituted 6-membered aryl or 5- or 6-membered heteroaryl moiety.

In other, more suitable compounds of general formula (I), X² is NH. In this case, the compounds of general formula (I) are compounds of general formula (Ia):

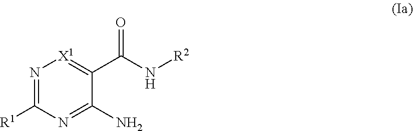

wherein X¹, R¹, R² and R⁴ are as defined for general formula (I).

In other compounds of general formula (I), X² is O. In this case, the compounds of general formula (I) are compounds of general formula (Ib):

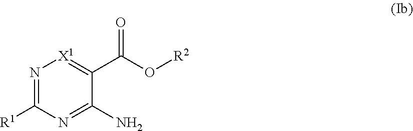

wherein X¹, R¹ and R² are as defined above for general formula (I).

In some compounds of general formula (Ib), R² is a group R^{2b}, where R^{2b} is C₁₋₆ alkyl.

In suitable compounds of general formula (I), R¹ is phenyl or 5- or 6-membered heteroaryl optionally substituted as described above.

In other suitable compounds of general formula (I), X¹ is CR³. In this case, the compounds of general formula (I) are compounds of general formula (Ic):

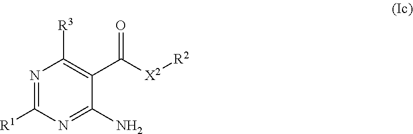

wherein R¹, R², R³ and X² are as defined above for general formula (I).

In other suitable compounds of general formula (Ic), X² is NH. In this case, the compounds of general formula (I) are compounds of general formula (Id):

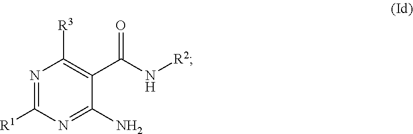

wherein R¹, R², R³ and X² are as defined above for general formula (I).

Compounds of general formula (II):

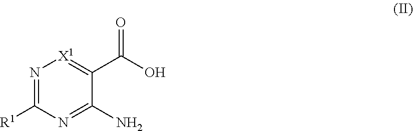

wherein X¹ and R¹ are as defined for general formula (I) and which are as compounds of general formula (Ib) except that R² is H are of use as intermediates in the synthesis of compounds of general formula (Ia).

More suitably, in any of formulae (I), (Ia), (Ib), (Ic), (Id) or (II) above, R¹ is phenyl or 5-membered heteroaryl optionally substituted as described above.

In suitable compounds of general formula (I) (or (Ia), (Ib), (Ic), (Id) or (II) above), R¹ is phenyl, furanyl, oxazolyl or pyrazolyl, more suitably, phenyl, furan-2-yl, oxazol-2-yl, pyrazol-1-yl, m-cyanophenyl or thiazolyl, any of which may be unsubstituted or substituted as described above.

In still more suitable compounds of general formula (a) (or (Ia), (Ib), (Ic), (Id) or (II) above), R¹ is phenyl, furanyl, oxazolyl or pyrazolyl, more suitably, phenyl, furan-2-yl, oxazol-2-yl, m-cyanophenyl or pyrazol-1-yl, any of which may be unsubstituted or substituted as described above.

Suitably, in any of formulae (I), (Ia), (Ib), (Ic), (Id) or (II) above, R¹ is phenyl, furanyl, oxazolyl, or thiazolyl.

Still more suitably, in any of formulae (I), (Ia), (Ib), (Ic), (Id) or (II) above, R¹ is phenyl, for example m-cyanophenyl, or furanyl and more particularly furanyl, for example furan-2-yl.

Suitable substituents for R¹ include one or more substituents selected from halo, OH, CN, R⁵, OR⁵ and NR⁵R⁶, wherein each R⁵ and R⁶ is independently H or C₁₋₆ alkyl which is optionally substituted with one or more substituents selected from halo and OH.

More suitable substituents for R¹ include halo and cyano.

When R¹ is phenyl, it is suitably substituted as described above, particularly with halo and cyano. Examples of such R¹ groups include 3-halophenyl, for example 3-fluorophenyl and 3-chlorophenyl, 2-halophenyl, for example 2-fluorophenyl, and 2-chlorophenyl, 4-halophenyl, for example 4-fluorophenyl and 4-chlorophenyl, 3-cyanophenyl, 2-cyanophenyl and 4-cyanophenyl.

More suitable examples of such R¹ groups include 3-fluorophenyl, 2-fluorophenyl, 4-fluorophenyl and 3-cyanophenyl, especially 3-fluorophenyl and 3-cyanophenyl.

When R¹ is 5-membered heteroaryl such as furanyl, oxazolyl or pyrazolyl, it is suitably unsubstituted. Suitable examples of such R¹ groups include furan-2-yl, oxazol-2-yl and pyrazol-1-yl, especially unsubstituted furan-2-yl.

Suitably, R¹ is selected from furanyl, oxazolyl, thiazolyl or phenyl optionally substituted with fluoro or cyano.

More suitably, R¹ is selected from unsubstituted furan-2-yl, 3-fluorophenyl and 3-cyanophenyl.

In suitable compounds of general formula (I), (Ia), (Ib), (Ic), (Id) or (II) above, R² is C₁₋₆ alkyl optionally substituted with one or more substituents selected from halo, OH, O—C₁₋₆ alkyl and R⁸, wherein each R⁸ is independently as defined above for general formula (I).

More suitably, R² is C₁₋₆ alkyl substituted with one or more substituents R⁸, wherein R⁸ is as defined above.

In some suitable compounds, when:

X¹ is CH;

X² is NH; and

R² is C₁₋₆ alkyl substituted with R⁸;

R¹ and R⁸ are not both selected from unsubstituted phenyl, phenyl substituted with methyl, unsubstituted pyridyl, unsubstituted furyl and unsubstituted thienyl.

Still more suitably, R² is C₁₋₄ alkyl or C₁₋₃ substituted with one or more substituents R⁸, wherein R⁸ is as defined above.

In particularly suitable compounds of formulae (I), (Ia), (Ib), (Ic), (Id) or (II) above, R² is CH₂—R⁸, CH₂—CH₂—R³ or CH(CH₃)—R⁸, especially CH₂R⁸, where R³ is as defined above.

More particularly, R⁸ is phenyl, naphthyl, indanyl, pyridyl, pyrimidinyl, quinolinyl, pyrrolyl, imidazolyl, pyrazolyl or triazolyl, any of which is optionally substituted as described above for aryl and heteroaryl groups of R².

Example structures of R⁸ groups include but are not limited to the following:

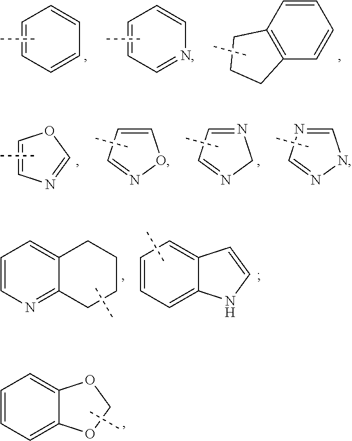

wherein “ [symbol] ” denotes the point of attachment to R² and R⁸ may be optionally substituted as defined herein.

Still more particularly, R⁸ is phenyl, pyridyl, pyrimidinyl, quinolinyl, imidazolyl or triazolyl, any of which is optionally substituted as described above for general formula (I).

Particularly suitable R⁸ groups include phenyl, indan-1-yl, indan-2-yl, pyridin-2-yl, imidazol-2-yl, quinolin-8-yl and triazol-3-yl, any of which is optionally substituted as described above for general formula (I).

More suitably, R⁸ may be substituted with one or more substituents selected from halo; OH; C₁₋₆ alkyl and —O(C₁₋₆ alkyl), either of which is optionally substituted with halo, OH or —O(C₁₋₆ alkyl).

In some particularly suitable compounds, R⁸ is unsubstituted or is substituted with one or more substituents selected from methyl, ethyl, n-propyl, iso-propyl, hydroxy, methoxy, ethyoxy, fluoro or chloro.

In some suitable compounds, R⁸ is:

pyridyl optionally substituted with Me, OH, OMe, OEt, CF₃, F; or

quinolinyl optionally substituted with Me, OH, OMe, OEt, CF₃, F; or

phenyl optionally substituted with Me, OH, OMe, OEt, CF₃, F.

When R⁸ is pyridine-2-yl, it suitably has a single substituent at the pyridine 3-position.

An example of a particularly suitable R⁸ group is 3-methyl-pyridin-2-yl.

In particularly suitable compounds of general formula (I), R¹ is furan-2-yl and R² is CH₂-(3-methylpyridin-2-yl). In such compounds, X² is suitably NH.

In a particular embodiment, the compounds have the structural formula (Ic) or (Id) above, in which R¹, R² and R³ each have any one of the definitions set out herein.

In suitable compounds of formula (Ic) or (Id), R¹ is selected from phenyl optionally substituted as described above or 5- or 6-membered heteroaryl optionally substituted as described above. More suitably, in either of formulae (Ic) or (Id) above, R¹ is phenyl or 5-membered heteroaryl optionally substituted as described above.

In still more suitable compounds of general formula (Ic) or (Id) above, R¹ is phenyl, furanyl, thiazole, oxazolyl or pyrazolyl any of which may be unsubstituted or substituted as described above. More suitably, R¹ is phenyl, furan-2-yl, oxazol-2-yl, oxazol-5-yl or pyrazol-1-yl, any of which may be unsubstituted or substituted as described above.

Still more suitably, in either of formulae (Ic) or (Id) above, R¹ is: phenyl optionally substituted with one or more substituents selected from halo, OH, CN, R⁵, OR⁵ and NR⁵R⁶, wherein each R⁵ and R⁶ is independently H or C₁₋₆ alkyl which is optionally substituted with one or more substituents selected from halo and OH; or furanyl (and more particularly furanyl, for example furan-2-yl), oxazolyl or pyrazolyl. When R¹ is phenyl, it is suitably substituted as described above, particularly with halo and cyano. Examples of such R¹ groups include 3-halophenyl, for example 3-fluorophenyl and 3-chlorophenyl, 2-halophenyl, for example 2-fluorophenyl, and 2-chlorophenyl, 4-halophenyl, for example 4-fluorophenyl and 4-chlorophenyl, or 3-cyanophenyl, 2-cyanophenyl, 4-cyanophenyl, furanyl, oxazolyl or pyrazolyl.

In formula (Ic) or (Id), R¹ is suitably selected from furanyl, or phenyl optionally substituted with fluoro or cyano. More suitably, R¹ is selected from unsubstituted furan-2-yl, 3-fluorophenyl and 3-cyanophenyl.

In suitable compounds of formula (Ic) or (Id), R² is:
(i) (C₁₋₆ alkyl) optionally substituted with one or more substituents selected from halo, OH, O—C₁₋₆ alkyl, NH(C₁₋₆ alkyl), N(C₁₋₆ alkyl)₂ and R⁸; or
(ii) R² is R⁸, wherein each R⁸ is independently aryl or heteroaryl, either or which may optionally be substituted with one or more substituents selected from halo, OH, NH₂, CN, NO₂, R⁷, OR⁷ NHR⁷ or N(R⁷)₂;
and wherein each R⁷ is independently C₁₋₆ alkyl optionally substituted with one or more substituents selected from halo, OH, —O(C₁₋₆ alkyl) and —O(C₁₋₆ haloalkyl).

Suitably, in compounds of formula (Ic) or (Id), R² is either aryl, heteroaryl or (C₁₋₆ alkyl) optionally substituted with one or more substituents selected from OH, N(C₁₋₆ alkyl)₂, aryl and heteroaryl;
wherein each aryl or heteroaryl may optionally be independently substituted with one or more substituents selected from halo, OH, NH₂, CN, NO₂, R⁷, OR NHR⁷ or N(R⁷)₂; wherein each R⁷ is independently C₁₋₆ alkyl optionally substituted with one or more substituents selected from halo, OH, —O(C₁₋₆ alkyl) and —O(C₁₋₆ haloalkyl).

Suitably, in compounds of formula (Ic) or (Id), R² is either aryl, heteroaryl or (C₁₋₂ alkyl) substituted with one or more aryl or heteroaryl substituents, wherein each aryl or heteroaryl may optionally be independently substituted with one or more substituents selected from halo, OH, NH₂, CN, NO₂, R⁷, OR⁷ NHR⁷ or N(R)₂;
wherein each R⁷ is independently C₁₋₆ alkyl optionally substituted with one or more substituents selected from halo, OH, —O(C₁₋₆ alkyl) and —O(C₁₋₆ haloalkyl).

In suitable compounds of formula (Ic) and (Id), R³ is:
(i) —O(C₁₋₆ alkyl), optionally substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl; or
(ii) heteroaryl;
wherein any carbocyclyl, heterocyclyl, aryl and heteroaryl groups are optionally substituted with one or more substituents selected from halo, C₁₋₆ alkyl, C₁₋₆ haloalkyl and —NR¹¹R¹²; wherein each R¹¹ and R¹² is independently selected from H, C₁₋₆ alkyl and C₁₋₆ haloalkyl.

In suitable compounds of formula (Ic) and (Id), R³ is —O(C₁₋₆ alkyl), optionally substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl;
wherein any carbocyclyl, heterocyclyl, aryl and heteroaryl groups are optionally substituted with one or more substituents selected from halo, C₁₋₆ alkyl, C₁₋₆ haloalkyl and —NR¹¹R¹²; wherein each R¹¹ and R¹² is independently selected from H, C₁₋₆ alkyl and C₁₋₆ haloalkyl.

In suitable compounds of formula (Id),
R¹ is selected from phenyl, furanyl, thiazole, oxazolyl or pyrazolyl (e.g. phenyl, furan-2-yl, oxazol-2-yl, oxazol-5-yl or pyrazol-1-yl unsubstituted furan-2-yl, 3-fluorophenyl and 3-cyanophenyl;
R² is (C₁₋₆ alkyl) optionally substituted with one or more substituents selected from halo, OH, N(C₁₋₆ alkyl)₂ and R⁸; or R² is R⁸, wherein each R⁸ is independently aryl or heteroaryl, either of which may optionally be substituted with one or more substituents selected from halo, OH, NH₂, CN, NO₂, R⁷, OR⁷ NHR⁷ or N(R⁷)₂; wherein each R⁷ is independently C₁₋₆ alkyl optionally substituted with one or more substituents selected from halo, OH, —O(C₁₋₆ alkyl) and —O(C₁₋₆ haloalkyl); and
R³ is —O(C₁₋₆ alkyl), optionally substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl;
wherein any carbocyclyl, heterocyclyl, aryl and heteroaryl groups are optionally substituted with one or more substituents selected from halo, C₁₋₆ alkyl, C₁₋₆ haloalkyl and —NR¹¹R¹²; wherein each R¹¹ and R¹² is independently selected from H, C₁₋₆ alkyl and C₁₋₆ haloalkyl.

In further suitable compounds of formula (Id),
R¹ is selected from unsubstituted furan-2-yl, 3-fluorophenyl and 3-cyanophenyl;
R² is (C₁₋₆ alkyl) optionally substituted with one or more substituents selected from halo, OH, N(C₁₋₆ alkyl)₂ and R⁸; or R² is R⁸, wherein each R⁸ is independently aryl or heteroaryl, either of which may optionally be substituted with one or more substituents selected from halo, OH, NH₂, CN, NO₂, R⁷, OR⁷ NHR⁷ or N(R⁷)₂; wherein each R⁷ is independently C₁₋₆ alkyl optionally substituted with one or more substituents selected from halo, OH, —O(C₁₋₆ alkyl) and —O(C₁₋₆ haloalkyl); and
R³ is —O(C₁₋₆ alkyl), optionally substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl;
wherein any carbocyclyl, heterocyclyl, aryl and heteroaryl groups are optionally substituted with one or more substituents selected from halo, C₁₋₆ alkyl, C₁₋₆ haloalkyl and —NR¹¹R¹²; wherein each R¹¹ and R¹² is independently selected from H, C₁₋₆ alkyl and C₁₋₆ haloalkyl.

In further suitable compounds of formula (Id),
R¹ is selected from unsubstituted furan-2-yl, 3-fluorophenyl and 3-cyanophenyl;
R² is (C₁₋₆ alkyl) optionally substituted with one or more substituents selected from halo, OH, N(C₁₋₆ alkyl)₂ and R⁸; or R² is R⁸, wherein each R⁸ is independently aryl or heteroaryl, either of which may optionally be substituted with one or more substituents selected from halo, OH, NH₂, CN, NO₂, R⁷, OR⁷ NHR⁷ or N(R⁷)₂; wherein each R⁷ is independently C₁₋₆ alkyl optionally substituted with one or more substituents selected from halo, OH, —O(C₁₋₆ alkyl) and —O(C₁₋₆ haloalkyl); and
R³ is —O(C₁₋₆ alkyl) optionally substituted with one or more substituents selected from heterocyclyl, NH₂, halo, OH and —O(C₁₋₆ alkyl).

Specific examples of compounds of general formula (I) include any one of the following:
Ethyl 5-amino-3-(2-furyl)-1,2,4-triazine-6-carboxylate;
5-Amino-3-(2-furyl)-N-[(3-methyl-2-pyridyl)methyl]-1,2,4-triazine-6-carboxamide;
4-Amino-2-(2-furyl)-N-[(3-methyl-2-pyridyl)methyl]pyrimidine-5-carboxamide;
4-Amino-N-benzyl-2-(3-fluorophenyl)pyrimidine-5-carboxamide;
4-Amino-2-(3-fluorophenyl)-N-(2-phenylethyl)pyrimidine-5-carboxamide;
4-Amino-2-(3-fluorophenyl)-N-phenyl-pyrimidine-5-carboxamide;
4-amino-2-(3-fluorophenyl)-N-[(3-methyl-2-pyridyl)methyl]pyrimidine-5-carboxamide;
4-Amino-2-(3-fluorophenyl)-N-[(1R)-1-phenylethyl]pyrimidine-5-carboxamide;
4-Amino-2-(3-fluorophenyl)-N-[(2-fluorophenyl)methyl]pyrimidine-5-carboxamide;
4-Amino-N-[(2-fluoro-6-methoxy-phenyl)methyl]-2-(3-fluorophenyl)pyrimidine-5-carboxamide;
4-Amino-N-[(2-ethoxy-6-fluoro-phenyl)methyl]-2-(3-fluorophenyl)pyrimidine-5-carboxamide;
4-Amino-N-[(2,6-difluorophenyl)methyl]-2-(3-fluorophenyl)pyrimidine-5-carboxamide;
4-Amino-2-(3-fluorophenyl)-N-(pyrimidin-2-ylmethyl)pyrimidine-5-carboxamide;
Amino-2-(3-fluorophenyl)-N-[[3-(trifluoromethyl)-2-pyridyl]methyl]pyrimidine-5-carboxamide;
4-Amino-2-(3-fluorophenyl)-N-[2-(4-hydroxyphenyl)ethyl]pyrimidine-5-carboxamide;
4-Amino-N-[(3-ethoxy-2-pyridyl)methyl]-2-(3-fluorophenyl)pyrimidine-5-carboxamide;
4-Amino-2-(3-fluorophenyl)-N-(2-pyridyl methyl)pyrimidine-5-carboxamide;
4-Amino-2-(3-fluorophenyl)-N-(o-tolylmethyl) pyrimidine-5-carboxamide;
4-Amino-2-(2-furyl)-N-[(4-methoxyphenyl) methyl]pyrimidine-5-carboxamide;
4-Amino-N-[(3-chlorophenyl)methyl]-2-(2-furyl)pyrimidine-5-carboxamide;
4-Amino-N-[(3-fluorophenyl)methyl]-2-(2-furyl)pyrimidine-5-carboxamide;
4-Amino-N-[(4-fluorophenyl)methyl]-2-(2-furyl)pyrimidine-5-carboxamide;
4-Amino-N-[(2-chlorophenyl)methyl]-2-(2-furyl)pyrimidine-5-carboxamide;
4-Amino-N-[(2,6-dichlorophenyl)methyl]-2-(2-furyl)pyrimidine-5-carboxamide;
4-Amino-N-[(3,5-difluorophenyl)methyl]-2-(2-furyl)pyrimidine-5-carboxamide;
4-Amino-N-[(2-fluorophenyl)methyl]-2-(2-furyl)pyrimidine-5-carboxamide;
4-Amino-N-[(2,4-dichlorophenyl)methyl]-2-(2-furyl)pyrimidine-5-carboxamide;
4-Amino-2-(2-furyl)-N-[(2-methoxyphenyl)methyl]pyrimidine-5-carboxamide;
4-Amino-N-[(3-chloro-2-pyridyl)methyl]-2-(2-furyl)pyrimidine-5-carboxamide;
4-Amino-2-(2-furyl)-N-indan-1-yl-pyrimidine-5-carboxamide;
4-Amino-2-(2-furyl)-N-indan-2-yl-pyrimidine-5-carboxamide;
4-amino-2-(2-furyl)-N-(8-quinolylmethyl) pyrimidine-5-carboxamide;
4-Amino-N-[(1-ethylimidazol-2-yl)methyl]-2-(2-furyl)pyrimidine-5-carboxamide;
4-amino-2-(2-furyl)-N-[(2-isopropyl-1,2,4-triazol-3-yl)methyl]pyrimidine-5-carboxamide;
4-amino-2-(2-furyl)-N-[[4-(trifluoromethyl)phenyl]methyl]pyrimidine-5-carboxamide;
4-amino-2-(2-furyl)-N-[[3-(trifluoro methyl)phenyl]methyl]pyrimidine-5-carboxamide;
4-amino-2-(2-furyl)-N-[[3-(trifluoromethyl)-2-pyridyl]methyl]pyrimidine-5-carboxamide;
ethyl 5-amino-3-(3-fluorophenyl)-1,2,4-triazine-6-carboxylate;
5-Amino-3-(3-fluorophenyl)-N-[(3-methyl-2-pyridyl)methyl]-1,2,4-triazine-6-carboxamide;
5-Amino-3-(4-fluorophenyl)-N-[(3-methyl-2-pyridyl)methyl]-1,2,4-triazine-6-carboxamide;
4-Amino-2-(2-fluorophenyl)-N-[(3-methyl-2-pyridyl)methyl]pyrimidine-5-carboxamide;
4-Amino-2-(3-fluorophenyl)-N-(p-tolylmethyl)pyrimidine-5-carboxamide;
4-Amino-2-(3-fluorophenyl)-N-[(2-hydroxyphenyl)methyl]pyrimidine-5-carboxamide;
4-Amino-N-[(3-chlorophenyl)methyl]-2-(3-fluorophenyl)pyrimidine-5-carboxamide;
4-Amino-2-(3-fluorophenyl)-N-[(3-fluorophenyl)methyl]pyrimidine-5-carboxamide;
4-Amino-2-(3-fluorophenyl)-N-[(4-fluorophenyl) methyl]pyrimidine-5-carboxamide;
4-Amino-N-[(2-chlorophenyl)methyl]-2-(3-fluorophenyl)pyrimidine-5-carboxamide;
4-Amino-N-[(2,6-dichlorophenyl)methyl]-2-(3-fluorophenyl)pyrimidine-5-carboxamide;
4-Amino-N-[(3,5-difluorophenyl)methyl]-2-(3-fluorophenyl)pyrimidine-5-carboxamide;
4-Amino-2-(3-fluorophenyl)-N-[(2-methoxyphenyl)methyl]pyrimidine-5-carboxamide;
4-Amino-N-[(3,5-dimethoxyphenyl)methyl]-2-(3-fluorophenyl)pyrimidine-5-carboxamide;
4-Amino-N-[(2,3-dimethoxyphenyl)methyl]-2-(3-fluorophenyl)pyrimidine-5-carboxamide;
4-Amino-2-(3-fluorophenyl)-N-[[3-(trifluoromethyl)phenyl]methyl]pyrimidine-5-carboxamide;
4-Amino-N-[(3-chloro-2-pyridyl)methyl]-2-(3-fluorophenyl)pyrimidine-5-carboxamide;
4-Amino-2-(3-fluorophenyl)-N-(8-quinolylmethyl)pyrimidine-5-carboxamide;
4-Amino-N-[(1-ethylimidazol-2-yl)methyl]-2-(3-fluorophenyl)pyrimidine-5-carboxamide;
4-Amino-2-(3-fluorophenyl)-N-[(2-isopropyl-1,2,4-triazol-3-yl)methyl]pyrimidine-5-carboxamide;
4-amino-2-(2-furyl)-N-(p-tolylmethyl)pyrimidine-5-carboxamide;
4-amino-2-(2-furyl)-N-[(2-hydroxyphenyl) methyl]pyrimidine-5-carboxamide;
4-Amino-2-(3-cyanophenyl)-N-[(3-methyl-2-pyridyl)methyl]pyrimidine-5-carboxamide;
4-Amino-6-chloro-2-(2-furyl)-N-[(3-methyl-2-pyridyl)methyl]pyrimidine-5-carboxamide;
4-Amino-2-(4-fluorophenyl)-N-[(3-methyl-2-pyridyl)methyl]pyrimidine-5-carboxamide;
4-Amino-N-[(3-methyl-2-pyridyl)methyl]-2-pyrazol-1-yl-pyrimidine-5-carboxamide;
4-Amino-N-[(3-methyl-2-pyridyl)methyl]-2-oxazol-2-yl-pyrimidine-5-carboxamide;
5-Amino-3-(3-cyanophenyl)-N-[(3-methyl-2-pyridyl)methyl]-1,2,4-triazine-6-carboxamide;
4-Amino-2-(2-furyl)-6-methoxy-N-[(3-methyl-2-pyridyl)methyl]pyrimidine-5-carboxamide;
4-Amino-2-(2-furyl)-N-[(3-methyl-2-pyridyl)methyl]-6-pyrazol-1-yl-pyrimidine-5-carboxamide;
   and pharmaceutically acceptable salts and solvates thereof.

Specific examples of compounds of general formula (I) include the following compounds;
Ethyl 5-amino-3-(2-furyl)-1,2,4-triazine-6-carboxylate;
5-Amino-3-(2-furyl)-N-[(3-methyl-2-pyridyl)methyl]-1,2,4-triazine-6-carboxamide;
4-Amino-2-(2-furyl)-N-[(3-methyl-2-pyridyl)methyl]pyrimidine-5-carboxamide;
4-Amino-2-(5-methyl-2-furyl)-N-[(3-methyl-2-pyridyl)methyl]pyrimidine-5-carboxamide;
4-Amino-N-benzyl-2-(3-fluorophenyl)pyrimidine-5-carboxamide;
4-Amino-2-(3-fluorophenyl)-N-(2-phenylethyl)pyrimidine-5-carboxamide;
4-Amino-2-(3-fluorophenyl)-N-phenyl-pyrimidine-5-carboxamide;
4-Amino-2-(3-fluorophenyl)-N-[(3-methyl-2-pyridyl)methyl]pyrimidine-5-carboxamide;
4-Amino-2-(3-fluorophenyl)-N-[(1R)-1-phenylethyl]pyrimidine-5-carboxamide;
4-Amino-2-(3-fluorophenyl)-N-[(2-fluorophenyl) methyl]pyrimidine-5-carboxamide;
4-Amino-N-[(2-fluoro-6-methoxy-phenyl) methyl]-2-(3-fluorophenyl)pyrimidine-5-carboxamide;
4-Amino-N-[(2-ethoxy-6-fluoro-phenyl)methyl]-2-(3-fluorophenyl)pyrimidine-5-carboxamide;
4-Amino-N-[(2,6-difluorophenyl)methyl]-2-(3-fluorophenyl)pyrimidine-5-carboxamide;
4-Amino-2-(3-fluorophenyl)-N-(pyrimidin-2-ylmethyl)pyrimidine-5-carboxamide;
4-Amino-2-(3-fluorophenyl)-N-[[3-(trifluoromethyl)-2-pyridyl]methyl]pyrimidine-5-carboxamide;
4-Amino-2-(3-fluorophenyl)-N-[2-(4-hydroxyphenyl)ethyl]pyrimidine-5-carboxamide;
4-Amino-N-[(3-ethoxy-2-pyridyl)methyl]-2-(3-fluorophenyl)pyrimidine-5-carboxamide;
4-Amino-2-(3-fluorophenyl)-N-(2-pyridyl methyl)pyrimidine-5-carboxamide;
4-Amino-2-(3-fluorophenyl)-N-(o-tolylmethyl) pyrimidine-5-carboxamide;
4-Amino-2-(2-furyl)-N-[(4-methoxyphenyl) methyl]pyrimidine-5-carboxamide;
4-Amino-N-[(3-chlorophenyl)methyl]-2-(2-furyl)pyrimidine-5-carboxamide;
4-Amino-N-[(3-fluorophenyl)methyl]-2-(2-furyl)pyrimidine-5-carboxamide;
4-Amino-N-[(4-fluorophenyl)methyl]-2-(2-furyl)pyrimidine-5-carboxamide;
4-Amino-N-[(2-chlorophenyl)methyl]-2-(2-furyl)pyrimidine-5-carboxamide;
4-Amino-N-[(2,6-dichlorophenyl)methyl]-2-(2-furyl)pyrimidine-5-carboxamide;
4-Amino-N-[(3,5-difluorophenyl)methyl]-2-(2-furyl)pyrimidine-5-carboxamide;
4-Amino-N-[(2-fluorophenyl)methyl]-2-(2-furyl)pyrimidine-5-carboxamide;
4-Amino-N-[(2,4-dichlorophenyl)methyl]-2-(2-furyl)pyrimidine-5-carboxamide;
4-Amino-2-(2-furyl)-N-[(2-methoxyphenyl)methyl]pyrimidine-5-carboxamide;
4-Amino-N-[(3-chloro-2-pyridyl)methyl]-2-(2-furyl)pyrimidine-5-carboxamide;
4-Amino-2-(2-furyl)-N-indan-1-yl-pyrimidine-5-carboxamide;
4-Amino-2-(2-furyl)-N-indan-2-yl-pyrimidine-5-carboxamide;
4-Amino-2-(2-furyl)-N-(8-quinolylmethyl) pyrimidine-5-carboxamide;
4-Amino-N-[(1-ethylimidazol-2-yl)methyl]-2-(2-furyl)pyrimidine-5-carboxamide;
4-Amino-2-(2-furyl)-N-[(2-isopropyl-1,2,4-triazol-3-yl)methyl]pyrimidine-5-carboxamide;
4-Amino-2-(2-furyl)-N-[[4-(trifluoromethyl) phenyl]methyl]pyrimidine-5-carboxamide;
4-Amino-2-(2-furyl)-N-[[3-(trifluoro methyl)phenyl]methyl]pyrimidine-5-carboxamide;
4-Amino-2-(2-furyl)-N-[[3-(trifluoromethyl)-2-pyridyl]methyl]pyrimidine-5-carboxamide;
4-Amino-N-[(2,6-dimethoxyphenyl)methyl]-2-(2-furyl)pyrimidine-5-carboxamide;
4-Amino-2-(2-furyl)-N-[(6-methyl-2-pyridyl)methyl]pyrimidine-5-carboxamide;
4-Amino-2-(2-furyl)-N-[(1-isopropylimidazol-2-yl)methyl]pyrimidine-5-carboxamide;
4-Amino-2-(2-furyl)-N-(m-tolylmethyl) pyrimidine-5-carboxamide;
4-Amino-N-[[2-(difluoromethyl)phenyl]methyl]-2-(2-furyl)pyrimidine-5-carboxamide;
4-Amino-2-(2-furyl)-N-(3-isoquinolylmethyl) pyrimidine-5-carboxamide;
4-Amino-2-(2-furyl)-N-[(1-methylimidazol-2-yl)methyl]pyrimidine-5-carboxamide;
4-Amino-N-[(2,3-dimethoxyphenyl)methyl]-2-(2-furyl)pyrimidine-5-carboxamide;
4-Amino-N-[(3,5-dimethylphenyl)methyl]-2-(2-furyl)pyrimidine-5-carboxamide;
4-Amino-2-(2-furyl)-N-[[3-(trifluoromethoxy) phenyl]methyl]pyrimidine-5-carboxamide;
4-Amino-2-(2-furyl)-N-(1H-indol-5-ylmethyl) pyrimidine-5-carboxamide;
4-Amino-N-[(1S)-6-fluoroindan-1-yl]-2-(2-furyl)pyrimidine-5-carboxamide;
4-Amino-N-[(3,5-dimethoxyphenyl)methyl]-2-(2-furyl)pyrimidine-5-carboxamide;
4-Amino-N-[[3-fluoro-5-(trifluoromethyl) phenyl]methyl]-2-(2-furyl)pyrimidine-5-carboxamide;
4-Amino-2-(2-furyl)-N-(oxazol-2-ylmethyl)pyrimidine-5-carboxamide;
4-Amino-2-(2-furyl)-N-(6-isoquinolylmethyl) pyrimidine-5-carboxamide;
4-Amino-N-[(3-fluoro-2-pyridyl)methyl]-2-(2-furyl)pyrimidine-5-carboxamide;
4-Amino-2-(2-furyl)-N-(5,6,7,8-tetrahydroquinolin-8-yl)pyrimidine-5-carboxamide;
4-Amino-2-(2-furyl)-N-(isoxazol-5-ylmethyl)pyrimidine-5-carboxamide;
4-Amino-N-(1,3-benzodioxol-4-ylmethyl)-2-(2-furyl)pyrimidine-5-carboxamide;
4-Amino-N-[(3-amino-2-pyridyl)methyl]-2-(2-furyl)pyrimidine-5-carboxamide;
4-Amino-2-(2-furyl)-N-[[2-(trifluoromethyl)phenyl]methyl]pyrimidine-5-carboxamide;
4-Amino-N-[(2-fluoro-6-methoxy-phenyl)methyl]-2-(2-furyl)pyrimidine-5-carboxamide;
4-Amino-2-(2-furyl)-N-[1-(3-methyl-2-pyridyl)ethyl]pyrimidine-5-carboxamide;
4-Amino-2-(2-furyl)-N-(pyrimidin-2-ylmethyl)pyrimidine-5-carboxamide;
4-Amino-2-(2-furyl)-N-[[4-(trifluoromethyl)pyrimidin-2-yl]methyl]pyrimidine-5-carboxamide;
4-Amino-2-(2-furyl)-N-(3-hydroxypropyl)pyrimidine-5-carboxamide;
4-Amino-2-(2-furyl)-N-[(1S)-2-hydroxy-1-phenyl-ethyl]pyrimidine-5-carboxamide;
5-Amino-3-(3-fluorophenyl)-N-[(3-methyl-2-pyridyl)methyl]-1,2,4-triazine-6-carboxamide;
5-Amino-3-(4-fluorophenyl)-N-[(3-methyl-2-pyridyl)methyl]-1,2,4-triazine-6-carboxamide;
4-Amino-2-(2-fluorophenyl)-N-[(3-methyl-2-pyridyl)methyl]pyrimidine-5-carboxamide;
4-Amino-2-(3-fluorophenyl)-N-(p-tolylmethyl)pyrimidine-5-carboxamide;
4-Amino-2-(3-fluorophenyl)-N-[(2-hydroxyphenyl)methyl]pyrimidine-5-carboxamide;
4-Amino-N-[(3-chlorophenyl)methyl]-2-(3-fluorophenyl)pyrimidine-5-carboxamide;
4-Amino-2-(3-fluorophenyl)-N-[(3-fluorophenyl)methyl]pyrimidine-5-carboxamide;
4-Amino-2-(3-fluorophenyl)-N-[(4-fluorophenyl)methyl]pyrimidine-5-carboxamide;
4-Amino-N-[(2-chlorophenyl)methyl]-2-(3-fluorophenyl)pyrimidine-5-carboxamide;
4-Amino-N-[(2,6-dichlorophenyl)methyl]-2-(3-fluorophenyl)pyrimidine-5-carboxamide;
4-Amino-N-[(3,5-difluorophenyl)methyl]-2-(3-fluorophenyl)pyrimidine-5-carboxamide;
4-Amino-2-(3-fluorophenyl)-N-[(2-methoxyphenyl)methyl]pyrimidine-5-carboxamide;
4-Amino-N-[(3,5-dimethoxyphenyl)methyl]-2-(3-fluorophenyl)pyrimidine-5-carboxamide;
4-Amino-N-[(2,3-dimethoxyphenyl)methyl]-2-(3-fluorophenyl)pyrimidine-5-carboxamide;
4-Amino-2-(3-fluorophenyl)-N-[[3-(trifluoromethyl)phenyl]methyl]pyrimidine-5-carboxamide;
4-Amino-N-[(3-chloro-2-pyridyl)methyl]-2-(3-fluorophenyl)pyrimidine-5-carboxamide;
4-Amino-2-(3-fluorophenyl)-N-(8-quinolylmethyl)pyrimidine-5-carboxamide;
4-Amino-N-[(1-ethylimidazol-2-yl)methyl]-2-(3-fluorophenyl)pyrimidine-5-carboxamide;
4-Amino-2-(3-fluorophenyl)-N-[(2-isopropyl-1,2,4-triazol-3-yl)methyl]pyrimidine-5-carboxamide;
4-Amino-2-(2-furyl)-N-(p-tolylmethyl)pyrimidine-5-carboxamide;
4-amino-2-(2-furyl)-N-[(2-hydroxyphenyl)methyl]pyrimidine-5-carboxamide;
4-Amino-2-(3-cyanophenyl)-N-[(3-methyl-2-pyridyl)methyl]pyrimidine-5-carboxamide;
4-Amino-6-chloro-2-(2-furyl)-N-[(3-methyl-2-pyridyl)methyl]pyrimidine-5-carboxamide;
4-Amino-2-(4-fluorophenyl)-N-[(3-methyl-2-pyridyl)methyl]pyrimidine-5-carboxamide;
4-Amino-N-[(3-methyl-2-pyridyl)methyl]-2-pyrazol-1-yl-pyrimidine-5-carboxamide;
4-Amino-N-[(3-methyl-2-pyridyl)methyl]-2-oxazol-2-yl-pyrimidine-5-carboxamide;
4-Amino-N-[(3-methyl-2-pyridyl)methyl]-2-thiazol-2-yl-pyrimidine-5-carboxamide;
5-Amino-3-(3-cyanophenyl)-N-[(3-methyl-2-pyridyl)methyl]-1,2,4-triazine-6-carboxamide;
4-Amino-2-(2-furyl)-6-methoxy-N-[(3-methyl-2-pyridyl)methyl]pyrimidine-5-carboxamide;
4-Amino-2-(2-furyl)-6-methoxy-N-[[3-(trifluoromethyl)-2-pyridyl]methyl]pyrimidine-5-carboxamide;
4-Amino-N-[(2,6-dichlorophenyl)methyl]-2-(2-furyl)-6-methoxy-pyrimidine-5-carboxamide;
4-Amino-2-(2-furyl)-N-[(3-methyl-2-pyridyl)methyl]-6-pyrazol-1-yl-pyrimidine-5-carboxamide;
4-(2-Acetamidoethoxy)-6-amino-2-(2-fury)-N-[(3-methyl-2-pyridyl)methyl]pyrimidine-5-carboxamide;
4-Amino-2-(2-furyl)-6-(2-hydroxyethoxy)-N-[(3-methyl-2-pyridyl)methyl]pyrimidine-5-carboxamide;
4-Amino-2-(2-furyl)-N-[(3-methyl-2-pyridyl)methyl]-6-(2,2,2-trifluoroethoxy)pyrimidine-5-carboxamide;
4-Amino-2-(2-furyl)-6-isobutoxy-N-[(3-methyl-2-pyridyl)methyl]pyrimidine-5-carboxamide;
4-Amino-2-(2-furyl)-6-(2-methoxyethoxy)-N-[(3-methyl-2-pyridyl)methyl]pyrimidine-5-carboxamide;
4-Amino-6-(2-fluoroethoxy)-2-(2-furyl)-N-[(3-methyl-2-pyridyl)methyl]pyrimidine-5-carboxamide;
4-Amino-2-(2-furyl)-6-(3-hydroxypropoxy)-N-[(3-methyl-2-pyridyl)methyl]pyrimidine-5-carboxamide;
4-Amino-2-(2-furyl)-N-[(3-methyl-2-pyridyl)methyl]-6-propoxy-pyrimidine-5-carboxamide;
4-Amino-6-(2,2-dimethylpropoxy)-2-(2-furyl)-N-[(3-methyl-2-pyridyl)methyl]pyrimidine-5-carboxamide;
4-Amino-2-(2-furyl)-6-[(2S)-2-hydroxypropoxy]-N-[(3-methyl-2-pyridyl)methyl]pyrimidine-5-carboxamide;
4-Amino-2-(2-furyl)-6-(2-methoxy-1-methyl-ethoxy)-N-[(3-methyl-2-pyridyl)methyl]pyrimidine-5-carboxamide;
4-Amino-2-(2-furyl)-6-[(2R)-2-hydroxypropoxy]-N-[(3-methyl-2-pyridyl)methyl]pyrimidine-5-carboxamide;
4-(2-Acetamidoethylamino)-6-amino-2-(2-furyl)-N-[(3-methyl-2-pyridyl)methyl]pyrimidine-5-carboxamide;
4-Amino-2-(2-furyl)-N-[(3-methyl-2-pyridyl)methyl]-6-[2-(2-piperidyl)ethoxy]pyrimidine-5-carboxamide;
4-Amino-6-(2-aminoethoxy)-2-(2-furyl)-N-[(3-methyl-2-pyridyl)methyl]pyrimidine-5-carboxamide;
4-Amino-2-(2-furyl)-N-[(3-methyl-2-pyridyl)methyl]-6-(2-piperazin-1-ylethoxy)pyrimidine-5-carboxamide;
4-Amino-6-(3-aminopropoxy)-2-(2-furyl)-N-[(3-methyl-2-pyridyl)methyl]pyrimidine-5-carboxamide;
4-Amino-2-(3-cyanophenyl)-6-(2-fluoroethoxy)-N-isopropyl-pyrimidine-5-carboxamide;
4-Amino-2-(3-cyanophenyl)-6-(2-fluoroethoxy)-N-propyl-pyrimidine-5-carboxamide;
4-Amino-2-(3-cyanophenyl)-6-(2-fluoroethoxy)-N-isobutyl-pyrimidine-5-carboxamide;
4-Amino-N-butyl-2-(3-cyanophenyl)-6-(2-fluoroethoxy)pyrimidine-5-carboxamide;
4-Amino-2-(3-cyanophenyl)-6-(2-fluoroethoxy)-N-[(2-isopropyl-1,2,4-triazol-3-yl)methyl]pyrimidine-5-carboxamide;
4-Amino-2-(3-cyanophenyl)-N-[2-(dimethylamino)ethyl]-6-(2-fluoroethoxy)pyrimidine-5-carboxamide;
4-Amino-2-(3-cyanophenyl)-6-(2-fluoroethoxy)-N-(3-hydroxybutyl)pyrimidine-5-carboxamide;
4-Amino-2-(3-cyanophenyl)-6-(2-fluoroethoxy)-N-[(2-methyl-1,2,4-triazol-3-yl) methyl]pyrimidine-5-carboxamide;
4-Amino-2-(3-cyanophenyl)-6-(2-fluoroethoxy)-N-[(3-methyl-2-pyridyl)methyl]pyrimidine-5-carboxamide;
4-Amino-6-(2-fluoroethoxy)-N-[(3-methyl-2-pyridyl)methyl]-2-oxazol-2-yl-pyrimidine-5-carboxamide;
   and pharmaceutically acceptable salts and solvates thereof.

In an embodiment, the compound of the present invention (i.e. a compound of formula (I), (Ia), (Ib), (Ic) or (Id)) is not one of the following compounds:
ethyl 4-amino-2-(2-methoxyphenyl)pyrimidine-5-carboxylate;
ethyl 4-amino-2-(o-tolyl)pyrimidine-5-carboxylate;
ethyl 4-amino-2-(2-chlorophenyl)pyrimidine-5-carboxylate;
ethyl 4-amino-2-phenylpyrimidine-5-carboxylate;
ethyl 4-amino-2-(4-chlorophenyl)pyrimidine-5-carboxylate;
ethyl 4-amino-2-(2-hydroxyphenyl)pyrimidine-5-carboxylate;
2-(3,5-dimethyl-pyrazol-1-yl)-4-amino-5-carbethoxypyrimidine (ethyl 4-amino-2-(3,5-dimethyl-1H-pyrazol-1-yl)pyrimidine-5-carboxylate);
ethyl 4-amino-2-(2-isopropoxyphenyl)pyrimidine-5-carboxylate;
ethyl-4-amino-6-methyl-2-(p-chloro-phenyl)pyrimidin-5-carboxylate;
ethyl-5-amino-3-phenyl-1,2,4-triazine-6-carboxylate;
ethyl-5-amino-3-(pyridin-2-yl)-1,2,4-triazine-6-carboxylate;
ethyl-5-amino-3-(pyrimidin-2-yl)-1,2,4-triazine-6-carboxylate;
ethyl-5-amino-3-(pyrazin-2-yl)-1,2,4-triazine-6-carboxylate;
5-Pyrimidinecarboxamide, 4-amino-2-(2-furanyl)-N-(3-methylbutyl)-;
5-Pyrimidinecarboxamide, 4-amino-N-hexyl-2-(2-thienyl)-;
5-Pyrimidinecarboxamide, 4-amino-N-(1,1-dimethylethyl)-2-(2-furanyl)-;
5-Pyrimidinecarboxamide, 4-amino-N-(1-methylpropyl)-2-(2-thienyl)-;
5-Pyrimidinecarboxamide, 4-amino-N-propyl-2-(2-thienyl)-;
5-Pyrimidinecarboxamide, 4-amino-N-(4,6-dimethyl-2-pyrimidinyl)-2-(2-furanyl)-;
5-Pyrimidinecarboxamide, 4-amino-2-(2-furanyl)-N-(5-methyl-1,3,4-thiadiazol-2-yl)-;
5-Pyrimidinecarboxamide, 4-amino-N-2-pyrimidinyl-2-(2-thienyl)-;
5-Pyrimidinecarboxamide, 4-amino-N-[3-(dimethylamino)propyl]-2-(2-thienyl)-;
5-Pyrimidinecarboxamide, 4-amino-2-(2-furanyl)-N-(4-pyridinylmethyl)-;
5-Pyrimidinecarboxamide, 4-amino-N-(2,4-dimethylphenyl)-2-(2-furanyl)-;
5-Pyrimidinecarboxamide, 4-amino-2-(2-furanyl)-N-2-thiazolyl-;
5-Pyrimidinecarboxamide, 4-amino-2-(2-furanyl)-N-(6-methyl-2-pyridinyl)-;
5-Pyrimidinecarboxamide, 4-amino-N-[2-(dimethylamino)ethyl]-2-(2-furanyl)-;
5-Pyrimidinecarboxamide, 4-amino-2-(2-furanyl)-N-2H-tetrazol-5-yl-;
5-Pyrimidinecarboxamide, 4-amino-N-(3,5-dimethylphenyl)-2-(2-furanyl)-;
5-Pyrimidinecarboxamide, 4-amino-2-(2-furanyl)-N-4-pyridinyl-;
5-Pyrimidinecarboxamide, 4-amino-2-(2-furanyl)-N-(3-methylphenyl)-;
5-Pyrimidinecarboxamide, 4-amino-2-(2-furanyl)-N-(2-pyridinylmethyl)-;
5-Pyrimidinecarboxamide, 4-amino-2-(5-bromo-2-furanyl)-N-methyl-;
5-Pyrimidinecarboxamide, 4-amino-2-(2-furanyl)-N-(4-methoxyphenyl)-;
5-Pyrimidinecarboxamide, 4-amino-2-(2-furanyl)-N-3-pyridinyl-;
5-Pyrimidinecarboxamide, 4-amino-2-(2-furanyl)-N-methyl-;
5-Pyrimidinecarboxamide, 4-amino-N-[3-(dimethylamino)propyl]-2-(2-furanyl)-;
5-Pyrimidinecarboxamide, 4-amino-N-(5-methyl-3-isoxazolyl)-2-(2-thienyl)-;
5-Pyrimidinecarboxamide, 4-amino-N-2-thiazolyl-2-(2-thienyl)-;
5-Pyrimidinecarboxamide, 4-amino-N-(2-fluorophenyl)-2-(2-furanyl)-;
5-Pyrimidinecarboxamide, 4-amino-N-(2-furanylmethyl)-2-(2-thienyl)-;
5-Pyrimidinecarboxamide, 4-amino-N-2H-tetrazol-5-yl-2-(2-thienyl)-;
5-Pyrimidinecarboxamide, 4-amino-N-(4-methylphenyl)-2-(2-thienyl)-;
5-Pyrimidinecarboxamide, 4-amino-2-(2-furanyl)-N-pentyl-;
5-Pyrimidinecarboxamide, 4-amino-N-(3-methoxypropyl)-2-(2-thienyl)-;
5-Pyrimidinecarboxamide, 4-amino-N-butyl-2-(2-furanyl)-;
5-Pyrimidinecarboxamide, 4-amino-N-ethyl-2-(2-furanyl)-;
5-Pyrimidinecarboxamide, 4-amino-N-cyclohexyl-2-(2-thienyl)-;
5-Pyrimidinecarboxamide, 4-amino-2-(2-furanyl)-N-phenyl-;
5-Pyrimidinecarboxamide, 4-amino-N-2-pyridinyl-2-(2-thienyl)-;
5-Pyrimidinecarboxamide, 4-amino-N-ethyl-2-(2-thienyl)-;
5-Pyrimidinecarboxamide, 4-amino-N-3-pyridinyl-2-(2-thienyl)-;
5-Pyrimidinecarboxamide, 4-amino-2-(2-furanyl)-N-(1-phenylethyl)-;
5-Pyrimidinecarboxamide, 4-amino-N-phenyl-2-(2-thienyl)-;
5-Pyrimidinecarboxamide, 4-amino-2-(2-furanyl)-N-(3-pyridinylmethyl)-;
5-Pyrimidinecarboxamide, 4-amino-2-(2-furanyl)-N-(1-methylpropyl)-;
5-Pyrimidinecarboxamide, 4-amino-N-(2-ethylphenyl)-2-(2-furanyl)-;
5-Pyrimidinecarboxamide, 4-amino-2-(2-furanyl)-N-(2-methylphenyl)-;
5-Pyrimidinecarboxamide, 4-amino-N-(2,3-dimethylphenyl)-2-(2-furanyl)-;
5-Pyrimidinecarboxamide, 4-amino-2-(2-furanyl)-N-(5-methyl-2-pyridinyl)-;
5-Pyrimidinecarboxamide, 4-amino-2-(2-furanyl)-N-(phenylmethyl)-;
5-Pyrimidinecarboxamide, 4-amino-2-(2-furanyl)-N-(5-methyl-3-isoxazolyl)-;
5-Pyrimidinecarboxamide, 4-amino-2-(2-furanyl)-N-(2-furanylmethyl)-;
5-Pyrimidinecarboxamide, 4-amino-2-(2-furanyl)-N-(4-methylphenyl)-;
5-Pyrimidinecarboxamide, 4-amino-2-(2-furanyl)-N-hexyl-;
5-Pyrimidinecarboxamide, 4-amino-N-(3-ethoxypropyl)-2-(2-furanyl)-;
5-Pyrimidinecarboxamide, 4-amino-N-(phenylmethyl)-2-(2-thienyl)-;
5-Pyrimidinecarboxamide, 4-amino-N-(3-ethoxypropyl)-2-(2-thienyl)-;
5-Pyrimidinecarboxamide, 4-amino-N-(3,4-dimethylphenyl)-2-(2-furanyl)-;
5-Pyrimidinecarboxamide, 4-amino-N-(1,1-dimethylethyl)-2-(2-thienyl)-;
5-Pyrimidinecarboxamide, 4-amino-2-(2-furanyl)-N-(2-methylpropyl)-;
5-Pyrimidinecarboxamide, 4-amino-N-4-pyridinyl-2-(2-thienyl)-;
5-Pyrimidinecarboxamide, 4-amino-2-(2-furanyl)-N-(4-methyl-2-pyridinyl)-;
5-Pyrimidinecarboxamide, 4-amino-N-[2-(dimethylamino)ethyl]-2-(2-thienyl)-;
5-Pyrimidinecarboxamide, 4-amino-2-(2-furanyl)-N-2-pyridinyl-;
5-Pyrimidinecarboxamide, 4-amino-2-(2-furanyl)-N-propyl-;
5-Pyrimidinecarboxamide, 4-amino-2-(2-furanyl)-N-(1-methylethyl)-;
5-Pyrimidinecarboxamide, 4-amino-2-(2-furanyl)-N-(3-methyl-2-pyridinyl)-;
5-Pyrimidinecarboxamide, 4-amino-N-(3-methylbutyl)-2-(2-thienyl)-;
5-Pyrimidinecarboxamide, 4-amino-N-(4-ethylphenyl)-2-(2-furanyl)-;
5-Pyrimidinecarboxamide, 4-amino-N-(2-methylpropyl)-2-(2-thienyl)-;
5-Pyrimidinecarboxamide, 4-amino-2-(2-furanyl)-N-(2-methoxyphenyl)-;
5-Pyrimidinecarboxamide, 4-amino-N-butyl-2-(2-thienyl)-;
5-Pyrimidinecarboxamide, 4-amino-N-methyl-2-(2-thienyl)-;
5-Pyrimidinecarboxamide, 4-amino-2-(2-furanyl)-N-(3-methoxypropyl)-;
5-Pyrimidinecarboxamide, 4-amino-N-pentyl-2-(2-thienyl)-;
5-Pyrimidinecarboxamide, 4-amino-N-(2-methoxyethyl)-2-(2-thienyl)-;
5-Pyrimidinecarboxamide, 4-amino-2-(2-furanyl)-N-2-pyrimidinyl-;
5-Pyrimidinecarboxamide, 4-amino-N-(2-methylphenyl)-2-(2-thienyl)-;
5-Pyrimidinecarboxamide, 4-amino-N-[2-(diethylamino)ethyl]-2-(2-furanyl)-;
5-Pyrimidinecarboxamide, 4-amino-2-(2-furanyl)-N-(2-methoxyethyl)-;
5-Pyrimidinecarboxamide, 4-amino-N-(4-fluorophenyl)-2-(2-furanyl)-;
5-Pyrimidinecarboxamide, 4-amino-2-(2-furanyl)-N-(2-phenylethyl)-;
5-Pyrimidinecarboxamide, 4-amino-N-(2,6-dimethylphenyl)-2-(2-furanyl)-;
5-Pyrimidinecarboxamide, 4-amino-N-(3-fluorophenyl)-2-(2-furanyl)-;
5-Pyrimidinecarboxamide, 4-amino-2-(2-furanyl)-N-(4-methyl-2-thiazolyl)-;
5-Pyrimidinecarboxamide, 4-amino-N-(1-methylethyl)-2-(2-thienyl)-;
5-Pyrimidinecarboxamide, 4-amino-2-(2-furanyl)-N-[(4-methylphenyl)methyl]-;
5-Pyrimidinecarboxamide, 4-amino-N-(3-methylphenyl)-2-(2-thienyl)-;
5-Pyrimidinecarboxamide, 4-amino-N-(2,5-dimethylphenyl)-2-(2-furanyl)-;
5-Pyrimidinecarboxamide, 4-amino-2-(2-furanyl)-N-[3-(1-methylethoxy)propyl]-;
5-Pyrimidinecarboxamide, 4-amino-N-methyl-2-phenyl-;
5-Pyrimidinecarboxamide, 4-amino-N-methyl-2-(2-pyridinyl)-;
5-Pyrimidinecarboxamide, 4-amino-N-methyl-2-(4-pyridinyl)-;
5-Pyrimidinecarboxamide, 4-amino-N-methyl-2-(3-pyridinyl)-;
5-Pyrimidinecarboxamide, 4-amino-N-ethyl-2-phenyl-;
5-Pyrimidinecarboxamide, 4-amino-N-methyl-2-(2-methylphenyl)-;
5-Pyrimidinecarboxamide, 4-amino-N-methyl-2-(3-methylphenyl)-;
5-Pyrimidinecarboxamide, 4-amino-N-methyl-2-(4-methylphenyl)-;
5-Pyrimidinecarboxamide, 4-amino-N-ethyl-2-(3-pyridinyl)-;
5-Pyrimidinecarboxamide, 4-amino-N-ethyl-2-(4-pyridinyl)-;
5-Pyrimidinecarboxamide, 4-amino-N-ethyl-2-(2-pyridinyl)-;
5-Pyrimidinecarboxamide, 4-amino-2-(4-fluorophenyl)-N-methyl-;
5-Pyrimidinecarboxamide, 4-amino-2-(3-fluorophenyl)-N-methyl-;
5-Pyrimidinecarboxamide, 4-amino-2-(2-fluorophenyl)-N-methyl-;
5-Pyrimidinecarboxamide, 4-amino-N-(1-methylethyl)-2-phenyl-;
5-Pyrimidinecarboxamide, 4-amino-N-ethyl-2-(2-methylphenyl)-;
5-Pyrimidinecarboxamide, 4-amino-2-(4-ethylphenyl)-N-methyl-;
5-Pyrimidinecarboxamide, 4-amino-N-ethyl-2-(3-methylphenyl)-;
5-Pyrimidinecarboxamide, 4-amino-2-phenyl-N-propyl-;
5-Pyrimidinecarboxamide, 4-amino-N-ethyl-2-(4-methylphenyl)-;
5-Pyrimidinecarboxamide, 4-amino-N-(1-methylethyl)-2-(2-pyridinyl)-;
5-Pyrimidinecarboxamide, 4-amino-N-propyl-2-(3-pyridinyl)-;
5-Pyrimidinecarboxamide, 4-amino-N-(1-methylethyl)-2-(4-pyridinyl)-;
5-Pyrimidinecarboxamide, 4-amino-N-propyl-2-(2-pyridinyl)-;
5-Pyrimidinecarboxamide, 4-amino-N-propyl-2-(4-pyridinyl)-;
5-Pyrimidinecarboxamide, 4-amino-N-(1-methylethyl)-2-(3-pyridinyl)-;
5-Pyrimidinecarboxamide, 4-amino-2-(3-methoxyphenyl)-N-methyl-;
5-Pyrimidinecarboxamide, 4-amino-2-(4-methoxyphenyl)-N-methyl-;
5-Pyrimidinecarboxamide, 4-amino-2-(2-methoxyphenyl)-N-methyl-;
5-Pyrimidinecarboxamide, 4-amino-N-ethyl-2-(4-fluorophenyl)-;
5-Pyrimidinecarboxamide, 4-amino-N-ethyl-2-(2-fluorophenyl)-;
5-Pyrimidinecarboxamide, 4-amino-N-ethyl-2-(3-fluorophenyl)-;
5-Pyrimidinecarboxamide, 4-amino-2-(2-chlorophenyl)-N-methyl-;
5-Pyrimidinecarboxamide, 4-amino-2-(3-chlorophenyl)-N-methyl-;
5-Pyrimidinecarboxamide, 4-amino-2-(4-chlorophenyl)-N-methyl-;
5-Pyrimidinecarboxamide, 4-amino-N-ethyl-2-(4-ethylphenyl)-;
5-Pyrimidinecarboxamide, 4-amino-2-(3-methylphenyl)-N-propyl-;
5-Pyrimidinecarboxamide, 4-amino-N-(1-methylethyl)-2-(4-methylphenyl)-;
5-Pyrimidinecarboxamide, 4-amino-N-(1-methylethyl)-2-(2-methylphenyl)-;
5-Pyrimidinecarboxamide, 4-amino-2-(2-methylphenyl)-N-propyl-;
5-Pyrimidinecarboxamide, 4-amino-N-butyl-2-phenyl-;
5-Pyrimidinecarboxamide, 4-amino-N-(1-methylpropyl)-2-phenyl-;
5-Pyrimidinecarboxamide, 4-amino-2-(4-methylphenyl)-N-propyl-;
5-Pyrimidinecarboxamide, 4-amino-N-(1-methylethyl)-2-(3-methylphenyl)-;
5-Pyrimidinecarboxamide, 4-amino-N-methyl-2-[4-(1-methylethyl)phenyl]-;
5-Pyrimidinecarboxamide, 4-amino-N-(1,1-dimethylethyl)-2-phenyl-;
5-Pyrimidinecarboxamide, 4-amino-N-(2-methylpropyl)-2-phenyl-;
5-Pyrimidinecarboxamide, 4-amino-N-(2-methylpropyl)-2-(4-pyridinyl)-;
5-Pyrimidinecarboxamide, 4-amino-N-(1,1-dimethylethyl)-2-(3-pyridinyl)-;
5-Pyrimidinecarboxamide, 4-amino-N-(1-methylpropyl)-2-(3-pyridinyl)-;
5-Pyrimidinecarboxamide, 4-amino-N-(1,1-dimethylethyl)-2-(4-pyridinyl)-;
5-Pyrimidinecarboxamide, 4-amino-N-(1,1-dimethylethyl)-2-(2-pyridinyl)-;
5-Pyrimidinecarboxamide, 4-amino-N-(2-methylpropyl)-2-(3-pyridinyl)-;
5-Pyrimidinecarboxamide, 4-amino-N-(1-methylpropyl)-2-(2-pyridinyl)-;
5-Pyrimidinecarboxamide, 4-amino-N-(2-methylpropyl)-2-(2-pyridinyl)-;
5-Pyrimidinecarboxamide, 4-amino-N-butyl-2-(2-pyridinyl)-;
5-Pyrimidinecarboxamide, 4-amino-N-(1-methylpropyl)-2-(4-pyridinyl)-;
5-Pyrimidinecarboxamide, 4-amino-N-butyl-2-(4-pyridinyl)-;
5-Pyrimidinecarboxamide, 4-amino-N-butyl-2-(3-pyridinyl)-;
5-Pyrimidinecarboxamide, 4-amino-2-(3-ethoxyphenyl)-N-methyl-;
5-Pyrimidinecarboxamide, 4-amino-2-(4-ethoxyphenyl)-N-methyl-;
5-Pyrimidinecarboxamide, 4-amino-N-ethyl-2-(2-methoxyphenyl)-;
5-Pyrimidinecarboxamide, 4-amino-2-(2-ethoxyphenyl)-N-methyl-;
5-Pyrimidinecarboxamide, 4-amino-N-ethyl-2-(4-methoxyphenyl)-;
5-Pyrimidinecarboxamide, 4-amino-N-(2-methoxyethyl)-2-phenyl-;
5-Pyrimidinecarboxamide, 4-amino-N-ethyl-2-(3-methoxyphenyl)-;
5-Pyrimidinecarboxamide, 4-amino-N-methyl-2-(3-nitrophenyl)-;
5-Pyrimidinecarboxamide, 4-amino-N-methyl-2-(4-nitrophenyl)-;
5-Pyrimidinecarboxamide, 4-amino-N-(2-methoxyethyl)-2-(2-pyridinyl)-;
5-Pyrimidinecarboxamide, 4-amino-N-(2-methoxyethyl)-2-(3-pyridinyl)-;
5-Pyrimidinecarboxamide, 4-amino-N-(2-methoxyethyl)-2-(4-pyridinyl)-;
5-Pyrimidinecarboxamide, 4-amino-2-(3-fluorophenyl)-N-(1-methylethyl)-;
5-Pyrimidinecarboxamide, 4-amino-2-(4-fluorophenyl)-N-propyl-;
5-Pyrimidinecarboxamide, 4-amino-2-(2-fluorophenyl)-N-(1-methylethyl)-;
5-Pyrimidinecarboxamide, 4-amino-2-(4-fluorophenyl)-N-(1-methylethyl)-;
5-Pyrimidinecarboxamide, 4-amino-2-(3-fluorophenyl)-N-propyl-;
5-Pyrimidinecarboxamide, 4-amino-2-(2-fluorophenyl)-N-propyl-;
5-Pyrimidinecarboxamide, 4-amino-2-(2-chlorophenyl)-N-ethyl-;
5-Pyrimidinecarboxamide, 4-amino-2-(4-chlorophenyl)-N-ethyl-;
5-Pyrimidinecarboxamide, 4-amino-2-(3-chlorophenyl)-N-ethyl-;
5-Pyrimidinecarboxamide, 4-amino-2-(2-chloro-6-fluorophenyl)-N-methyl-;
5-Pyrimidinecarboxamide, 4-amino-2-phenyl-N-2H-tetrazol-5-yl-;
5-Pyrimidinecarboxamide, 4-amino-2-(3-pyridinyl)-N-2H-tetrazol-5-yl-;
5-Pyrimidinecarboxamide, 4-amino-2-(2-pyridinyl)-N-2H-tetrazol-5-yl-;
5-Pyrimidinecarboxamide, 4-amino-2-(4-pyridinyl)-N-2H-tetrazol-5-yl-;
5-Pyrimidinecarboxamide, 4-amino-2-(2-methylphenyl)-N-(2-methylpropyl)-;
5-Pyrimidinecarboxamide, 4-amino-N-butyl-2-(3-methylphenyl)-;
5-Pyrimidinecarboxamide, 4-amino-2-(4-methylphenyl)-N-(2-methylpropyl)-;
5-Pyrimidinecarboxamide, 4-amino-N-butyl-2-(4-methylphenyl)-;
5-Pyrimidinecarboxamide, 4-amino-N-butyl-2-(2-methylphenyl)-;
5-Pyrimidinecarboxamide, 4-amino-N-ethyl-2-[4-(1-methylethyl)phenyl]-;
5-Pyrimidinecarboxamide, 4-amino-2-[4-(1,1-dimethylethyl)phenyl]-N-methyl-;
5-Pyrimidinecarboxamide, 4-amino-2-(4-ethylphenyl)-N-propyl-;
5-Pyrimidinecarboxamide, 4-amino-N-(1,1-dimethylethyl)-2-(2-methylphenyl)-;
5-Pyrimidinecarboxamide, 4-amino-N-pentyl-2-phenyl-;
5-Pyrimidinecarboxamide, 4-amino-2-(3-methylphenyl)-N-(1-methylpropyl)-;
5-Pyrimidinecarboxamide, 4-amino-N-(3-methylbutyl)-2-phenyl-;
5-Pyrimidinecarboxamide, 4-amino-2-(4-ethylphenyl)-N-(1-methylethyl)-;
5-Pyrimidinecarboxamide, 4-amino-2-(2-methylphenyl)-N-(1-methylpropyl)-;
5-Pyrimidinecarboxamide, 4-amino-N-(1,1-dimethylethyl)-2-(4-methylphenyl)-;
5-Pyrimidinecarboxamide, 4-amino-N-(1,1-dimethylethyl)-2-(3-methylphenyl)-;
5-Pyrimidinecarboxamide, 4-amino-2-(3-methylphenyl)-N-(2-methylpropyl)-;
5-Pyrimidinecarboxamide, 4-amino-2-(4-methylphenyl)-N-(1-methylpropyl)-;
5-Pyrimidinecarboxamide, 4-amino-N-[2-(dimethylamino)ethyl]-2-phenyl-;
5-Pyrimidinecarboxamide, 4-amino-N-(3-methylbutyl)-2-(3-pyridinyl)-;
5-Pyrimidinecarboxamide, 4-amino-N-(3-methylbutyl)-2-(2-pyridinyl)-;
5-Pyrimidinecarboxamide, 4-amino-N-pentyl-2-(2-pyridinyl)-;
5-Pyrimidinecarboxamide, 4-amino-N-(3-methylbutyl)-2-(4-pyridinyl)-;
5-Pyrimidinecarboxamide, 4-amino-N-pentyl-2-(4-pyridinyl)-;
5-Pyrimidinecarboxamide, 4-amino-N-pentyl-2-(3-pyridinyl)-;
5-Pyrimidinecarboxamide, 4-amino-2-(2-methoxyphenyl)-N-propyl-;
5-Pyrimidinecarboxamide, 4-amino-N-(3-methoxypropyl)-2-phenyl-;
5-Pyrimidinecarboxamide, 4-amino-2-(2-methoxyphenyl)-N-(1-methylethyl)-;
5-Pyrimidinecarboxamide, 4-amino-2-(4-ethoxyphenyl)-N-ethyl-;
5-Pyrimidinecarboxamide, 4-amino-N-methyl-2-[2-(1-methylethoxy)phenyl]-;
5-Pyrimidinecarboxamide, 4-amino-2-(3-methoxyphenyl)-N-(1-methylethyl)-;
5-Pyrimidinecarboxamide, 4-amino-2-(4-methoxyphenyl)-N-(1-methylethyl)-;
5-Pyrimidinecarboxamide, 4-amino-N-methyl-2-(4-propoxyphenyl)-;
5-Pyrimidinecarboxamide, 4-amino-2-(3-methoxyphenyl)-N-propyl-;
5-Pyrimidinecarboxamide, 4-amino-2-(4-methoxyphenyl)-N-propyl-;
5-Pyrimidinecarboxamide, 4-amino-N-methyl-2-[4-(1-methylethoxy)phenyl]-;
5-Pyrimidinecarboxamide, 4-amino-N-methyl-2-[3-(1-methylethoxy)phenyl]-;
5-Pyrimidinecarboxamide, 4-amino-N-(2-methoxyethyl)-2-(4-methylphenyl)-;
5-Pyrimidinecarboxamide, 4-amino-N-(2-methoxyethyl)-2-(3-methylphenyl)-;
5-Pyrimidinecarboxamide, 4-amino-2-(3-ethoxyphenyl)-N-ethyl-;
5-Pyrimidinecarboxamide, 4-amino-N-methyl-2-(2-propoxyphenyl)-;
5-Pyrimidinecarboxamide, 4-amino-2-(2-ethoxyphenyl)-N-ethyl-;
5-Pyrimidinecarboxamide, 4-amino-N-(2-methoxyethyl)-2-(2-methylphenyl)-;
5-Pyrimidinecarboxamide, 4-amino-N-methyl-2-(3-propoxyphenyl)-;
5-Pyrimidinecarboxamide, 4-amino-N-[2-(dimethylamino)ethyl]-2-(3-pyridinyl)-;
5-Pyrimidinecarboxamide, 4-amino-N-[2-(dimethylamino)ethyl]-2-(2-pyridinyl)-;
5-Pyrimidinecarboxamide, 4-amino-N-[2-(dimethylamino)ethyl]-2-(4-pyridinyl)-;
5-Pyrimidinecarboxamide, 4-amino-N-(3-methoxypropyl)-2-(4-pyridinyl)-;
5-Pyrimidinecarboxamide, 4-amino-N-(3-methoxypropyl)-2-(2-pyridinyl)-;
5-Pyrimidinecarboxamide, 4-amino-N-(3-methoxypropyl)-2-(3-pyridinyl)-;
5-Pyrimidinecarboxamide, 4-amino-2-(2,3-dimethoxyphenyl)-N-methyl-;
5-Pyrimidinecarboxamide, 4-amino-2-(2,5-dimethoxyphenyl)-N-methyl-;
5-Pyrimidinecarboxamide, 4-amino-2-(3,4-dimethoxyphenyl)-N-methyl-;
5-Pyrimidinecarboxamide, 4-amino-2-(2,4-dimethoxyphenyl)-N-methyl-;
5-Pyrimidinecarboxamide, 4-amino-2-(2-fluorophenyl)-N-(2-methylpropyl)-;
5-Pyrimidinecarboxamide, 4-amino-N-(1,1-dimethylethyl)-2-(2-fluorophenyl)-;
5-Pyrimidinecarboxamide, 4-amino-N-butyl-2-(2-fluorophenyl)-;
5-Pyrimidinecarboxamide, 4-amino-N-butyl-2-(4-fluorophenyl)-;
5-Pyrimidinecarboxamide, 4-amino-N-(1,1-dimethylethyl)-2-(3-fluorophenyl)-;
5-Pyrimidinecarboxamide, 4-amino-2-(4-fluorophenyl)-N-(1-methylpropyl)-;
5-Pyrimidinecarboxamide, 4-amino-2-(3-fluorophenyl)-N-(2-methylpropyl)-;
5-Pyrimidinecarboxamide, 4-amino-2-(4-fluorophenyl)-N-(2-methylpropyl)-;
5-Pyrimidinecarboxamide, 4-amino-2-(3-fluorophenyl)-N-(1-methylpropyl)-;
5-Pyrimidinecarboxamide, 4-amino-N-(1,1-dimethylethyl)-2-(4-fluorophenyl)-;
5-Pyrimidinecarboxamide, 4-amino-N-butyl-2-(3-fluorophenyl)-;
5-Pyrimidinecarboxamide, 4-amino-2-(2-fluorophenyl)-N-(1-methylpropyl)-;
5-Pyrimidinecarboxamide, 4-amino-2-(4-fluorophenyl)-N-(2-methoxyethyl)-;
5-Pyrimidinecarboxamide, 4-amino-2-(3-fluorophenyl)-N-(2-methoxyethyl)-;
5-Pyrimidinecarboxamide, 4-amino-2-(2-fluorophenyl)-N-(2-methoxyethyl)-;
5-Pyrimidinecarboxamide, 4-amino-N,2-diphenyl-;
5-Pyrimidinecarboxamide, 4-amino-2-(2-chlorophenyl)-N-propyl-;
5-Pyrimidinecarboxamide, 4-amino-2-(2-chlorophenyl)-N-(1-methylethyl)-;
5-Pyrimidinecarboxamide, 4-amino-2-(3-chlorophenyl)-N-propyl-;
5-Pyrimidinecarboxamide, 4-amino-2-(3-chlorophenyl)-N-(1-methylethyl)-;
5-Pyrimidinecarboxamide, 4-amino-2-(4-chlorophenyl)-N-(1-methylethyl)-;
5-Pyrimidinecarboxamide, 4-amino-2-(4-chlorophenyl)-N-propyl-;
5-Pyrimidinecarboxamide, 4-amino-N-phenyl-2-(4-pyridinyl)-;
5-Pyrimidinecarboxamide, 4-amino-N-phenyl-2-(3-pyridinyl)-;
5-Pyrimidinecarboxamide, 4-amino-N-phenyl-2-(2-pyridinyl)-;
5-Pyrimidinecarboxamide, 4-amino-2-phenyl-N-2-pyridinyl-;
5-Pyrimidinecarboxamide, 4-amino-2-phenyl-N-3-pyridinyl-;
5-Pyrimidinecarboxamide, 4-amino-2-phenyl-N-4-pyridinyl-;
5-Pyrimidinecarboxamide, 4-amino-N-3-pyridinyl-2-(4-pyridinyl)-;
5-Pyrimidinecarboxamide, 4-amino-N,2-di-4-pyridinyl-;
5-Pyrimidinecarboxamide, 4-amino-2-(2-pyridinyl)-N-3-pyridinyl-;
5-Pyrimidinecarboxamide, 4-amino-2-(2-pyridinyl)-N-4-pyridinyl-;
5-Pyrimidinecarboxamide, 4-amino-N,2-di-3-pyridinyl-;
5-Pyrimidinecarboxamide, 4-amino-N-2-pyridinyl-2-(3-pyridinyl)-;
5-Pyrimidinecarboxamide, 4-amino-N-2-pyridinyl-2-(4-pyridinyl)-;
5-Pyrimidinecarboxamide, 4-amino-2-(3-pyridinyl)-N-4-pyridinyl-;
5-Pyrimidinecarboxamide, 4-amino-2-phenyl-N-2-pyrimidinyl-;
5-Pyrimidinecarboxamide, 4-amino-N,2-di-2-pyridinyl-;
5-Pyrimidinecarboxamide, 4-amino-2-(3-chloro-4-methoxyphenyl)-N-methyl-;
5-Pyrimidinecarboxamide, 4-amino-2-(5-chloro-2-methoxyphenyl)-N-methyl-;
5-Pyrimidinecarboxamide, 4-amino-2-(2-pyridinyl)-N-2-pyrimidinyl-;
5-Pyrimidinecarboxamide, 4-amino-2-(4-pyridinyl)-N-2-pyrimidinyl-;
5-Pyrimidinecarboxamide, 4-amino-2-(3-pyridinyl)-N-2-pyrimidinyl-;
5-Pyrimidinecarboxamide, 4-amino-N-(2-furanylmethyl)-2-phenyl-;
5-Pyrimidinecarboxamide, 4-amino-2-(2-chloro-6-fluorophenyl)-N-ethyl-;
5-Pyrimidinecarboxamide, 4-amino-N-(2-furanylmethyl)-2-(2-pyridinyl)-;
5-Pyrimidinecarboxamide, 4-amino-N-(2-furanylmethyl)-2-(3-pyridinyl)-;
5-Pyrimidinecarboxamide, 4-amino-N-(5-methyl-3-isoxazolyl)-2-phenyl-;
5-Pyrimidinecarboxamide, 4-amino-N-(2-furanylmethyl)-2-(4-pyridinyl)-;
5-Pyrimidinecarboxamide, 4-amino-N-(5-methyl-3-isoxazolyl)-2-(4-pyridinyl)-;
5-Pyrimidinecarboxamide, 4-amino-N-(5-methyl-3-isoxazolyl)-2-(2-pyridinyl)-;
5-Pyrimidinecarboxamide, 4-amino-N-(5-methyl-3-isoxazolyl)-2-(3-pyridinyl)-;
5-Pyrimidinecarboxamide, 4-amino-2-(2-methylphenyl)-N-2H-tetrazol-5-yl-;
5-Pyrimidinecarboxamide, 4-amino-2-(4-methylphenyl)-N-2H-tetrazol-5-yl-;
5-Pyrimidinecarboxamide, 4-amino-2-(3-methylphenyl)-N-2H-tetrazol-5-yl-;
5-Pyrimidinecarboxamide, 4-amino-2-(2,4-dichlorophenyl)-N-methyl-;
5-Pyrimidinecarboxamide, 4-amino-2-(2,6-dichlorophenyl)-N-methyl-;
5-Pyrimidinecarboxamide, 4-amino-2-(3,4-dichlorophenyl)-N-methyl-;
5-Pyrimidinecarboxamide, 4-amino-2-phenyl-N-2-thiazolyl-;
5-Pyrimidinecarboxamide, 4-amino-2-(2-pyridinyl)-N-2-thiazolyl-;
5-Pyrimidinecarboxamide, 4-amino-2-(4-pyridinyl)-N-2-thiazolyl-;
5-Pyrimidinecarboxamide, 4-amino-2-(3-pyridinyl)-N-2-thiazolyl-;
5-Pyrimidinecarboxamide, 4-amino-2-phenyl-N-[(tetrahydro-2-furanyl)methyl]-;
5-Pyrimidinecarboxamide, 4-amino-2-(4-ethylphenyl)-N-(2-methylpropyl)-;
5-Pyrimidinecarboxamide, 4-amino-N-(1,1-dimethylethyl)-2-(4-ethylphenyl)-;
5-Pyrimidinecarboxamide, 4-amino-2-(2-methylphenyl)-N-pentyl-;
5-Pyrimidinecarboxamide, 4-amino-2-[4-(1-methylethyl)phenyl]-N-propyl-;
5-Pyrimidinecarboxamide, 4-amino-N-(1-methylethyl)-2-[4-(1-methylethyl)phenyl]-;
5-Pyrimidinecarboxamide, 4-amino-2-(4-ethylphenyl)-N-(1-methylpropyl)-;
5-Pyrimidinecarboxamide, 4-amino-2-[4-(1,1-dimethylethyl)phenyl]-N-ethyl-;
5-Pyrimidinecarboxamide, 4-amino-N-hexyl-2-phenyl-;
5-Pyrimidinecarboxamide, 4-amino-N-butyl-2-(4-ethylphenyl)-;
5-Pyrimidinecarboxamide, 4-amino-N-(3-methylbutyl)-2-(2-methylphenyl)-;
5-Pyrimidinecarboxamide, 4-amino-N-(3-methylbutyl)-2-(3-methylphenyl)-;
5-Pyrimidinecarboxamide, 4-amino-N-(3-methylbutyl)-2-(4-methylphenyl)-;
5-Pyrimidinecarboxamide, 4-amino-2-(4-methylphenyl)-N-pentyl-;
5-Pyrimidinecarboxamide, 4-amino-2-(3-methylphenyl)-N-pentyl-;
5-Pyrimidinecarboxamide, 4-amino-2-(3-pyridinyl)-N-[(tetrahydro-2-furanyl)methyl]-;
5-Pyrimidinecarboxamide, 4-amino-2-(4-pyridinyl)-N-[(tetrahydro-2-furanyl)methyl]-;
5-Pyrimidinecarboxamide, 4-amino-2-(2-pyridinyl)-N-[(tetrahydro-2-furanyl)methyl]-;
5-Pyrimidinecarboxamide, 4-amino-N-[2-(dimethylamino)ethyl]-2-(4-methylphenyl)-;
5-Pyrimidinecarboxamide, 4-amino-N-hexyl-2-(4-pyridinyl)-;
5-Pyrimidinecarboxamide, 4-amino-N-[2-(dimethylamino)ethyl]-2-(3-methylphenyl)-;
5-Pyrimidinecarboxamide, 4-amino-N-[3-(dimethylamino)propyl]-2-phenyl-;
5-Pyrimidinecarboxamide, 4-amino-N-hexyl-2-(3-pyridinyl)-;
5-Pyrimidinecarboxamide, 4-amino-N-[2-(dimethylamino)ethyl]-2-(2-methylphenyl)-;
5-Pyrimidinecarboxamide, 4-amino-N-hexyl-2-(2-pyridinyl)-;
5-Pyrimidinecarboxamide, 4-amino-2-(4-fluorophenyl)-N-2H-tetrazol-5-yl-;
5-Pyrimidinecarboxamide, 4-amino-2-(3-fluorophenyl)-N-2H-tetrazol-5-yl-;
5-Pyrimidinecarboxamide, 4-amino-2-(2-fluorophenyl)-N-2H-tetrazol-5-yl-;
5-Pyrimidinecarboxamide, 4-amino-2-(2-ethoxyphenyl)-N-(1-methylethyl)-;
5-Pyrimidinecarboxamide, 4-amino-N-ethyl-2-[4-(1-methylethoxy)phenyl]-;
5-Pyrimidinecarboxamide, 4-amino-N-(1,1-dimethylethyl)-2-(2-methoxyphenyl)-;
5-Pyrimidinecarboxamide, 4-amino-N-(3-methoxypropyl)-2-(3-methylphenyl)-;
5-Pyrimidinecarboxamide, 4-amino-N-methyl-2-[3-(1-methylpropoxy)phenyl]-;
5-Pyrimidinecarboxamide, 4-amino-N-ethyl-2-[2-(1-methylethoxy)phenyl]-;
5-Pyrimidinecarboxamide, 4-amino-N-methyl-2-[4-(2-methylpropoxy)phenyl]-;
5-Pyrimidinecarboxamide, 4-amino-N-ethyl-2-(4-propoxyphenyl)-;
5-Pyrimidinecarboxamide, 4-amino-N-(1,1-dimethylethyl)-2-(4-methoxyphenyl)-;
5-Pyrimidinecarboxamide, 4-amino-2-(4-ethoxyphenyl)-N-propyl-;
5-Pyrimidinecarboxamide, 4-amino-N-(3-methoxypropyl)-2-(2-methylphenyl)-;
5-Pyrimidinecarboxamide, 4-amino-2-(4-ethoxyphenyl)-N-(1-methylethyl)-;
5-Pyrimidinecarboxamide, 4-amino-N-ethyl-2-(2-propoxyphenyl)-;
5-Pyrimidinecarboxamide, 4-amino-N-ethyl-2-(3-propoxyphenyl)-;
5-Pyrimidinecarboxamide, 4-amino-2-(4-butoxyphenyl)-N-methyl-;
5-Pyrimidinecarboxamide, 4-amino-2-(2-butoxyphenyl)-N-methyl-;
5-Pyrimidinecarboxamide, 4-amino-2-(4-methoxyphenyl)-N-(1-methylpropyl)-;
5-Pyrimidinecarboxamide, 4-amino-2-(2-methoxyphenyl)-N-(1-methylpropyl)-;
5-Pyrimidinecarboxamide, 4-amino-N-methyl-2-[2-(2-methylpropoxy)phenyl]-;
5-Pyrimidinecarboxamide, 4-amino-2-(2-ethoxyphenyl)-N-propyl-;
5-Pyrimidinecarboxamide, 4-amino-N-ethyl-2-[3-(1-methylethoxy)phenyl]-;
5-Pyrimidinecarboxamide, 4-amino-2-(4-ethylphenyl)-N-(2-methoxyethyl)-;
5-Pyrimidinecarboxamide, 4-amino-N-(3-methoxypropyl)-2-(4-methylphenyl)-;
5-Pyrimidinecarboxamide, 4-amino-N-butyl-2-(3-methoxyphenyl)-;
5-Pyrimidinecarboxamide, 4-amino-2-(3-ethoxyphenyl)-N-(1-methylethyl)-;
5-Pyrimidinecarboxamide, 4-amino-2-(3-butoxyphenyl)-N-methyl-;
5-Pyrimidinecarboxamide, 4-amino-N-butyl-2-(4-methoxyphenyl)-;
5-Pyrimidinecarboxamide, 4-amino-N-butyl-2-(2-methoxyphenyl)-;
5-Pyrimidinecarboxamide, 4-amino-2-(3-ethoxyphenyl)-N-propyl-;
5-Pyrimidinecarboxamide, 4-amino-N-(3-ethoxypropyl)-2-phenyl-;
5-Pyrimidinecarboxamide, 4-amino-2-(3-methoxyphenyl)-N-(2-methylpropyl)-;
5-Pyrimidinecarboxamide, 4-amino-2-(4-methoxyphenyl)-N-(2-methylpropyl)-;
5-Pyrimidinecarboxamide, 4-amino-N-methyl-2-[3-(2-methylpropoxy)phenyl]-;
5-Pyrimidinecarboxamide, 4-amino-2-(3-methoxyphenyl)-N-(1-methylpropyl)-;
5-Pyrimidinecarboxamide, 4-amino-N-(1,1-dimethylethyl)-2-(3-methoxyphenyl)-;
5-Pyrimidinecarboxamide, 4-amino-2-(2-methoxyphenyl)-N-(2-methylpropyl)-;
5-Pyrimidinecarboxamide, 4-amino-N-methyl-2-[4-(1-methylpropoxy)phenyl]-;
5-Pyrimidinecarboxamide, 4-amino-N-methyl-2-[2-(1-methylpropoxy)phenyl]-;
5-Pyrimidinecarboxamide, 4-amino-N-[3-(dimethylamino)propyl]-2-(2-pyridinyl)-;
5-Pyrimidinecarboxamide, 4-amino-N-[3-(dimethylamino)propyl]-2-(4-pyridinyl)-;
5-Pyrimidinecarboxamide, 4-amino-N-[3-(dimethylamino)propyl]-2-(3-pyridinyl)-;
5-Pyrimidinecarboxamide, 4-amino-N-(3-ethoxypropyl)-2-(3-pyridinyl)-;
5-Pyrimidinecarboxamide, 4-amino-N-(3-ethoxypropyl)-2-(4-pyridinyl)-;
5-Pyrimidinecarboxamide, 4-amino-N-(3-ethoxypropyl)-2-(2-pyridinyl)-;
5-Pyrimidinecarboxamide, 4-amino-2-(3-ethoxy-4-methoxyphenyl)-N-methyl-;
5-Pyrimidinecarboxamide, 4-amino-2-(2,4-dimethoxyphenyl)-N-ethyl-;
5-Pyrimidinecarboxamide, 4-amino-2-(4-ethoxy-3-methoxyphenyl)-N-methyl-;
5-Pyrimidinecarboxamide, 4-amino-N-(2-methoxyethyl)-2-(2-methoxyphenyl)-;
5-Pyrimidinecarboxamide, 4-amino-N-(2-methoxyethyl)-2-(3-methoxyphenyl)-;
5-Pyrimidinecarboxamide, 4-amino-2-(2,5-dimethoxyphenyl)-N-ethyl-;
5-Pyrimidinecarboxamide, 4-amino-2-(2-ethoxy-3-methoxyphenyl)-N-methyl-;
5-Pyrimidinecarboxamide, 4-amino-2-(2,3-dimethoxyphenyl)-N-ethyl-;
5-Pyrimidinecarboxamide, 4-amino-N-(2-methoxyethyl)-2-(4-methoxyphenyl)-;
5-Pyrimidinecarboxamide, 4-amino-2-(3,4-dimethoxyphenyl)-N-ethyl-;
5-Pyrimidinecarboxamide, 4-amino-2-(3-fluorophenyl)-N-pentyl-;
5-Pyrimidinecarboxamide, 4-amino-2-(3-fluorophenyl)-N-(3-methylbutyl)-;
5-Pyrimidinecarboxamide, 4-amino-2-(2-fluorophenyl)-N-pentyl-;
5-Pyrimidinecarboxamide, 4-amino-2-(2-fluorophenyl)-N-(3-methylbutyl)-;
5-Pyrimidinecarboxamide, 4-amino-2-(4-fluorophenyl)-N-pentyl-;
5-Pyrimidinecarboxamide, 4-amino-2-(4-fluorophenyl)-N-(3-methylbutyl)-;
5-Pyrimidinecarboxamide, 4-amino-2-(2-methoxy-5-nitrophenyl)-N-methyl-;
5-Pyrimidinecarboxamide, 4-amino-2-(4-methoxy-3-nitrophenyl)-N-methyl-;
5-Pyrimidinecarboxamide, 4-amino-N-[2-(dimethylamino)ethyl]-2-(4-fluorophenyl)-;
5-Pyrimidinecarboxamide, 4-amino-N-[2-(dimethylamino)ethyl]-2-(3-fluorophenyl)-;
5-Pyrimidinecarboxamide, 4-amino-N-[2-(dimethylamino)ethyl]-2-(2-fluorophenyl)-;
5-Pyrimidinecarboxamide, 4-amino-2-(2-fluorophenyl)-N-(3-methoxypropyl)-;
5-Pyrimidinecarboxamide, 4-amino-2-(4-fluorophenyl)-N-(3-methoxypropyl)-;
5-Pyrimidinecarboxamide, 4-amino-2-(3-fluorophenyl)-N-(3-methoxypropyl)-;
5-Pyrimidinecarboxamide, 4-amino-N-(3-methylphenyl)-2-phenyl-;
5-Pyrimidinecarboxamide, 4-amino-2-(2-methylphenyl)-N-phenyl-;
5-Pyrimidinecarboxamide, 4-amino-N-(4-methylphenyl)-2-phenyl-;
5-Pyrimidinecarboxamide, 4-amino-2-(4-methylphenyl)-N-phenyl-;
5-Pyrimidinecarboxamide, 4-amino-2-phenyl-N-(phenylmethyl)-;
5-Pyrimidinecarboxamide, 4-amino-2-(3-methylphenyl)-N-phenyl-;
5-Pyrimidinecarboxamide, 4-amino-N-(2-methylphenyl)-2-phenyl-;
5-Pyrimidinecarboxamide, 4-amino-2-(4-chlorophenyl)-N-(1-methylpropyl)-;
5-Pyrimidinecarboxamide, 4-amino-N-butyl-2-(3-chlorophenyl)-;
5-Pyrimidinecarboxamide, 4-amino-2-(2-chlorophenyl)-N-(1,1-dimethylethyl)-;
5-Pyrimidinecarboxamide, 4-amino-2-(3-chlorophenyl)-N-(1-methylpropyl)-;
5-Pyrimidinecarboxamide, 4-amino-N-butyl-2-(2-chlorophenyl)-;
5-Pyrimidinecarboxamide, 4-amino-N-butyl-2-(4-chlorophenyl)-;
5-Pyrimidinecarboxamide, 4-amino-2-(2-chlorophenyl)-N-(1-methylpropyl)-;
5-Pyrimidinecarboxamide, 4-amino-2-(4-chlorophenyl)-N-(1,1-dimethylethyl)-;
5-Pyrimidinecarboxamide, 4-amino-2-(2-chlorophenyl)-N-(2-methylpropyl)-;
5-Pyrimidinecarboxamide, 4-amino-2-(3-chlorophenyl)-N-(1,1-dimethylethyl)-;
5-Pyrimidinecarboxamide, 4-amino-2-(4-chlorophenyl)-N-(2-methylpropyl)-;
5-Pyrimidinecarboxamide, 4-amino-2-(3-chlorophenyl)-N-(2-methylpropyl)-;
5-Pyrimidinecarboxamide, 4-amino-N-(6-methyl-2-pyridinyl)-2-phenyl-;
5-Pyrimidinecarboxamide, 4-amino-2-(4-methylphenyl)-N-4-pyridinyl-;
5-Pyrimidinecarboxamide, 4-amino-2-(3-methylphenyl)-N-4-pyridinyl-;
5-Pyrimidinecarboxamide, 4-amino-N-(3-methylphenyl)-2-(3-pyridinyl)-;
5-Pyrimidinecarboxamide, 4-amino-N-(3-methyl-2-pyridinyl)-2-phenyl-;
5-Pyrimidinecarboxamide, 4-amino-N-(phenylmethyl)-2-(3-pyridinyl)-;
5-Pyrimidinecarboxamide, 4-amino-N-(3-methylphenyl)-2-(2-pyridinyl)-;
5-Pyrimidinecarboxamide, 4-amino-N-(phenylmethyl)-2-(4-pyridinyl)-;
5-Pyrimidinecarboxamide, 4-amino-N-(2-methylphenyl)-2-(3-pyridinyl)-;
5-Pyrimidinecarboxamide, 4-amino-N-(3-methylphenyl)-2-(4-pyridinyl)-;
5-Pyrimidinecarboxamide, 4-amino-2-phenyl-N-(3-pyridinylmethyl)-;
5-Pyrimidinecarboxamide, 4-amino-2-(2-methylphenyl)-N-4-pyridinyl-;
5-Pyrimidinecarboxamide, 4-amino-2-(3-methylphenyl)-N-3-pyridinyl-;
5-Pyrimidinecarboxamide, 4-amino-N-(4-methylphenyl)-2-(3-pyridinyl)-;
5-Pyrimidinecarboxamide, 4-amino-N-(4-methylphenyl)-2-(4-pyridinyl)-;
5-Pyrimidinecarboxamide, 4-amino-N-(4-methylphenyl)-2-(2-pyridinyl)-;
5-Pyrimidinecarboxamide, 4-amino-2-phenyl-N-(4-pyridinylmethyl)-;
5-Pyrimidinecarboxamide, 4-amino-2-(4-methylphenyl)-N-3-pyridinyl-;
5-Pyrimidinecarboxamide, 4-amino-N-(2-methylphenyl)-2-(2-pyridinyl)-;
5-Pyrimidinecarboxamide, 4-amino-2-phenyl-N-(2-pyridinylmethyl)-;
5-Pyrimidinecarboxamide, 4-amino-2-(2-methylphenyl)-N-3-pyridinyl-;
5-Pyrimidinecarboxamide, 4-amino-N-(phenylmethyl)-2-(2-pyridinyl)-;
5-Pyrimidinecarboxamide, 4-amino-N-(2-methylphenyl)-2-(4-pyridinyl)-;
5-Pyrimidinecarboxamide, 4-amino-N-(4-methyl-2-pyridinyl)-2-phenyl-;
5-Pyrimidinecarboxamide, 4-amino-2-(2-methylphenyl)-N-2-pyridinyl-;
5-Pyrimidinecarboxamide, 4-amino-2-(4-methylphenyl)-N-2-pyridinyl-;
5-Pyrimidinecarboxamide, 4-amino-2-(3-methylphenyl)-N-2-pyridinyl-;
5-Pyrimidinecarboxamide, 4-amino-N-(5-methyl-2-pyridinyl)-2-phenyl-;
5-Pyrimidinecarboxamide, 4-amino-N-(5-methyl-2-pyridinyl)-2-(2-pyridinyl)-;
5-Pyrimidinecarboxamide, 4-amino-N-(3-methyl-2-pyridinyl)-2-(4-pyridinyl)-;
5-Pyrimidinecarboxamide, 4-amino-2-(4-methylphenyl)-N-2-pyrimidinyl-;
5-Pyrimidinecarboxamide, 4-amino-2-(3-pyridinyl)-N-(4-pyridinylmethyl)-;
5-Pyrimidinecarboxamide, 4-amino-2-(2-pyridinyl)-N-(4-pyridinylmethyl)-;
5-Pyrimidinecarboxamide, 4-amino-2-(3-pyridinyl)-N-(3-pyridinylmethyl)-;
5-Pyrimidinecarboxamide, 4-amino-N-(6-methyl-2-pyridinyl)-2-(3-pyridinyl)-;
5-Pyrimidinecarboxamide, 4-amino-N-(4-methyl-2-pyridinyl)-2-(4-pyridinyl)-;
5-Pyrimidinecarboxamide, 4-amino-N-(3-methyl-2-pyridinyl)-2-(2-pyridinyl)-;
5-Pyrimidinecarboxamide, 4-amino-2-(4-pyridinyl)-N-(2-pyridinylmethyl)-;
5-Pyrimidinecarboxamide, 4-amino-2-(4-pyridinyl)-N-(4-pyridinylmethyl)-;
5-Pyrimidinecarboxamide, 4-amino-N-(4-methyl-2-pyridinyl)-2-(2-pyridinyl)-;
5-Pyrimidinecarboxamide, 4-amino-2-(2-methylphenyl)-N-2-pyrimidinyl-;
5-Pyrimidinecarboxamide, 4-amino-N-(6-methyl-2-pyridinyl)-2-(2-pyridinyl)-;
5-Pyrimidinecarboxamide, 4-amino-N-(3-methyl-2-pyridinyl)-2-(3-pyridinyl)-;
5-Pyrimidinecarboxamide, 4-amino-2-(2-pyridinyl)-N-(3-pyridinylmethyl)-;
5-Pyrimidinecarboxamide, 4-amino-2-(4-pyridinyl)-N-(3-pyridinylmethyl)-;
5-Pyrimidinecarboxamide, 4-amino-N-(4-methyl-2-pyridinyl)-2-(3-pyridinyl)-;
5-Pyrimidinecarboxamide, 4-amino-2-(3-pyridinyl)-N-(2-pyridinylmethyl)-;
5-Pyrimidinecarboxamide, 4-amino-N-(5-methyl-2-pyridinyl)-2-(4-pyridinyl)-;
5-Pyrimidinecarboxamide, 4-amino-N-(5-methyl-2-pyridinyl)-2-(3-pyridinyl)-;
5-Pyrimidinecarboxamide, 4-amino-2-(2-pyridinyl)-N-(2-pyridinylmethyl)-;
5-Pyrimidinecarboxamide, 4-amino-2-(3-methylphenyl)-N-2-pyrimidinyl-;
5-Pyrimidinecarboxamide, 4-amino-N-(6-methyl-2-pyridinyl)-2-(4-pyridinyl)-;
5-Pyrimidinecarboxamide, 4-amino-2-(2-chloro-6-fluorophenyl)-N-2-propen-1-yl-;
5-Pyrimidinecarboxamide, 4-amino-2-(2-chlorophenyl)-N-(2-methoxyethyl)-;
5-Pyrimidinecarboxamide, 4-amino-2-(5-chloro-2-ethoxyphenyl)-N-methyl-;
5-Pyrimidinecarboxamide, 4-amino-2-(3-chloro-4-methoxyphenyl)-N-ethyl-;
5-Pyrimidinecarboxamide, 4-amino-2-(4-chlorophenyl)-N-(2-methoxyethyl)-;
5-Pyrimidinecarboxamide, 4-amino-2-(3-chloro-4-ethoxyphenyl)-N-methyl-;
5-Pyrimidinecarboxamide, 4-amino-2-(5-chloro-2-methoxyphenyl)-N-ethyl-;
5-Pyrimidinecarboxamide, 4-amino-2-(3-chlorophenyl)-N-(2-methoxyethyl)-;
5-Pyrimidinecarboxamide, 4-amino-N-(3-fluorophenyl)-2-phenyl-;
5-Pyrimidinecarboxamide, 4-amino-N-(2-fluorophenyl)-2-phenyl-;
5-Pyrimidinecarboxamide, 4-amino-N-(4-fluorophenyl)-2-phenyl-;
5-Pyrimidinecarboxamide, 4-amino-2-(4-fluorophenyl)-N-phenyl-;
5-Pyrimidinecarboxamide, 4-amino-2-(2-fluorophenyl)-N-phenyl-;
5-Pyrimidinecarboxamide, 4-amino-2-(3-fluorophenyl)-N-phenyl-;
5-Pyrimidinecarboxamide, 4-amino-N-(2-furanylmethyl)-2-(3-methylphenyl)-;
5-Pyrimidinecarboxamide, 4-amino-N-(2-furanylmethyl)-2-(4-methylphenyl)-;
5-Pyrimidinecarboxamide, 4-amino-N-(2-furanylmethyl)-2-(2-methylphenyl)-;
5-Pyrimidinecarboxamide, 4-amino-2-(2-chloro-6-fluorophenyl)-N-propyl-;
5-Pyrimidinecarboxamide, 4-amino-2-(2-chloro-6-fluorophenyl)-N-(1-methylethyl)-;
5-Pyrimidinecarboxamide, 4-amino-N-(3-fluorophenyl)-2-(3-pyridinyl)-;
5-Pyrimidinecarboxamide, 4-amino-2-(4-fluorophenyl)-N-4-pyridinyl-;
5-Pyrimidinecarboxamide, 4-amino-N-(2-fluorophenyl)-2-(4-pyridinyl)-;
5-Pyrimidinecarboxamide, 4-amino-2-(2-fluorophenyl)-N-3-pyridinyl-;
5-Pyrimidinecarboxamide, 4-amino-2-(2-fluorophenyl)-N-4-pyridinyl-;
5-Pyrimidinecarboxamide, 4-amino-N-(3-fluorophenyl)-2-(4-pyridinyl)-;
5-Pyrimidinecarboxamide, 4-amino-2-(3-fluorophenyl)-N-2-pyridinyl-;
5-Pyrimidinecarboxamide, 4-amino-2-(2-fluorophenyl)-N-2-pyridinyl-;
5-Pyrimidinecarboxamide, 4-amino-N-(2-fluorophenyl)-2-(3-pyridinyl)-;
5-Pyrimidinecarboxamide, 4-amino-2-(4-fluorophenyl)-N-3-pyridinyl-;
5-Pyrimidinecarboxamide, 4-amino-2-(4-fluorophenyl)-N-2-pyridinyl-;
5-Pyrimidinecarboxamide, 4-amino-N-(2-fluorophenyl)-2-(2-pyridinyl)-;
5-Pyrimidinecarboxamide, 4-amino-2-(3-fluorophenyl)-N-3-pyridinyl-;
5-Pyrimidinecarboxamide, 4-amino-N-(4-fluorophenyl)-2-(2-pyridinyl)-;
5-Pyrimidinecarboxamide, 4-amino-N-(4-fluorophenyl)-2-(3-pyridinyl)-;
5-Pyrimidinecarboxamide, 4-amino-2-(3-fluorophenyl)-N-4-pyridinyl-;
5-Pyrimidinecarboxamide, 4-amino-N-(4-fluorophenyl)-2-(4-pyridinyl)-;
5-Pyrimidinecarboxamide, 4-amino-N-(3-fluorophenyl)-2-(2-pyridinyl)-;
5-Pyrimidinecarboxamide, 4-amino-N-(5-methyl-3-isoxazolyl)-2-(3-methylphenyl)-;
5-Pyrimidinecarboxamide, 4-amino-N-(5-methyl-3-isoxazolyl)-2-(2-methylphenyl)-;
5-Pyrimidinecarboxamide, 4-amino-N-(5-methyl-3-isoxazolyl)-2-(4-methylphenyl)-;
5-Pyrimidinecarboxamide, 4-amino-2-(3-fluorophenyl)-N-2-pyrimidinyl-;
5-Pyrimidinecarboxamide, 4-amino-2-(2-fluorophenyl)-N-2-pyrimidinyl-;
5-Pyrimidinecarboxamide, 4-amino-2-(4-fluorophenyl)-N-2-pyrimidinyl-;
5-Pyrimidinecarboxamide, 4-amino-2-(4-ethylphenyl)-N-2H-tetrazol-5-yl-;
5-Pyrimidinecarboxamide, 4-amino-N-[(2-chlorophenyl)methyl]-2-(4-fluorophenyl)-.

In an embodiment, the present invention relates to a compound of the present invention as defined herein (i.e. a compound of formula (I), (Ia), (Ib), (Ic) or (Id)) for use in therapy, wherein the compound is not one of the following compounds:
ethyl 4-amino-2-(2-methoxyphenyl)pyrimidine-5-carboxylate;
ethyl 4-amino-2-(o-tolyl)pyrimidine-5-carboxylate;
ethyl 4-amino-2-(2-chlorophenyl)pyrimidine-5-carboxylate;
ethyl 4-amino-2-phenylpyrimidine-5-carboxylate;
ethyl 4-amino-2-(4-chlorophenyl)pyrimidine-5-carboxylate; or
ethyl 4-amino-2-(2-hydroxyphenyl)pyrimidine-5-carboxylate.

In an embodiment, the present invention relates to a pharmaceutical composition comprising a compound of the present invention (i.e. a compound of formula (I), (Ia), (Ib), (Ic) or (Id)) in admixture with a one or more pharmaceutically acceptable excipients, wherein the compound is not one of the following compounds:
ethyl 4-amino-2-(2-methoxyphenyl)pyrimidine-5-carboxylate;
ethyl 4-amino-2-(o-tolyl)pyrimidine-5-carboxylate;
ethyl 4-amino-2-(2-chlorophenyl)pyrimidine-5-carboxylate;
ethyl 4-amino-2-phenylpyrimidine-5-carboxylate;
ethyl 4-amino-2-(4-chlorophenyl)pyrimidine-5-carboxylate; or
ethyl 4-amino-2-(2-hydroxyphenyl)pyrimidine-5-carboxylate.

Compounds of formula (I) may be prepared as described below and the methods for their preparation form a further aspect of the invention.

Compounds of general formula (Ia) as defined above may be prepared from compounds of general formula (II):

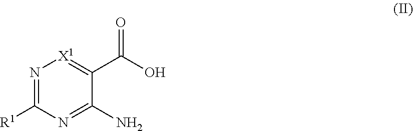

wherein X¹ and R¹ are as defined for general formula (I) (Ia) or (Ib); by reaction with a compound of general formula (III):

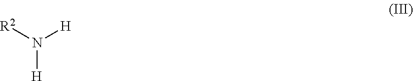

wherein R² is as defined for general formula (I), (Ia), (Ib), (Ic) or (Id).

In some cases, a coupling agent may be used for this reaction. Suitable coupling agents include (1-[bis(dimethylamino)methylene]-1H-1,2,3-triazolo[4,5-b]pyridinium 3-oxid hexafluorophosphate) (HATU), O-(benzotriazol-1-yl)-N,N,N′,N′-tetramethyluronium tetrafluoroborate (TBTU), propylphosphonic anhydride (T3P®), dicyclohexylcarbodiimide (DCI), diisopropylcarbodiimide (DIC) and carbonyl diimidazole (CDI). Other coupling agents are well known in the art.

Compounds of general formula (II) as defined above may be prepared from compounds of general formula (Ib) as defined above an in which R² is R^{2b} (i.e. C₋₆ alkyl) by hydrolysis, suitably base hydrolysis, for example using an alkali metal hydroxide, for example lithium, sodium or potassium hydroxide. Lithium hydroxide is particularly suitable.

There are several ways in which compounds of general formula (Ib) can be prepared. In a first method, a compound of general formula (Ib) in which X¹ is N or C—R³ can be prepared from a compound of general formula (IV):

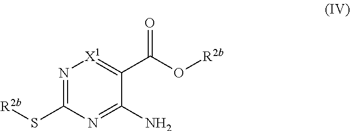

wherein each R^{2b} is independently C₁₋₆ alkyl;
X¹ is N or CR³, wherein R³ is as defined for general formula (I);
by reaction with a compound of general formula (V):

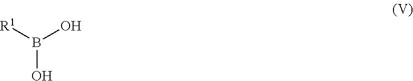

wherein R¹ is as defined for general formula (I).

Suitably, the reaction is carried out in the presence of a palladium catalyst and is a Liebeskind-Srogl type coupling reaction.

Compounds of general formula (IV) in which X¹ is N may be prepared from compounds of general formula (VI):

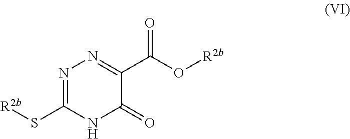

wherein R^{2b} is as defined for general formula (IV);
by reaction with thionyl chloride followed by ammonium hydroxide as described in WO 2013/192049.

Compounds of general formula (VI) may be prepared by reacting a compound of general formula (VII):

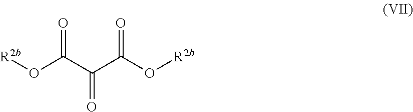

wherein R^{2b} is as defined for general formula (IV);
with aminothiourea, which has the formula:

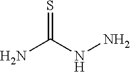

The reaction may be carried out according to the procedure described in JP2009007341. Thus, the reagents may be heated in a suitable solvent, typically an alcoholic solvent, following which an alkali metal alkoxide is added to the mixture.

Typically, in this reaction, R^{2b} is ethyl, the alkali metal alkoxide is sodium ethoxide and the solvent is ethanol.

Compounds of general formula (VII) are known and several are commercially available. One example of a commercially available compound of general formula (VII) is diethyl 2-oxopropanedioate, in which R^{2b} is ethyl.

Compounds of general formula (IV) in which X¹ is C—Cl may be prepared by reacting a compound of general formula (VIII):

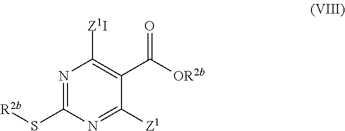

wherein R^{2b} is as defined for general formula (IV) and each Z¹ is independently halo, for example chloro;
with aqueous ammonium hydroxide.

Suitably the reaction is carried out at room temperature (about 15 to 25° C.) in a solvent such as tetrahydrofuran.

Compounds of general formula (VIII) may be prepared by reacting a compound of general formula (IX):

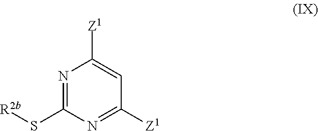

wherein R^{2b} is as defined for general formula (IV) and Z¹ is as defined for general formula (VIII) and is suitably chloro;
with a compound of general formula (X):

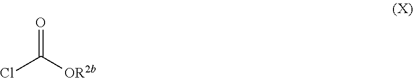

wherein R^{2b} is as defined for general formula (IV).

The procedure may be carried out as described in US 2010/0249110. Suitably, therefore, the compound of formula (IX) is treated with a strong base such as lithium diisopropylamide (LDA) under an inert atmosphere such as nitrogen and at reduced temperature, for example −78° C. The compound of formula (X) is subsequently added.

Compounds of general formulae (IX) and (X) are known and many are commercially available, for example the compounds in which R^{2b} is ethyl. In this case, the compound of general formula (IX) is 4,6-dichloro-2-methylsulfanyl-pyrimidine and the compound of formula (X) is ethyl chloroformate.

Compounds of general formula (IV) in which X¹ is C—R³, where R³ is halo may be converted to compounds of general formula (IV) in which X¹ is CR³ and R³ is other than halo.

Examples of such reactions are given in Examples 18 and 19 below and other such reactions will be familiar to those of skill in the art.

A compound of general formula (Ib) in which X¹ is CH may be synthesised by reacting a compound of general formula (V) as defined above with a compound of general formula (XV):

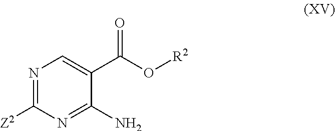

wherein R^{2b} is as defined for general formula (IV) and Z² is halo, suitably chloro.

The reaction may be carried out in the presence of a palladium catalyst as described above for the reaction of the compound of general formula (V) with the compound of general formula (IV). Alternatively the boronic acid (V) can be replaced by a boronate ester analogue or stannane derivative.

Compounds of general formula (XV) are known in the art. Compounds where Z² is chloro are commercially available and other compounds may be synthesised by methods known to those of skill in the art. Suitably, in the compound of general formula (XV), R^{2b} is ethyl.

As described above, the compound of general formula (XV) may be reacted with the compound of general formula (V) to give a compound of general formula (Ib) in which R^{2b} is C₁₋₆ alkyl. This may be hydrolysed to give a compound of general formula (II). The compound of general formula (II) may then undergo a coupling reaction with a compound of general formula (III) to give a compound of general formula (Ia) as illustrated in the scheme below.

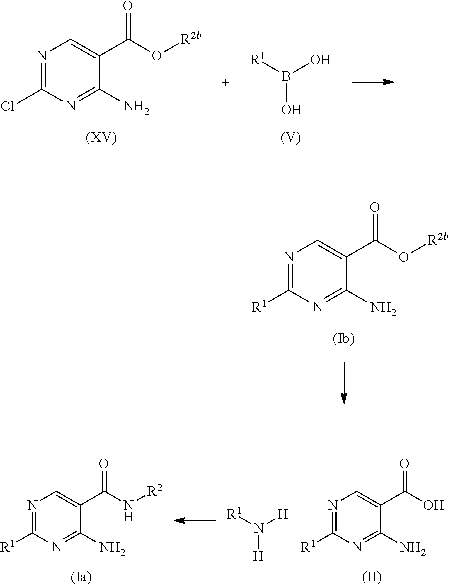

However, compounds of general formula (Ia) may also be prepared from compounds of general formula (XV) by carrying out similar steps in a different order.

Thus, for example, a compound of general formula (XV) may be hydrolysed to give 4-amino-2-chloro-pyrimidine-5-carboxylicacid:

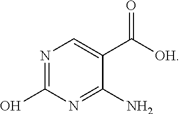

Suitably, the hydrolysis is carried out using a base, particularly an alkali metal hydroxide, for example lithium, sodium or potassium hydroxide, with lithium hydroxide being particularly suitable. The reaction may be conducted at room temperature (about 15 to 25° C.) in a mixed solvent, typically THF and water.

4-Amino-2-chloro-pyrimidine-5-carboxylic acid may be reacted with an amine of general formula (III) as defined above to give a compound of general formula (XVI):

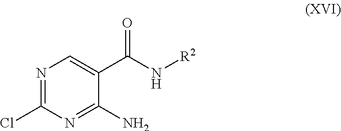

wherein R² is as defined for general formula (I).

The reaction is suitably carried out in the presence of a coupling agent as described above for the reaction between the compound of general formula (Ib) in which R² is H and the compound of general formula (III).

The compound of general formula (XVI) may then be reacted with a compound of general formula (V) as defined above to give a compound of general formula (Ia) as defined above. The reaction is suitably carried out in the presence of a palladium catalyst as described above for the reaction between the compounds of general formulae (IV) and (V).

This is illustrated in the scheme below:

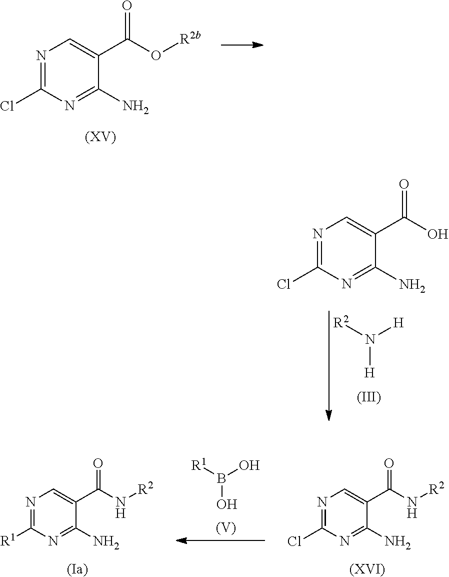

and an example of this type of synthesis is provided in Example 12.

An alternative method for the synthesis of a compound of general formula (Ib) in which R² is R^{2b} and X¹ is N is by reacting a compound of general formula (XX):

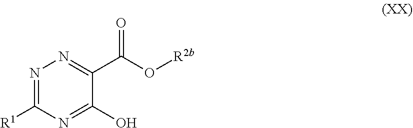

wherein R¹ is as defined for general formula (I) and R^{2b} is as defined for general formula (IV);
with ammonium hydroxide.

Suitably, the compound of general formula (XX) is first activated, for example using phosphoryl chloride.

The product of general formula (Ib) is formed as a mixture with a product similar to a compound of general formula (Ia) in which X¹-R² is NH₂.

Compounds of general formula (XX) may be formed from the reaction between a compound of general formula (VII) as defined above and a compound of general formula (XXII):

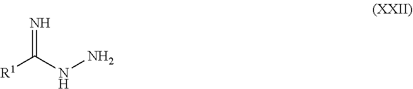

wherein R¹ is as defined for general formula (I).

The reaction is suitably carried out in an organic solvent such as toluene and at elevated temperature, for example the reflux temperature of the solvent.

The product of general formula (XX) may be obtained in admixture with a compound of the formula:

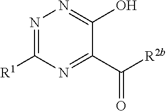

wherein R¹ and R^{2b} are as defined above.

Compounds of general formula (XXII) may be synthesised by reacting a compound of general formula (XXII):

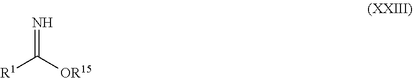

wherein R¹ is as defined above and R¹⁵ is C₁₋₆ alkyl;
with hydrazine hydrate. Typically, the reaction is carried out at room temperature (about 15 to 25° C.) in an alcoholic solvent such as ethanol.

A compound of general formula (XXIII) may be prepared by reacting a compound of general formula (XXIV):

R¹—CN (XXIV)

wherein R¹ is as defined for general formula (I);
with a compound of general formula R¹⁵OH (XXV) and acetyl chloride:
wherein R¹⁵ is as defined for general formula (XXIII).

The reaction may be carried out according to the procedure of Ningning L, Zhengkai C, Yue L, Zhanxiang L, Yuhong Z, (Org. Lett., 2017, 19 (10), pp 2588-2591) as described below in Example 6.

A compound of general formula (II) in which X¹ is CH may be prepared from a compound of general formula (XXX):

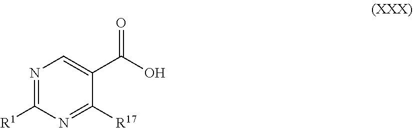

wherein R¹ is as defined for general formula (I) and R¹⁷ is halo, especially chloro;
by reaction with aqueous ammonium hydroxide.

Suitably, the reaction is carried out in an aqueous solvent, typically methanol and water and at elevated temperature, for example about 100-140° C., typically about 120° C. with microwave irradiation.

A compound of general formula (XXX) may also be converted to a compound of general formula (Ia) in which X¹ is CH via a compound of general formula (XXXI):

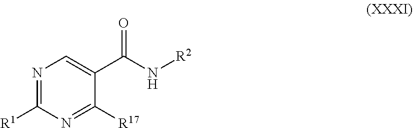

wherein R¹ and R² are as defined for general formula (I) and R¹⁷ is as defined for general formula (XXX).

The compound of general formula (XXXI) may be obtained by conversion of the compound of general formula (XXX) to its acid chloride, for example by reaction with oxalyl chloride, followed by reaction of the acid chloride with a compound of general formula (III). Suitable reaction conditions for these reactions are familiar to those of skill in the art.

The compound of general formula (XXXI) may be converted to a compound of general formula (Ia) by reaction with sodium azide followed by reduction with triphenylphosphine.

The reaction with sodium azide typically takes place in an organic solvent such as N,N-dimethylformamide at a temperature of about 30-50° C. The temperature may then be reduced to room temperature (about 15-25° C.) for the reaction with triphenylphosphine.

A compound of general formula (XXX) may be prepared by hydrolysis of a compound of general formula (XXXII):

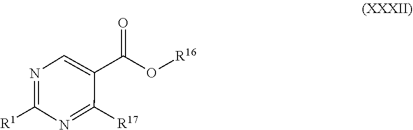

wherein R¹ is as defined for general formula (I), R¹⁷ is as defined for general formula (XXX) and R¹⁶ is C₁₋₆ alkyl.

Typically, the hydrolysis is alkaline hydrolysis, suitably with an alkali metal hydroxide, for example lithium, sodium or potassium hydroxide, most suitably lithium hydroxide.

A compound of general formula (XXXII) may be prepared from a compound of general formula (XXXII):

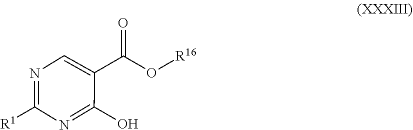

wherein R¹ is as defined for general formula (I) and R¹⁶ is as defined for general formula (XXXIII);
by reaction with a halogenating agent. When R¹⁷ is chloro, a suitable halogenating agent is phosphoryl chloride. In this case, the compound of general formula (XXXIII) and the phosphoryl chloride may be mixed with cooling, for example to 0° C., following which the reaction mixture may be heated to 100-120° C.

A compound of general formula (XXXIII) may be prepared by reacting a compound of general formula (XXXIV):

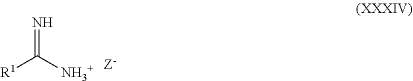

wherein R¹ is as defined for general formula (I) and Z⁻ is a suitable counter ion, particularly a halide such as chloride;
with a compound of general formula (XXXV):

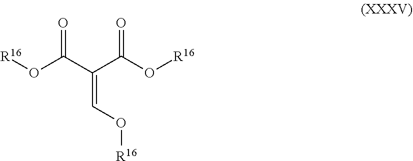

wherein R¹⁶ is as defined for general formula (XXXII);
according to the procedure set out in WO 2006/097220.

Compounds of general formulae (XXXIV) and (XXXV) are known and are commercially available or may be prepared by methods known to those of skill in the art.

Compounds of general formulae (I), (la), (Ib), (Ic) or (Id), in which X¹ is CR³ and R³ is O(C₁₋₆ alkyl), optionally substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl; may be prepared by reacting a compound of Formula (Ya):

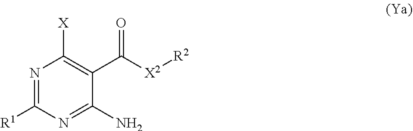

wherein R¹, R², and X² are as defined above for general formula (I), (Ia), (Ib), (Ic) or (Id), and X is a leaving group, for example halo (e.g. chloro, fluoro, bromo);
with a compound of general formula (Za);

R₃ₐ—OH (Za)

wherein R₃ₐ is (C₁₋₆ alkyl), optionally substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl.

If either R⁹ or R¹⁰ are H in R₃ₐ above, then they may be protected with a suitable protecting group. Examples of suitable protecting groups for amines are well known in the art. If a protecting group is present, then the process further comprises a step of removing any protecting groups present to provide the compound of formula I defined herein.

The reaction may include dissolving the compound of Formula (Za) in a suitable solvent and adding the compound of formula (Ya) to the solvent, or vice versa. In an embodiment, the solvent is a suspension of sodium hydride in anhydrous DMF or THF. In another embodiment, the compound of Formula (Za) is used as the solvent.

Compounds of general formulae (I), (Ia), (Ib), (Ic) or (Id), in which X¹ is CR³ and R³ is —NH(C₁₋₆ alkyl), optionally substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl; may be prepared by reacting a compound of Formula (Ya):

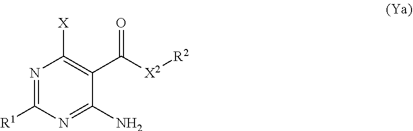

wherein R¹, R², and X² are as defined above for general formula (I), (Ia), (Ib), (Ic) or (Id), and X_{b} is a leaving group, for example halo (e.g. chloro, fluoro, bromo);
with a compound of general formula (Zb);

R₃ₐ—NH₂ (Zb)
wherein R₃ₐ is (C₁₋₆ alkyl), optionally substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl, wherein R⁴ is H or methyl and each R⁹ and R¹⁰ is independently selected from H, C₁₋₆ alkyl and C₁₋₆ haloalkyl; and
wherein any carbocyclyl, heterocyclyl, aryl and heteroaryl groups are optionally substituted with one or more substituents selected from halo, C₁₋₆ alkyl, C₁₋₆ haloalkyl and —NR¹¹R¹²; wherein each R¹¹ and R¹² is independently selected from H, C₁₋₆ alkyl and C₁₋₆ haloalkyl.

If either R⁹ or R¹⁰ are H in R₃ₐ above, then they may be protected with a suitable protecting group. Examples of suitable protecting groups for amines are well known in the art. If a protecting group is present, then the process further comprises a step of removing any protecting groups present to provide the compound of formula I defined herein.

The reaction may include dissolving the compound of Formula (Ya) in a suitable solvent and adding the compound of formula (Zb) to the solvent, or vice versa. In an embodiment, the solvent is THF or DMF.

Compounds of general formulae (I), (Ia), (Ib), (Ic) or (Id) in which R¹ is as defined herein may be prepared by reacting a compound of Formula (Yb):

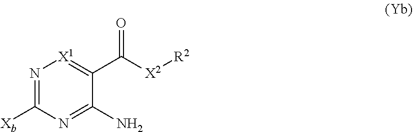

wherein R², X¹ and X² are as defined above for general formula (I), (Ia), (Ib), (Ic) or (Id), and X_{b} is a leaving group, for example —S(C₁₋₆ alkyl), halo (e.g. chloro, fluoro, bromo); with a compound of general formula (W);

R¹-L (W)
wherein R¹ is as defined herein and L is a displaceable group, e.g. H, B(OH)₂. Preferably, R¹ is furanyl, oxazolyl, thiazolyl or phenyl optionally substituted with fluoro or cyano. The reaction may include dissolving the compound of Formula (Yb) in a suitable solvent and adding the compound of formula (W) to the solvent, or vice versa. In an embodiment, the solvent is THF or DMF.

Compounds of general formulae (I), (Ia), (Ib), (Ic) or (Id) in which X² is NH and R² is as defined herein, may be prepared by reacting a compound of Formula (Yc):

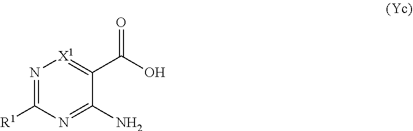

Wherein R¹ and X¹ are as defined above for general formula (I), (Ia), (Ib), (Ic) or (Id);
with a compound according to Formula Q:

H₂N—R² (Q)
wherein R² is as defined herein. The reaction may include dissolving the compound of Formula (Yc) in a suitable solvent and adding the compound of formula (Q) to the solvent, or vice versa. In an embodiment, the solvent is THF or DMF.

An example reaction scheme to make a compound according to general formulae (I), (Ia), (Ib), (Ic) or (Id) defined herein is shown in Scheme 1 below.

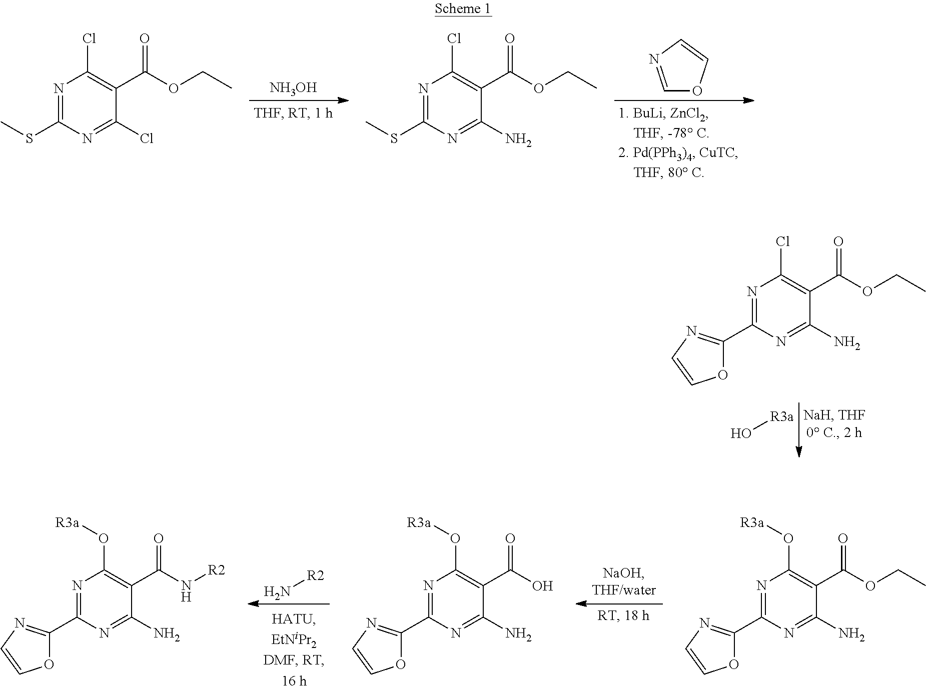

An alternative reaction scheme to make a compound according to general formulae (I), (Ia), (Ib), (Ic) or (Id) defined herein is shown in Scheme 2 below.

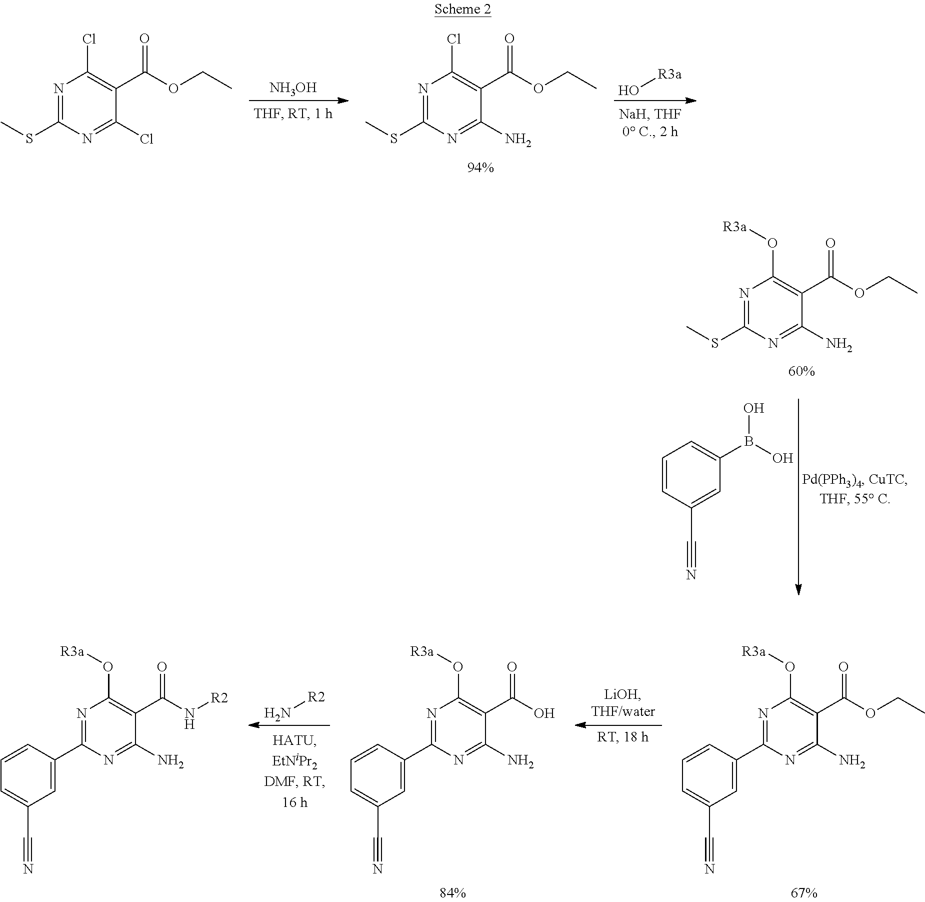

As discussed above, the compounds of general formula (I) are potent and selective adenosine A2a antagonists and are therefore useful in the treatment of cancer.

Therefore, in a further aspect of the invention there is provided a compound of general formula (I) for use in medicine.

More specifically, there is provided a compound of general formula (I) for use in the treatment of cancer, particularly solid tumours, for example non-small cell lung cancer, head and neck squamous cancer and urothelial cancer.

There is also provided the use of a compound of general formula (I) in the manufacture of a medicament for the treatment of cancer, particularly solid tumours, for example non-small cell lung cancer, head and neck squamous cancer and urothelial cancer.

The invention further provides a method for the treatment of cancer, particularly solid tumours, for example non-small cell lung cancer, head and neck squamous cancer and urothelial cancer, the method comprising administering to a patient in need of such treatment an effective amount of a compound of general formula (I).

The patient to be treated is suitably a mammal and more suitably a human.

The compounds of general formula (I) may be administered in a pharmaceutical composition and therefore in a further aspect of the invention there is provided a pharmaceutical composition comprising a compound of general formula (I) and a pharmaceutically acceptable excipient. Other pharmacologically active materials may also be present, as considered appropriate or advisable for the disease or condition being treated or prevented.

The carrier, or, if more than one be present, each of the carriers, must be acceptable in the sense of being compatible with the other ingredients of the formulation and not deleterious to the recipient.

The formulations include those suitable for oral, rectal, nasal, bronchial (inhaled), topical (including dermal, transdermal, eye drops, buccal and sublingual), vaginal or parenteral (including subcutaneous, intramuscular, intravenous and intradermal) administration and may be prepared by any methods well known in the art of pharmacy.

The route of administration will depend upon the condition to be treated but preferred compositions are formulated for oral administration.

The composition may be prepared by bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) in conjunction or association with a pharmaceutically acceptable carrier or vehicle.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Some formulations may comprise a mucoadherent, for example a mucopolysaccharide such as sodium hyaluronate. Such compositions may be formulated as, for example, liquids, liquid syrups, soft gels, liquid gels, flowable gels or aqueous suspensions and may, in addition to the active agent and the mucoadherent, also contain one or more additional excipients as set out above. Liquid formulations will usually also contain a liquid carrier, which may be a solvent or suspending agent, for example water or saline solution and may also contain a substance to increase their viscosity, for example sodium carboxymethylcellulose, sorbitol or dextran.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For topical application to the skin, compounds of general formula (I) may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

The composition defined above may be used for the treatment of the respiratory tract by nasal, bronchial or buccal administration of, for example, aerosols or sprays which can disperse the pharmacological active ingredient in the form of a powder or in the form of drops of a solution or suspension. Pharmaceutical compositions with powder-dispersing properties (e.g., dry powder inhalers) usually contain, in addition to the active ingredient, a suitable carrier such lactose and, if desired, adjuncts, such as surfactants and/or diluents and/or flow aids and/or lubricants. Pharmaceutical compositions with powder-dispersing properties (e.g., metered dose inhalers) usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution (e.g., either solution for nebulisation or metered dose inhalers) contain, in addition to this, a suitable propellant, and furthermore, if necessary, an additional solvent and/or a stabiliser. Instead of the propellant, compressed air can also be used, it being possible for this to be produced as required by means of a suitable compression and expansion device.

The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the compound of general formula (I) will be about 0.0001 to less than 4.0% (w/w).

Preferably, for topical ocular administration, the compositions administered according to general formula (I) will be formulated as solutions, suspensions, emulsions and other dosage forms. Aqueous solutions are generally preferred, based on ease of formulation, as well as a patient's ability to administer such compositions easily by means of instilling one to two drops of the solutions in the affected eyes. However, the compositions may also be suspensions, viscous or semi-viscous gels, or other types of solid or semi-solid compositions. Suspensions may be preferred for compounds that are sparingly soluble in water.

An alternative for administration to the eye is intravitreal injection of a solution or suspension of the compound of general formula (I). In addition, the compound of general formula (I) may also be introduced by means of ocular implants or inserts.

The compositions administered according to general formula (I) may also include various other ingredients, including, but not limited to, tonicity agents, buffers, surfactants, stabilizing polymer, preservatives, co-solvents and viscosity building agents. Suitable pharmaceutical compositions of general formula (I) include a compound of the invention formulated with a tonicity agent and a buffer. The pharmaceutical compositions of general formula (I) may further optionally include a surfactant and/or a palliative agent and/or a stabilizing polymer.

Parenteral formulations will generally be sterile.

The medical practitioner, or other skilled person, will be able to determine a suitable dosage for the compound of general formula (I), and hence the amount of the compound of the invention that should be included in any particular pharmaceutical formulation (whether in unit dosage form or otherwise).

Compounds of general formula (I) may be used in combination with one or more other active agents which are useful in the treatment or prophylaxis of cancer.

An additional active agent of this type may be included in the pharmaceutical composition described above but alternatively it may be administered separately, either at the same time as the compound of general formula (I) or at an earlier or later time.

Therefore, in a further aspect of the present invention there is provided a product comprising a compound of general formula (I) and an additional agent useful in the treatment or prevention of cancer as a combined preparation for simultaneous, sequential or separate use in the treatment of cancer, in particular solid tumours for example non-small cell lung cancer, head and neck squamous cancer and urothelial cancer.

There is also provided a compound of general formula (I) in combination with an additional agent useful in the treatment of cancer as a combined preparation for simultaneous, sequential or separate use in the treatment of treatment of cancer, in particular solid tumours for example non-small cell lung cancer, head and neck squamous cancer and urothelial cancer.

Suitable additional active agents which may be included in a pharmaceutical composition or a combined preparation with the compounds of general formula (I) include:

other forms of cancer immunotherapy and anti-cancer chemotherapeutic agents;

A2b antagonists;

anti-PD-1 and PDL-1 antibodies including, but not limited to, pembrolizumab, nivolumab, durvalumab, avelumab and atezolizumab; and

anti-CTLA4 antibodies including, but not limited to, ipilimumab.

The A2a antagonists of general formula (I) can also be used in combination with cell-based immunotherapy and cancer vaccines that include, but are not limited to CAR T cell therapy.

Examples of anti-cancer chemotherapeutic agents include, but are not limited to, MEK (e.g. MEK1, MEK2, or MEK1 and MEK2) inhibitors (e.g. XL518, CI-1040, PD035901, selumetinib/AZD6244, GSK1 120212/trametinib, GDC-0973, ARRY-162, ARRY-300, AZD8330, PD0325901, U0126, PD98059, TAK-733, PD3 18088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, arboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-I, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fiudarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatinA; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzyl guanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-basedimmune modulator; protein kinase C inhibitor; protein kinase C inhibitors, microalgal; proteintyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAPinhibitor; retelliptine demethylated; rhenium Re 186 etidronate; rhizoxin; ribozymes; RII retinamide; rogletimide; rohitukine; romurtide; roquinimex; rubiginone E1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stemcell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin 2 (including recombinant interleukin 2, or rIL.sub.2), interferon alfa-2a; interferon alfa-2b; interferon alfa-n1; interferon alfa-n3; interferon beta-la; interferon gamma-b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-3 10705), 21-hydroxyepothilone D (i.e. Desoxyepothilone F and dEpoF), 26-fluoroepothilone, Auristatin PE (i.e. NSC-654663), Soblidotin (i.e. TZT-1027), Vincristine sulfate, Cryptophycin 52 (i.e. LY-355703), Vitilevuamide, Tubulysin A, Canadensol, Centaureidin (i.e. NSC-106969), Oncocidin A1 (i.e. BTO-956 and DF E), Fijianolide B, Laulimalide, Narcosine (also known as NSC-5366), Nascapine, Hemiasterlin, Vanadocene acetylacetonate, Monsatrol, Inanocine (i.e. NSC-698666), Eleutherobins (such as Desmethyleleutherobin, Desaetyleleutherobin, Isoeleutherobin A, and ZEleutherobin), Caribaeoside, Caribaeolin, Halichondrin B, Diazonamide A, Taccalonolide A, Diozostatin, (−)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-F£ER2, anti-CD52, anti-ULA-DR, and anti-VEGF monoclonal antibodies), immunotoxins (e.g., anti-CD33 monoclonal

antibody-calicheamicin conjugate, anti-CD22 monoclonal antibody-pseudomonas exotoxin conjugate, etc.), radioimmunotherapy (e.g., anti-CD20 monoclonal antibody conjugated to In, 0Y, or I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, or the like.

The invention will now be further described with reference to the examples.

### EXAMPLES

General Conditions:

Mass spectra were run on LC-MS systems using electrospray ionization These were run using either a Waters Acquity H-Class UPLC with PDA and QDa mass detection, Acquity UPLC (binary pump/PDA detector)+ZQ Mass Spectrometer or Acquity i-Class (quarternary pump/PDA detector)+Quattro Micro Mass Spectrometer. [M+H]+ refers to mono-isotopic molecular weights.

NMR spectra were run on either a Bruker Ultrashield 500 MHz NMR spectrometer, Bruker Avance III HD 400 MHz NMR spectrometer or a Bruker Avance DPX 300 MHz NMR spectrometer. Spectra were recorded at 298K and were referenced using the solvent peak.

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, and NMR. Abbreviations used are those conventional in the art. If not defined, the terms have their generally accepted meanings.

### ABBREVIATION

App apparent
Br broad
D doublet
dd doublet of doublets
DCM dichloromethane
DIEA diethylisopropylamine
DIPEA diisopropylethylamine
DMF N,N-dimethylformamide
EtOAc ethyl acetate
h hour(s)
HATU 2-(7-Aza-1H-benzotriazole-1-yl)-1,1,3,3-tetramethyluronium hexafluorophosphate
HPLC high pressure liquid chromatography
LC-MS liquid chromatography and mass spectrometry
MeOH methanol
MS mass spectrometry
m multiplet
min minute(s)
mL milliliter(s)
m/z mass to charge ratio
NMR nuclear magnetic resonance
Pd(amphos)₂C₂ Bis(triphenylphosphine)palladium(II) dichloride
ppm parts per million
PS polymer supported
rac- racemate
rt retention time
s singlet
t triplet
TEA triethylamine
TFA trifluoroacetic acid
THF tetrahydrofuran

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

It should be understood that the organic compounds according to the preferred embodiments may exhibit the phenomenon of tautomerism. As the chemical structures within this specification can only represent one of the possible tautomeric forms, it should be understood that the preferred embodiments encompass any tautomeric form of the drawn structure.

If not indicated otherwise, the analytical HPLC conditions are as follows:

Method 3A

Column: Acquity UPLC CSH C18 2.1×50 mm 1.7 μm

Column Temp 50° C.

Eluents: A: H20, B: acetonitrile, 0.1% formic acid

Flow Rate: 1 mL/min

Gradient: 0.2-2.5 mins 2-98% B, 2.5-3.0 mins 98% B

Method 3B

Column: Acquity UPLC BEH C18 2.1×50 mm 1.7 μm

Column Temp 50° C.

Eluents: A: H20, B: acetonitrile, 0.1% ammonia

Flow Rate: 1 mL/min

Gradient: 0.2-2.5 mins 2-98% B, 2.5-3.0 mins 98% B

Method 8A

Column: Acquity UPLC CSH C18 2.1×100 mm 1.7 μm

Column Temp 50° C.

Eluents: A: H20, B: acetonitrile, 0.1% formic acid

Flow Rate: 0.6 mL/min

Gradient: 0.5-6.5 mins 2-98% B 6.5-7.5 mins 98% B

Method 8B

Column: Acquity UPLC BEH C18 2.1×100 mm 1.7 μm

Column Temp 50° C.

Eluents: A: H20, B: acetonitrile, 0.1% ammonia

Flow Rate: 0.6 mL/min

Gradient: 0.5-6.5 mins 2-98% B 6.5-7.5 mins 98% B

Method CP-QC1_acidic

Column Acquity UPLC® HSS C18 1.8 μm MVK 2.1×100 mm

Column Temp 40° C.

Eluents A: H2O, B:MeCN, both containing 0.1% formic acid

Flow Rate 0.5 mL/min

Gradient 5%-100% B over 2.3 min

Method CP-QC5_basic

Column Acquity UPLC® BEH C18 1.7 μm MV Kit 2.1×100 mm

Column Temp 40° C.

Eluents A: H2O, B:MeCN, both 10 mM ammonium bicarbonate

Flow Rate 0.5 mL/min

Gradient 5%-100% B over 2.3 min

Method ARG-QC1_acidic

Column Acquity UPLC BEH C18 1.7 μm, 100×2.1 mm

Column Temp 40° C.

Eluents A: H2O, B:MeCN, both containing 0.1% formic acid

Flow Rate 0.4 mL/min

Gradient 0.40 min 5% B, 5% to 95% B in 6.60 min, 1.00 min 95% B

Method ARG-QC2_acidic

Column Acquity UPLC BEH C18 1.7 μm, 100×2.1 mm

Column Temp 40° C.

Eluents A: H2O, B:MeCN, both containing 0.1% formic acid

Flow Rate 0.4 mL/min

Gradient 0.40 min 5% B, 5% to 95% B in 6.60 min, 1.00 min 95% B

Both ARG-QC1_acidic and ARG-QC2_acidic are the same method, but run on different machines

Method ARG-QC5_basic

Column Acquity UPLC BEH C18 1.7 μm, 100×2.1 mm

Column Temp 40° C.

Eluents A: H2O, B:MeCN, both containing 0.1% ammonia

Flow Rate 0.4 mL/min

Gradient 0.40 min 5% B, 5% to 95% B in 6.60 min, 1.00 min 95% B

Method Acidic

Column Acquity UPLC BEH C18 1.7 μm, 50×2.1 mm

Column Temp 40° C.

Eluents A: H2O, B:MeCN, both containing 0.1% formic acid

Flow Rate 0.4 mL/min

Gradient 3% to 97% B in 1.50 min, 0.40 min 97% B

### PREPARATION OF EXAMPLES

### Example 1

### Ethyl 5-amino-3-(2-furyl)-1,2,4-triazine-6-carboxylate

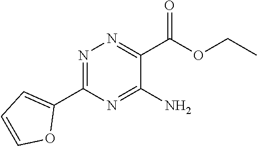 [SMILES: CCOC(=O)c1nnc(-c2ccco2)nc1N]

### Step 1: Ethyl 3-methylsulfanyl-5-oxo-4H-1,2,4-triazine-6-carboxylate

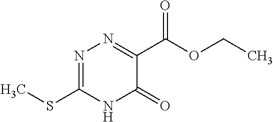

The titled compound was prepared from aminothiourea and diethyl 2-oxopropanedioate according to the procedure detailed by Kokubo, S and Miyazaki, H (Jpn. Kokai Tokkyo Koho, 2009007341—English translation provided in Scifinder).

A mixture of aminothiourea (1 g, 10.97 mmol) and diethyl 2-oxopropanedioate (1.76 mL, 11.52 mmol) in EtOH (20 mL) was heated to 70° C. for 5 h after which time the solution was cooled to room temperature. Sodium ethoxide (21 wt % EtOH) (4.1 mL, 10.97 mmol) was added and the mixture heated at 70° C. for 2 h.

After cooling to 0° C., a solution of potassium carbonate (1.52 g, 10.97 mmol) in water (7 mL) was added followed by methyl iodide (956 μL, 15.36 mmol). The resulting mixture was stirred for 4 h after which time 1M citric acid was added dropwise and the mixture was concentrated in vacuo. The crude mixture was partitioned between EtOAc (100 mL) and H₂O (100 mL), the organic portion separated and the aqueous further extracted with EtOAc (2×50 mL). The combined organic extracts were washed with brine, dried over MgSO₄ and the solvent removed in vacuo. Purification by column chromatography on silica eluting with 3% MeOH in DCM afforded the titled compound as a pale yellow solid.

LC-MS (Method 3A): Rt 0.98 mins; MS m/z 216.1=[M+H]+

1H NMR (500 MHz, Chloroform-d) δ 4.44 (q, J=7.1 Hz, 2H), 2.58 (s, 3H), 1.39 (t, J=7.1 Hz, 3H).

1H NMR (500 MHz, DMSO-d6) δ 4.29 (q, J=7.1 Hz, 2H), 1.27 (t, J=7.1 Hz, 3H).

### Step 2: Ethyl 5-amino-3-methylsulfanyl-1,2,4-triazine-6-carboxylate

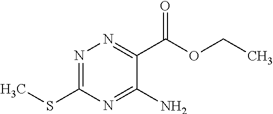

The titled compound was prepared from ethyl 3-methylsulfanyl-5-oxo-4H-1,2,4-triazine-6-carboxylate (step 1) according to the procedure of Jia, Z J; Kane, B; Rose, J; Bauer, Shawn M; Song, Y; Xu, Q; Pandey, A (WO2013/192049 A2 pages 43 and 89).

A mixture of ethyl 3-methylsulfanyl-5-oxo-4H-1,2,4-triazine-6-carboxylate (step 1)(400 mg, 1.86 mmol) in thionyl chloride (2.02 mL, 27.88 mmol) was heated to reflux for 3 h. After cooling to room temperature, the solvent was removed in vacuo and the mixture re-dissolved in toluene and concentrated in vacuo (×3) to afford a viscous yellow oil. The oil was dissolved in 1,4-dioxane (2 mL) and the mixture cooled to 0° C. before NH₄OH (35 wt %) (1.5 mL, 1.86 mmol) was added dropwise.

The mixture was stirred for 30 mins and then diluted with water. The resulting solid was collected by filtration and dried to afford the titled compound as a cream solid.

LC-MS (Method 3B): Rt 1.14 mins; MS m/z 215.2=[M+H]+

1H NMR (500 MHz, Chloroform-d) δ 7.93 (br s, 1H), 5.65 (br s, 1H), 4.48 (q, J=7.1 Hz, 2H), 2.61 (s, 3H), 1.45 (t, J=7.1 Hz, 3H).

### Step 3: Ethyl 5-amino-3-(2-furyl)-1,2,4-triazine-6-carboxylate

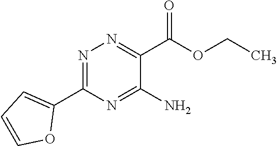 [SMILES: CCOC(=O)c1nnc(-c2ccco2)nc1N]

To a degassed solution of ethyl 5-amino-3-methylsulfanyl-1,2,4-triazine-6-carboxylate (step 2) (160 mg, 0.75 mmol) in THF (4 mL) was added copper(I) 3-methylsalicylate (321 mg, 1.49 mmol), Pd(PPhs)₄ (86 mg, 0.07 mmol) and commercially available 2-furylboronic acid (167 mg, 1.49 mmol). The resulting mixture was heated to reflux for 90 mins and allowed to cool to room temperature. The mixture was partitioned between H₂O (10 mL) and DCM (10 mL), the organic portion separated and the aqueous further extracted with DCM (2×15 mL). The combined organic extracts were dried over MgSO₄ and the solvent removed in vacuo. Purification by column chromatography on silica eluting with a gradient of 1 to 2% MeOH in DCM afforded the titled compound as a yellow solid.

LC-MS (Method 3B): Rt 1.76 mins; MS m/z 235.1=[M+H]+

1H NMR (500 MHz, Chloroform-d) δ 8.05 (s, 1H), 7.70 (d, J=1.8 Hz, 1H), 7.63 (d, J=3.6 Hz, 1H), 6.63 (dd, J=3.6, 1.8 Hz, 1H), 5.99 (s, 1H), 4.51 (q, J=7.1 Hz, 2H), 1.47 (t, J=7.1 Hz, 3H).

### Example 2

### 5-Amino-3-(2-furyl)-N-[(3-methyl-2-pyridyl)methyl]-1,2,4-triazine-6-carboxamide

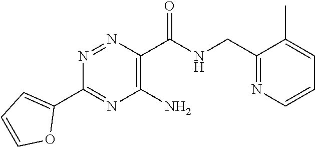 [SMILES: Cc1cccnc1CNC(=O)c1nnc(-c2ccco2)nc1N]

### Step 1:5-Amino-3-(2-furyl)-1,2,4-triazine-6-carboxylic Acid

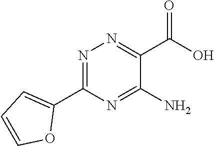 [SMILES: Nc1nc(-c2ccco2)nnc1C(=O)O]

To a solution of ethyl 5-amino-3-(2-furyl)-1,2,4-triazine-6-carboxylate (Example 1) (32 mg, 0.14 mmol) in THF (2 mL) was added a solution of LiOH (4 mg, 0.16 mmol) in water (2 mL) and the mixture was stirred for 1 h at room temperature. A drop of 2M HCl was added to neutralize the mixture and the solvent was removed in vacuo to afford the titled compound as a cream solid. The material was taken through to the next step without further analysis or purification.

LC-MS (Method 3A): Rt 0.80 mins; MS m/z 207.2=[M+H]+

### Step 2: 5-Amino-3-(2-furyl)-N-[(3-methyl-2-pyridyl)methyl]-1,2,4-triazine-6-carboxamide

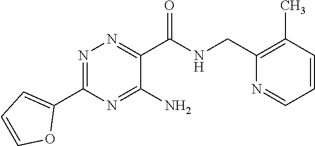 [SMILES: Cc1cccnc1CNC(=O)c1nnc(-c2ccco2)nc1N]

To a solution of 5-amino-3-(2-furyl)-1,2,4-triazine-6-carboxylic acid (step 1) (28 mg, 0.14 mmol) in DMF (1 mL) was added (3-methyl-2-pyridyl)methanamine (24 μL, 0.2 mmol), HATU (103 mg, 0.27 mmol) and DIPEA (118 μL, 0.68 mmol). The mixture was stirred at room temperature for 1 h and the resulting mixture partitioned between EtOAc (10 mL) and H₂O (10 mL). The organic portion was separated and the aqueous further extracted with EtOAc (2×10 mL). The combined organic extracts were washed with H₂O (3×10 mL), brine, dried over MgSO₄ and the solvent removed in vacuo. Purification by column chromatography on silica eluting with 70% EtOAc in petrol afforded the titled compound as a cream solid.

LC-MS (Method 8B): 3.63 mins; MS 311.1=[M+H]+

1H NMR (500 MHz, Chloroform-d) δ 9.65 (br t, J=4.4 Hz, 1H), 8.79 (br s, 1H), 8.44 (dd, J=4.9, 1.7 Hz, 1H), 7.69 (dd, J=1.8, 0.8 Hz, 1H), 7.54 (dd, J=3.5, 0.8 Hz, 1H), 7.52-7.49 (m, 1H), 7.17 (dd, J=7.6, 4.9 Hz, 1H), 6.63 (dd, J=3.5, 1.8 Hz, 1H), 5.84 (br s, 1H), 4.71 (d, J=4.4 Hz, 2H), 2.35 (s, 3H).

### Example 3

### 4-Amino-2-(2-furyl)-N-[(3-methyl-2-pyridyl)methyl]pyrimidine-5-carboxamide

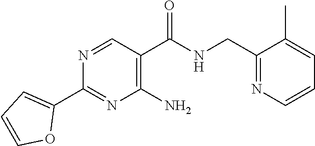 [SMILES: Cc1cccnc1CNC(=O)c1cnc(-c2ccco2)nc1N]

### Step 1: Ethyl 4-amino-2-(2-furyl)pyrimidine-5-carboxylate

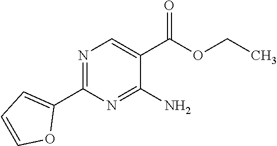 [SMILES: CCOC(=O)c1cnc(-c2ccco2)nc1N]

A vial was charged with commercially available ethyl 4-amino-2-chloro-pyrimidine-5-carboxylate (1 g, 4.96 mmol), potassium carbonate (1.37 g, 9.92 mmol), 1,4-dioxane (12 mL) and water (2 mL). The resulting mixture was stirred vigorously until a cloudy solution was obtained and then placed under an atmosphere of nitrogen. Pd(amphos)₂C2 (176 mg, 0.25 mmol) was added followed by 2-furylboronic acid (833 mg, 7.44 mmol), the vial sealed, the contents evacuated and backfilled with nitrogen (3× cycles) and the mixture heated using microwave radiation at 100° C. for 20.5 hours. More potassium carbonate (1028 mg, 7.44 mmol), Pd(amphos)₂C2 (88 mg, 0.12 mmol) and 2-furylboronic acid (555 mg, 4.96 mmol) were added and stirring continued at 100° C. for a further 5 h. The mixture was allowed to cool, diluted with DCM (100 mL) and extracted with water (100 mL). The aqueous layer was further extracted with DCM (100 mL) and EtOAc (200 mL). The combined organic extracts were dried over MgSO₄ and the solvent removed in vacuo. Purification by column chromatography on silica eluting with a gradient of 0.5 to 4% MeOH in DCM afforded the titled compound as a pale yellow solid.

LC-MS (Method 3B): Rt 1.49 mins; MS m/z 234.1=[M+H]+

1H NMR (500 MHz, Chloroform-d) δ 8.92 (s, 1H), 7.93 (s, 1H), 7.63 (dd, J=1.7, 0.9 Hz, 1H), 7.35 (dd, J=3.5, 0.8 Hz, 1H), 6.57 (dd, J=3.5, 1.7 Hz, 1H), 5.81 (s, 1H), 4.37 (q, J=7.1 Hz, 2H), 1.40 (t, J=7.1 Hz, 3H).

### Step 2: 4-Amino-2-(2-furyl)pyrimidine-5-carboxylic Acid

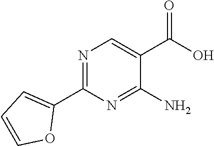 [SMILES: Nc1nc(-c2ccco2)ncc1C(=O)O]

LiOH (21 mg, 0.86 mmol) was added to a suspension of ethyl 4-amino-2-(2-furyl)pyrimidine-5-carboxylate (step 1) (100 mg, 0.43 mmol) in THF (2 mL) and water (2 mL) and the mixture stirred at room temperature for 1 h. The resulting mixture was acidified with 2M HCl, and the solvent removed in vacuo to afford the titled compound as a cream solid which was used in the next step without further purification.

LC-MS (Method 3A): Rt 0.91 mins; MS m/z 206.1=[M+H]+

### Step 3: 4-Amino-2-(2-furyl)-N-[(3-methyl-2-pyridyl)methyl]pyrimidine-5-carboxamide

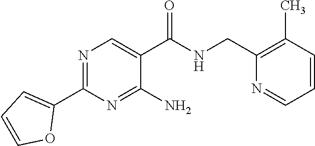 [SMILES: Cc1cccnc1CNC(=O)c1cnc(-c2ccco2)nc1N]

To a suspension of 4-amino-2-(2-furyl)pyrimidine-5-carboxylic acid (step 2) (88 mg, 0.43 mmol) in DMF (3 mL) was added (3-methyl-2-pyridyl)methanamine (77 μL, 0.65 mmol), HATU (326 mg, 0.86 mmol) and DIPEA (374 μL, 2.15 mmol) in quick succession and the mixture stirred at room temperature for 15 mins. The resulting mixture was diluted with EtOAc (25 mL), washed with 50% brine (4×25 mL), dried over MgSO₄ and the solvent removed in vacuo. Purification by column chromatography on silica eluting with a gradient of 0.5 to 4% MeOH in DCM afforded a solid which was triturated with CHCl₃/Et₂O/EtOAc to afford the titled compound as an off-white solid.

LC-MS (Method 8B): Rt 3.31 mins; MS m/z 310.2=[M+H]+

1H NMR (500 MHz, Chloroform-d) δ 8.78 (s, 1H), 8.51-8.45 (m, 1H), 8.40 (dd, J=4.9, 1.5 Hz, 1H), 7.62 (dd, J=1.7, 0.9 Hz, 1H), 7.53 (ddd, J=7.6, 1.7, 0.9 Hz, 1H), 7.32 (dd, J=3.5, 0.9 Hz, 1H), 7.19 (dd, J=7.6, 4.8 Hz, 1H), 7.03 (br s, 2H), 6.57 (dd, J=3.5, 1.7 Hz, 1H), 4.63 (d, J=3.8 Hz, 2H), 2.34 (s, 3H).

### Example 3.1

### 4-Amino-2-(5-methyl-2-furyl)-N-[(3-methyl-2-pyridyl)methyl]pyrimidine-5-carboxamide

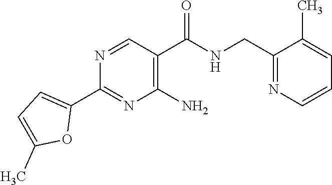 [SMILES: Cc1ccc(-c2ncc(C(=O)NCc3ncccc3C)c(N)n2)o1]

### Step 1: Ethyl 4-amino-2-(5-methyl-2-furyl)pyrimidine-5-carboxylate

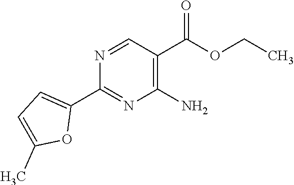 [SMILES: CCOC(=O)c1cnc(-c2ccc(C)o2)nc1N]

The titled compound was prepared from ethyl 4-amino-2-chloro-pyrimidine-5-carboxylate and (5-methyl-2-furyl)boronic acid analogously to Example 3 step 1.

LC-MS (Method 3B): Rt 1.50 mins; MS m/z 248.3=[M+H]+

1H NMR (500 MHz, Chloroform-d) δ 8.90 (s, 1H), 7.93 (s, 1H), 7.29 (br d, J=2.0 Hz, 1H), 6.19 (dq, J=3.2, 1.0 Hz, 1H), 5.85 (s, 1H), 4.36 (q, J=7.1 Hz, 2H), 2.46-2.44 (m, 3H), 1.39 (t, J=7.1 Hz, 3H).

### Step 2: 4-Amino-2-(5-methyl-2-furyl)pyrimidine-5-carboxylic Acid

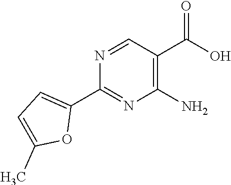 [SMILES: Cc1ccc(-c2ncc(C(=O)O)c(N)n2)o1]

The titled compound was prepared from ethyl 4-amino-2-(5-methyl-2-furyl)pyrimidine-5-carboxylate (step 1) analogously to Example 3 step 2.

LC-MS (Method 3A): Rt 0.94 mins; MS m/z 220.2=[M+H]+

### Step 3: 4-Amino-2-(5-methyl-2-furyl)-N-[(3-methyl-2-pyridyl)methyl]pyrimidine-5-carboxamide

The titled compound was prepared from 4-amino-2-(5-methyl-2-furyl)pyrimidine-5-carboxylic acid and (3-methyl-2-pyridyl)methanamine analogously to Example 3 step 3.

LC-MS (Method 8B): Rt 3.72 mins; MS m/z 324.1=[M+H]+

1H NMR (500 MHz, Chloroform-d) δ 8.75 (s, 1H), 8.43 (br t, J=3.8 Hz, 1H), 8.39 (dd, J=4.8, 1.6 Hz, 1H), 7.51 (ddd, J=7.6, 1.6, 0.9 Hz, 1H), 7.24 (d, J=3.4 Hz, 1H), 7.18 (dd, J=7.6, 4.8 Hz, 1H), 7.06 (v br s, 2H), 6.18 (dq, J=3.4, 1.0 Hz, 1H), 4.61 (d, J=3.8 Hz, 2H), 2.45 (s, 3H), 2.33 (s, 3H).

### Example 4

### 4-Amino-N-benzyl-2-(3-fluorophenyl)pyrimidine-5-carboxamide

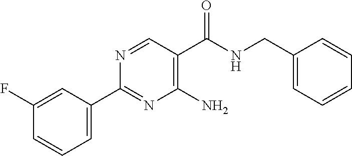 [SMILES: Nc1nc(-c2cccc(F)c2)ncc1C(=O)NCc1ccccc1]

### Step 1: Ethyl 2-(3-fluorophenyl)-4-hydroxy-pyrimidine-5-carboxylate

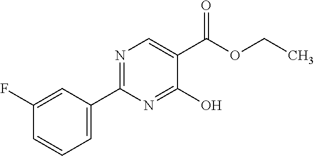 [SMILES: CCOC(=O)c1cnc(-c2cccc(F)c2)nc1O]

The titled compound was prepared from (3-fluorobenzenecarboximidoyl) ammonium chloride and diethyl 2-(ethoxy methylene)propanedioate according to the procedure of Woltering E; Tuch A; Dittrich-Wengenroth E; Kretschmer A; Baerfacker L; Bauser M; Ellinghaus P; Lustig K; Pook E; Weber O (WO2006/097220 A1 page 33).

To a flask containing commercially available (3-fluorobenzenecarboximidoyl) ammonium chloride (5 g, 28.64 mmol) was added sodium ethoxide in ethanol (21%) (21.4 mL, 57.27 mmol) followed by a solution of diethyl 2-(ethoxy methylene)propanedioate (5.79 mL, 28.64 mmol) in ethanol (10 mL). The mixture was heated to reflux for 2 hours and after cooling to room temperature, treated with 6N HCl aq. (50 mL). The precipitate was filtered and washed with water (2×200 mL). The filtered solid was dissolved in EtOAc (1 litre) and dried over MgSO₄. The filtrate was re-filtered and the solid washed with water (2×25 mL) before being dissolved in EtOAc (500 mL), dried over MgSO₄, combined with the other EtOAc fraction and concentrated in vacuo to a volume of 50 mL at which point the formed solid was collected by filtration to afford the titled compound.

1H NMR (500 MHz, Chloroform-d) δ 11.73 (s, 1H), 9.04 (s, 1H), 8.26 (d, J=7.9 Hz, 1H), 8.18 (dt, J=10.1, 2.2 Hz, 1H), 7.49 (td, J=7.9, 5.7 Hz, 1H), 7.30-7.22 (m, 1H), 4.49 (q, J=7.1 Hz, 2H), 1.45 (t, J=7.1 Hz, 3H).

### Step 2: Ethyl 4-chloro-2-(3-fluorophenyl)pyrimidine-5-carboxylate

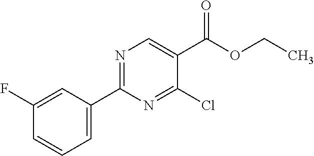 [SMILES: CCOC(=O)c1cnc(-c2cccc(F)c2)nc1Cl]

Ethyl 2-(3-fluorophenyl)-4-hydroxy-pyrimidine-5-carboxylate (step 1) (2.6 g, 9.91 mmol) was added portionwise to phosphorus oxychloride (60.81 g, 396.6 mmol) at 0° C. and the mixture was heated to 110° C. for 16 h. The reaction mixture was cooled to room temperature and added dropwise to a vigorously stirred mixture of ice water (500 mL). The solution was stirred for 15 minutes and extracted with Et₂O (3×100 mL). The combined organic extracts were washed with sat. NaHCO₃ (1×100 mL), dried over MgSO₄ and concentrated in vacuo to afford the titled compound as a white solid.

LC-MS (Method 3B): Rt 2.18 mins; MS m/z 280.9=[M+H]+

1H NMR (500 MHz, Chloroform-d) δ 9.19 (s, 1H), 8.32-8.28 (m, 1H), 8.20 (ddd, J=10.0, 2.6, 1.5 Hz, 1H), 7.48 (dt, J=8.0, 5.6 Hz, 1H), 7.28-7.22 (m, 1H), 4.47 (q, J=7.1 Hz, 2H), 1.45 (t, J=7.1 Hz, 3H).

### Step 3: 4-Chloro-2-(3-fluorophenyl)pyrimidine-5-carboxylic Acid

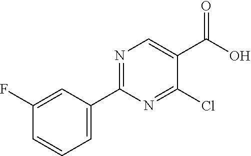 [SMILES: O=C(O)c1cnc(-c2cccc(F)c2)nc1Cl]

To a solution of ethyl 4-chloro-2-(3-fluorophenyl)pyrimidine-5-carboxylate (step 2) (100 mg, 0.36 mmol) in THF (1 mL) was added LiOH (18 mg, 0.43 mmol) in water (1 mL) and the mixture was stirred for 1 h at room temperature. The solvent was removed in vacuo and the crude mixture was dissolved in water and acidified with 2N HCl. The resulting solid was collected by filtration, dissolved in EtOAc, dried over MgSO₄ and concentrated in vacuo to afford the titled compound as a white solid. The solid was used in the next step without further purification.

LC-MS (Method 3A): Rt 1.83 mins; MS m/z 251.0/253.0=[M−H]

1H NMR (500 MHz, DMSO-d6) δ 9.27 (s, 1H), 8.24 (dt, J=8.0, 1.4 Hz, 1H), 8.07 (ddd, J=10.2, 2.8, 1.4 Hz, 1H), 7.64 (dt, J=8.0, 5.9 Hz, 1H), 7.49 (tdd, J=8.4, 2.8, 1.0 Hz, 1H).

### Step 4: N-Benzyl-4-chloro-2-(3-fluorophenyl)pyrimidine-5-carboxamide

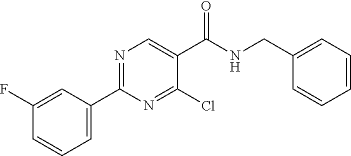 [SMILES: O=C(NCc1ccccc1)c1cnc(-c2cccc(F)c2)nc1Cl]

To a mixture of 4-chloro-2-(3-fluorophenyl)pyrimidine-5-carboxylic acid (step 3) (80 mg, 0.32 mmol) in DCM (3 mL) at 0° C. was added DMF (2 drops) followed by dropwise addition of oxalyl chloride (80 μL, 0.95 mmol) and the mixture was stirred for 1 h. The solvent was removed in vacuo, the solid re-dissolved in DCM (1 mL) and added dropwise to a cooled (0° C.) solution of benzylamine (38 μL, 0.35 mmol) and triethylamine (221 μL, 1.58 mmol) in DCM (2 mL). The reaction mixture was stirred at room temperature for 16 h and then partitioned between DCM (5 mL) and H₂O (5 ml). The organic portion was separated and the aqueous further extracted with DCM (2×10 mL). The combined organic extracts were dried over MgSO₄ and the solvent removed in vacuo. The crude material was suspended in MeCN and the solid collected by filtration to afford the titled compound as a white solid.

LC-MS (Method 3B): Rt 2.00 mins; MS m/z 340.2/342.2=[M+H]+

1H NMR (500 MHz, Chloroform-d) δ 9.19 (s, 1H), 8.29-8.24 (m, 1H), 8.17 (ddd, J=10.1, 2.7, 1.6 Hz, 1H), 7.47 (dt, J=8.0, 5.7 Hz, 1H), 7.41-7.36 (m, 4H), 7.36-7.31 (m, 1H), 7.25-7.21 (m, 1H), 6.90 (br t, 1H), 4.70 (d, J=5.6 Hz, 2H).

### Step 5: 4-Amino-N-benzyl-2-(3-fluorophenyl)pyrimidine-5-carboxamide

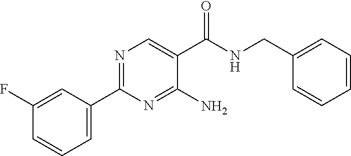 [SMILES: Nc1nc(-c2cccc(F)c2)ncc1C(=O)NCc1ccccc1]

To a solution of N-benzyl-4-chloro-2-(3-fluorophenyl)pyrimidine-5-carboxamide (step 4) (45 mg, 0.13 mmol) in DMF (1 mL) was added sodium azide (26 mg, 0.4 mmol) and the mixture was stirred at 40° C. for 2 h. After cooling to room temperature, the mixture was treated with triphenylphosphine (45 mg, 0.17 mmol) and stirred for 1 h. The resulting mixture was partitioned between EtOAc (5 mL) and H₂O (5 mL). The organic portion was separated, the aqueous portion further extracted with EtOAc (3×5 mL) and the combined organic extracts concentrated in vacuo. The crude material was dissolved in THF (2 mL), 2M HCL (1 mL) was added and the mixture warmed to 40° C. for 1 h. The solvent was removed in vacuo and the resulting solid suspended in EtOAc and collected by filtration. The solid was partitioned between 14% NH₄OH (10 mL) and EtOAc (10 mL). The organic portion was separated and the aqueous further extracted with EtOAc (3×10 mL). The combined organic extracts were washed with brine (1×20 mL), dried over MgSO₄ and the solvent was removed in vacuo. Purification by column chromatography on silica eluting with 30% EtOAc in hexane afforded the titled compound as a white solid.

LC-MS (Method 8B): Rt 4.88 mins; MS m/z 323.0=[M+H]+

1H NMR (500 MHz, Chloroform-d) δ 8.54 (s, 1H), 8.18-8.15 (m, 1H), 8.07 (ddd, J=10.3, 2.7, 1.6 Hz, 1H), 7.42 (dt, J=8.0, 5.7 Hz, 1H), 7.41-7.29 (m, 5H), 7.20-7.14 (m, 1H), 6.93 (br s, 2H), 6.38 (br t, 1H), 4.64 (d, J=5.6 Hz, 2H).

The compounds of the following tabulated Examples (Table 1) were prepared analogously to Example 4 from 4-chloro-2-(3-fluorophenyl)pyrimidine-5-carboxylic acid (Ex. 4 step 3) and the appropriate amine.

TABLE 1

| Ex. | Structure and Name | Retention time, [M + H]+, 1H NMR |
|---|---|---|
| 4.1 | 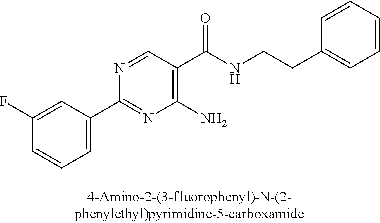 [SMILES: Nc1nc(-c2cccc(F)c2)ncc1C(=O)NCCc1ccccc1] | LC-MS (Method 8B): Rt 5.06 mins; MS m/z 337.1 = [M + H]+ 1H NMR (500 MHz, Chloroform-d) δ 8.46 (s, 1H), 8.16 (dt, J = 8.0, 1.4 Hz, 1H), 8.06 (ddd, J = 10.3, 2.8, 1.4 Hz, 1H), 7.42 (dt, J = 8.0, 5.8 Hz, 1H), 7.37- 7.32 (m, 2H), 7.29-7.22 (m, 3H), 7.18 (tdd, J = 8.3, 2.8, 1.0 Hz, 1H), 6.98 (br s, 2H), 6.32 (s, 1H), 3.71 (apr q, J = 6.5 Hz, 2H), 2.95 (t, J = 6.9 Hz, 2H). |
| 4.2 | 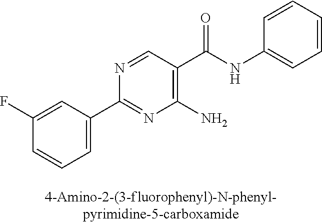 [SMILES: Nc1nc(-c2cccc(F)c2)ncc1C(=O)Nc1ccccc1] | LCMS (Method 8B): 4.96 mins; MS m/z 309.0 = [M + H]+ 1H NMR (500 MHz, DMSO-d6) δ 10.35 (s, 1H), 8.91 (s, 1H), 8.21 (dt, J = 7.8, 1.2 Hz, 1H), 8.07 (ddd, J = 10.5, 2.8, 14 Hz, 1H), 7.86 (s, 2H), 7.73-7.68 (m, 2H), 7.58 (dt, J = 8.0, 5.9 Hz, 1H), 7.42- 7.33 (m, 3H), 7.16-7.09 (m, 1H). |
| 4.3 | 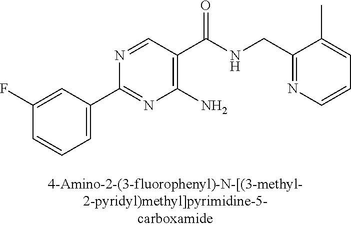 [SMILES: Cc1cccnc1CNC(=O)c1cnc(-c2cccc(F)c2)nc1N] | LC-MS (Method 8B): Rt 4.62 mins; MS m/z 338.1 = [M + H]+ 1H NMR (500 MHz, DMSO-d6) δ 9.03 (t, J = 5.5 Hz, 1H), 8.82 (s, 1H), 8.36 (dd, J = 4.8, 1.7 Hz, 1H), 8.20-8.16 (m, 1H), 8.03 (ddd, J = 10.6, 2.8, 1.5 Hz, 1H), 7.96 (br s, 2H), 7.60 (d, J = 7.6 Hz, 1H), 7.55 (dt, J = 8.0, 5.9 Hz, 1H), 7.37 (td, J = 8.6, 2.8 Hz, 1H), 7.23 (dd, J = 7.6, 4.8 Hz, 1H), 4.58 (d, J = 5.5 Hz, 2H), 2.35 (s, 3H). |

### Example 5

### 4-Amino-2-(3-fluorophenyl)-N-[(1R)-1-phenylethyl]pyrimidine-5-carboxamide

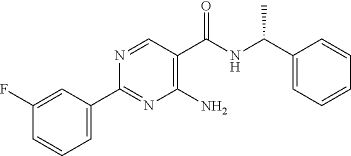 [SMILES: C[C@@H](NC(=O)c1cnc(-c2cccc(F)c2)nc1N)c1ccccc1]

### Step 1: 4-Amino-2-(3-fluorophenyl)pyrimidine-5-carboxylic Acid

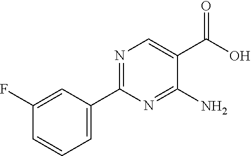 [SMILES: Nc1nc(-c2cccc(F)c2)ncc1C(=O)O]

4-Chloro-2-(3-fluorophenyl)pyrimidine-5-carboxylic acid (Example 4, step 3)(100 mg, 0.39 mmol) was dissolved in a mixture of MeOH (0.5 mL) and NH₄OH (35% in H₂O) (1 mL, 0.39 mmol) and the mixture was heated using microwave radiation at 120° C. for 30 mins. The solvent was removed in vacuo to afford the titled compound as a white solid.

LC-MS (Method 3A): Rt 1.21 mins; MS m/z 234.0=[M+H]+

1H NMR (500 MHz, DMSO-d6) δ 8.82 (s, 1H), 8.73 (s, 1H), 8.19-8.13 (m, 1H), 8.02 (ddd, J=10.7, 2.8, 1.4 Hz, 1H), 7.52 (dt, J=8.0, 6.0 Hz, 1H), 7.32 (td, J=8.5, 2.8 Hz, 1H).

Broad signal at 7.44 ppm—presumed NH₂ protons under this signal.

### Step 2: 4-Amino-2-(3-fluorophenyl)-N-[(1R)-1-phenylethyl]pyrimidine-5-carboxamide

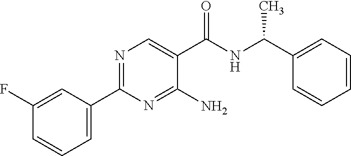 [SMILES: C[C@@H](NC(=O)c1cnc(-c2cccc(F)c2)nc1N)c1ccccc1]

To a solution of 4-amino-2-(3-fluorophenyl)pyrimidine-5-carboxylic acid (step 1) (50 mg, 0.16 mmol) in DMF (2 mL) was added commercially available (1R)-1-phenylethanamine (31 μL, 0.24 mmol), HATU (123 mg, 0.32 mmol) and finally DIPEA (140 μL, 0.8 mmol) and the mixture was stirred for 3 h. The resulting mixture was partitioned between H₂O (10 mL) and EtOAc (10 mL), the organic portion separated and the aqueous further extracted with EtOAc (2×15 mL). The combined organic extracts were washed with water, brine, dried over MgSO₄ and the solvent removed in vacuo. Purification by column chromatography on silica eluting with 15% EtOAc in hexane the titled compound as a white solid.

LC-MS (Method 8B): Rt 4.79 mins; MS m/z 337.1=[M+H]+

1H NMR (500 MHz, Chloroform-d) δ 8.55 (s, 1H), 8.18-8.14 (m, 1H), 8.07 (ddd, J=10.3, 2.7, 1.5 Hz, 1H), 7.42 (dt, J=8.0, 5.7 Hz, 1H), 7.39-7.36 (m, 4H), 7.34-7.29 (m, 1H), 7.17 (tdd, J=8.3, 2.7, 1.0 Hz, 1H), 6.88 (s, 2H), 6.29 (d, J=7.0 Hz, 1H), 5.27 (p, J=7.0 Hz, 1H), 1.62 (d, J=7.0 Hz, 3H).

The compounds of the following tabulated Examples (Table 2) were prepared analogously to Example 5 from either 4-chloro-2-(3-fluorophenyl)pyrimidine-5-carboxylic acid (Ex. 4 step 3) or 4-amino-2-(2-furyl)pyrimidine-5-carboxylic acid (Example 3 step 2) and the appropriate commercially available amine (Example 5 step 2). In Table 2, 5.22 is a comparative example.

TABLE 2

| Ex. | Structure and Name | Retention time, [M + H]+, 1H NMR |
|---|---|---|
| 5.1 | 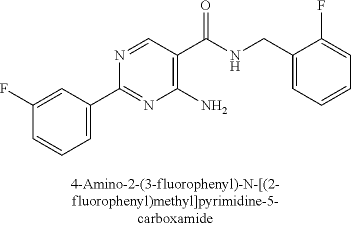 [SMILES: Nc1nc(-c2cccc(F)c2)ncc1C(=O)NCc1ccccc1F] | LC-MS (Method 8B): Rt 4.54 mins; MS m/z 341.1 = [M + H]+ 1H NMR (500 MHz, Chloroform-d) δ 8.55 (s, 1H), 8.18-8.13 (m, 1H), 8.07 (ddd, J = 10.3, 2.7. 1.5 Hz, 1H), 7.45-7.37 (m, 2H), 7.34-7.28 (m, 1H), 7.19-7.13 (m, 2H), 7.12-7.07 (m, 1H), 6.89 (s, 2H), 6.50 (t, J = 5.9 Hz, 1H), 4.68 (d, J = 5.8 Hz, 2H). |
| 5.2 | 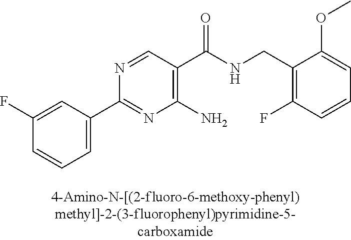 [SMILES: COc1cccc(F)c1CNC(=O)c1cnc(-c2cccc(F)c2)nc1N] | LC-MS (Method 8B): Rt 4.57 mins; MS m/z 371.1 = [M + H]+ 1H NMR (500 MHz, DMSO-d6) δ 8.72 (t, J = 4.8 Hz, 1H), 8.71 (s, 1H), 8.18-8.13 (m, 1H), 8.01 (ddd, J = 10.6, 2.7, 1.4 Hz, 1H), 7.90 (s, 2H), 7.54 (dt, J = 8.0, 5.9 Hz, 1H), 7.39- 7.29 (m, 2H), 6.89 (d, J = 8.4 Hz, 1H), 6.81 (t, J = 8.9 Hz, 1H), 4.46 (d, J = 4.8 Hz, 2H), 3.84 (s, 3H). |
| 5.3 | 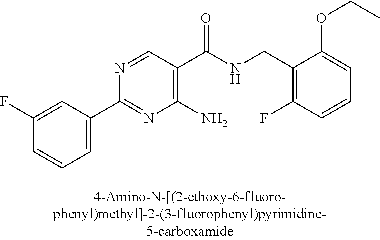 [SMILES: CCOc1cccc(F)c1CNC(=O)c1cnc(-c2cccc(F)c2)nc1N] | LC-MS (Method 8B): Rt 4.91 mins; MS m/z 385.1 = [M + H]+ 1H NMR (500 MHz, DMSO-d6) δ 8.71 (s, 1H), 8.69 (t, J = 4.9 Hz, 1H), 8.19-8.14 (m, 1H), 8.02 (dt, J = 10.3, 2.2 Hz, 1H), 7.92 (s, 2H), 7.55 (dt, J = 8.0, 5.9 Hz, 1H), 7.37 (td, J = 8.5, 2.8 Hz, 1H), 7.31 (td, J = 8.3, 6.7 Hz, 1H), 6.87 (d, J = 8.3 Hz, 1H), 6.80 (t, J = 8.9 Hz, 1H), 4.49 (d, J = 4.9 Hz, 2H), 4.09 (q, J = 6.9 Hz, 2H), 1.33 (t, J = 6.9 Hz, 3H). |
| 5.4 | 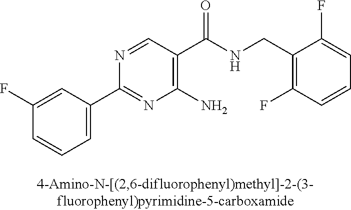 [SMILES: Nc1nc(-c2cccc(F)c2)ncc1C(=O)NCc1c(F)cccc1F] | LC-MS (Method 8B): Rt 3.78 mins; MS m/z 359.1 = [M + H]+ 1H NMR (500 MHz, DMSO-d6) δ 9.05 (t, J = 5.1 Hz, 1H), 8.75 (s, 1H), 8.18-8.14 (m, 1H), 8.01 (ddd, J = 10.5, 2.7, 1.3 Hz, 1H), 7.92 (br s, 2H), 7.54 (dt, J = 8.1, 6.0 Hz, 1H), 7.45-7.38 (m, 1H), 7.36 (td, J = 8.1, 6.0 Hz, 1H), 7.14-7.08 (m, 2H), 4.51 (d, J = 5.1 Hz, 2H). |
| 5.5 | 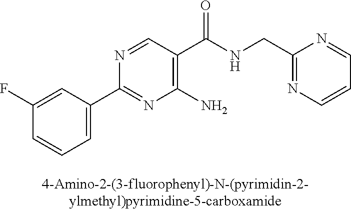 [SMILES: Nc1nc(-c2cccc(F)c2)ncc1C(=O)NCc1ncccn1] | LC-MS (Method 8B): Rt 3.37 mins; MS m/z 325.0 = [M + H]+ 1H NMR (500 MHz, DMSO-d6) δ 9.26 (t, J = 5.6 Hz, 1H), 8.89 (s, 1H), 8.79 (d, J = 4.9 Hz, 2H), 8.19 (apr d, J = 7.8 Hz, 1H), 8.07-8.02 (m, 1H), 7.93 (br s, 2H), 7.56 (dt, J = 8.0, 6.0 Hz, 1H), 7.41 (t, J = 4.9 Hz, 1H), 7.38 (td, J = 8.5, 2.8 Hz, 1H), 4.66 (d, J = 5.6 Hz, 2H). |
| 5.6 | 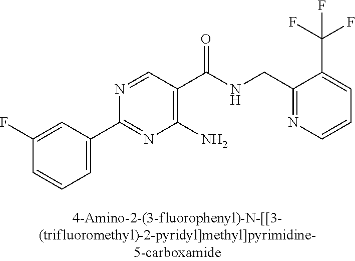 [SMILES: Nc1nc(-c2cccc(F)c2)ncc1C(=O)NCc1ncccc1C(F)(F)F] | LC-MS (Method 8B): Rt 4.46 mins; MS m/z 392.1 = [M + H]+ 1H NMR (500 MHz, DMSO-d6) δ 9.21 (t, J = 5.4 Hz, 1H), 8.87 (s, 1H), 8.82 (d, J = 4.5 Hz, 1H), 8.22-8.17 (m, 2H), 8.04 (ddd, J = 10.6, 2.8, 1.5 Hz, 1H), 7.92 (s, 2H), 7.59-7.53 (m, 2H), 7.37 (td, J = 8.5, 2.8 Hz, 1H), 4.75 (d, J = 5.4 Hz, 2H). |
| 5.7 | 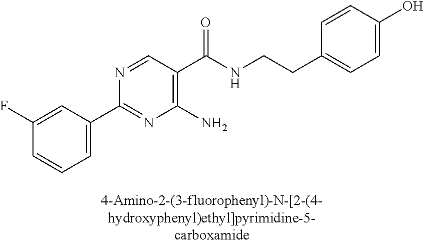 [SMILES: Nc1nc(-c2cccc(F)c2)ncc1C(=O)NCCc1ccc(O)cc1] | LC-MS (Method 8B): Rt 3.97mins; MS m/z 353.1 = [M + H]+ 1H NMR (500 MHz, DMSO-d6) δ 9.20 (s, 1H), 8.69 (s, 1H), 8.68 (t, J = 5.6 Hz, 1H), 8.17 (dt, J = 8.0, 1.1 Hz, 1H), 8.02 (ddd, J = 10.6, 2.7, 1.5 Hz, 1H), 7.91 (br s, 2H), 7.55 (dt, J = 8.0, 6.0 Hz, 1H), 7.36 (tdd, J = 8.5, 2.7, 1.1 Hz, 1H), 7.06-6.99 (m, 2H), 6.71-6.65 (m, 2H), 3.44-3.37 (m, 2H), 2.75-2.69 (m, 2H). |
| 5.8 | 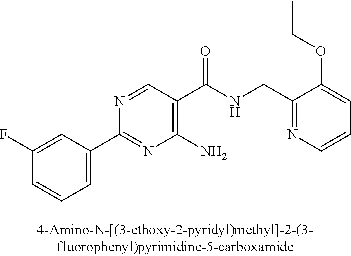 [SMILES: CCOc1cccnc1CNC(=O)c1cnc(-c2cccc(F)c2)nc1N] | LC-MS (Method 8B): Rt 4.36 mins; MS m/z 368.1 = [M + H]+ 1H NMR (500 MHZ, DMSO-d6) δ 8.93 (t, J = 5.7 Hz, 1H), 8.82 (s, 1H), 8.21-8.15 (m, 1H), 8.08 (dd, J = 4.8, 1.2 Hz, 1H), 8.06-8.01 (m, 1H), 7.95 (br s, 2H), 7.55 (dt, J = 8.0, 5.9 Hz, 1H), 7.41 (dd, J = 8.4, 1.2 Hz, 1H), 7.37 (td, J = 8.4, 2.8 Hz, 1H), 7.28 (dd, J = 8.3, 4.8 Hz, 1H), 4.58 (d, J = 5.7 Hz, 2H), 4.12 (q, J = 6.9 Hz, 2H), 1.37 (t, J = 6.9 Hz, 3H). |
| 5.9 | 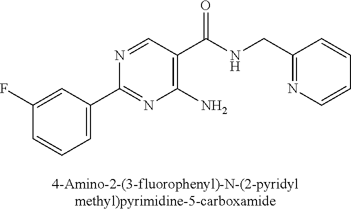 [SMILES: Nc1nc(-c2cccc(F)c2)ncc1C(=O)NCc1ccccn1] | LC-MS (Method 8B): Rt 3.78 mins; MS m/z 324.1 = [M + H]+ 1H NMR (500 MHz, DMSO-d6) δ 9.28 (t, J = 5.9 Hz, 1H), 8.88 (s, 1H), 8.52 (ddd, J = 4.9, 1.8, 0.9 Hz, 1H), 8.19 (dt, J = 7.8, 1.2 Hz, 1H), 8.04 (ddd, J = 10.6, 2.7, 1.5 Hz, 1H), 7.94 (br s, 2H), 7.77 (td, J = 7.8, 1.8 Hz, 1H), 7.56 (dt, J = 8.0, 6.0 Hz, 1H), 7.40-7.35 (m, 2H), 7.28 (ddd, J = 7.5, 4.9, 1.2 Hz, 1H), 4.57 (d, J = 5.9 Hz, 2H). |
| 5.10 | 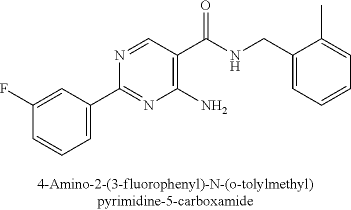 [SMILES: Cc1ccccc1CNC(=O)c1cnc(-c2cccc(F)c2)nc1N] | LC-MS (Method 8B): Rt 4.69 mins; MS m/z 337.1 = [M + H]+ 1H NMR (500 MHz, DMSO-d6) δ 9.07 (t, J = 5.7 Hz, 1H), 8.85 (s, 1H), 8.20-8.16 (m, 1H), 8.03 (ddd, J = 10.6, 2.8, 1.5 Hz, 1H), 7.95 (br s, 2H), 7.56 (dt, J = 8.0, 6.0 Hz, 1H), 7.37 (tdd, J = 8.5, 2.8, 1.0 Hz, 1H), 7.29-7.24 (m, 1H), 7.20-7.14 (m, 3H), 4.45 (d, J = 5.7 Hz, 2H), 2.33 (s, 3H). |
| 5.11 | 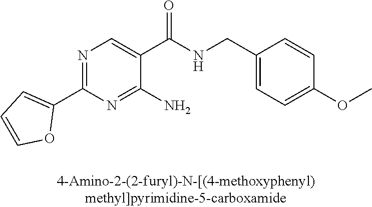 [SMILES: COc1ccc(CNC(=O)c2cnc(-c3ccco3)nc2N)cc1] | LC-MS (CP-QC1_acidic) Rt 2.83 mins; MS m/z 325.3 [M + H]+ ¹H NMR (400 MHz, DMSO) δ 9.06 (t, J = 5.9 Hz, 1H), 8.72 (s, 1H), 7.94- 7.87 (m, 3H), 7.28-7.22 (m, 3H), 6.90 (td, J = 2.5, 9.6 Hz, 2H), 6.66 (dd, J = 1.7, 3.4 Hz, 1H), 4.39 (d, J = 5.8 Hz, 2H), 3.73 (s, 3H). |
| 5.12 | 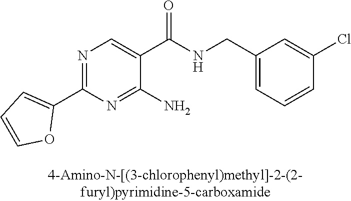 [SMILES: Nc1nc(-c2ccco2)ncc1C(=O)NCc1cccc(Cl)c1] | LC-MS (CP-QC5_basic) Rt 3.24 mins; MS m/z 329.2/331.2 [M + H]+ ¹H NMR (400 MHz, DMSO) δ 9.15 (t, J = 5.9 Hz, 1H), 8.75 (s, 1H), 8.01- 7.89 (m, 3H), 7.40-7.36 (m, 2H), 7.31 (tt, J = 1.5, 7.8 Hz, 2H), 7.25 (dd, J = 0.8, 3.4 Hz, 1H), 6.68 (dd, J = 1.8, 3.4 Hz, 1H), 4.47 (d, J = 5.9 Hz, 2H) |
| 5.13 | 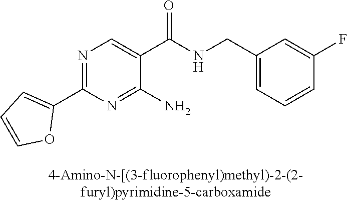 [SMILES: Nc1nc(-c2ccco2)ncc1C(=O)NCc1cccc(F)c1] | LC-MS (CP-QC5_basic) Rt 3.14 mins; MS m/z 313.2 [M + H]+ ¹H NMR (400 MHz, DMSO) δ 9.16 (t, J = 5.9 Hz, 1H), 8.76 (s, 1H), 7.98- 7.89 (m, 3H), 7.42-7.35 (m, 1H), 7.25 (dd, J = 0.8, 3.4 Hz, 1H), 7.19- 7.13 (m, 2H), 7.11-7.05 (m, 1H), 6.68 (dd, J = 1.7, 3.4 Hz, 1H), 4.48 (d, J = 5.9 Hz, 2H) |
| 5.14 | 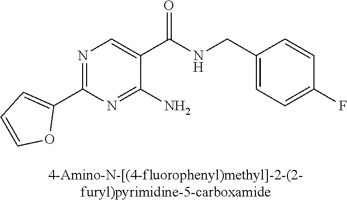 [SMILES: Nc1nc(-c2ccco2)ncc1C(=O)NCc1ccc(F)cc1] | LC-MS (CP-QC5_basic) Rt 3.14 mins; MS m/z 313.2 [M + H]+ ¹H NMR (400 MHz, DMSO) δ 9.13 (t, J = 5.9 Hz, 1H), 8.74 (s, 1H), 7.95- 7.88 (m, 3H), 7.40-7.34 (m, 2H), 7.23 (dd, J = 0.9, 3.4 Hz, 1H), 7.16 (tt, J = 2.5, 9.4 Hz, 2H), 6.67 (dd, J = 1.7, 3.4 Hz, 1H), 4.44 (d, J = 5.9 Hz, 2H) |
| 5.15 | 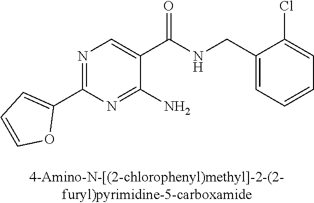 [SMILES: Nc1nc(-c2ccco2)ncc1C(=O)NCc1ccccc1Cl] | LC-MS (CP-QC5_basic) Rt 3.43 mins; MS m/z 329.1/331.2 [M + H]+ ¹H NMR (400 MHz, DMSO) δ 9.14 (t, J = 5.8 Hz, 1H), 8.80 (s, 1H), 7.95- 7.89 (m, 3H), 7.48-7.45 (m, 1H), 7.42-7.38 (m, 1H), 7.37-7.28 (m, 2H), 7.25 (dd, J = 0.8, 3.4 Hz, 1H), 6.68 (dd, J = 1.7, 3.4 Hz, 1H), 4.53 (d, J = 5.7 Hz, 2H). |
| 5.16 | 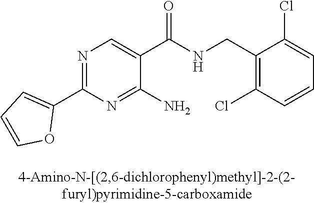 [SMILES: Nc1nc(-c2ccco2)ncc1C(=O)NCc1c(Cl)cccc1Cl] | LC-MS (CP-QC5_basic) Rt 3.30 mins; MS m/z 363.2/365.2/367.2 [M + H]+ ¹H NMR (400 MHz, DMSO) δ 8.77 (t, J = 4.4 Hz, 1H), 8.64 (s, 1H), 7.90- 7.86 (m, 3H), 7.53-7.50 (m, 2H), 7.39 (dd, J = 7.4, 8.7 Hz, 1H), 7.21 (dd, J = 0.8, 3.4 Hz, 1H), 6.66 (dd, J = 1.7, 3.4 Hz, 1H), 4.68 (d, J = 4.4 Hz, 2H) |
| 5.17 | 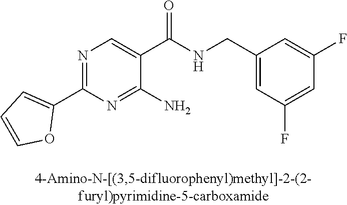 [SMILES: Nc1nc(-c2ccco2)ncc1C(=O)NCc1cc(F)cc(F)c1] | LC-MS (CP-QC5_basic) Rt 3.19 mins; MS m/z 331.2 [M + H]+ ¹H NMR (400 MHz, DMSO) δ 9.16 (t, J = 5.7 HZ, 1H), 8.76 (s, 1H), 7.96- 7.89 (m, 3H), 7.25 (dd, J = 0.8, 3.4 Hz, 1H), 7.15-7.02 (m, 3H), 6.68 (dd, J = 1.7, 3.4 Hz, 1H), 4.48 (d, J = 5.9 Hz, 2H) |
| 5.18 | 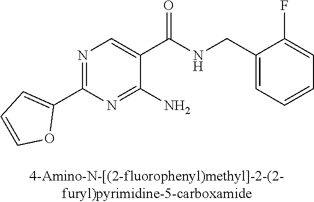 [SMILES: Nc1nc(-c2ccco2)ncc1C(=O)NCc1ccccc1F] | LC-MS (CP-QC5_basic) Rt 3.13 mins; MS m/z 313.2 [M + H]+ ¹H NMR (400 MHz, DMSO) δ 9.11 (t, J = 5.7 Hz, 1H), 8.76 (s, 1H), 7.93- 7.88 (m, 3H), 7.42-7.37 (m, 1H), 7.36-7.29 (m, 1H), 7.24-7.16 (m, 3H), 6.67 (dd, J = 1.7, 3.4 Hz, 1H), 4.50 (d, J = 5.7 Hz, 2H) |
| 5.19 | 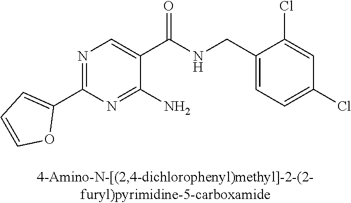 [SMILES: Nc1nc(-c2ccco2)ncc1C(=O)NCc1ccc(Cl)cc1Cl] | LC-MS (CP-QC1_acidic) Rt 3.22 mins; MS m/z 363.2 /365.2/367.2 [M + H]+ ¹H NMR (400 MHz, DMSO) δ 9.15 (t, J = 5.7 Hz, 1H), 8.79 (s, 1H), 7.93- 7.89 (m, 3H), 7.63 (dd, J = 0.6, 1.7 Hz, 1H), 7.45-7.40 (m, 2H), 7.25 (dd, J = 0.8, 3.4 Hz, 1H), 6.68 (dd, J = 1.7, 3.4 Hz, 1H), 4.50 (d, J = 5.7 Hz, 2H). |
| 5.20 | 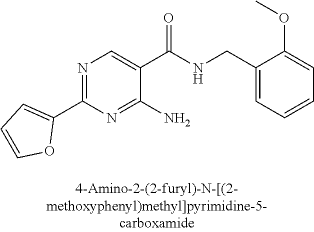 [SMILES: COc1ccccc1CNC(=O)c1cnc(-c2ccco2)nc1N] | LC-MS (CP-QC1_acidic) Rt 2.89 mins; MS m/z 325.2 [M + H]+ ¹H NMR (400 MHz, DMSO) δ 8.95 (t, J = 5.8 Hz, 1H), 8.77 (s, 1H), 7.96- 7.88 (m, 3H), 7.28-7.19 (m, 3H), 7.02-6.99 (m, 1H), 6.92 (dt, J = 0.9, 7.4 Hz, 1H), 6.67 (dd, J = 1.7, 3.4 Hz, 1H), 4.43 (d, J = 5.8 Hz, 2H), 3.83 (s, 3H) |
| 5.21 | 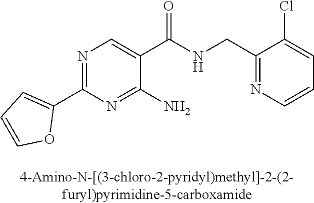 [SMILES: Nc1nc(-c2ccco2)ncc1C(=O)NCc1ncccc1Cl] | LC-MS (CP-QC5_basic) Rt 3700 mins; MS m/z 330.2/332.2 [M + H]+ ¹H NMR (400 MHz, DMSO) δ 9.07 (t, J = 5.5 Hz, 1H), 8.77 (s, 1H), 8.51 (dd, J = 1.4, 4.6 Hz, 1H), 7.96-7.88 (m, 4H), 7.38 (dd, J = 4.7, 8.0 Hz, 1H), 7.24 (dd, J = 0.8, 3.4 Hz, 1H), 6.67 (dd, J = 1.8, 3.4 Hz, 1H), 4.67 (d, J = 5.5 Hz, 2H). |
| comp 5.22 | 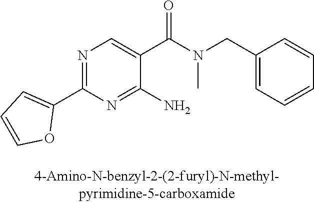 [SMILES: CN(Cc1ccccc1)C(=O)c1cnc(-c2ccco2)nc1N] | LC-MS (CP-QC5_basic) Rt 3.08 mins; MS m/z 309.2 [M + H]+ ¹H NMR (400 MHz, DMSO) δ 8.22 (s, 1H), 7.85 (dd, J = 0.8, 1.7 Hz, 1H), 7.40-7.14 (m, 8H), 6.64 (dd, J = 1.8, 3.3 Hz, 1H), 4.57 (s, 2H), 2.88 (s, 3H) |
| 5.23 | 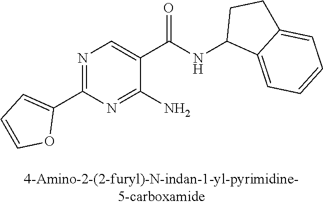 [SMILES: Nc1nc(-c2ccco2)ncc1C(=O)NC1CCc2ccccc21] | LC-MS (CP-QC5_basic) Rt 3.24 mins; MS m/z 321.3 [M + H]+ ¹H NMR (400 MHz, DMSO) δ 8.85 (d, J = 8.2 Hz, 1H), 8.72 (s, 1H), 7.96- 7.87 (m, 3H), 7.29-7.18 (m, 5H), 6.66 (dd, J = 1.7, 3.4 Hz, 1H), 5.54 (dd, J = 8.0, 16.1 Hz, 1H), 3.00 (ddd, J = 3.1, 9.0, 15.8 Hz, 1H), 2.86 (ddd, J = 8.2, 8.2, 16.1 Hz, 1H), 2.49-2.42 (m, 1H), 2.03-1.92 (m, 1H) |
| 5.24 | 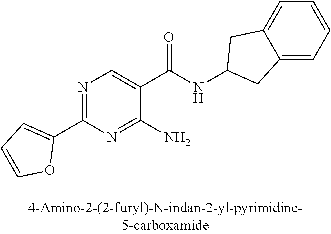 [SMILES: Nc1nc(-c2ccco2)ncc1C(=O)NC1Cc2ccccc2C1] | LC-MS (CP-QC1_acidic) Rt 2.99 mins; MS m/z 321.2 [M + H]+ ¹H NMR (400 MHz, DMSO) δ 8.74 (d, J = 6.9 Hz, 1H), 8.69 (s, 1H), 7.92- 7.87 (m, 3H), 7.27-7.21 (m, 3H), 7.19-7.13 (m, 2H), 6.67-6.65 (m, 1H), 4.73-4.63 (m, 1H), 3.29-3.22 (m, 2H), 2.96 (dd, J = 6.7, 16.0 Hz, 2H) |
| 5.25 | 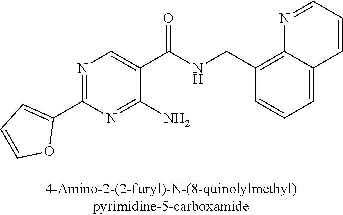 [SMILES: Nc1nc(-c2ccco2)ncc1C(=O)NCc1cccc2cccnc12] | LC-MS (CP-QC1_acidic) Rt 2.66 mins; MS m/z 346.2 [M + H]+ ¹H NMR (400 MHz, DMSO) δ 9.18 (t, J = 5.8 Hz, 1H), 8.98 (dd, J = 1.8, 4.2 Hz, 1H), 8.84 (s, 1H), 8.41 (dd, J = 1.8, 8.3 Hz, 1H), 7.97-7.89 (m, 4H), 7.68 (dd, J = 1.2, 7.1 Hz, 1H), 7.62-7.57 (m, 2H), 7.25 (dd, J = 0.8, 3.4 Hz, 1H), 6.68 (dd, J = 1.8, 3.4 Hz, 1H), 5.13 (d, J = 5.7 Hz, 2H) |
| 5.26 | 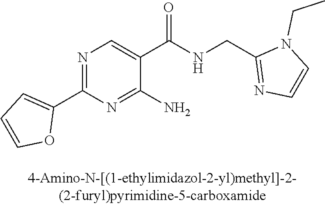 [SMILES: CCn1ccnc1CNC(=O)c1cnc(-c2ccco2)nc1N] | LC-MS (CP-QC5_basic) Rt 2.80 mins; MS m/z 313.2 [M + H]+ ¹H NMR (400 MHz, DMSO) δ 9.07 (t, J = 5.5 Hz, 1H), 8.69 (s, 1H), 7.95- 7.87 (m, 3H), 7.22 (dd, J = 0.9, 3.4 Hz, 1H), 7.16 (d, J = 1.2 Hz, 1H), 6.83 (d, J = 1.2 Hz, 1H), 6.66 (dd, J = 1.7, 3.4 Hz, 1H), 4.53 (d, J = 5.6 Hz, 2H), 4.01 (q, J = 7.2 Hz, 2H), 1.28 (t, J = 7.2 Hz, 3H) |
| 5.27 | 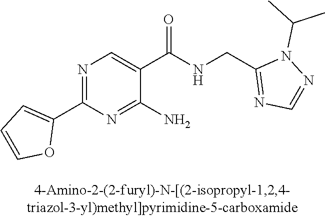 [SMILES: CC(C)n1ncnc1CNC(=O)c1cnc(-c2ccco2)nc1N] | LC-MS (CP-QC1_acidic) Rt 2.52 mins; MS m/z 328.3 [M + H]+ ¹H NMR (400 MHz, DMSO) δ 9.22 (t, J = 5.7 Hz, 1H), 8.71 (s, 1H), 7.92- 7.87 (m, 4H), 7.24 (dd, J = 0.8, 3.4 Hz, 1H), 6.67 (dd, J = 1.7, 3.4 Hz, 1H), 4.87-4.77 (m, 1H), 4.62 (d, J = 5.6 Hz, 2H), 1.39 (d, J = 6.6 Hz, 6H). |
| 5.28 | 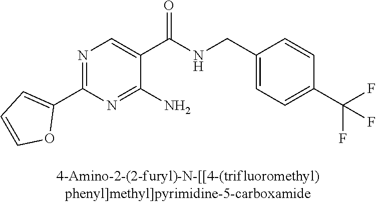 [SMILES: Nc1nc(-c2ccco2)ncc1C(=O)NCc1ccc(C(F)(F)F)cc1] | LC-MS (CP-QC1_acidic) Rt mins; MS m/z 363.2 [M + H]+ ¹H NMR (400 MHz, DMSO) δ 9.22 (t, J = 5.8 Hz, 1H), 8.77 (s, 1H), 7.96- 7.88 (m, 3H), 7.71 (d, J = 8.0 Hz, 2H), 7.55 (d, J = 8.0 Hz, 2H), 7.24 (dd, J = 0.8, 3.4 Hz, 1H), 6.68 (dd, J = 1.7, 3.4 Hz, 1H), 4.55 (d, J = 5.6 Hz, 2H). |
| 5.29 | 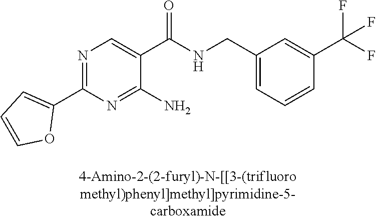 [SMILES: Nc1nc(-c2ccco2)ncc1C(=O)NCc1cccc(C(F)(F)F)c1] | LC-MS (CP-QC1_acidic) Rt 3.12 mins; MS m/z 363.2 [M + H]+ ¹H NMR (400 MHz, DMSO) δ 9.21 (t, J = 5.9 Hz, 1H), 8.76 (s, 1H), 8.01- 7.90 (m, 3H), 7.70-7.56 (m, 4H), 7.26 (dd, J = 0.7, 3.4 Hz, 1H), 6.68 (dd, J = 1.7, 3.4 Hz, 1H), 4.55 (d, J = 5.9 Hz, 2H). |
| 5.30 | 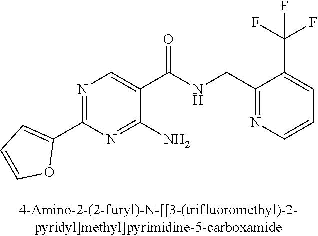 [SMILES: Nc1nc(-c2ccco2)ncc1C(=O)NCc1ncccc1C(F)(F)F] | LC-MS (Method 8B): Rt 3.87 mins; MS m/z 364.1 = [M + H]+ 1H NMR (500 MHz, Chloroform-d) δ 8.75 (dd, J = 4.9, 0.8 Hz, 1H), 8.74 (s, 1H), 8.14 (t, J = 3.9 Hz, 1H), 8.03 (ddd, J = 7.9, 1.7, 0.8 Hz, 1H), 7.63 (dd, J = 1.8, 0.9 Hz, 1H), 7.43 (ddq, J = 7.9, 4.9, 0.8 Hz, 1H), 7.33 (dd, J = 3.5, 0.9 Hz, 1H), 7.14 (br s, 2H), 6.57 (dd, J = 3.5, 1.8 Hz, 1H), 4.92 (d, J = 3.9 Hz, 2H). |
| 5.31 | 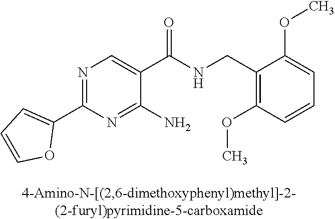 [SMILES: COc1cccc(OC)c1CNC(=O)c1cnc(-c2ccco2)nc1N] | LC-MS (ARG-QC1_acidic) Rt 3.46 min MS m/z 355.1 = [M + H]+ ¹H NMR (400 MHz, DMSO) δ 8.59 (s, 1H), 8.31 (dd, J = 3.9, 3.9 Hz, 1H), 7.86 (s, 3H), 7.27 (t, J = 8.3 Hz, 1H), 7.18 (d, J = 3.2 Hz, 1H), 6.70-6.63 (m, 3H), 4.43 (d, J = 4.3 Hz, 2H), 3.79 (s, 6H). |
| 5.32 | 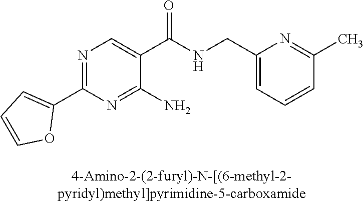 [SMILES: Cc1cccc(CNC(=O)c2cnc(-c3ccco3)nc2N)n1] | LC-MS (ARG-QC1_acidic) Rt 1.89 mins; MS m/z 310.2= [M + H]+; ¹H NMR (400 MHz, DMSO) δ 9.18 (t, J = 5.8 HZ, 1H), 8.78 (s, 1H), 7.95- 7.88 (m, 3H), 7.65 (t, J = 7.7 Hz, 1H), 7.24 (dd, J = 0.9, 3.4 Hz, 1H), 7.16- 7.11 (m, 2H), 6.67 (dd, J = 1.8, 3.4 Hz, 1H), 4.50 (d, J = 5.7 Hz, 2H), 2.46 (s, 3H). |
| 5.33 | 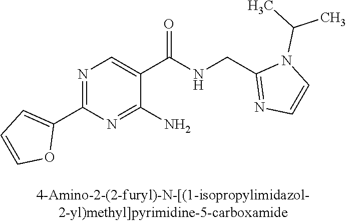 [SMILES: CC(C)n1ccnc1CNC(=O)c1cnc(-c2ccco2)nc1N] | LC-MS (ARG-QC1_acidic) Rt Z03 mins; MS m/z 327.1 = [M + H]+ ¹H NMR (400 MHz, DMSO) δ 9.06 (dd, J = 5.5, 5.5 Hz, 1H), 8.68 (s, 1H), 7.94-7.94 (m, 2H), 7.88 (d, J = 0.9 Hz, 1H), 7.27 (d, J = 1.2 Hz, 1H), 7.23- 7.21 (m, 1H), 6.85 (d, J = 1.0 Hz, 1H), 6.66 (dd, J = 1.8, 3.4 Hz, 1H), 4.55 (d, J = 5.7 Hz, 2H), 1.34 (d, J = 6.7 Hz, 6H). |
| 5.34 | 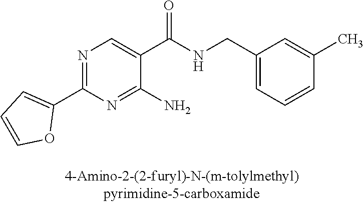 [SMILES: Cc1cccc(CNC(=O)c2cnc(-c3ccco3)nc2N)c1] | LC-MS (CP-QC1_acidic) Rt 2.98 mins; MS m/z 309.2 = [M + H]+ ¹H NMR (400 MHz, DMSO) δ 9.09 (t, J = 5.9 Hz, 1H), 8.75 (s, 1H), 7.96- 7.88 (m, 3H), 7.25-7.20 (m, 2H), 7.14-7.11 (m, 2H), 7.06 (d, J = 7.5 Hz, 1H), 6.67 (dd, J = 1.8. 3.4 Hz, 1H), 4.43 (d, J = 5.9 Hz, 2H), 2.30 (s, 3H) |
| 5.35 | 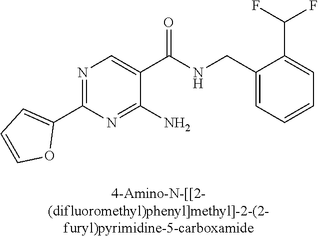 [SMILES: Nc1nc(-c2ccco2)ncc1C(=O)NCc1ccccc1C(F)F] | LC-MS (CP-QC1_acidic) Rt 3709 mins; MS m/z 345.2 = [M + H]+ ¹H NMR (400 MHz, DMSO) δ 9.15 (t, J = 5.8 Hz, 1H), 8.77 (s, 1H), 7.90 (tt, J = 2.7, 3.7 Hz, 3H), 7.60 (d, J = 7.6 Hz, 1H), 7.56-7.51 (m, 1H), 7.48-7.18 (m, 4H), 6.67 (dd, J = 1.8, 3.4 Hz, 1H), 4.62 (d, J = 5.6 Hz, 2H) |
| 5.36 | 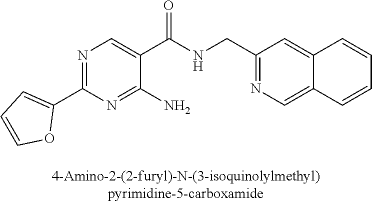 [SMILES: Nc1nc(-c2ccco2)ncc1C(=O)NCc1cc2ccccc2cn1] | LC-MS (CP-QC5_basic) Rt 3.05 mins; MS m/z 346.2= [M + H]+ ¹H NMR (400 MHz, DMSO) δ 9.33- 9.29 (m, 2H), 8.86 (s, 1H), 8.14-8.10 (m, 1H), 8.00-7.89 (m, 4H), 7.78- 7.74 (m, 2H). 7.67-7.62 (m, 1H). 7.25 (dd, J = 0.9, 3.4 Hz, 1H), 6.68 (dd, J = 1.7, 3.4 Hz, 1H), 4.72 (d, J = 5.8 Hz, 2H). |
| 5.37 | 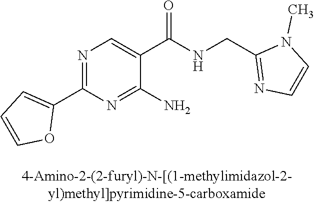 [SMILES: Cn1ccnc1CNC(=O)c1cnc(-c2ccco2)nc1N] | LC-MS (ARG-QC1_acidic) Rt 1.68 mins MS m/z 299.1= [M + H]+ ¹H NMR (400 MHz, DMSO) δ 9.06- 9.01 (m, 1H), 8.70 (s, 1H), 7.94-7.94 (m, 2H), 7.88 (dd, J = 0.9, 1.7 Hz, 1H), 7.22 (dd, J = 0.9, 3.4 Hz, 1H), 7.09 (d, J = 1.1 Hz, 1H), 6.80 (d, J = 1.2 Hz, 1H), 6.66 (dd, J = 1.7. 3.4 Hz, 1H), 4.50 (d, J = 5.5 Hz, 2H), 3.65 (s, 3H). |
| 5.38 | 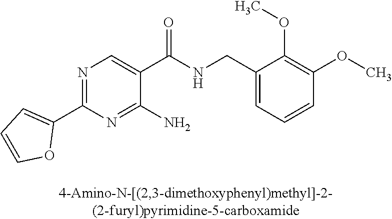 [SMILES: COc1cccc(CNC(=O)c2cnc(-c3ccco3)nc2N)c1OC] | LC-MS (CP-QC5_basic) Rt 3.11 mins; MS m/z 355.2 = [M + H]+ ¹H NMR (400 MHz, DMSO) δ 9.00 (t, J = 5.8 Hz, 1H), 8.75 (s, 1H), 7.98- 7.89 (m, 3H), 7.24 (dd, J = 0.8, 3.4 Hz, 1H), 7.06-7.01 (m, 1H), 6.96 (dd, J = 1.6, 8.2 Hz, 1H), 6.87 (dd, J = 1.5, 7.5 Hz, 1H), 6.68 (dd, J = 1.7, 3.4 Hz, 1H), 4.47 (d, J = 5.8 Hz, 2H), 3.81 (s, 3H), 3.77 (s, 3H) |
| 5.39 | 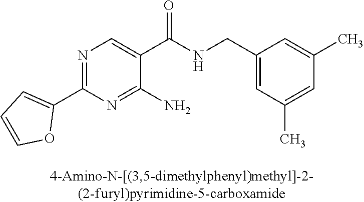 [SMILES: Cc1cc(C)cc(CNC(=O)c2cnc(-c3ccco3)nc2N)c1] | LC-MS (CP-QC1_acidic) Rt 3.12 mins; MS m/z 323.2 = [M + H]+ ¹H NMR (400 MHz, DMSO) δ 9.07 (t, J = 5.8 Hz, 1H), 8.74 (s, 1H), 8.00- 7.89 (m, 3H), 7.24 (dd, J = 0.8, 3.4 Hz, 1H), 6.93 (s, 2H), 6.88 (s, 1H), 6.68 (dd, J = 1.7, 3.4 Hz, 1H), 4.39 (d, J = 5.9 Hz, 2H), 2.25 (s, 6H) |
| 5.40 | 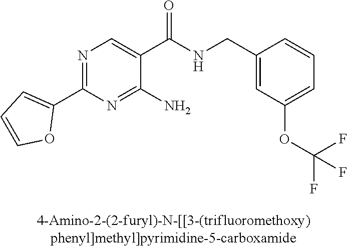 [SMILES: Nc1nc(-c2ccco2)ncc1C(=O)NCc1cccc(OC(F)(F)F)c1] | LC-MS (CP-QC5_basic) Rt 3.36 mins; MS m/z 379.2 = [M + H]+ ¹H NMR (400 MHz, DMSO) δ 9.19 (t, J = 5.9 Hz, 1H), 8.75 (s, 1H), 7.98- 7.89 (m, 3H), 7.48 (t, J = 7.9 Hz, 1H), 7.39-7.36 (m, 1H), 7.31 (s, 1H), 7.28- 7.24 (m, 2H), 6.68 (dd, J = 1.7, 3.4 Hz, 1H), 4.51 (d, J = 5.8 Hz, 2H) |
| 5.41 | 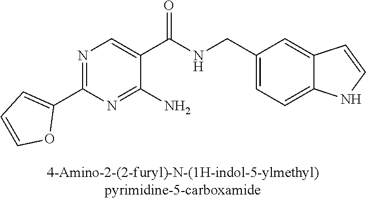 [SMILES: Nc1nc(-c2ccco2)ncc1C(=O)NCc1ccc2[nH]ccc2c1] | LC-MS (CP-QC5_basic) Rt 3.04 mins; MS m/z 334.2= [M + H]+ ¹H NMR (400 MHz, DMSO) δ 11.05 (s, 1H), 9.08 (t, J = 5.9 Hz, 1H), 8.74 (s, 1H), 7.96-7.87 (m, 3H), 7.49 (s, 1H), 7.37-7.30 (m, 2H), 7.22 (dd, J = 0.8, 3.4 Hz, 1H), 7.08 (dd, J = 1.6, 8.4 Hz, 1H), 6.66 (dd, J = 1.8. 3.4 Hz, 1H), 6.41-6.38 (m, 1H), 4.52 (d, J = 5.9 Hz, 2H) |
| 5.42 | 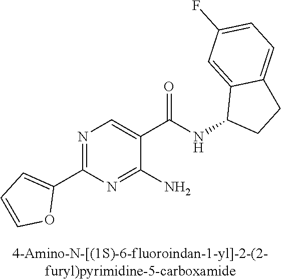 [SMILES: Nc1nc(-c2ccco2)ncc1C(=O)N[C@H]1CCc2ccc(F)cc21] | LC-MS (ARG-QC1_acidic) Rt 3.79 mins MS m/z 339.1= [M + H]+ ¹H NMR (400 MHz, DMSO) δ 8.87 (d, J = 8.0 Hz, 1H), 8.72 (s, 1H), 7.95- 7.95 (m, 2H), 7.88 (d, J = 0.9 Hz, 1H), 7.29 (dd, J = 5.2, 8.0 Hz, 1H), 7.23-7.22 (m, 1H), 7.09-7.02 (m, 2H), 6.67 (dd, J = 1.7. 3.4 Hz, 1H), 5.51 (q, J = 8.0 Hz, 1H), 2.96 (ddd, J = 2.8, 8.8, 15.5 Hz, 1H), 2.86-2.78 (m, 1H), 2.52-2.49 (m, 1H), 2.02 (ddd, J = 8.8, 12.6, 17.4 Hz, 1H). |
| 5.43 | 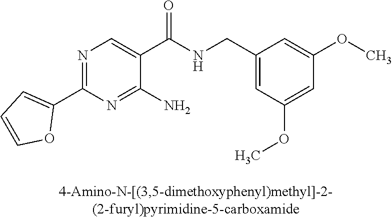 [SMILES: COc1cc(CNC(=O)c2cnc(-c3ccco3)nc2N)cc(OC)c1] | LC-MS (ARG-QC1_acidic) Rt 3.38 mins; MS m/z 355.1= [M + H]+ ¹H NMR (400 MHz, DMSO) δ 9.09 (t, J = 5.9 Hz, 1H), 8.74 (s, 1H), 8.02- 7.90 (m, 3H), 7.25 (dd, J = 0.7, 3.4 Hz, 1H), 6.68 (dd, J = 1.7, 3.4 Hz, 1H), 6.49 (d, J = 2.3 Hz, 2H), 6.39 (t, J = 2.3 Hz, 1H), 4.39 (d, J = 5.8 Hz, 2H), 3.72 (s, 6H) |
| 5.44 | 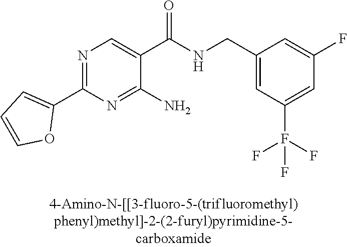 | LC-MS (CP-QC5_basic) Rt 3.37 mins; MS m/z 381.2 = [M + H]+ ¹H NMR (400 MHz, DMSO) δ 9.21 (t, J = 5.7 Hz, 1H), 8.77 (s, 1H), 7.93- 7.88 (m, 3H), 7.59-7.55 (m, 2H), 7.54-7.50 (m, 1H), 7.24 (dd, J = 0.9, 3.4 Hz, 1H), 6.67 (dd, J = 1.8, 3.4 Hz, 1H), 4.56 (d, J = 5.6 Hz, 2H) |
| 5.45 | 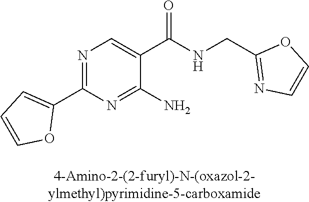 [SMILES: Nc1nc(-c2ccco2)ncc1C(=O)NCc1ncco1] | LC-MS (ARG-QC1_acidic) Rt 2.21 mins; MS m/z 286.1= [M + H]+ ¹H NMR (400 MHz, DMSO) δ 9.25 (t, J = 5.6 Hz, 1H), 8.74 (s, 1H), 8.07 (s, 2H), 7.89-7.89 (m, 2H), 7.25 (d, J = 3.4 Hz, 1H), 7.17 (s, 1H), 6.67 (dd, J = 1.8, 3.4 Hz, 1H), 4.57 (d, J = 5.7 Hz, 2H). |
| 5.47 | 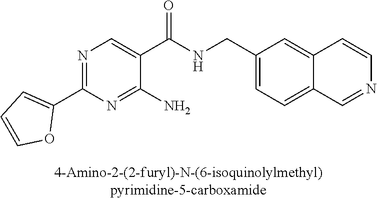 [SMILES: Nc1nc(-c2ccco2)ncc1C(=O)NCc1ccc2cnccc2c1] | LC-MS (CP-QC1_acidic) Rt 2.93 mins; MS m/z 346.2= [M + H]+ ¹H NMR (400 MHz, DMSO) δ 9.32- 9.25 (m, 2H), 8.82 (s, 1H), 8.48 (d, J = 5.6 Hz, 1H), 8.04-7.88 (m, 5H), 7.82-7.76 (m, 2H), 7.24 (dd, J = 0.9, 3.4 Hz, 1H), 6.67 (dd, J = 1.7, 3.4 Hz, 1H), 4.68 (d, J = 5.6 Hz, 2H) |
| 5.48 | 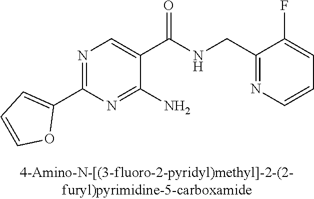 [SMILES: Nc1nc(-c2ccco2)ncc1C(=O)NCc1ncccc1F] | LC-MS (CP-QC1_acidic) Rt 2.54 mins; MS m/z 314.2 = [M + H]+ ¹H NMR (400 MHz, DMSO) δ 9.11 (t, J = 5.6 Hz, 1H), 8.75 (s, 1H), 8.39 (td, J = 1.5, 4.6 Hz, 1H), 7.91-7.87 (m, 3H), 7.71 (ddd, J = 1.3, 8.4, 10.3 Hz, 1H), 7.44-7.39 (m, 1H), 7.23 (dd, J = 0.8, 3.4 Hz, 1H), 6.67 (dd, J = 1.8, 3.4 Hz, 1H), 4.63 (dd, J = 1.4, 5.7 Hz, 2H) |
| 5.49 | 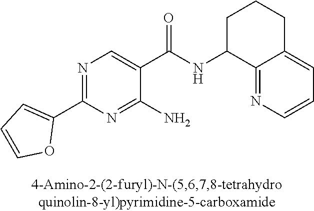 [SMILES: Nc1nc(-c2ccco2)ncc1C(=O)NC1CCCc2cccnc21] | LC-MS (CP-QC5_basic) Rt 2.93 mins; MS m/z 336.3 = [M + H]+ ¹H NMR (400 MHz, DMSO) δ 8.81 (d, J = 8.0 Hz, 1H), 8.66 (s, 1H), 8.39 (dd, J = 1.7, 4.6 Hz, 1H), 7.95-7.87 (m, 3H), 7.55 (dd, J = 1.5, 7.7 Hz, 1H), 7.24-7.20 (m, 2H), 6.66 (dd, J = 1.7, 3.4 Hz, 1H), 5.20-5.14 (m, 1H), 2.88- 2.75 (m, 2H), 2.10-2.03 (m, 1H), 1.99-1.75 (m, 3H) |
| 5.50 | 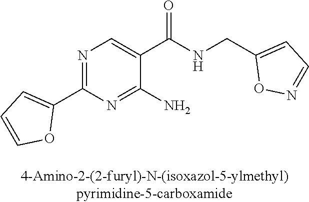 [SMILES: Nc1nc(-c2ccco2)ncc1C(=O)NCc1ccno1] | LC-MS (CP-QC1_acidic) Rt 2.47 mins; MS m/z 286.1 = [M + H]+ ¹H NMR (400 MHz, DMSO) δ 9.24 (t, J = 5.8 Hz, 1H), 8.74 (s, 1H), 8.50 (d, J = 1.8 Hz, 1H), 7.94-7.88 (m, 3H), 7.24 (dd, J = 0.9, 3.4 Hz, 1H), 6.67 (dd, J = 1.8, 3.4 Hz, 1H), 6.42-6.40 (m, 1H), 4.61 (d, J = 5.7 Hz, 2H) |
| 5.52 | 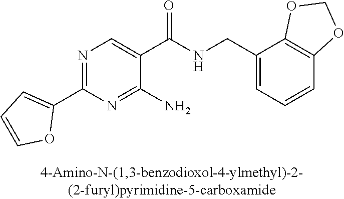 [SMILES: Nc1nc(-c2ccco2)ncc1C(=O)NCc1cccc2c1OCO2] | LC-MS (ARG-QC1_acidic) Rt 3.26 mins; MS m/z 339.1 = [M + H]+ ¹H NMR (400 MHz, DMSO) δ 9.05 (t, J = 5.7 Hz, 1H), 8.74 (s, 1H), 7.92- 7.87 (m, 3H), 7.23 (dd, J = 0.8, 3.4 Hz, 1H), 6.84-6.80 (m, 3H), 6.67 (dd, J = 1.8, 3.4 Hz, 1H), 6.03 (s, 2H), 4.42 (d, J = 5.7 Hz, 2H) |
| 5.53 | 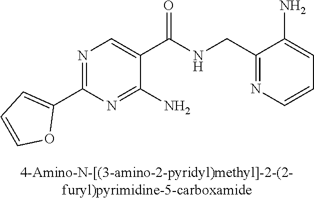 [SMILES: Nc1cccnc1CNC(=O)c1cnc(-c2ccco2)nc1N] | LC-MS (ARG-QC1_acidic) Rt 1.76 mins; MS m/z 311.1 = [M + H]+ ¹H NMR (400 MHz, DMSO) δ 9.21 (t, J = 5.2 Hz, 1H), 8.77 (s, 1H), 8.16 (s, 2H), 7.98-7.95 (m, 2H), 7.68-7.64 (m, 2H), 7.32 (d, J = 3.4 Hz, 1H), 6.72 (dd, J = 1.7, 3.4 Hz, 1H), 6.49 (s, 2H), 4.57 (d, J = 5.2 Hz, 2H) |
| 5.55 | 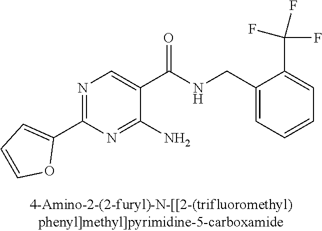 [SMILES: Nc1nc(-c2ccco2)ncc1C(=O)NCc1ccccc1C(F)(F)F] | LC-MS (ARG-QC1_acidic) Rt 3.94min MS m/z 363.1= [M + H]+ ¹H NMR (400 MHz, DMSO) δ 9.19 (t, J = 5.6 Hz, 1H), 8.81 (s, 2H), 7.90- 7.89 (m, 2H), 7.75 (d, J = 7.7 Hz, 1H), 7.68 (dd, J = 7.6, 7.6 Hz, 1H), 7.57 (d, J = 7.7 Hz, 1H), 7.49 (dd, J = 7.6, 7.6 Hz, 1H), 7.25 (d, J = 2.8 Hz, 1H), 6.68 (dd, J = 1.7, 3.4 Hz, 1H), 4.65 (d, J = 5.5 Hz, 2H). |
| 5.56 | 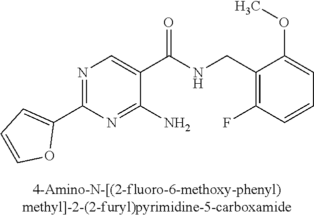 [SMILES: COc1cccc(F)c1CNC(=O)c1cnc(-c2ccco2)nc1N] | LC-MS (CP-QC1_acidic) Rt 3.02 mins; MS m/z 343.2 [M + H]+; ¹H NMR (400 MHz, DMSO) δ 8.67- 8.62 (m, 2H), 7.90-7.86 (m, 3H), 7.36-7.29 (m, 1H), 7.21 (dd, J = 0.9, 3.4 Hz, 1H), 6.89 (d, J = 8.4 Hz, 1H), 6.84-6.78 (m, 1H), 6.66 (dd, J = 1.8, 3.4 Hz, 1H), 4.46 (d, J = 4.5 Hz, 2H), 3.84 (s, 3H) |
| 5.57 | 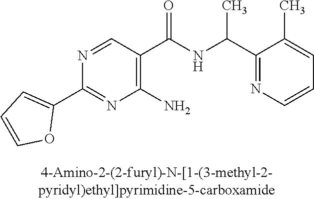 [SMILES: Cc1cccnc1C(C)NC(=O)c1cnc(-c2ccco2)nc1N] | LC-MS (Method 8A): Rt 2.46 mins; MS m/z 324.2 = [M + H]+ 1H NMR (500 MHz, DMSO-d6) δ 8.87 (d, J = 7.5 Hz, 1H), 8.76 (s, 1H), 8.39 (dd, J = 4.6, 1.7 Hz, 1H), 7.88 (dd, J = 1.7, 0.9 Hz, 1H), 7.86 (br. s, 2H), 7.57 (ddd, J = 7.6, 1.7, 0.9 Hz, 1H), 7.24-7.16 (m, 2H), 6.66 (dd, J = 3.5, 1.7 Hz, 1H), 5.37 (dq, J = 7.5, 6.8 Hz, 1H), 2.38 (s, 3H), 1.45 (d, J = 6.8 Hz, 3H). |
| 5.58 | 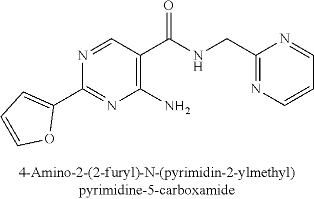 [SMILES: Nc1nc(-c2ccco2)ncc1C(=O)NCc1ncccn1] | LC-MS (Method 8B): Rt 2.68/2.72 mins; MS m/z 297.1 = [M + H]+ 1H NMR (500 MHz, DMSO-d6) δ 9.20 (t, J = 5.8 Hz, 1H), 8.80 (s, 1H), 8.78 (d, J = 4.9 Hz, 2H), 7.93 (s, 2H), 7.89 (dd, J = 1.8, 0.9 Hz, 1H), 7.41 (t, J = 4.9 Hz, 1H), 7.24 (dd, J = 3.4, 0.9 Hz, 1H), 6.67 (dd, J = 3.4, 1.8 Hz, 1H), 4.64 (d, J = 5.8 Hz, 2H). |
| 5.59 | 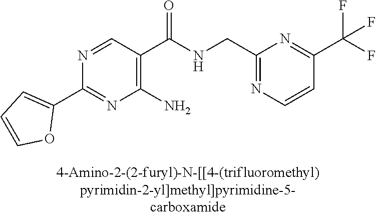 [SMILES: Nc1nc(-c2ccco2)ncc1C(=O)NCc1nccc(C(F)(F)F)n1] | LC-MS (Method 8B): Rt 3.62 mins; MS m/z 365.2 = [M + H]+ 1H NMR (500 MHz, DMSO-d6) δ 9.33 (t, J = 5.8 Hz, 1H), 9.18 (d, J = 5.1 Hz, 1H), 8.79 (s, 1H), 7.94 (d, J = 5.1 Hz, 1H), 7.89 (dd, J = 1.8, 0.9 Hz, 1H), 7.88 (s, 2H), 7.25 (dd, J = 3.4, 0.9 Hz, 1H), 6.68 (dd, J = 3.5, 1.8 Hz, 1H), 4.74 (d, J = 5.8 Hz, 2H). |
| 5.60 | 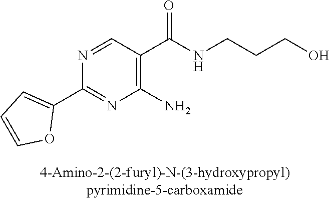 [SMILES: Nc1nc(-c2ccco2)ncc1C(=O)NCCCO] | LCMS (Method 8B): Rt 2.33, 2.38 mins; MS m/z 263.2 = [M + H]+ 1H NMR (500 MHz, DMSO-d6) δ 8.65 (s, 1H), 8.54 (t, J = 5.5 Hz, 1H), 7.93 (br.s, 2H) 7.88 (dd, J = 1.8, 0.9 Hz, 1H), 7.21 (dd, J = 3.4, 0.9 Hz, 1H), 6.66 (dd, J = 3.4, 1.8 Hz, 1H), 4.49 (t, J = 5.0 Hz, 1H), 3.50-3.42 (m, 2H), 3.32-3.26 (m, 2H), 1.72- 1.62 (m, 2H). |
| 5.61 | 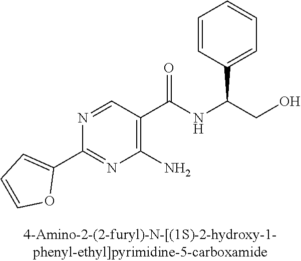 [SMILES: Nc1nc(-c2ccco2)ncc1C(=O)N[C@H](CO)c1ccccc1] | LC-MS (Method 8B): Rt 3.37 mins; MS m/z 325.3 = [M + H]+ 1H NMR (500 MHz, DMSO-d6) δ 8.85 (s, 1H), 8.80 (d, J = 8.0 Hz, 1H), 7.89 (dd, J = 1.7, 0.9 Hz, 1H), 7.84 (br s, 2H), 7.40-7.37 (m, 2H), 7.35- 7.30 (m, 2H), 7.25 (dt, J = 8.0, 1.7 Hz, 1H), 7.23 (dd, J = 3.4, 0.9 Hz, 1H), 6.67 (dd, J = 3.4, 1.7 Hz, 1H), 5.04 (td, J = 8.0, 5.4 Hz, 1H), 4.99 (t, J = 5.9 Hz, 1H), 3.73-3.67 (m, 1H), 3.66-3.61 (m, 1H) |

### Comparative Example 6

### 5-Amino-3-(3-fluorophenyl)-1,2,4-triazine-6-carboxamide

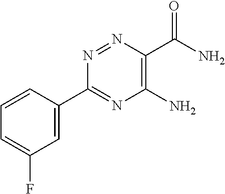 [SMILES: NC(=O)c1nnc(-c2cccc(F)c2)nc1N]

### Step 1: Ethyl 3-fluorobenzenecarboximidate

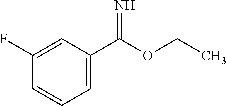

The titled compound was prepared from 3-fluorobenzonitrile according to the procedure of Ningning L, Zhengkai C, Yue L, Zhanxiang L, Yuhong Z, (Org. Lett., 2017, 19 (10), pp 2588-2591).

To a solution of commercially available 3-fluorobenzonitrile (883 μL, 8.26 mmol) in EtOH (5 mL) at 0° C. was added acetyl chloride (5.87 mL, 82.57 mmol) dropwise over 20 min and the resulting mixture was warmed to room temperature and stirred for 3.5 h. The solvent was removed in vacuo, the crude material suspended in Et₂O and the solid collected by filtration. The solid was partitioned between EtOAc (20 mL) and NaHCO₃ (20 mL). The organic portion was separated and the aqueous further extracted with EtOAc (2×10 mL). The combined organic extracts were dried over MgSO₄ and concentrated in vacuo to afford the titled compound as a clear oil.

LC-MS (Method 3B): Rt 1.59 mins; MS m/z 168.1=[M+H]+

1H NMR (500 MHz, Chloroform-d) δ 7.70 (br s, 1H), 7.53 (br d, J=7.8 Hz, 1H), 7.46 (br d, J=9.8 Hz, 1H), 7.38 (dt, J=8.0, 5.7 Hz, 1H), 7.15 (tdd, J=8.2, 2.6, 0.9 Hz, 1H), 4.31 (q, J=7.1 Hz, 2H), 1.42 (t, J=7.1 Hz, 3H).

### Step 2: N-Amino-3-fluoro-benzamidine

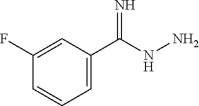 [SMILES: N=C(NN)c1cccc(F)c1]

To a solution of ethyl 3-fluorobenzenecarboximidate (step 1) (118 mg, 0.56 mmol) in EtOH (1 mL) was added hydrazine hydrate (30 μL, 0.62 mmol) and the reaction mixture was stirred for 3 h. Additional hydrazine hydrate (8 μL, 0.17 mmol) was added and the mixture stirred for 1.5 h. The solvent was removed in vacuo to afford the titled compound as a yellow semi solid.

LC-MS (Method 3B): Rt 0.85 mins; MS m/z 154.2=[M+H]+

1H NMR (500 MHz, Chloroform-d) δ 7.42-7.32 (m, 3H), 7.08 (tdd, J=8.3, 2.6, 1.1 Hz, 1H), 4.62 (br s, 2H), 3.81 (br s, 2H).

### Step 3: Ethyl 3-(3-fluorophenyl)-5-hydroxy-1,2,4-triazine-6-carboxylate

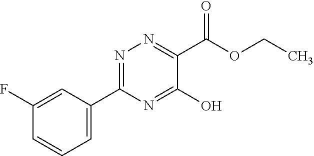 [SMILES: CCOC(=O)c1nnc(-c2cccc(F)c2)nc1O]

N-Amino-3-fluoro-benzamidine (step 2) (85 mg, 0.55 mmol) and diethyl 2-oxopropanedioate (93 μL, 0.61 mmol) were stirred in toluene (2 mL) for 2 h and then heated to reflux for 16 h. On cooling, a precipitate formed which was collected by filtration to afford the the titled compound as the major component of the crude reaction mixture as a pale yellow solid.

LC-MS (Method 3A): Rt 1.31 mins; MS m/z 264.0=[M+H]+

¹H NMR (500 MHz, DMSO-d₆) δ 14.51 (br s, 1H), 7.92 (ddd, J=8.0, 1.7, 0.9 Hz, 1H), 7.86 (ddd, J=9.9, 2.7, 1.7 Hz, 1H), 7.68 (dt, J=8.0, 5.8 Hz, 1H), 7.56 (tdd, J=8.5, 2.7, 0.9 Hz, 1H), 4.35 (q, J=7.1 Hz, 2H), 1.30 (t, J=7.1 Hz, 3H).

### Step 4: Ethyl 5-amino-3-(3-fluorophenyl)-1,2,4-triazine-6-carboxylate and 5-amino-3-(3-fluorophenyl)-1,2,4-triazine-6-carboxamide

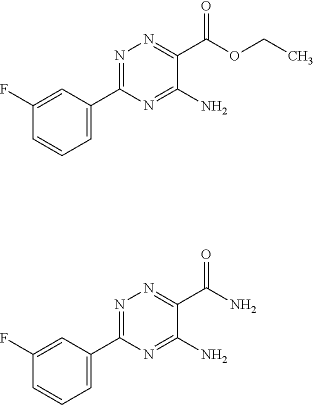 [SMILES: CCOC(=O)c1nnc(-c2cccc(F)c2)nc1N ; NC(=O)c1nnc(-c2cccc(F)c2)nc1N]

A solution of ethyl 3-(3-fluorophenyl)-5-hydroxy-1,2,4-triazine-6-carboxylate (step 3)(50 mg, 0.19 mmol) in POCl₃ (0.89 mL, 9.5 mmol) was heated to 110° C. for 3 h and allowed to cool to room temperature. The solvent was removed in vacuo and the crude product was dissolved in 1,4-dioxane (2 mL) and cooled to 0° C. NH₄OH (1 mL) was added dropwise and the resulting mixture allowed to warm to room temperature over 1 h. The mixture was partitioned between EtOAc (10 mL) and H₂O (10 mL), the organics separated and the aqueous further extracted with EtOAc (2×10 mL). The combined organic extracts were washed with brine, dried over MgSO₄ and concentrated in vacuo. Purification by column chromatography on silica eluting with 3% MeOH in DCM afforded 2 products:

### Product 4(i) Ethyl 5-amino-3-(3-fluorophenyl)-1,2,4-triazine-6-carboxylate

Yellow Solid

LC-MS (Method 3B): Rt 1.57 mins; MS m/z 263.2=[M+H]+

1H NMR (500 MHz, Chloroform-d) δ 8.32 (ddd, J=7.8, 1.5, 1.0 Hz, 1H), 8.20 (ddd, J=10.1, 2.7, 1.5 Hz, 1H), 7.95 (br s, 1H), 7.48 (dt, J=8.2, 5.7 Hz, 1H), 7.24 (tdd, J=8.2, 2.7, 1.0 Hz, 1H—overlapping with solvent peak), 5.71 (br s, 1H), 4.53 (q, J=7.1 Hz, 2H), 1.49 (t, J=7.1 Hz, 3H).

### Product 4(i): 5-Amino-3-(3-fluorophenyl)-1,2,4-triazine-6-carboxamide

Cream Solid.

LC-MS (Method 8B): Rt 3.36 mins; MS m/z 234.1=[M+H]+

1H NMR (500 MHz, DMSO-d6) δ 8.58 (br s, 1H), 8.53-8.47 (br m, 1H), 8.45-8.38 (br m, 1H), 8.23-8.19 (m, 1H), 8.06 (ddd, J=10.4, 2.8, 1.5 Hz, 1H), 7.92 (br s, 1H), 7.62 (dt, J=8.1, 6.0 Hz, 1H), 7.45 (tdd, J=8.5, 2.8, 1.0 Hz, 1H).

### Example 7

### 5-Amino-3-(3-fluorophenyl)-N-[(3-methyl-2-pyridyl)methyl]-1,2,4-triazine-6-carboxamide

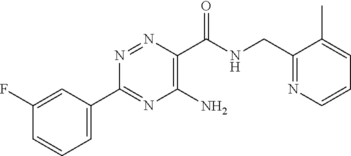 [SMILES: Cc1cccnc1CNC(=O)c1nnc(-c2cccc(F)c2)nc1N]

### Step 1: 5-Amino-3-(3-fluorophenyl)-1,2,4-triazine-6-carboxylic Acid

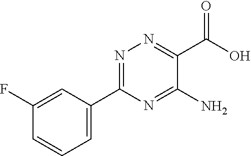 [SMILES: Nc1nc(-c2cccc(F)c2)nnc1C(=O)O]

To a solution of ethyl 5-amino-3-(3-fluorophenyl)-1,2,4-triazine-6-carboxylate (Ex 6, product 4(i)) (33 mg, 0.13 mmol) in THF (2 mL) was added a solution of LiOH (4 mg, 0.15 mmol) in water (2 mL) and the mixture was stirred for 1 hour. The solvent was removed in vacuo, the crude material re-dissolved in H₂O (10 mL) and extracted with Et₂O (15 mL). The organic portion was discarded and the aqueous layer acidified with 2M HCl and extracted with EtOAc (3×15 mL). The combined organic extracts were dried over MgSO₄ and the solvent removed in vacuo to afford the titled compound as a pale yellow solid.

LC-MS (Method 3A): Rt 1.28 mins; MS m/z 235.2=[M+H]+

1H NMR (500 MHz, Methanol-d4) δ 8.26 (ddd, J=7.8, 1.4, 1.1 Hz, 1H), 8.13 (ddd, J=10.2, 2.7, 1.4 Hz, 1H), 7.56 (dt, J=8.2, 5.7 Hz, 1H), 7.33 (tdd, J=8.2, 2.7, 1.1 Hz, 1H).

### Step 2: 5-Amino-3-(3-fluorophenyl)-N-[(3-methyl-2-pyridyl)methyl]-1,2,4-triazine-6-carboxamide

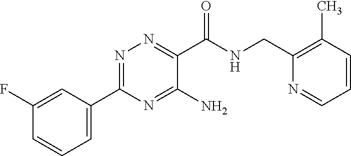 [SMILES: Cc1cccnc1CNC(=O)c1nnc(-c2cccc(F)c2)nc1N]

To a solution of 5-amino-3-(3-fluorophenyl)-1,2,4-triazine-6-carboxylic acid (step 1)(18 mg, 0.08 mmol) in DMF (1 mL) was added commercially available (3-methyl-2-pyridyl)methanamine (14 μL, 0.12 mmol), HATU (58 mg, 0.15 mmol) and DIPEA (67 μL, 0.38 mmol) and the mixture was stirred at room temperature for 1 hour. The mixture was partitioned between EtOAc (10 mL) and H₂O (10 mL), the organic separated and the aqueous further extracted with EtOAc (2×10 mL). The combined organic extracts were washed with brine, dried over MgSO₄ and the solvent removed in vacuo. Purification by column chromatography on silica eluting with 20% EtOAc in hexane afforded a yellow solid. The material was suspended in Et₂O and filtered to afford the titled compound as a yellow solid.

LC-MS (Method 8B): Rt 4.53 mins; MS m/z 339.2=[M+H]+

1H NMR (500 MHz, Chloroform-d) δ 9.69 (br t, J=4.4 Hz, 1H), 8.74 (br s, 1H), 8.46 (dd, J=4.8, 1.0 Hz, 1H), 8.34-8.27 (m, 1H), 8.19 (ddd, J=10.2, 2.7, 1.5 Hz, 1H), 7.53-7.44 (m, 2H), 7.24 (tdd, J=8.3, 2.7, 1.0 Hz, 1H), 7.17 (dd, J=7.6, 4.8 Hz, 1H), 5.70 (br s, 1H), 4.72 (d, J=4.4 Hz, 2H), 2.36 (s, 3H).

### Example 8

### 5-Amino-3-(4-fluorophenyl)-N-[(3-methyl-2-pyridyl)methyl]-1,2,4-triazine-6-carboxamide

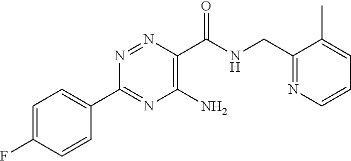 [SMILES: Cc1cccnc1CNC(=O)c1nnc(-c2ccc(F)cc2)nc1N]

### Step 1: Ethyl 5-amino-3-(4-fluorophenyl)-1,2,4-triazine-6-carboxylate

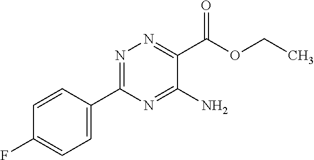 [SMILES: CCOC(=O)c1nnc(-c2ccc(F)cc2)nc1N]

To a degassed solution of ethyl 5-amino-3-methylsulfanyl-1,2,4-triazine-6-carboxylate (Example 1 step 2)(150 mg, 0.7 mmol) in THF (5 mL) was added commercially available 4-fluorophenylboronic acid (147 mg, 1.05 mmol), copper(I) thiophene-2-carboxylate (267 mg, 1.4 mmol) and Pd(PPh₃)₄ (40 mg, 0.04 mmol). The resulting mixture was heated to reflux for 18.5 h. The mixture was diluted with EtOAc (10 mL) and filtered through a pad of Celite® (filter material). The organic solution was washed with 1M NaHSO₄ solution (50 mL), sat. NaHCO₃ (50 mL), brine, dried over MgSO₄ and the solvent removed in vacuo. Purification by column chromatography on silica eluting with a gradient of 25 to 40% EtOAc in petrol afforded a solid which was suspended in Et₂O, filtered and dried to afford the titled compound as a yellow solid.

LC-MS (Method 3B): Rt 1.48 mins; MS m/z 263.2=[M+H]+

1H NMR (500 MHz, Chloroform-d) δ 8.53 (dd, J=8.4, 5.4 Hz, 2H), 7.94 (br s, 1H), 7.19 (apr t, J=8.4 Hz, 2H), 5.72 (br s, 1H), 4.53 (q, J=7.1 Hz, 2H), 1.49 (t, J=7.1 Hz, 3H).

### Step 2: 5-Amino-3-(4-fluorophenyl)-1,2,4-triazine-6-carboxylic Acid

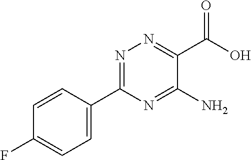 [SMILES: Nc1nc(-c2ccc(F)cc2)nnc1C(=O)O]

To a solution of ethyl 5-amino-3-(4-fluorophenyl)-1,2,4-triazine-6-carboxylate (step 1) (45 mg, 0.17 mmol) in THF (2 mL) was added a solution of LiOH (5 mg, 0.21 mmol) in water (2 mL) and the mixture was stirred for 1 hour. A drop of 2M HCl was added to neutralize the mixture and the solvent was removed in vacuo to afford the titled compound as a white solid.

LC-MS (Method 3A): Rt 1.19 mins; MS m/z 235.2=[M+H]+

The material was taken into the coupling reaction without further analysis or purification.

### Step 3: 5-Amino-3-(4-fluorophenyl)-N-[(3-methyl-2-pyridyl)methyl]-1,2,4-triazine-6-carboxamide

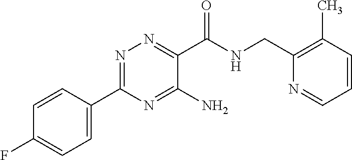 [SMILES: Cc1cccnc1CNC(=O)c1nnc(-c2ccc(F)cc2)nc1N]

The titled compound was prepared from 5-amino-3-(4-fluorophenyl)-1,2,4-triazine-6-carboxylic acid (step 2) and commercially available (3-methyl-2-pyridyl)methanamine analogously to Example 2, step 2.

LC-MS (Method 8B): Rt 4.47 mins; MS m/z 339.1=[M+H]+

1H NMR (500 MHz, Chloroform-d) δ 9.65 (br t, J=4.6 Hz, 1H), 8.68 (br s, 1H), 8.53-8.48 (m, 2H), 8.47 (dd, J=5.2, 1.6 Hz, 1H), 7.54 (d, J=7.6 Hz, 1H), 7.23-7.16 (m, 3H), 5.67 (br s, 1H), 4.74 (d, J=4.6 Hz, 2H), 2.38 (s, 3H).

### Example 9

### 4-Amino-2-(2-fluorophenyl)-N-[(3-methyl-2-pyridyl)methyl]pyrimidine-5-carboxamide

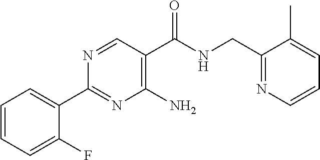 [SMILES: Cc1cccnc1CNC(=O)c1cnc(-c2ccccc2F)nc1N]

### Step 1: Ethyl 4-amino-2-(2-fluorophenyl)pyrimidine-5-carboxylate

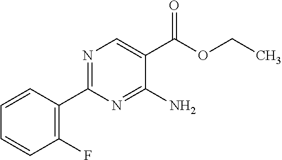 [SMILES: CCOC(=O)c1cnc(-c2ccccc2F)nc1N]

A solution of commercially available ethyl 4-amino-2-chloro-pyrimidine-5-carboxylate (500 mg, 2.48 mmol) and potassium carbonate (686 mg, 4.96 mmol) in a mixture of 1,4-dioxane (10 mL) and water (2 mL) was de-gassed via nitrogen sparging for 10 mins. Pd(dppf)Cl₂ CH₂Cl₂ (203 mg, 0.25 mmol) was added followed by commercially available (2-fluorophenyl)boronic acid (521 mg, 3.72 mmol) and placed under an atmosphere of nitrogen. The resulting mixture was stirred at 100° C. for 2.75 hours. The mixture was allowed to cool, partitioned between DCM (50 mL) and water (50 mL), the layers separated and the aqueous portion further extracted with DCM (50 mL). The combined organic portions were dried over MgSO₄ and the solvent removed in vacuo. Purification by column chromatography on silica eluting with a gradient of 10 to 30% EtOAc in petrol afforded the titled compound as a cream solid.

LC-MS (Method 3B): Rt 1.62 mins; MS m/z 262.1=[M+H]+

1H NMR (500 MHz, Chloroform-d) δ 9.03 (s, 1H), 8.00 (td, J=7.8, 1.8 Hz, 1H), 7.89 (s, 1H), 7.44 (dddd, J=8.3, 7.4, 4.9, 1.9 Hz, 1H), 7.24 (td, J=7.6, 1.2 Hz, 1H), 7.17 (ddd, J=11.2, 8.3, 1.1 Hz, 1H), 5.78 (s, 1H), 4.40 (q, J=7.1 Hz, 2H), 1.41 (t, J=7.1 Hz, 3H)

### Step 2: 4-Amino-2-(2-fluorophenyl)pyrimidine-5-carboxylic Acid

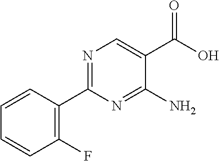 [SMILES: Nc1nc(-c2ccccc2F)ncc1C(=O)O]

The titled compound was prepared from ethyl 4-amino-2-(2-fluorophenyl) pyrimidine-5-carboxylate (step 1) and LiOH analogously to Example 3 step 2.

LC-MS (Method 3A): Rt 0.75 mins; MS m/z 234.1=[M+H]+

1H NMR (500 MHz, DMSO-d6) δ 9.05 (s, 1H), 8.82 (s, 1H), 8.49 (s, 1H), 7.92 (td, J=7.7, 1.9 Hz, 1H), 7.71-7.62 (m, 1H), 7.45-7.36 (m, 2H). Acid proton not observed.

### Step 3: 4-Amino-2-(2-fluorophenyl)-N-[(3-methyl-2-pyridyl)methyl]pyrimidine-5-carboxamide

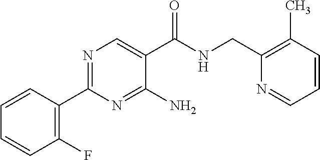 [SMILES: Cc1cccnc1CNC(=O)c1cnc(-c2ccccc2F)nc1N]

The titled compound was prepared from 4-amino-2-(2-fluorophenyl)pyrimidine-5-carboxylic acid (step 2) and commercially available (3-methyl-2-pyridyl)methanamine analogously to Example 3 step 3.

LC-MS (Method 8B): Rt 3.70 mins; MS m/z 338.1=[M+H]+

1H NMR (500 MHz, Chloroform-d) δ 8.88 (s, 1H), 8.52 (br t, J=4.0 Hz, 1H), 8.43-8.37 (m, 1H), 8.02 (td, J=7.8, 1.8 Hz, 1H), 7.52 (ddd, J=7.6, 1.5, 0.7 Hz, 1H), 7.48-7.40 (m, 1H), 7.24 (td, J=7.7, 1.3 Hz, 1H), 7.24-7.14 (m, 2H), 7.01 (br s, 2H), 4.63 (d, J=3.8 Hz, 2H), 2.33 (s, 3H)

### Example 10

### 4-Amino-2-(3-fluorophenyl)-N-(p-tolylmethyl)pyrimidine-5-carboxamide

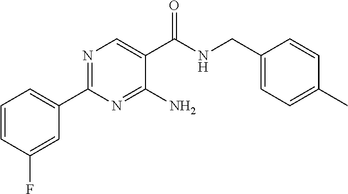 [SMILES: Cc1ccc(CNC(=O)c2cnc(-c3cccc(F)c3)nc2N)cc1]

To a suspension of 4-amino-2-(3-fluorophenyl)pyrimidine-5-carboxylic acid (Example 5 step 1) (76 mg, 0.33 mmol) in DCM (3 mL) at room temperature was added triethylamine (68 μL, 0.49 mmol), p-tolylmethanamine (46 μL, 0.36 mmol) and T3P® (propylphosphonic anhydride solution 50% in EtOAc) (291 μL, 0.49 mmol) and the resulting mixture was stirred at room temperature for 1 h 45 min. The mixture was diluted with DCM and washed with H₂O. The organic portion was passed through a phase separator and concentrated in vacuo. Purification of the crude residue by mass directed HPLC afforded the titled compound as a white solid.

LC-MS (Method acidic): Rt 3.45 min; MS m/z 337.3=[M+H]+

¹H NMR (400 MHz, DMSO) δ 9.15 (t, J=5.8 Hz, 1H), 8.83 (s, 1H), 8.18 (ddd, J=1.2, 1.2, 7.8 Hz, 1H), 8.06-7.94 (m, 3H), 7.55 (ddd, J=8.0, 8.0, 6.0 Hz, 1H), 7.40-7.34 (m, 1H), 7.23 (d, J=8.1 Hz, 2H), 7.15 (d, J=7.7 Hz, 2H), 4.43 (d, J=5.8 Hz, 2H), 2.28 (s, 3H) The compounds of the following tabulated Examples (Table 3) were prepared analogously to Example 10 from 4-amino-2-(3-fluorophenyl)pyrimidine-5-carboxylic acid (Example 5 step 1) and the appropriate commercially available amine.

TABLE 3

| Ex. | Structure and Name | Retention Time, [M + H]+, 1H NMR |
|---|---|---|
| 10.1 | 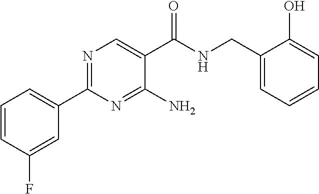 [SMILES: Nc1nc(-c2cccc(F)c2)ncc1C(=O)NCc1ccccc1O] | LCMS (ARG-QC1_acidic) Rt 4.10 min; MS m/z 339.2 [M + H]+ ¹H NMR (400 MHz, DMSO) δ 9.55 (s, 1H), 9.02 (t, J = 5.6 Hz, 1H), 8.86 (s, 1H), 8.18 (ddd, J = 1.2, 1.2, 7.9 Hz, 1H), 8.06-7.93 (m, 3H), 7.56 (ddd, J = 8.0, 8.0, 6.0 Hz, 1H), 7.40-7.34 (m, 1H), 7.15 (dd, J = 1.5, 7.5 Hz, 1H), 7.08 (dt, J = 1.5, 7.7 Hz, 1H), 6.82 (dd, J = 1.1, 8.0 Hz, 1H), 6.77 (dt, J = 1.1, 7.4 Hz, 1H), 4.42 (d, J = 5.4 Hz, 2H) |
| | 4-Amino-2-(3-fluorophenyl)-N-[(2- | |
| | hydroxyphenyl)methyl]pyrimidine-5- | |
| | carboxamide | |
| 10.2 | 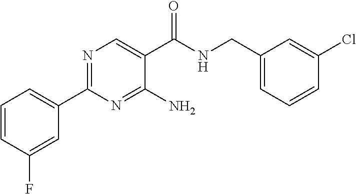 [SMILES: Nc1nc(-c2cccc(F)c2)ncc1C(=O)NCc1cccc(Cl)c1] | LCMS (CP-QC1_acidic): Rt 3.49 min; MS m/z 357.2/359.2 [M + H]+ ¹H NMR (400 MHz, DMSO) δ 9.23 (t, J = 5.8 Hz, 1H), 8.85 (s, 1H), 8.18 (ddd, J = 1.2, 1.2, 7.9 Hz, 1H), 8.06- 7.94 (m, 3H), 7.56 (ddd, J = 8.0, 8.0, 6.1 Hz, 1H), 7.41-7.29 (m, 5H), 4.48 (d, J = 5.8 Hz, 2H) |
| | 4-Amino-N-[(3-chlorophenyl)methyl]-2-(3- | |
| | fluorophenyl)pyrimidine-5-carboxamide | |
| 10.3 | 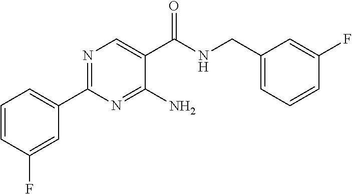 [SMILES: Nc1nc(-c2cccc(F)c2)ncc1C(=O)NCc1cccc(F)c1] | LCMS (CP-QC5_basic) Rt 3.42 min; MS m/z 341.3 [M + H]+ ¹H NMR (400 MHz, DMSO) δ 9.22 (t, J = 5.9 Hz, 1H), 8.85 (s, 1H), 8.18 (ddd, J = 1.1, 1.1, 7.9 Hz, 1H), 8.07- 7.94 (m, 3H), 7.56 (ddd, J = 8.0, 8.0, 6.0 Hz, 1H), 7.42-7.34 (m, 2H), 7.20-7.14 (m, 2H), 7.12-7.05 (m, 1H), 4.50 (d, J = 5.9 Hz, 2H) |
| | 4-Amino-2-(3-fluorophenyl)-N-[(3- | |
| | fluorophenyl)methyl]pyrimidine-5- | |
| | carboxamide | |
| 10.4 | 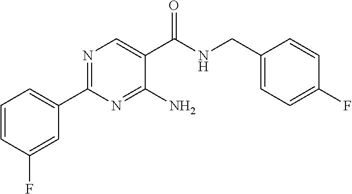 [SMILES: Nc1nc(-c2cccc(F)c2)ncc1C(=O)NCc1ccc(F)cc1] | LCMS (CP-QC1_acidic) Rt 3.35 min; MS m/z 341.3 [M + H]+ ¹H NMR (400 MHz, DMSO) δ 9.19 (t, J = 5.9 Hz, 1H), 8.83 (s, 1H), 8.18 (ddd, J = 1.2, 1.2, 7.8 Hz, 1H), 8.06- 7.94 (m, 3H), 7.55 (ddd, J = 8.1, 8.1, 6.1 Hz, 1H), 7.41-7.34 (m, 3H), 7.17 (tt, J = 2.5, 9.4 Hz, 2H), 4.46 (d, J = 5.8 Hz, 2H) |
| | 4-Amino-2-(3-fluorophenyl)-N-[(4- | |
| | fluorophenyl)methyl]pyrimidine-5- | |
| | carboxamide | |
| 10.5 | 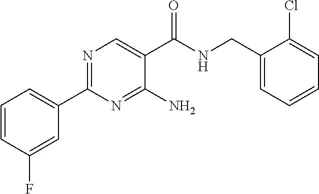 [SMILES: Nc1nc(-c2cccc(F)c2)ncc1C(=O)NCc1ccccc1Cl] | LCMS (CP-QC1_acidic) Rt 3.48 min; MS m/z 357.2/359.2 [M + H]+ ¹H NMR (400 MHz, DMSO) δ 9.20 (t, J = 5.8 Hz, 1H), 8.89 (s, 1H), 8.19 (ddd, J = 1.1, 1.1, 7.9 Hz, 1H), 8.07- 7.93 (m, 3H), 7.56 (ddd, J = 8.0, 8.0, 6.0 Hz, 1H), 7.48-7.45 (m, 1H), 7.43-7.29 (m, 4H), 4.55 (d, J = 5.7 Hz, 2H) |
| | 4-Amino-N-[(2-chlorophenyl)methyl]-2-(3- | |
| | fluorophenyl)pyrimidine-5-carboxamide | |
| 10.6 | 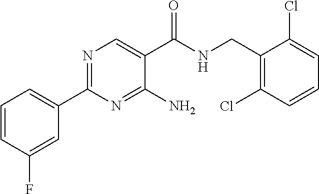 [SMILES: Nc1nc(-c2cccc(F)c2)ncc1C(=O)NCc1c(Cl)cccc1Cl] | LCMS (CP-QC1_acidic) Rt 3.58 min; MS m/z 391.2/393.2/395.2 [M + H]+ ¹H NMR (400 MHz, DMSO) δ 8.84 (t, J = 4.4 Hz, 1H), 8.73 (s, 1H), 8.16 (ddd, J = 1.2, 1.2, 7.9 Hz, 1H), 8.01 (ddd, J = 1.4, 2.7, 10.6 Hz, 1H), 7.90 (s, 2H), 7.58-7.51 (m, 3H), 7.41- (m, 2H), 4.70 (d, J = 4.4 Hz, 2H) |
| | 4-Amino-N-[(2,6-dichlorophenyl)methyl]-2-(3- | |
| | fluorophenyl)pyrimidine-5-carboxamide | |
| 10.7 | 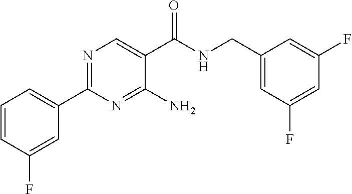 [SMILES: Nc1nc(-c2cccc(F)c2)ncc1C(=O)NCc1cc(F)cc(F)c1] | LCMS (CP-QC1_acidic) Rt 3.46 min; MS m/z 359.2 [M + H]+ ¹H NMR (400 MHz, DMSO) δ 9.22 (s, 1H), 8.86 (s, 1H), 8.19 (ddd, J = 1.2, 1.2, 7.8 Hz, 1H), 8.07-7.93 (m, 3H), 7.56 (ddd, J = 8.0, 8.0, 6.0 Hz, 1H), 7.40-7.34 (m, 1H), 7.15-7.03 (m, 3H), 4.49 (s, 2H) |
| | 4-Amino-N-[(3,5-difluorophenyl)methyl]-2-(3- | |
| | fluorophenyl)pyrimidine-5-carboxamide | |
| 10.8 | 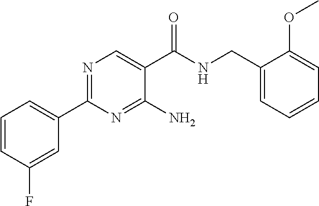 [SMILES: COc1ccccc1CNC(=O)c1cnc(-c2cccc(F)c2)nc1N] | LCMS (CP-QC5_basic) Rt 3.34 min; MS m/z 353.2 [M + H]+ ¹H NMR (400 MHz, DMSO) δ 9.00 (t, J = 5.8 Hz, 1H), 8.87 (s, 1H), 8.18 (ddd, J = 1.2, 1.2, 7.8 Hz, 1H), 8.06- 7.93 (m, 3H), 7.56 (ddd, J = 8.0, 8.0, 6.0 Hz, 1H), 7.40-7.34 (m, 1H), 7.28-7.21 (m, 2H), 7.00 (dd, J = 0.7, 8.2 Hz, 1H), 6.92 (dt, J = 1.0, 7.4 Hz, 1H), 4.45 (d, J = 5.7 Hz, 2H), 3.84 (s, 3H) |
| | 4-Amino-2-(3-fluorophenyl)-N-[(2- | |
| | methoxyphenyl)methyl]pyrimidine-5- | |
| | carboxamide | |
| 10.9 | 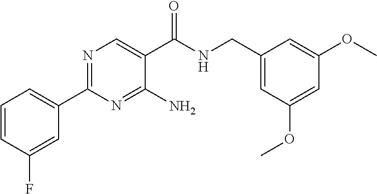 [SMILES: COc1cc(CNC(=O)c2cnc(-c3cccc(F)c3)nc2N)cc(OC)c1] | LCMS (CP-QC5_basic) Rt 3.40; MS m/z 383.3 [M + H]+ ¹H NMR (400 MHz, DMSO) δ 9.14 (t, J = 5.9 Hz, 1H), 8.84 (s, 1H), 8.18 (ddd, J = 1.2, 1.2, 7.9 Hz, 1H), 8.06- 7.94 (m, 3H), 7.56 (ddd, J = 8.1, 8.1, 6.0 Hz, 1H), 7.40-7.34 (m, 1H), 6.50 (d, J = 2.3 Hz, 2H), 6.39 (t, J = 2.3 Hz, 1H), 4.41 (d, J = 5.9 Hz, 2H), 3.73 (s, 6H) |
| | 4-Amino-N-[(3,5-dimethoxyphenyl)methyl]-2- | |
| | (3-fluorophenyl)pyrimidine-5-carboxamide | |
| 10.10 | 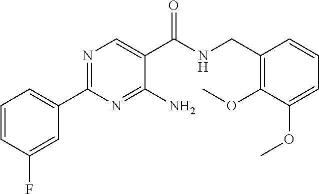 [SMILES: COc1cccc(CNC(=O)c2cnc(-c3cccc(F)c3)nc2N)c1OC] | LCMS (CP-QC5_basic) Rt 3.40; MS m/z 383.3 [M + H]+ ¹H NMR (400 MHz, DMSO) δ 9.06 (t, J = 5.7 Hz, 1H), 8.85 (s, 1H), 8.18 (ddd, J = 1.1, 1.1, 7.9 Hz, 1H), 8.06- 7.94 (m, 3H), 7.55 (ddd, J = 8.0, 8.0, 6.0 Hz, 1H), 7.40-7.34 (m, 1H), 7.04 (t, J = 7.8 Hz, 1H), 6.96 (dd, J = 1.6, 8.2 Hz, 1H), 6.88 (dd, J = 1.6, 7.6 Hz, 1H), 4.48 (d, J = 5.8 Hz, 2H), 3.81 (s, 3H), 3.78 (s, 3H) |
| | 4-Amino-N-[(2,3-dimethoxyphenyl)methyl]-2- | |
| | (3-fluorophenyl)pyrimidine-5-carboxamide | |
| 10.11 | 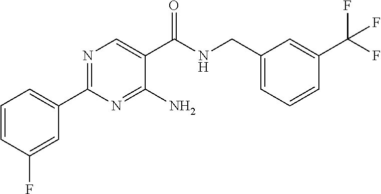 [SMILES: Nc1nc(-c2cccc(F)c2)ncc1C(=O)NCc1cccc(C(F)(F)F)c1] | LCMS (ARG-QC1_acidic) Rt 4.96 min; MS m/z 391.1 [M + H]+ ¹H NMR (400 MHz, DMSO) δ 9.26 (t, J = 6.0 Hz, 1H), 8.85 (s, 1H), 8.18 (ddd, J = 1.2, 1.2, 7.8 Hz, 1H), 8.06- 7.94 (m, 3H), 7.71-7.53 (m, 5H), 7.40-7.34 (m, 1H), 4.57 (d, J = 5.8 Hz, 2H). |
| | 4-Amino-2-(3-fluorophenyl)-N-[[3- | |
| | (trifluoromethyl)phenyl]methyl]pyrimidine-5- | |
| | carboxamide | |
| 10.12 | 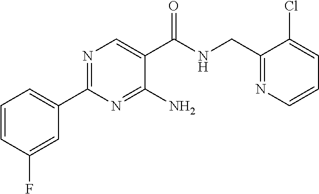 [SMILES: Nc1nc(-c2cccc(F)c2)ncc1C(=O)NCc1ncccc1Cl] | LCMS (ARG-QC1_acidic) Rt 4.07 min; MS m/z 358.1 [M + H]+ ¹H NMR (400 MHz, DMSO) δ 9.13 (t, J = 5.5 Hz, 1H), 8.86 (s, 1H), 8.51 (dd, J = 1.4, 4.7 Hz, 1H), 8.19 (ddd, J = 1.2, 1.2, 7.9 Hz, 1H), 8.04 (ddt, J = 1.5, 4.4, 5.3 Hz, 1H), 7.97-7.90 (m, 3H), 7.56 (ddd, J = 8.0, 8.0, 6.0 Hz, 1H), 7.41-7.34 (m, 2H), 4.69 (d, J = 5.6 Hz, 2H) |
| | 4-Amino-N-[(3-chloro-2-pyridyl)methyl]-2-(3- | |
| | fluorophenyl)pyrimidine-5-carboxamide | |
| 10.13 | 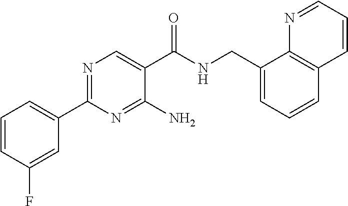 [SMILES: Nc1nc(-c2cccc(F)c2)ncc1C(=O)NCc1cccc2cccnc12] | LCMS (CP-QC5_basic) Rt 3.41 min; MS m/z 374.3 [M + H]+ ¹H NMR (400 MHz, DMSO) J = 5.8 Hz, 1H), 8.99 (dd, J = 1.8, 4.2 Hz, 1H), 8.94 (s, 1H), 8.41 (dd, J = 1.7, 8.3 Hz, 1H), 8.20 (ddd, J = 1.2, 1.2, 7.8 Hz, 1H), 8.08-7.89 (m, 4H), 7.70 (dd, J = 1.1, 7.2 Hz, 1H), 7.62- 7.53 (m, 3H), 7.41-7.34 (m, 1H), 5.14 (d, J = 5.8 Hz, 2H) |
| | 4-Amino-2-(3-fluorophenyl)-N-(8- | |
| | quinolylmethyl)pyrimidine-5-carboxamide | |
| 10.14 | 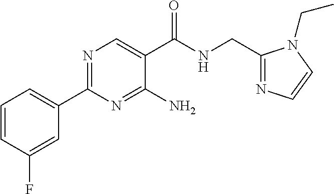 [SMILES: CCn1ccnc1CNC(=O)c1cnc(-c2cccc(F)c2)nc1N] | LCMS (CP-QC1_acidic) Rt 2.52 min; MS m/z 341.3 [M + H]+ ¹H NMR (400 MHz, DMSO) δ 9.13 (t, J = 5.6 Hz, 1H), 8.79 (s, 1H), 8.18 (ddd, J = 1.2, 1.2, 7.9 Hz, 1H), 8.06- 7.96 (m, 3H), 7.55 (ddd, J = 8.0, 8.0, 6.0 Hz, 1H), 7.40-7.34 (m, 1H), 7.18 (d, J = 1.2 Hz, 1H), 6.84 (d, J = 1.2 Hz, 1H), 4.55 (d, J = 5.6 Hz, 2H), 4.03 (q, J = 7.2 Hz, 2H), 1.30 (t, J = 7.2 Hz, 3H) |
| | 4-Amino-N-[(1-ethylimidazol-2-yl)methyl]-2- | |
| | (3-fluorophenyl)pyrimidine-5-carboxamide | |
| 10.15 | 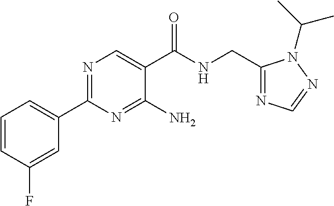 [SMILES: CC(C)n1ncnc1CNC(=O)c1cnc(-c2cccc(F)c2)nc1N] | LCMS (CP-QC5_basic) Rt 3.12 min; MS m/z 356.3 [M + H]+ ¹H NMR (400 MHz, DMSO) δ 9.29 (t, J = 5.6 Hz, 1H), 8.80 (s, 1H), 8.18 (ddd, J = 1.2, 1.2, 7.9 Hz, 1H), 8.05- 7.87 (m, 4H), 7.55 (ddd, J = 8.0, 8.0, 6.1 Hz, 1H), 7.40-7.34 (m, 1H), 4.87-4.78 (m, 1H), 4.64 (d, J = 5.5 Hz, 2H), 1.40 (d, J = 6.5 Hz, 6H). |
| | 4-Amino-2-(3-fluorophenyl)-N-[(2-isopropyl- | |
| | 1,2,4-triazol-3-yl)methyl]pyrimidine-5- | |
| | carboxamide | |

The compounds of the following tabulated Examples (Table 4) were prepared analogously to Example 10 from 4-Amino-2-(2-furyl)pyrimidine-5-carboxylic acid (Example 3 step 2) and the appropriate commercially available amine.

TABLE 4

| Ex. | Structure and Name | Retention Time, [M + H]+, 1H NMR |
|---|---|---|
| 11.1 | 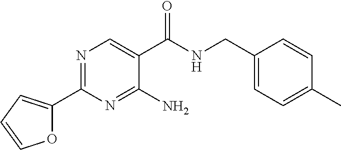 [SMILES: Cc1ccc(CNC(=O)c2cnc(-c3ccco3)nc2N)cc1] | LC-MS (CP-QC1_acidic) Rt 2.98 mins; MS m/z 309.2 [M + H]+; ¹H NMR (400 MHz, DMSO) d 9.08 (t, J = 5.9 Hz, 1H), 8.73 (s, 1H), 7.94- 7.87 (m, 3H), 7.23-7.20 (m, 3H), 7.14 (d, J = 7.9 Hz, 2H), 6.67 (dd, J = 1.7, 3.4 Hz, 1H), 4.41 (d, J = 5.9 Hz, 2H), 2.28 (s, 3H) |
| | 4-Amino-2-(2-furyl)-N-(p-tolylmethyl) | |
| | pyrimidine-5-carboxamide | |
| 11.2 | 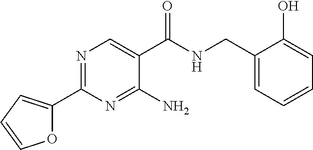 [SMILES: Nc1nc(-c2ccco2)ncc1C(=O)NCc1ccccc1O] 4-amino-2-(2-furyl)-N-[(2-hydroxyphenyl) | LC-MS (CP-QC1_acidic) Rt 2.72 mins; MS m/z 311.2 [M + H]+ ¹H NMR (400 MHz, DMSO) d 9.57 (s, 1H), 8.97 (t, J = 5.7 Hz, 1H), 8.76 (s, 1H), 7.92-7.87 (m, 3H), 7.23 (dd, J = 0.8, 3.4 Hz, 1H), 7.14 (dd, J = 1.5, 7.5 Hz, 1H), 7.07 (dt, J = 1.5, 7.7 Hz, 1H), 6.82 (dd, J = 1.0, 8.0 Hz, 1H), 6.76 (dt, J = 1.1, 7.4 Hz, 1H), 6.67 (dd, J = 1.7, 3.4 Hz, 1H), 4.40 (d, J = 5.7 Hz, 2H) |
| | methyl]pyrimidine-5-carboxamide | |

### Example 12

### 4-Amino-2-(3-cyanophenyl)-N-[(3-methyl-2-pyridyl)methyl]pyrimidine-5-carboxamide

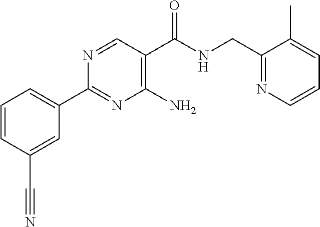 [SMILES: Cc1cccnc1CNC(=O)c1cnc(-c2cccc(C#N)c2)nc1N]

### Step 1: 4-Amino-2-chloro-pyrimidine-5-carboxylic Acid

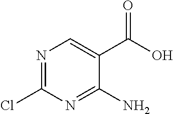 [SMILES: Nc1nc(Cl)ncc1C(=O)O]

LiOH (238 mg, 9.94 mmol) was added to a solution of commercially available ethyl 4-amino-2-chloro-pyrimidine-5-carboxylate (1 g, 4.96 mmol) in THF (20 mL) and water (20 mL) and the mixture stirred at room temperature for 30 mins. The volatile solvent was removed in vacuo and the remaining aqueous solution was washed with Et₂O (20 mL). The aqueous portion was acidified with 2M HCl and the resulting white precipitate was collected by filtration, washed with water and dried in vacuo to afford the titled compound as a white solid.

LC-MS (Method 3A): Rt 1.00 mins; MS m/z 174.0=[M+H]+

1H NMR (500 MHz, DMSO-d6) δ 13.55 (s, 1H), 8.58 (s, 1H), 8.47 (s, 1H), 7.98 (s, 1H).

### Step 2:4-Amino-2-chloro-N-[(3-methyl-2-pyridyl)methyl]pyrimidine-5-carboxamide

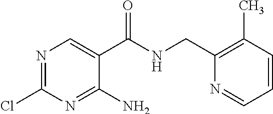 [SMILES: Cc1cccnc1CNC(=O)c1cnc(Cl)nc1N]

To a solution of 4-amino-2-chloro-pyrimidine-5-carboxylic acid (step 1) (400 mg, 2.3 mmol) in DMF (11.5 mL) was added commercially available (3-methyl-2-pyridyl)methanamine (411 μL, 3.45 mmol), HATU (1753 mg, 4.61 mmol) and DIPEA (2 mL, 11.48 mmol) and the mixture stirred at room temperature for 16.25 hours. The resulting mixture was diluted with EtOAc (50 mL) and washed with 50% brine (4×50 mL). The organic portion was dried over MgSO₄ and the solvent removed in vacuo. Purification by column chromatography on silica eluting with a gradient of 1 to 2% MeOH in CHCI3 afforded the titled compound as a cream solid.

LC-MS (Method 8B): Rt 3.15 mins; MS m/z 278.0=[M+H]+

1H NMR (500 MHz, DMSO-d6) δ 9.04 (t, J=5.6 Hz, 1H), 8.56 (s, 1H), 8.34 (ddd, J=4.8, 1.7, 0.8 Hz, 1H), 8.27 (br s, 2H), 7.59 (ddd, J=7.6, 1.7, 0.8 Hz, 1H), 7.22 (dd, J=7.6, 4.8 Hz, 1H), 4.55 (d, J=5.5 Hz, 2H), 2.32 (s, 3H).

### Step 3: 4-Amino-2-(3-cyanophenyl)-N-[(3-methyl-2-pyridyl)methyl]pyrimidine-5-carboxamide

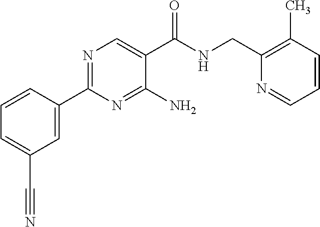 [SMILES: Cc1cccnc1CNC(=O)c1cnc(-c2cccc(C#N)c2)nc1N]

The titled compound was prepared from 4-amino-2-chloro-N-[(3-methyl-2-pyridyl)methyl]pyrimidine-5-carboxamide (step 2) and commercially available (3-cyanophenyl)boronic acid analogously to Example 3 step 1

LC-MS (Method 8B): Rt 4.00 mins; MS m/z 345.1=[M+H]+

1H NMR (500 MHz, Chloroform-d) δ 8.85 (s, 1H), 8.74 (td, J=1.7, 0.4 Hz, 1H), 8.65 (dt, J=8.0, 1.5 Hz, 1H), 8.53 (t, J=3.7 Hz, 1H), 8.42 (dd, J=4.8, 1.6 Hz, 1H), 7.75 (dt, J=7.7, 1.5 Hz, 1H), 7.58 (t, J=7.8 Hz, 1H), 7.54 (ddd, J=7.6, 1.7, 0.9 Hz, 1H), 7.20 (dd, J=7.6, 4.9 Hz, 1H), 7.03 (s, 2H), 4.64 (d, J=3.7 Hz, 2H), 2.35 (s, 3H).

### Example 13

### 4-Amino-6-chloro-2-(2-furyl)-N-[(3-methyl-2-pyridyl)methyl]pyrimidine-5-carboxamide

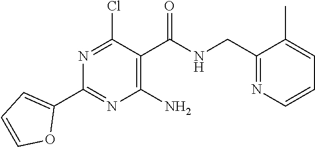 [SMILES: Cc1cccnc1CNC(=O)c1c(N)nc(-c2ccco2)nc1Cl]

### Step 1: Ethyl 4,6-dichloro-2-methylsulfanyl-pyrimidine-5-carboxylate

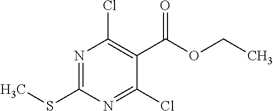

The titled compound was prepared from 4,6-dichloro-2-methylsulfanyl-pyrimidine and ethyl chloroformate according to the procedure of Tandon M; Wang J; Namdev N (US20100249110 A1 page 36).

LDA (2M in THF/n-heptane/ethylbenzene) (3.72 mL, 28.2 mmol) was added dropwise to a solution of commercially available 4,6-dichloro-2-methylsulfanyl-pyrimidine (5 g, 25.63 mmol) in anhydrous THF (30 mL) at −78° C. under a nitrogen atmosphere and the mixture stirred for 1 hour. Ethyl chloroformate (2.7 mL, 28.24 mmol) was added via syringe and the mixture stirred at −78° C. for a further 2 hours and then allowed to warm to room temperature. The reaction was quenched by addition of saturated aqueous ammonium chloride solution (50 mL) and extracted with EtOAc (2×50 mL). The combined organic portions were dried over MgSO₄ and the solvent removed in vacuo. Purification by column chromatography on silica eluting with a gradient of 1 to 2% EtOAc in petrol afforded the titled compound as a yellow solid.

LC-MS (Method 3B): Rt 2.52 mins; MS m/z N/A [does not ionise]

1H NMR (500 MHz, Chloroform-d) δ 4.45 (q, J=7.1 Hz, 2H), 2.58 (s, 3H), 1.41 (t, J=7.1 Hz, 3H).

### Step 2: Ethyl 4-amino-6-chloro-2-methylsulfanyl-pyrimidine-5-carboxylate

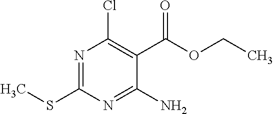

Ammonium hydroxide (35% in water) (5.98 mL, 18.42 mmol) was added to a solution of ethyl 4,6-dichloro-2-methylsulfanyl-pyrimidine-5-carboxylate (step 1)(492 mg, 1.84 mmol) in THF (10 mL) and the mixture stirred vigorously at room temperature for 1 hour. The resulting mixture was diluted with EtOAc (125 mL) and washed with water (2×125 mL) and brine (125 mL). The organic portion was dried over MgSO₄ and the solvent removed in vacuo to afford the titled compound as a pale yellow solid.

LC-MS (Method 3B): Rt 1.69 mins; MS m/z 248.1=[M+H]+

1H NMR (500 MHz, Chloroform-d) δ 7.83 (br s, 1H), 5.69 (br s, 1H), 4.38 (q, J=7.1 Hz, 2H), 2.50 (s, 3H), 1.40 (t, J=7.1 Hz, 3H).

### Step 3-5: 4-Amino-6-chloro-2-(2-furyl)-N-[(3-methyl-2-pyridyl)methyl]pyrimidine-5-carboxamide

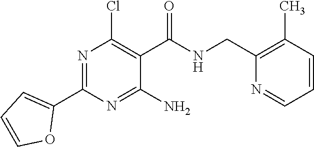 [SMILES: Cc1cccnc1CNC(=O)c1c(N)nc(-c2ccco2)nc1Cl]

The titled compound is prepared from ethyl 4-amino-6-chloro-2-methylsulfanyl-pyrimidine-5-carboxylate (step 2) and 2-furylboronic acid analogously to Example 8 (steps 1-3).

LC-MS (Method 8B): Rt 3.58 mins; MS m/z 344.0=[M+H]+

1H NMR (500 MHz, Chloroform-d) δ 8.55-8.47 (m, 1H), 8.32 (ddd, J=4.9, 1.6, 0.8 Hz, 1H), 7.55 (dd, J=1.7, 0.8 Hz, 1H), 7.48 (d, J=7.6 Hz, 1H), 7.28 (dd, J=3.5, 0.8 Hz, 1H), 7.14 (dd, J=7.6, 4.9 Hz, 1H), 6.50 (dd, J=3.5, 1.7 Hz, 1H), 4.65 (d, J=4.3 Hz, 2H), 2.30 (s, 3H). Amine signals not observed.

### Example 14

### 4-Amino-2-(4-fluorophenyl)-N-[(3-methyl-2-pyridyl)methyl]pyrimidine-5-carboxamide

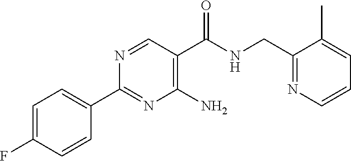 [SMILES: Cc1cccnc1CNC(=O)c1cnc(-c2ccc(F)cc2)nc1N]

### Step 1: 4-Amino-2-(4-fluorophenyl)pyrimidine-5-carboxylic Acid

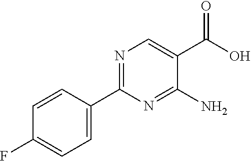 [SMILES: Nc1nc(-c2ccc(F)cc2)ncc1C(=O)O]

The titled compound was prepared from commercially available ethyl 4-amino-2-chloro-pyrimidine-5-carboxylate and (4-fluorophenyl)boronic acid analogously to Example 3 step 1 and 2.

LC-MS (Method 3A): Rt 1.10 mins; MS m/z 234.0=[M+H]+

### Step 2: 4-Amino-2-(4-fluorophenyl)-N-[(3-methyl-2-pyridyl)methyl]pyrimidine-5-carboxamide

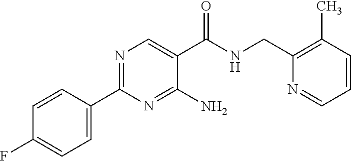 [SMILES: Cc1cccnc1CNC(=O)c1cnc(-c2ccc(F)cc2)nc1N]

The titled compound was prepared from 4-amino-2-(4-fluorophenyl)pyrimidine-5-carboxylic acid and commercially available (3-methyl-2-pyridyl)methanamine analogously to Example 3 step 3.

LC-MS (Method 8B): Rt 4.21 mins; MS m/z 338.1=[M+H]+

1H NMR (500 MHz, Chloroform-d) δ 8.85 (s, 1H), 8.52 (br t, J=3.8 Hz, 1H), 8.44-8.39 (m, 3H), 7.57-7.53 (m, 1H), 7.21 (dd, J=7.6, 4.9 Hz, 1H), 7.17-7.11 (m, 2H), 7.25-6.50 (br s, 2H), 4.65 (d, J=3.8 Hz, 2H), 2.36 (s, 3H).

### Example 15

### 4-Amino-N-[(3-methyl-2-pyridyl)methyl]-2-pyrazol-1-yl-pyrimidine-5-carboxamide

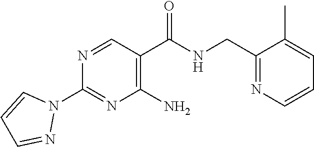 [SMILES: Cc1cccnc1CNC(=O)c1cnc(-n2cccn2)nc1N]

### Step 1: Ethyl 4-amino-2-pyrazol-1-yl-pyrimidine-5-carboxylate

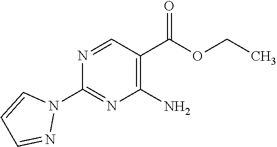 [SMILES: CCOC(=O)c1cnc(-n2cccn2)nc1N]

To a solution of commercially available ethyl 4-amino-2-chloro-pyrimidine-5-carboxylate (200 mg, 0.99 mmol) and 1H-pyrazole (135 mg, 1.98 mmol) in DMF (5 mL) was added Cs₂CO₃ (700 mg, 1.98 mmol) and the mixture was stirred for 3 hours. The resulting mixture was partitioned between EtOAc (25 mL) and H₂O (25 mL), the organic portion separated and the aqueous further extracted with EtOAc (2×25 mL). The combined organic extracts were washed with H₂O (3×25 mL), brine, dried over MgSO₄ and the solvent removed in vacuo to afford a solid. The solid was suspended in Et₂O, collected by filtration and dried to afford the titled compound as a white solid.

LC-MS (Method 3B): Rt 1.36 mins; MS m/z 234.1=[M+H]+

1H NMR (500 MHz, Chloroform-d) δ 8.90 (s, 1H), 8.57 (dd, J=2.7, 0.7 Hz, 1H), 8.08 (br s, 1H), 7.81 (dd, J=1.6, 0.7 Hz, 1H), 6.47 (dd, J=2.7, 1.6 Hz, 1H), 6.03 (br s, 1H), 4.38 (q, J=7.1 Hz, 2H), 1.41 (t, J=7.1 Hz, 3H).

### Step 2: 4-Amino-2-pyrazol-1-yl-pyrimidine-5-carboxylic Acid

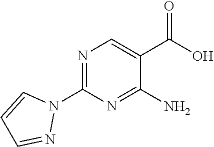 [SMILES: Nc1nc(-n2cccn2)ncc1C(=O)O]

To a solution of ethyl 4-amino-2-pyrazol-1-yl-pyrimidine-5-carboxylate (step 1) (70 mg, 0.3 mmol) in THF (3 mL) was added a solution of LiOH (9 mg, 0.36 mmol) in water (3 mL) and the mixture was stirred for 3 hours. Additional LiOH (9 mg, 0.36 mmol) in H₂O (0.5 mL) was added and the mixture was stirred at room temperature for 16 hours. 2M HCl was added to neutralize the mixture and the solvent was removed in vacuo to afford the titled compound as a white solid.

LC-MS (Method 3A): Rt 0.97 mins; MS m/z 206.1=[M+H]+

### Step 3:4-Amino-N-[(3-methyl-2-pyridyl)methyl]-2-pyrazol-1-yl-pyrimidine-5-carboxamide

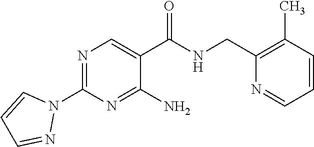 [SMILES: Cc1cccnc1CNC(=O)c1cnc(-n2cccn2)nc1N]

The titled compound was prepared from 4-amino-2-pyrazol-1-yl-pyrimidine-5-carboxylic acid (step 2) and commercially available (3-methyl-2-pyridyl)methanamine analogously to Example 3 step 3.

LC-MS (Method 8B): Rt 3.41 mins; MS m/z 310.1=[M+H]+

1H NMR (500 MHz, Chloroform-d) δ 8.78 (s, 1H), 8.57 (d, J=2.8 Hz, 1H), 8.55 (apr s, 1H), 8.44-8.39 (m, 1H), 7.83-7.78 (m, 1H), 7.58 (d, J=7.6 Hz, 1H), 7.24 (dd, J=7.6, 4.9 Hz, 1H), 6.48 (dd, J=2.8, 1.6 Hz, 1H), 4.66 (d, J=4.0 Hz, 2H), 2.38 (s, 3H). Amine signals not observed.

### Example 16

### 4-Amino-N-[(3-methyl-2-pyridyl)methyl]-2-oxazol-2-yl-pyrimidine-5-carboxamide

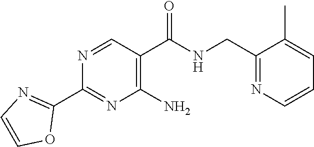 [SMILES: Cc1cccnc1CNC(=O)c1cnc(-c2ncco2)nc1N]

### Step 1: Ethyl 4-amino-2-oxazol-2-yl-pyrimidine-5-carboxylate

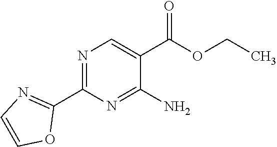 [SMILES: CCOC(=O)c1cnc(-c2ncco2)nc1N]

To a solution of commercially available ethyl 4-amino-2-chloro-pyrimidine-5-carboxylate (500 mg, 2.48 mmol) in 1,4-dioxane (10 mL) was added tributyl(oxazol-2-yl)stannane (779 μL, 3.72 mmol), Pd(PPh₃)₄ (287 mg, 0.25 mmol) and copper(I) iodide (118 mg, 0.62 mmol) and the mixture was heated at 100° C. for 18 hours. Additional tributyl(oxazol-2-yl)stannane (779 μL, 3.72 mmol) was added and the mixture heated at 100° C. for a further 3 hours and allowed to cool to room temperature. The mixture was partitioned between EtOAc (50 mL) and 1M KF solution (50 mL), the organic portion separated and the aqueous further extracted with EtOAc (2×25 mL). The combined organic extracts were washed with H₂O (50 mL), brine, dried over MgSO₄ and the solvent removed in vacuo. Purification by column chromatography on silica eluting with 3% MeOH in DCM afforded the titled compound as a cream solid.

LC-MS (Method 3B): Rt 1.17 mins; MS m/z 235.1=[M+H]+

1H NMR (500 MHz, Chloroform-d) δ 9.05 (br s, 1H), 8.10 (br s, 1H), 7.89 (br s, 1H), 7.43 (br s, 1H), 6.16 (br s, 1H), 4.41 (q, J=7.1 Hz, 2H), 1.42 (t, J=7.1 Hz, 3H).

### Step 2: 4-Amino-2-oxazol-2-yl-pyrimidine-5-carboxylic Acid

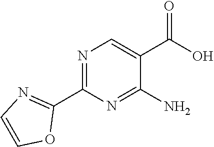 [SMILES: Nc1nc(-c2ncco2)ncc1C(=O)O]

The titled compound was prepared from ethyl 4-amino-2-oxazol-2-yl-pyrimidine-5-carboxylate (step 1) analogously to Example 15 step 2.

LC-MS (Method 3A): Rt 0.71/0.77 mins; MS m/z 207.1=[M+H]+

### Step 3: 4-Amino-N-[(3-methyl-2-pyridyl)methyl]-2-oxazol-2-yl-pyrimidine-5-carboxamide

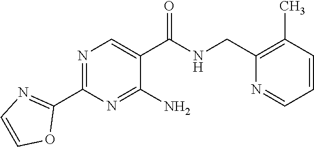 [SMILES: Cc1cccnc1CNC(=O)c1cnc(-c2ncco2)nc1N]

The titled compound was prepared from 4-amino-2-oxazol-2-yl-pyrimidine-5-carboxylic acid (step 2) and commercially available (3-methyl-2-pyridyl)methanamine analogously to Example 3 step 3.

LC-MS (Method 8B): Rt 3.17 mins; MS m/z 311.1=[M+H]+

1H NMR (500 MHz, Chloroform-d) δ 8.92 (s, 1H), 8.68 (apr s, 1H), 8.45 (dd, J=4.9. 0.9 Hz, 1H), 7.88 (d, J=0.7 Hz, 1H), 7.80-6.60 (v br s, 2H), 7.63 (d, J=7.6 Hz, 1H), 7.42 (d, J=0.7 Hz, 1H), 7.30-7.27 (m, 1H—overlapping with residual solvent peak), 4.70 (d, J=4.1 Hz, 2H), 2.42 (s, 3H).

### Example 16.1

### 4-Amino-N-[(3-methyl-2-pyridyl)methyl]-2-thiazol-2-yl-pyrimidine-5-carboxamide

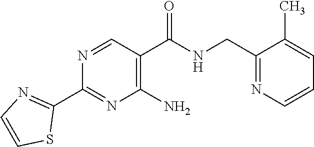 [SMILES: Cc1cccnc1CNC(=O)c1cnc(-c2nccs2)nc1N]

### Step 1: Ethyl 4-amino-2-thiazol-2-yl-pyrimidine-5-carboxylate

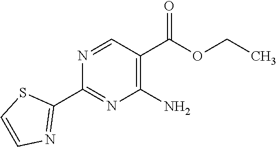 [SMILES: CCOC(=O)c1cnc(-c2nccs2)nc1N]

The titled compound was prepared from ethyl 4-amino-2-chloro-pyrimidine-5-carboxylate and tributyl(thiazol-2-yl)stannane analogously to Example 16 step 1.

LC-MS (Method 3B): Rt 1.34 mins; MS m/z 251.0=[M+H]+

1H NMR (500 MHz, Chloroform-d) δ 8.98 (s, 1H), 8.05 (d, J=3.1 Hz, 1H), 8.02 (br s, 1H), 7.57 (d, J=3.1 Hz, 1H), 6.01 (br s, 1H), 4.40 (q, J=7.1 Hz, 2H), 1.41 (t, J=7.1 Hz, 3H).

### Step 2: 4-Amino-2-thiazol-2-yl-pyrimidine-5-carboxylic Acid

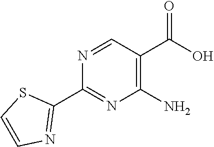 [SMILES: Nc1nc(-c2nccs2)ncc1C(=O)O]

The titled compound was prepared from Ethyl 4-amino-2-thiazol-2-yl-pyrimidine-5-carboxylate analogously to Example 16 step 2.

LC-MS (Method 3A): Rt 0.95 mins; MS m/z 223.2=[M+H]+

### Step 3: 4-Amino-N-[(3-methyl-2-pyridyl)methyl]-2-thiazol-2-yl-pyrimidine-5-carboxamide

The titled compound was prepared from 4-Amino-2-thiazol-2-yl-pyrimidine-5-carboxylic acid and (3-methyl-2-pyridyl)methanamine analogously to Example 16 step 3.

LC-MS (Method 8B): Rt 3.39 mins; MS m/z 327.0=[M+H]+

1H NMR (500 MHz, Chloroform-d) δ 8.82 (s, 1H), 8.53 (br t, J=3.8 Hz, 1H), 8.40 (dd, J=4.9, 1.0 Hz, 1H), 8.04 (d, J=3.1 Hz, 1H), 7.54 (d, J=3.1 Hz, 1H), 7.53 (ddd, J=7.6, 1.6, 1.0 Hz, 1H), 7.20 (dd, J=7.6, 4.9 Hz, 1H), 4.63 (d, J=3.8 Hz, 2H), 2.34 (s, 3H).

### Example 17

### 5-Amino-3-(3-cyanophenyl)-N-[(3-methyl-2-pyridyl)methyl]-1,2,4-triazine-6-carboxamide

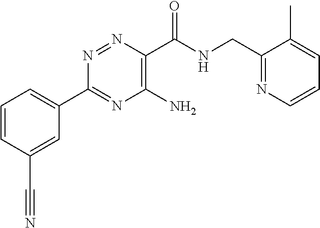 [SMILES: Cc1cccnc1CNC(=O)c1nnc(-c2cccc(C#N)c2)nc1N]

The title compound was prepared from ethyl 5-amino-3-methylsulfanyl-1,2,4-triazine-6-carboxylate (Example 1 step 1) and commercially available (3-cyanophenyl)boronic acid analogously to Example 8 steps 1-3.

LC-MS (Method 8B): Rt 4.28 mins; MS m/z 346.1=[M+H]+

1H NMR (500 MHz, Chloroform-d) δ 9.71 (br t, J=4.4 Hz, 1H), 8.81 (br s, 1H), 8.80 (t, t, J=1.6 Hz, 1H), 8.76 (dt, J=7.8, 1.6 Hz, 1H), 8.47 (dd, J=4.8, 1.5 Hz, 1H), 7.81 (dt, J=7.6, 1.5 Hz, 1H), 7.64 (t, J=7.8 Hz, 1H), 7.52 (d, J=7.8 Hz, 1H), 7.19 (dd, J=7.6, 4.8 Hz, 1H), 5.77 (s, 1H), 4.73 (d, J=4.4 Hz, 2H), 2.37 (s, 3H).

### Example 18

### 4-Amino-2-(2-furyl)-6-methoxy-N-[(3-methyl-2-pyridyl)methyl]pyrimidine-5-carboxamide

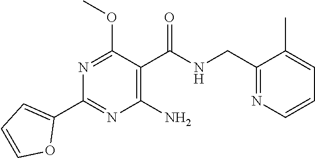 [SMILES: COc1nc(-c2ccco2)nc(N)c1C(=O)NCc1ncccc1C]

### Step 1: Methyl 4-amino-6-methoxy-2-methylsulfanyl-pyrimidine-5-carboxylate

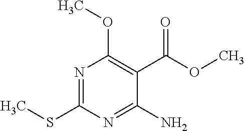

Sodium methoxide (132 mg, 2.44 mmol) was added to a solution of ethyl 4-amino-6-chloro-2-methylsulfanyl-pyrimidine-5-carboxylate (Example 13 step 2) (200 mg, 0.81 mmol) in MeOH (8 mL) and the mixture stirred at room temperature for 22.5 hours. The resulting mixture was concentrated in vacuo (water bath at 25° C.) to approximately half the initial volume. The suspension which formed was collected by vacuum filtration, washed with a small volume of MeOH and dried under vacuum to afford the titled compound as a white solid.

LC-MS (Method 3A): Rt 1.50 mins; MS m/z 230.1=[M+H]+

1H NMR (500 MHz, DMSO-d6) δ 7.79 (s, 2H), 3.88 (s, 3H), 3.73 (s, 3H), 2.46 (s, 3H).

### Step 2: Methyl 4-amino-2-(2-furyl)-6-methoxy-pyrimidine-5-carboxylate

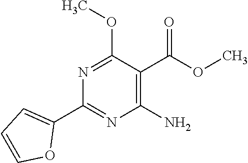 [SMILES: COC(=O)c1c(N)nc(-c2ccco2)nc1OC]

A vial comprising methyl 4-amino-6-methoxy-2-methylsulfanyl-pyrimidine-5-carboxylate (step 1)(145 mg, 0.63 mmol), commercially available 2-furylboronic acid (106 mg, 0.95 mmol), Pd(PPh₃)₄ (37 mg, 0.03 mmol) and copper(I) thiophene-2-carboxylate (241 mg, 1.27 mmol) was sealed and placed under an atmosphere of nitrogen. De-gassed THF (8 mL) was added via syringe and the resulting mixture heated using microwave radiation at 55° C. for 17.5 hours. More 2-furylboronic acid (71 mg, 0.63 mmol), Pd(PPh₃)₄ (73 mg, 0.06 mmol) and copper(I) thiophene-2-carboxylate (121 mg, 0.63 mmol) were added and stirring continued at 55° C. for a further 20.25 hours. The mixture was allowed to cool, diluted with EtOAc (50 mL) and washed sequentially with 1M aqueous NaHSO₄ (2×50 mL), saturated aqueous NaHCO₃ (2×50 mL) and brine (50 mL). The organic portion was dried over MgSO₄, filtered and concentrated in vacuo to afford a yellow residue. Purification by column chromatography on silica eluting with a gradient of 0.5 to 2% MeOH in DCM afforded the titled compound as a yellow solid.

LC-MS (Method 3B): Rt 1.48 mins; MS m/z 250.0=[M+H]+

1H NMR (500 MHz, Chloroform-d) δ 8.18 (br s, 1H), 7.64-7.62 (m, 1H), 7.42-7.37 (m, 1H), 6.56 (dd, J=3.5, 1.7 Hz, 1H), 6.17 (br s, 1H), 4.08 (s, 3H), 3.88 (s, 3H).

### Step 3: 4-Amino-2-(2-furyl)-6-methoxy-pyrimidine-5-carboxylic Acid

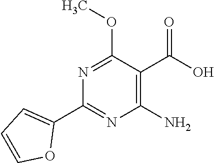 [SMILES: COc1nc(-c2ccco2)nc(N)c1C(=O)O]

A solution of LiOH (12 mg, 0.51 mmol) in water (3 mL) was added to a stirred suspension of methyl 4-amino-2-(2-furyl)-6-methoxy-pyrimidine-5-carboxylate (step 2) (80 mg, 0.25 mmol) in THF (3 mL) at room temperature for 2 hour. MeOH (1 mL) was added and the mixture stirred at room temperature for a further 19.5 hours. More LiOH (6 mg, 0.25 mmol) was added and the mixture stirred at 40° C. for 3 hours. The mixture was allowed to cool, diluted with water (3 mL) and extracted with DCM (3×5 mL). The combined organic portions were back extracted with water (5 mL) and the combined aqueous portions were acidified with 2M HCl (0.4 mL) and concentrated in vacuo to afford the titled compound as wet yellow solid.

LC-MS (Method 3A): Rt 1.24 mins; MS m/z 236.0=[M+H]+

### Step 4: 4-Amino-2-(2-furyl)-6-methoxy-N-[(3-methyl-2-pyridyl)methyl]pyrimidine-5-carboxamide

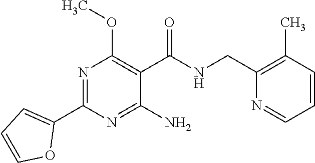 [SMILES: COc1nc(-c2ccco2)nc(N)c1C(=O)NCc1ncccc1C]

(3-Methyl-2-pyridyl)methanamine (45 μL, 0.38 mmol), HATU (190 mg, 0.5 mmol) and then DIPEA (218 μL, 1.25 mmol) were added in quick succession to a solution of 4-amino-2-(2-furyl)-6-methoxy-pyrimidine-5-carboxylic acid (step 3)(59 mg, 0.25 mmol) in DMF (3 mL) and the mixture stirred at room temperature for 17 hours. The resulting mixture was diluted with EtOAc (25 mL) and washed with 50% brine (4×25 mL). The organic portion was dried over MgSO₄ and concentrated in vacuo to afford a grey residue. Purification by column chromatography on silica eluting with a gradient of 50 to 100% EtOAc in petrol afforded a cream solid. The material was triturated with Et₂O/EtOAc to afford the titled compound as a pale cream solid.

LC-MS (Method 8B): Rt 4.54 mins; MS m/z 340.1=[M+H]+

1H NMR (500 MHz, Chloroform-d) δ 9.72-9.65 (m, 1H), 9.40 (br s, 1H), 8.48-8.43 (m, 1H), 7.61 (dd, J=1.8, 0.9 Hz, 1H), 7.57 (br d, J=7.5 Hz, 1H), 7.31 (d, J=3.4 Hz, 1H), 7.23 (br t, J=6.3 Hz, 1H), 6.55 (dd, J=3.4, 1.7 Hz, 1H), 5.73 (br s, 1H), 4.69 (d, J=4.1 Hz, 2H), 4.24 (s, 3H), 2.40 (s, 3H).

### Example 18.1

### 4-Amino-2-(2-furyl)-6-methoxy-N-[[3-(trifluoromethyl)-2-pyridyl]methyl]pyrimidine-5-carboxamide

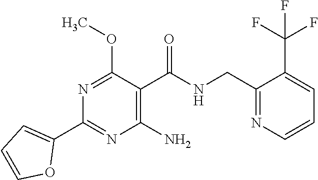 [SMILES: COc1nc(-c2ccco2)nc(N)c1C(=O)NCc1ncccc1C(F)(F)F]

The titled compound was prepared from 4-amino-2-(2-furyl)-6-methoxy-pyrimidine-5-carboxylic acid (Example 18 step 3) and commercially available [3-(trifluoromethyl)-2-pyridyl]methanamine hydrochloride analogously to Example 18 step 4.

LC-MS (Method 8B): Rt 4.97 mins; MS m/z 394.2=[M+H]+

1H NMR (500 MHz, Chloroform-d) δ 9.67-9.24 (m, 2H), 8.79 (d, J=4.8 Hz, 1H), 8.01 (d, J=7.9 Hz, 1H), 7.62 (s, 1H), 7.40 (dd, J=7.9, 4.8 Hz, 1H), 7.33 (br. s, 1H), 6.56 (s, 1H), 5.74 (br. s, 1H), 4.93 (d, J=4.3 Hz, 2H), 4.22 (s, 3H).

### Example 18.3

### 4-Amino-N-[(2,6-dichlorophenyl)methyl]-2-(2-furyl)-6-methoxy-pyrimidine-5-carboxamide

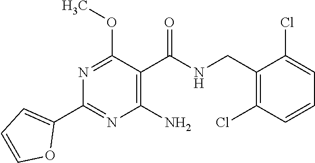 [SMILES: COc1nc(-c2ccco2)nc(N)c1C(=O)NCc1c(Cl)cccc1Cl]

The titled compound was prepared from 4-amino-2-(2-furyl)-6-methoxy-pyrimidine-5-carboxylic acid (Example 18 step 3) and commercially available (2,6-dichlorophenyl) methanamine analogously to Example 18 step 4.

LC-MS (Method 8B): Rt 5.46 mins; MS m/z 393.1=[M+H]+

1H NMR (500 MHz, Chloroform-d) δ 9.37 (br. s, 1H), 8.42 (s, 1H), 7.60 (s, 1H), 7.35 (d, J=8.0 Hz, 2H), 7.28 (br. s, 1H), 7.20 (t, J=8.0 Hz, 1H), 6.53 (s, 1H), 5.72 (br. s, 1H), 4.91 (d, J=5.7 Hz, 2H), 4.09 (s, 3H).

### Example 19

### 4-Amino-2-(2-furyl)-N-[(3-methyl-2-pyridyl)methyl]-6-pyrazol-1-yl-pyrimidine-5-carboxamide

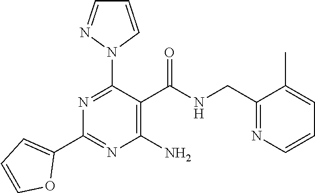 [SMILES: Cc1cccnc1CNC(=O)c1c(N)nc(-c2ccco2)nc1-n1cccn1]

A mixture comprising 4-amino-6-chloro-2-(2-furyl)-N-[(3-methyl-2-pyridyl)methyl]pyrimidine-5-carboxamide (Example 13) (25 mg, 0.07 mmol), commercially available 1H-pyrazole (10 mg, 0.15 mmol) and potassium carbonate (20 mg, 0.15 mmol) was stirred in DMF (1 mL) at 100° C. for 21 hours. After cooling to room temperature, the mixture was diluted with EtOAc (20 mL) and washed with water (4×20 mL). The organic portion was dried over MgSO₄, filtered and concentrated in vacuo. Purification by column chromatography on silica eluting with a gradient of 50 to 100% EtOAc in petrol afforded the titled compound as a pale tan solid.

LC-MS (Method 8B): 3.76 mins; MS m/z 376.1=[M+H]+

1H NMR (500 MHz, Chloroform-d) δ 8.50 (dd, J=2.7, 0.7 Hz, 1H), 8.22-8.19 (m, 1H), 7.60 (dd, J=1.7, 0.8 Hz, 1H), 7.56 (dd, J=1.6, 0.7 Hz, 1H), 7.50 (d, J=7.6 Hz, 1H), 7.36-7.32 (m, 1H), 7.31 (dd, J=3.4, 0.9 Hz, 1H), 7.13 (dd, J=7.6, 4.9 Hz, 1H), 6.67 (br s, 2H), 6.55 (dd, J=3.4, 1.7 Hz, 1H), 6.38 (dd, J=2.7, 1.6 Hz, 1H), 4.71 (d, J=5.0 Hz, 2H), 2.35 (s, 3H).

### Example 20

### 4-(2-Acetamidoethoxy)-6-amino-2-(2-fury)-N-[(3-methyl-2-pyridyl)methyl]pyrimidine-5-carboxamide

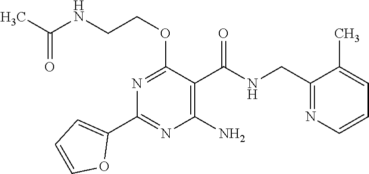 [SMILES: CC(=O)NCCOc1nc(-c2ccco2)nc(N)c1C(=O)NCc1ncccc1C]

N-Acetylethanolamine (10 μL, 0.11 mmol) was added to a suspension of sodium hydride (60% in oil) (5 mg, 0.12 mmol) in anhydrous DMF (0.5 mL) under an atmosphere of nitrogen and the resulting mixture stirred at room temperature for 30 mins. A solution of 4-amino-6-chloro-2-(2-furyl)-N-[(3-methyl-2-pyridyl)methyl]pyrimidine-5-carboxamide (Example 13) (25 mg, 0.07 mmol) in anhydrous DMF (0.5 mL) was added via syringe and the resulting mixture stirred at room temperature for 4 hours. The reaction was quenched by addition of MeOH (3 drops) and the resulting precipitate collected by filtration. The solid was washed with a small volume of MeOH then Et₂O and vacuum dried to afford the titled compound as a white solid.

LC-MS (Method 8B): Rt 3.86 mins; MS m/z 411.2=[M+H]+

1H NMR (500 MHz, DMSO-d6) δ 9.42 (t, J=4.3 Hz, 1H), 9.12 (br s, 1H), 8.43 (br dd, J=4.9, 1.6 Hz, 1H), 8.09 (t, J=5.6 Hz, 1H), 7.91 (dd, J=1.7, 0.9 Hz, 1H), 7.74 (br s, 1H), 7.66-7.62 (m, 1H), 7.31-7.25 (m, 2H), 6.69 (dd, J=3.4, 1.7 Hz, 1H), 4.57 (d, J=4.3 Hz, 2H), 4.55 (t, J=6.1 Hz, 2H), 3.64 (apr q, J=6.0 Hz, 2H), 2.31 (s, 3H), 1.73 (s, 3H).

The compounds of the following tabulated Examples (Table 5) were prepared analogously to Example 20 from 4-amino-6-chloro-2-(2-furyl)-N-[(3-methyl-2-pyridyl)methyl]pyrimidine-5-carboxamide (Example 13) and the appropriate commercially available alcohol.

TABLE 5

| Ex. | Structure and Name | Retention Time, [M + H]+, 1H NMR |
|---|---|---|
| 20.1 | 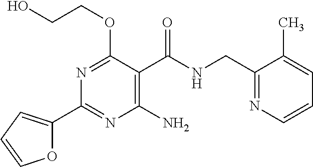 [SMILES: Cc1cccnc1CNC(=O)c1c(N)nc(-c2ccco2)nc1OCCO] 4-Amino-2-(2-furyl)-6-(2-hydroxyethoxy)-N- | LC-MS (Method 8B): Rt 4.06 mins; MS m/z 370.2 = [M + H]+ 1H NMR (500 MHz, DMSO-d6) δ 9.45 (t, J = 4.2 Hz, 1H), 9.10 (br s, 1H), 8.41 (br dd, J = 4.9, 1.7 Hz, 1H), 7.91 (dd, J = 1.7, 0.9 Hz, 1H), 7.73 (br s, 1H), 7.67-7.63 (m, 1H), 7.28 (dd, J = 7.5, 4.8 Hz, 1H), 7.25 (dd, J = 3.4, 0.8 Hz, 1H), 6.68 (dd, J = 3.4, 1.7 Hz, 1H), 4.90 (br t, J = 4.6 Hz, 1H), 4.61-4.53 (m, 4H), 3.97-3.90 (m, 2H), 2.31 (s, 3H) |
| | [(3-methyl-2-pyridyl)methyl]pyrimidine-5- | |
| | carboxamide | |
| 20.2 | 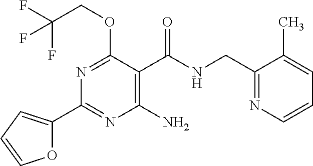 [SMILES: Cc1cccnc1CNC(=O)c1c(N)nc(-c2ccco2)nc1OCC(F)(F)F] 4-Amino-2-(2-furyl)-N-[(3-methyl-2- | LC-MS (Method 8B): Rt 5.07 mins; MS m/z 408.2 = [M + H]+ 1H NMR (500 MHz, Chloroform-d) δ 9.51 (br s, 1H), 9.35-9.26 (m, 1H), 8.42 (br d, J = 4.6 Hz, 1H), 7.65-7.60 (m, 1H), 7.50 (br d, J = 5.0 Hz, 1H), 7.29 (d, J = 3.5 Hz, 1H), 7.20-7.14 (m, 1H), 6.56 (dd, J = 3.5, 1.7 Hz, 1H), 5.82 (br s, 1H), 5.05 (q, J = 8.4 Hz, 2H), 4.68 (d, J = 3.8 Hz, 2H), 2.34 (s, 3H) |
| | pyridyl)methyl]-6-(2,2,2-trifluoroethoxy) | |
| | pyrimidine-5-carboxamide | |
| 20.3 | 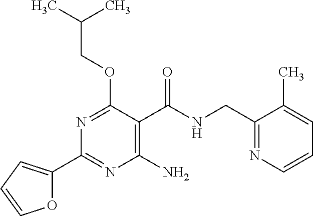 [SMILES: Cc1cccnc1CNC(=O)c1c(N)nc(-c2ccco2)nc1OCC(C)C] 4-Amino-2-(2-furyl)-6-isobutoxy-N-[(3-methyl- | LC-MS (Method 8B): Rt 5.60 mins; MS m/z 382.2 = [M + H]+ 1H NMR (500 MHz, Chloroform-d) δ 9.64-9.55 (m, 1H), 9.47 (br s, 1H), 8.41-8.35 (m, 1H), 7.63- 7.59 (m, 1H), 7.50 (br d, J = 7.6 Hz, 1H), 7.28 (br s, 1H), 7.16 (dd, J = 7.6, 4.9 Hz, 1H), 6.54 (dd, J = 3.4, 1.7 Hz, 1H), 5.70 (br s, 1H), 4.69 (d, J = 3.9 Hz, 2H), 4.39 (d, J = 6.8 Hz, 2H), 2.42 (apr hept, J = 6.7 Hz, 1H), 2.34 (s, 3H), 1.07 (d, J = 6.7 Hz, 6H). |
| | 2-pyridyl)methyl]pyrimidine-5-carboxamide | |
| 20.6 | 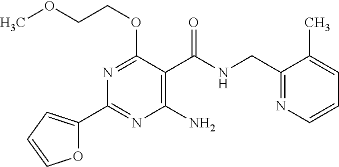 [SMILES: COCCOc1nc(-c2ccco2)nc(N)c1C(=O)NCc1ncccc1C] 4-Amino-2-(2-furyl)-6-(2-methoxyethoxy)-N- [(3-methyl-2-pyridyl)methyl]pyrimidine-5- carboxamide | LC-MS (Method 8B): Rt 4.51 mins; MS m/z 384.2 = [M + H]+ 1H NMR (500 MHz, DMSO-d6) δ 9.35 (t, J = 4.1 Hz, 1H), 9.10 (br s, 1H), 8.48-8.40 (m, 1H), 7.90 (dd, J = 1.7, 0.9 Hz, 1H), 7.75 (br s, 1H), 7.65 (ddd, J = 7.5, 1.7, 0.9 Hz, 1H), 7.29 (dd, J = 7.5, 4.8 Hz, 1H), 7.26 (dd, J = 3.3, 0.9 Hz, 1H), 6.68 (dd, J = 3.3, 1.7 Hz, 1H), 4.70-4.65 (m, 2H), 4.58 (d, J = 4.1 Hz, 2H), 3.90- 3.85 (m, 2H), 3.22 (s, 3H), 2.31 (s, 3H) |
| 20.7 | 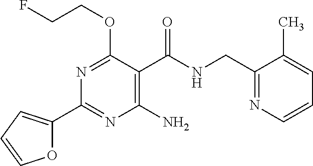 [SMILES: Cc1cccnc1CNC(=O)c1c(N)nc(-c2ccco2)nc1OCCF] 4-Amino-6-(2-fluoroethoxy)-2-(2-furyl)-N-[(3- | LC-MS (Method 8B): Rt 4.42 mins; MS m/z 372.3 = [M + H]+ 1H NMR (500 MHz, DMSO-d6) δ 9.51 (br t, J = 3.4 Hz, 1H), 9.14 (br s, 1H), 8.41 (d, J = 4.0 Hz, 1H), 7.91 (s, 1H), 7.79 (br s, 1H), 7.65 (d, J = 7.3 Hz, 1H), 7.34-7.24 (m, 2H), 6.72-6.67 (m, 1H), 4.99 (dt, J = 47.7, 3.4 Hz, 2H), 4.80 (dt, J = 30.6, 3.4 Hz, 2H), 4.58 (d, J = 3.4 Hz, 2H), 2.31 (s, 3H) |
| | methyl-2-pyridyl)methyl]pyrimidine-5- | |
| | carboxamide | |
| 20.8 | 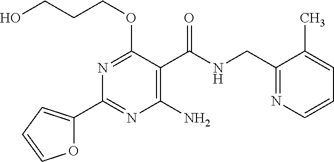 [SMILES: Cc1cccnc1CNC(=O)c1c(N)nc(-c2ccco2)nc1OCCCO] 4-Amino-2-(2-furyl)-6-(3-hydroxpropoxy)-N- [(3-methyl-2-pyridyl)methyl]pyrimidine-5- | LC-MS (Method 8B): Rt 4.07 mins; MS m/z = 384.2 [M + H]+ 1H NMR (500 MHz, DMSO-d6) δ 9.50 (br t, J = 3.4 Hz, 1H), 9.14 (br s, 1H), 8.41 (d, J = 4.1 Hz, 1H), 7.90 (s, 1H), 7.71 (br s, 1H), 7.65 (d, J = 7.2 Hz, 1H), 7.28 (dd, J = 7.2, 5.0 Hz, 1H), 7.25 (d, J = 3.1 Hz, 1H), 6.71-6.65 (m, 1H), 4.64-4.58 (m, 3H), 4.57 (d, J = 3.4 Hz, 2H), 3.67- 3.58 (m, 2H), 2.30 (s, 3H), 2.11 (p, J = 6.1 Hz, 2H) |
| | carboxamide | |
| 20.9 | 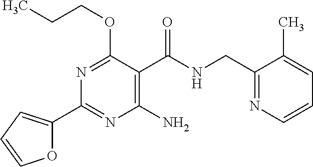 [SMILES: CCCOc1nc(-c2ccco2)nc(N)c1C(=O)NCc1ncccc1C] 4-Amino-2-(2-furyl)-N-[(3-methyl-2-pyridyl) methyl]-6-propoxy-pyrimidine-5-carboxamide | LC-MS (Method 8B): Rt 4.95 mins; MS m/z 368.3 = [M + H]+ 1H NMR (500 MHz, DMSO-d6) δ 9.51 (br t, J = 3.7 Hz, 1H), 9.15 (br s, 1H), 8.39 (d, J = 4.0 Hz, 1H), 7.93- 7.88 (m, 1H), 7.73 (br s, 1H), 7.65 (d, J = 7.3 Hz, 1H), 7.29 (dd, J = 7.3, 4.9 Hz, 1H), 7.25 (dd, J = 3.4, 0.8 Hz, 2H), 6.68 (dd, J = 3.4, 1.7 Hz, 1H), 4.57 (d, J = 3.7 Hz, 2H), 4.51 (t, J = 6.5 Hz, 2H), 2.30 (s, 3H), 1.96 (apr sext, J = 7.2 Hz, 2H), 1.02 (t, J = 7.4 Hz, 3H) |
| 20.10 | 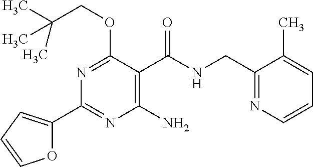 [SMILES: Cc1cccnc1CNC(=O)c1c(N)nc(-c2ccco2)nc1OCC(C)(C)C] | LC-MS (Method 8B): Rt 5.70 mins; MS m/z 396.3 = [M + H]+ 1H NMR (500 MHz, Chloroform-d) δ 9.42 (br s, 1H), 9.04 (br s, 1H), 8.38 (d, J = 4.3 Hz, 1H), 7.61 (s, 1H), 7.51 (br d, J = 6.4 Hz, 1H), 7.30 (br s, 1H), 7.21-7.13 (m, 1H), 6.58-6.52 (m, 1H), 5.73 (br s, 1H), 4.74 (d, J = 3.8 Hz, 2H), 4.35 (s, 2H), 2.37 (s, 3H), 1.04 (s, 9H). |
| | 4-Amino-6-(2,2-dimethylpropoxy)-2-(2-furyl)- | |
| | N-[(3-methyl-2-pyridyl)methyl]pyrimidine-5- | |
| | carboxamide | |
| 20.11 | 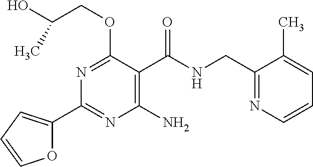 [SMILES: Cc1cccnc1CNC(=O)c1c(N)nc(-c2ccco2)nc1OC[C@H](C)O] 4-Amino-2-(2-furyl)-6-[(2S)-2-hydroxy propoxy]-N-[(3-methyl-2-pyridyl)methyl] pyrimidine-5-carboxamide | LC-MS (Method 8B): Rt 3.93 mins; MS m/z 384.3 = [M + H]+ 1H NMR (500 MHz, DMSO-d6) δ 9.34 (t, J = 4.1 Hz, 1H), 9.09 (br s, 1H), 8.41-8.36 (m, 1H), 7.91 (dd, J = 1.5, 0.8 Hz, 1H), 7.74 (br s, 1H), 7.84-7.61 (m, 1H), 7.29 (dd, J = 7.5, 4.9 Hz, 1H), 7.26 (dd, J = 3.3, 0.8 Hz, 1H), 6.69 (dd, J = 3.4, 1.7 Hz, 1H), 5.00 (br d, J = 3.4 Hz, 1H), 4.59 (d, J = 4.1 Hz, 2H), 4.44 (dd, J = 10.5, 6.3 Hz, 1H), 4.34 (dd, J = 10.5, 5.4 Hz, 1H), 4.28-4.20 (m, 1H), 2.32 (s, 3H), 1.20 (d, J = 6.3 |
| | | Hz, 3H) |
| 20.12 | 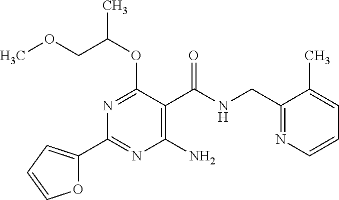 [SMILES: COCC(C)Oc1nc(-c2ccco2)nc(N)c1C(=O)NCc1ncccc1C] 4-Amino-2-(2-furyl)-6-(2-methoxy-1-methyl- | LC-MS (Method 8B): Rt 4.46 mins; MS m/z 398.3 = [M + H]+ 1H NMR (500 MHz, DMSO-d6) δ 9.53 (br t, J = 3.5 Hz, 1H), 9.17 (br s, 1H), 8.44 (br d, J = 4.0 Hz, 1H), 7.91 (s, 1H), 7.75 (br s, 1H), 7.66 (d, J = 7.2 Hz, 1H), 7.30 (dd, J = 7.2, 4.9 Hz, 1H), 7.25 (d, J = 3.2 Hz, 1H), 6.68 (dd, J = 3.2, 1.6 Hz, 1H), 5.83-5.75 (m, 1H), 4.57 (d, J = 3.5 Hz, 2H), 3.81 (dd, J = 10.5, 7.1 Hz, 1H), 3.60 (dd, J = 10.5, 3.9 Hz, 1H), 3.23 (s, 3H), 2.31 (s, 3H), 1.41 (d, J = 6.4 Hz, 3H) |
| | ethoxy)-N-[(3-methyl-2-pyridyl)methyl] | |
| | pyrimidine-5-carboxamide | |
| 20.13 | 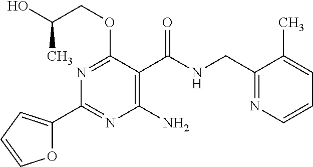 [SMILES: Cc1cccnc1CNC(=O)c1c(N)nc(-c2ccco2)nc1OC[C@@H](C)O] 4-Amino-2-(2-furyl)-6-[(2R)-2-hydroxy propoxy]-N-[(3-methyl-2-pyridyl)methyl] pyrimidine-5-carboxamide | LC-MS (Method 8B): Rt 4.38 mins; MS m/z 384.3 = [M + H]+ 1H NMR (500 MHz, DMSO-d6) δ 9.33 (t, J = 4.2 Hz, 1H), 9.07 (br s, 1H), 8.40-8.36 (m, 1H), 7.90 (dd, J = 1.6, 0.8 Hz, 1H), 7.73 (br s, 1H), 7.65 (d, J = 7.0 Hz, 1H), 7.28 (dd, J = 7.5, 4.9 Hz, 1H), 7.26 (dd, J = 3.4, 0.8 Hz, 1H), 6.68 (dd, J = 3.4, 1.6 Hz, 1H), 5.00 (d, J = 4.5 Hz, 1H), 4.58 (d, J = 4.2 Hz, 2H), 4.43 (dd, J = 10.5, 6.3 Hz, 1H), 4.33 (dd, J = 10.5, 5.4 Hz, 1H), 4.26-4.19 (m, 1H), 2.31 (s, 3H), 1.19 (d, J = 6.3 |
| | | Hz, 3H) |

### Example 22

### 4-(2-Acetamidoethylamino)-6-amino-2-(2-furyl)-N-[(3-methyl-2-pyridyl)methyl]pyrimidine-5-carboxamide

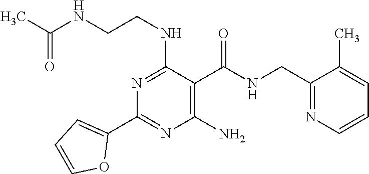 [SMILES: CC(=O)NCCNc1nc(-c2ccco2)nc(N)c1C(=O)NCc1ncccc1C]

### Step 1: Ethyl 4-(2-acetamidoethylamino)-6-amino-2-methylsulfanyl-pyrimidine-5-carboxylate

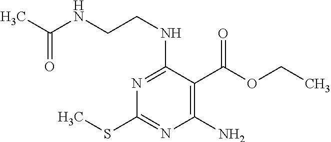 [SMILES: CCOC(=O)c1c(N)nc(SC)nc1NCCNC(C)=O]

N-(2-Aminoethyl)acetamide (290 μL, 3.03 mmol) was added to a solution of ethyl 4-amino-6-chloro-2-methylsulfanyl-pyrimidine-5-carboxylate (Example 13 step 2) (250 mg, 1.01 mmol) in THF (5 mL) and the mixture stirred at room temperature for 19 hours. The resulting mixture was diluted with EtOAc (25 mL) and washed with water (2×25 mL) and brine (20 mL). The organic portion was separated, dried over MgSO₄ and concentrated in vacuo. The crude product was triturated with Et₂O to afford the titled compound as a cream solid.

LC-MS (Method 3B): Rt 1.37 mins; MS m/z 314.2=[M+H]+

1H NMR (500 MHz, Chloroform-d) δ 8.59 (s, 1H), 7.45 (br s, 1H), 6.62 (s, 1H), 5.46 (br s, 1H), 4.37 (q, J=7.1 Hz, 2H), 3.70-3.65 (m, 2H), 3.47-3.42 (m, 2H), 2.48 (s, 3H), 1.97 (s, 3H), 1.40 (t, J=7.1 Hz, 3H).

### Step 2: Ethyl 4-(2-acetamidoethylamino)-6-amino-2-(2-furyl)pyrimidine-5-carboxylate

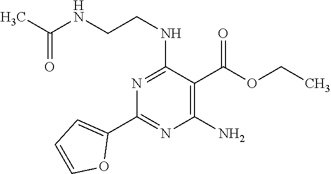 [SMILES: CCOC(=O)c1c(N)nc(-c2ccco2)nc1NCCNC(C)=O]

A microwave vial was charged with ethyl 4-(2-acetamidoethylamino)-6-amino-2-methylsulfanyl-pyrimidine-5-carboxylate (step 1) (220 mg, 0.7 mmol), copper(I) thiophene-2-carboxylate (268 mg, 1.4 mmol), Pd(PPh₃)₄ (81 mg, 0.07 mmol) and commercially available 2-furylboronic acid (118 mg, 1.05 mmol) and placed under an atmosphere of nitrogen. THF (5 mL) was added via syringe and the mixture heated using microwave radiation at 55° C. for 19.5 hours. More 2-furylboronic acid (79 mg, 0.7 mmol), Pd(PPh₃)₄ (81 mg, 0.07 mmol) and copper(I) thiophene-2-carboxylate (134 mg, 0.7 mmol) were added and stirring continued at 55° C. for a further 5 hours. After cooling to room temperature, the mixture was diluted with EtOAc (100 mL) and washed with 10% aqueous ammonia (3×50 mL). The combined aqueous portions were back extracted with EtOAc (50 mL) and the combined organic extracts were filtered, dried over MgSO₄ and concentrated in vacuo. Purification of the crude material by column chromatography on silica eluting with 1% MeOH in EtOAc afforded a pale yellow solid which was triturated with EtOAc/Et₂O to afford the titled compound as a cream solid.

LC-MS (Method 3A): Rt 1.14 mins; MS m/z 334.1=[M+H]+

1H NMR (500 MHz, Chloroform-d) δ 8.68 (s, 1H), 7.60 (dd, J=1.7, 0.9 Hz, 1H), 7.55 (br s, 1H), 7.29 (br d, J=3.4 Hz, 1H), 7.24 (s, 1H), 6.56 (dd, J=3.4, 1.8 Hz, 1H), 5.66 (br s, 1H), 4.40 (q, J=7.1 Hz, 2H), 3.79-3.74 (m, 2H), 3.51-3.46 (m, 2H), 1.86 (s, 3H), 1.43 (t, J=7.1 Hz, 3H).

### Step 3:4-(2-Acetamidoethylamino)-6-amino-2-(2-furyl)pyrimidine-5-carboxylic Acid

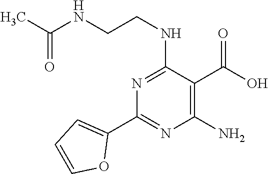 [SMILES: CC(=O)NCCNc1nc(-c2ccco2)nc(N)c1C(=O)O]

A solution of LiOH (10 mg, 0.42 mmol) in water (2 mL) was added to a suspension of ethyl 4-(2-acetamidoethylamino)-6-amino-2-(2-furyl)pyrimidine-5-carboxylate (step 2) (68 mg, 0.2 mmol) in THF (2 mL) and the mixture stirred at room temperature for 20 hours. More LiOH (5 mg, 0.2 mmol) was added and the mixture stirred at room temperature for 1 hour and heated to 40° C. for a further 2 hours. After cooling to room temperature, the mixture was acidified with 2M HCl (0.35 mL) and concentrated in vacuo to afford the titled compound as an off-white, gummy residue. This material was used in the next step without further purification.

LC-MS (Method 3A): Rt 0.92 mins; MS m/z 306.1=[M+H]+

1H NMR (500 MHz, DMSO-d6) δ 10.56 (t, J=5.8 Hz, 1H), 9.55 (br d, J=5.3 Hz, 1H), 8.11 (br t, J=5.5 Hz, 1H), 7.74 (dd, J=1.8, 0.9 Hz, 1H), 7.04 (dd, J=3.3, 0.9 Hz, 1H), 6.57 (dd, J=3.3, 1.8 Hz, 1H), 6.35 (br d, J=5.6 Hz, 1H), 3.47-3.39 (m, 2H), 3.19 (apr q, J=6.3 Hz, 2H), 1.78 (s, 3H).

### Step 4: 4-(2-Acetamidoethylamino)-6-amino-2-(2-furyl)-N-[(3-methyl-2-pyridyl)methyl]pyrimidine-5-carboxamide

The titled compound was prepared from commercially available (3-methyl-2-pyridyl)methanamine and 4-(2-acetamidoethylamino)-6-amino-2-(2-furyl)pyrimidine-5-carboxylic acid (step 3) analogously to Example 3 step 3.

LC-MS (Method 8B): Rt 3.43 mins; MS m/z 410.3=[M+H]+

1H NMR (500 MHz, Chloroform-d) δ 8.45 (br s, 1H), 8.33 (br dd, J=4.9, 1.6 Hz, 1H), 7.59 (dd, J=1.7, 0.9 Hz, 1H), 7.53-7.49 (m, 1H), 7.33 (br s, 1H), 7.26-7.24 (m, 2H), 7.17 (dd, J=7.6, 4.9 Hz, 1H), 6.55 (dd, J=3.4, 1.7 Hz, 1H), 6.42 (br s, 2H), 4.76 (d, J=5.7 Hz, 2H), 3.80-3.73 (m, 2H), 3.53-3.45 (m, 2H), 2.35 (s, 3H), 1.81 (s, 3H).

### Example 23

### 4-Amino-2-(2-furyl)-N-[(3-methyl-2-pyridyl)methyl]-6-[2-(2-piperidyl)ethoxy]pyrimidine-5-carboxamide

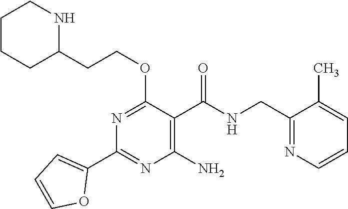 [SMILES: Cc1cccnc1CNC(=O)c1c(N)nc(-c2ccco2)nc1OCCC1CCCCN1]

### Step 1: tert-Butyl 2-[2-[6-amino-2-(2-furyl)-5-[(3-methyl-2-pyridyl)methylcarbamoyl]pyrimidin-4-yl]oxyethyl]piperidine-1-carboxylate

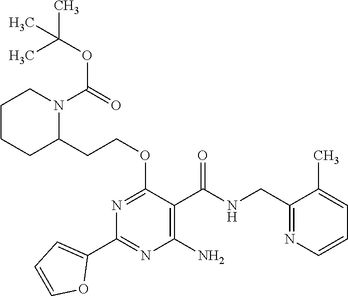 [SMILES: Cc1cccnc1CNC(=O)c1c(N)nc(-c2ccco2)nc1OCCC1CCCCN1C(=O)OC(C)(C)C]

The titled compound was prepared from 4-amino-6-chloro-2-(2-furyl)-N-[(3-methyl-2-pyridyl)methyl]pyrimidine-5-carboxamide (Example 13) and commercially available tert-butyl 2-(2-hydroxyethyl)piperidine-1-carboxylate analogously to Example 20.

LC-MS (Method 3B): Rt 2.17 mins; MS m/z 537.3=[M+H]+

1H NMR (500 MHz, Chloroform-d) δ 9.60 (br s, 1H), 9.41 (br s, 1H), 8.40 (br d, J=3.9 Hz, 1H), 7.60 (s, 1H), 7.54 (br s, 1H), 7.33-7.26 (m, 1H), 7.20 (br s, 1H), 6.54 (dd, J=3.4, 1.7 Hz, 1H), 5.69 (br s, 1H), 4.71 (br s, 2H), 4.61 (br s, 2H), 4.50 (br s, 1H), 4.04 (br s, 1H), 2.91-2.82 (m, 1H), 2.46 (br s, 1H), 2.38 (br s, 3H), 2.18-2.11 (m, 1H), 1.64-1.56 (m, 6H), 1.41 (s, 9H).

### Step 2: 4-Amino-2-(2-furyl)-N-[(3-methyl-2-pyridyl)methyl]-6-[2-(2-piperidyl)ethoxy]pyrimidine-5-carboxamide

tert-Butyl 2-[2-[6-amino-2-(2-furyl)-5-[(3-methyl-2-pyridyl)methylcarbamoyl]pyrimidin-4-yl]oxyethyl]piperidine-1-carboxylate (step 1) (15 mg, 0.02 mmol) was dissolved in methanolic HCl (3M, 2 mL, 6 mmol) and the mixture stirred at 40° C. for 3 hours. The resulting mixture was concentrated in vacuo and purification of the crude material by column chromatography on silica eluting with 5% MeOH in DCM followed by 5% 7M methanolic ammonia solution in DCM afforded the titled compound as a white solid.

LC-MS (Method 8B): Rt 4.92 mins; MS m/z 437.3=[M+H]+

1H NMR (500 MHz, DMSO-d6) δ 9.50 (t, J=4.0 Hz, 1H), 9.12 (br s, 1H), 8.44-8.41 (m, 1H), 7.90 (dd, J=1.7, 0.9 Hz, 1H), 7.71 (br s, 1H), 7.65 (ddd, J=7.5, 1.5, 0.7 Hz, 1H), 7.29 (dd, J=7.5, 4.9 Hz, 1H), 7.26 (dd, J=3.4, 0.9 Hz, 1H), 6.68 (dd, J=3.4, 1.7 Hz, 1H), 4.68-4.59 (m, 2H), 4.56 (d, J=4.0 Hz, 2H), 2.88-2.81 (m, 1H), 2.59-2.52 (m, 2H), 2.40-2.34 (m, 1H), 2.30 (s, 3H), 2.02-1.88 (m, 2H), 1.70-1.64 (m, 1H), 1.59 (br d, J=11.9 Hz, 1H), 1.46-1.40 (m, 1H), 1.28-1.17 (m, 2H), 1.12-1.03 (m, 1H).

### Example 23.1

### 4-Amino-6-(2-aminoethoxy)-2-(2-furyl)-N-[(3-methyl-2-pyridyl)methyl]pyrimidine-5-carboxamide

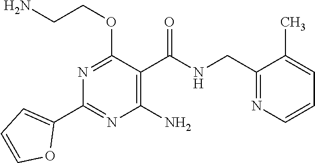 [SMILES: Cc1cccnc1CNC(=O)c1c(N)nc(-c2ccco2)nc1OCCN]

The titled compound was prepared from 4-amino-6-chloro-2-(2-furyl)-N-[(3-methyl-2-pyridyl)methyl]pyrimidine-5-carboxamide (Example 13) and commercially available N-Boc-ethanolamine analogously to Example 23 steps 1 and 2

LC-MS (Method 8B): Rt 3.77 mins; MS m/z 369.4=[M+H]+

1H NMR (500 MHz, DMSO-d6) δ 9.50 (br t, J=3.7 Hz, 1H), 9.15 (br s, 1H), 8.44 (d, J=4.1 Hz, 1H), 7.94-7.88 (m, 1H), 7.73 (br s, 1H), 7.66 (d, J=7.4 Hz, 1H), 7.30 (dd, J=7.4, 4.9 Hz, 1H), 7.28-7.23 (m, 1H), 6.69 (dd, J=3.3, 1.7 Hz, 1H), 4.59 (d, J=3.8 Hz, 2H), 4.49 (t, J=5.5 Hz, 2H), 3.08 (t, J=5.3 Hz, 2H), 2.31 (s, 3H), 1.78 (br s, 2H).

### Example 23.2

### 4-Amino-2-(2-furyl)-N-[(3-methyl-2-pyridyl)methyl]-6-(2-piperazin-1-ylethoxy) pyrimidine-5-carboxamide

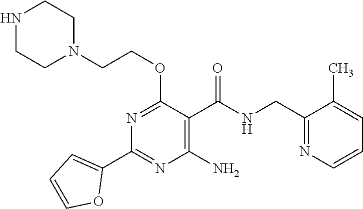 [SMILES: Cc1cccnc1CNC(=O)c1c(N)nc(-c2ccco2)nc1OCCN1CCNCC1]

The titled compound was prepared from tert-butyl 4-[2-[6-amino-2-(2-furyl)-5-[(3-methyl-2-pyridyl)methylcarbamoyl]pyrimidin-4-yl]oxyethyl]piperazine-1-carboxylate (Example 20.4) and methanolic 3M HCl and analogously to Example 23 step 2.

LC-MS (Method 8): Rt 3.86 mins; MS m/z 438.2=[M+H]+

1H NMR (500 MHz, Chloroform-d) 9.61-9.54 (i, 1H), 9.43 (brs, H), 8.46-8.41 (m, 1H), 7.60 (dd, J=1.7, 0.9 Hz, 1H), 7.50 (brddd, =7.5, 1.7, 0.9 Hz, 1H), 7.29-7.24 (m, 1H), 7.17 (dd, J=7.6, 4.8 Hz, 1H), 6.54 (dd, J=3.4, 1.8 Hz, 1H), 5.70 (br s, 1H), 4.76 (t, J=6.3 Hz, 2H), 4.68 (d, J=4.1 Hz, 2H), 3.03 (t, J=6.4 Hz, 2H), 2.86 (apr t, J=4.9 Hz, 4H), 2.57 (br s, 14H), 2.34 (7, 3H).

The compounds of the following tabulated Examples (Table 6) were prepared analogously to Example 23 steps 1 and 2 from 4-amino-6-chloro-2-(2-furyl)-N-[(3-methyl-2-pyridyl)methyl]pyrimidine-5-carboxamide (Example 13) and the appropriate commercially available alcohol.

TABLE 6

| Ex. | Structure and Name | Retention Time, [M + H]+, 1H NMR |
|---|---|---|
| 23.5 | Step 1: tert-butyl N-[3-[6-amino-2-(2-furyl)-5- | Step 1: LC-MS (Method 3B): Rt |
| | [(3-methyl-2-pyridyl)methylcarbamoyl] | 1.98 mins; MS m/z 483.3 = [M + H]+ |
| | pyrimidin-4-yl]oxypropyl]carbamate 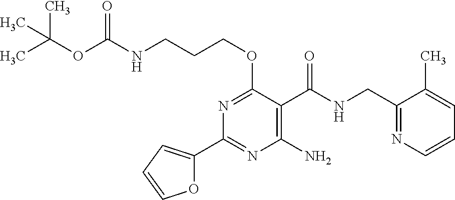 [SMILES: Cc1cccnc1CNC(=O)c1c(N)nc(-c2ccco2)nc1OCCCNC(=O)OC(C)(C)C] Step 2: 4-Amino-6-(3-aminopropoxy)-2-(2- furyl)-N-[(3-methyl-2- pyridyl)methyl]pyrimidine-5-carboxamide dihydrochloride 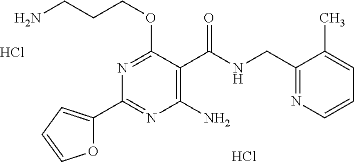 [SMILES: Cl ; Cc1cccnc1CNC(=O)c1c(N)nc(-c2ccco2)nc1OCCCN ; Cl] | 1H NMR (500 MHz, Chloroform-d) δ 9.60 (br s, 1H), 9.44 (br s, 1H), 8.46-8.40 (m, 1H), 7.60 (dd, J = 1.6, 0.7 Hz, 1H), 7.55 (br s, 1H), 7.29 (br s, 1H), 7.21 (br s, 1H), 6.54 (dd, J = 3.4, 1.6 Hz, 1H), 5.75 (br s, 1H), 5.54 (br s, 1H), 4.77 (t, J = 5.6 Hz, 2H), 4.69 (s, 2H), 3.33 (apr q, J = 5.8 Hz, 2H), 2.38 (br s, 3H), 2.19 (apr p, J = 5.8 Hz, 2H), 1.41 (s, 9H). Step 2: LC-MS (Method 8B): Rt 4.15 mins; MS m/z 383.3 = [M + H]+ (free base) 1H NMR (500 MHz, DMSO-d6) δ 9.07 (br s, 1H), 8.63 (d, J = 5.0 Hz, 1H), 8.25 (br s, 1H), 8.09 (br s, 3H), 7.92 (dd, J = 1.7, 0.8 Hz, 1H), 7.75 (br s, 1H), 7.30 (dd, J = 3.4, 0.8 Hz, 1H), 6.70 (dd, J = 3.4, 1.7 Hz, 1H), 4.83 (s, 2H), 4.67 (t, J = 6.0 Hz, 2H), 3.02-2.94 (m, 2H), 2.50 (s, 3H), 2.19 (apr p, J = 6.6 Hz, 2H). |

### Example 24

### 4-Amino-2-(3-cyanophenyl)-6-(2-fluoroethoxy)-N-isopropyl-pyrimidine-5-carboxamide

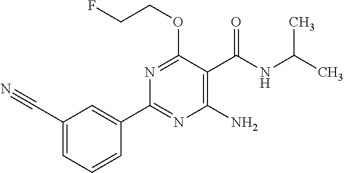 [SMILES: CC(C)NC(=O)c1c(N)nc(-c2cccc(C#N)c2)nc1OCCF]

### Step 1: Ethyl 4-amino-6-(2-fluoroethoxy)-2-methylsulfanyl-pyrimidine-5-carboxylate

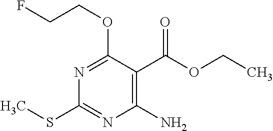 [SMILES: CCOC(=O)c1c(N)nc(SC)nc1OCCF]

Commercially available 2-fluoroethanol (520 μL, 8.86 mmol) was added dropwise to a stirred suspension of sodium hydride (60% in oil) (488 mg, 12.2 mmol) in anhydrous THF (16 mL) at room temperature under nitrogen. After stirring for 15 mins, the resulting gelatinous suspension was treated, rapidly via cannular, with a solution of ethyl 4-amino-6-chloro-2-methylsulfanyl-pyrimidine-5-carboxylate (Example 13 step 2) (2 g, 8.07 mmol) in anhydrous THF (40 mL) and the mixture stirred at room temperature for 75 mins. The reaction was quenched by addition of water (50 mL) and brine (20 mL) and the mixture was extracted with EtOAc (150 mL). The organic portion was washed with brine (25 mL), dried over MgSO₄ and concentrated in vacuo. Purification of the crude material by column chromatography on silica eluting with a gradient of 5 to 30% EtOAc in petrol afforded the titled compound as a pale yellow solid.

LC-MS (Method 3B): Rt 1.74 mins; MS m/z 276.0=[M+H]+

1H NMR (500 MHz, Chloroform-d) δ 8.21 (br s, 1H), 5.66 (br s, 1H), 4.79-4.76 (m, 1H), 4.70-4.67 (m, 1H), 4.67-4.64 (m, 1H), 4.61-4.58 (m, 1H), 4.30 (q, J=7.1 Hz, 2H), 2.49 (s, 3H), 1.37 (t, J=7.1 Hz, 3H).

### Step 2: Ethyl 4-amino-2-(3-cyanophenyl)-6-(2-fluoroethoxy)pyrimidine-5-carboxylate

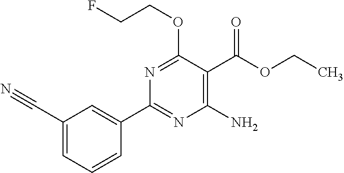 [SMILES: CCOC(=O)c1c(N)nc(-c2cccc(C#N)c2)nc1OCCF]

The titled compound was prepared from ethyl 4-amino-6-(2-fluoroethoxy)-2-methylsulfanyl-pyrimidine-5-carboxylate (step 1) and commercially available (3-cyanophenyl)boronic acid analogously to Example 22 step 2.

LC-MS (Method 8B): Rt 5.05 mins; MS m/z 331.1=[M+H]+

1H NMR (500 MHz, DMSO-d6) δ 8.66 (t, J=1.3 Hz, 1H), 8.60 (dt, J=8.0, 1.3 Hz, 1H), 8.03 (dt, J=7.6, 1.3 Hz, 1H), 7.87 (br s, 2H), 7.74 (t, J=7.8 Hz, 1H), 4.84-4.80 (m, 1H), 4.79-4.75 (m, 1H), 4.75-4.69 (m, 2H), 4.26 (q, J=7.1 Hz, 2H), 1.29 (t, J=7.1 Hz, 3H).

### Step 3:4-Amino-2-(3-cyanophenyl)-6-(2-fluoroethoxy)pyrimidine-5-carboxylic Acid

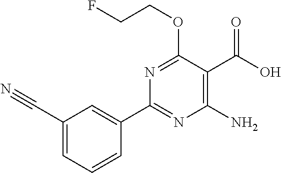 [SMILES: N#Cc1cccc(-c2nc(N)c(C(=O)O)c(OCCF)n2)c1]

The titled compound was prepared from ethyl 4-amino-2-(3-cyanophenyl)-6-(2-fluoroethoxy)pyrimidine-5-carboxylate (step 2) and LiOH analogously to Example 22 step 3.

LC-MS (Method 3A): Rt 1.64 mins; MS m/z 303.0=[M+H]+

1H NMR (500 MHz, DMSO-d6) δ 12.84 (br s, 1H), 8.65 (s, 1H), 8.60 (d, J=7.8 Hz, 1H), 8.02 (d, J=7.8 Hz, 1H), 7.95 (br s, 2H), 7.74 (t, J=7.8 Hz, 1H), 4.85-4.77 (m, 2H), 4.73 (s, 2H).

### Step 4: 4-Amino-2-(3-cyanophenyl)-6-(2-fluoroethoxy)-N-isopropyl-pyrimidine-5-carboxamide

The titled compound was prepared from 4-amino-2-(3-cyanophenyl)-6-(2-fluoroethoxy)pyrimidine-5-carboxylic acid (step 3) and commercially available isopropylamine analogously to Example 3 step 3.

LC-MS (Method 8B): Rt 5.07 mins; MS m/z 344.1=[M+H]+

1H NMR (500 MHz, DMSO-d6) δ 8.65 (td, J=1.7, 0.5 Hz, 1H), 8.59 (ddd, J=8.0, 1.7, 1.2 Hz, 1H), 8.52 (br s, 1H), 8.01 (ddd, J=7.7, 1.7, 1.2 Hz, 1H), 7.92 (d, J=7.4 Hz, 1H), 7.82 (br s, 1H), 7.77-7.71 (m, 1H), 4.91-4.88 (m, 1H), 4.84-4.82 (m, 1H), 4.81-4.75 (m, 2H), 4.09-3.99 (m, 1H), 1.16 (d, J=6.5 Hz, 6H).

The compounds of the following tabulated Examples (Table 7) were prepared analogously to Example 24 step 4 from 4-amino-2-(3-cyanophenyl)-6-(2-fluoroethoxy)pyrimidine-5-carboxylic acid (Example 24 step 3) and the appropriate commercially available amine.

TABLE 7

| | | Retention Time, [M + H]+, 1H |
|---|---|---|
| Ex. | Structure and Name | NMR |
| 24.1 | 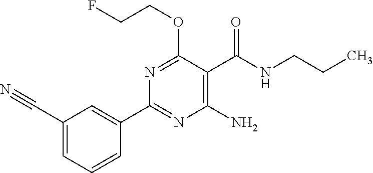 [SMILES: CCCNC(=O)c1c(N)nc(-c2cccc(C#N)c2)nc1OCCF] 4-Amino-2-(3-cyanophenyl)-6-(2-fluoro ethoxy)- N-propyl-pyrimidine-5-carboxamide | LC-MS (Method 8B): Rt 4.50 mins; MS m/z 344.2 = [M + H]+ 1H NMR (500 MHz, DMSO-d6) δ 8.65 (td, J = 1.7, 0.6 Hz, 1H), 8.59 (ddd, J = 8.0, 1.7, 1.2 Hz, 1H), 8.53 (br s, 1H), 8.08 (t, J = 5.6 Hz, 1H), 8.01 (ddd, J = 7.7, 1.7, 1.2 Hz, 1H), 7.80 (br s, 1H), 7.76- 7.71 (m, 1H), 4.92-4.88 (m, 1H), 4.86-4.83 (m, 1H), 4.82-4.77 (m, 2H), 3.24 (td, J = 6.9, 5.6 Hz, 2H), 1.53 (apr sext, J = 7.4 Hz, 2H), 0.92 (t, J = 7.4 Hz, 3H) |
| 24.2 | 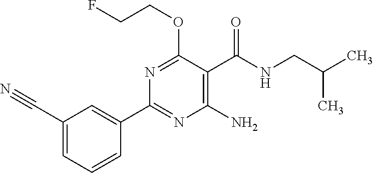 [SMILES: CC(C)CNC(=O)c1c(N)nc(-c2cccc(C#N)c2)nc1OCCF] 4-Amino-2-(3-cyanophenyl)-6-(2-fluoro ethoxy)- N-isobutyl-pyrimidine-5-carboxamide | LC-MS (Method 8B): Rt 5.28 mins; MS m/z 358.2 = [M + H]+ 1H NMR (500 MHz, DMSO-d6) δ 8.65 (td, J = 1.7, 0.6 Hz, 1H), 8.60 (ddd, J = 8.0, 1.7, 1.2 Hz, 1H), 8.46 (br s, 1H), 8.09 (t, J = 5.7 Hz, 1H), 8.02 (ddd, J = 7.7, 1.7, 1.2 Hz, 1H), 7.81 (br s, 1H), 7.76- 7.72 (m, 1H), 4.91-4.87 (m, 1H), 4.87-4.83 (m, 1H), 4.79 (s, 2H), 3.12 (dd, J = 6.7, 5.7 Hz, 2H), 1.80 (th, J = 6.7, 6.7 Hz, 1H), 0.92 (d, J = 6.7 Hz, 6H). |
| 24.3 | 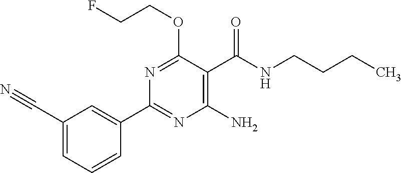 [SMILES: CCCCNC(=O)c1c(N)nc(-c2cccc(C#N)c2)nc1OCCF] 4-Amino-N-butyl-2-(3-cyanophenyl)-6-(2- fluoroethoxy)pyrimidine-5-carboxamide | LC-MS (Method 8B): Rt 5.05 mins; MS m/z 358.2 = [M + H]+ 1H NMR (500 MHz, DMSO-d6) δ 8.65 (td, J = 1.7, 0.5 Hz, 3H), 8.59 (ddd, J = 8.0, 1.7, 1.2 Hz, 1H), 8.48 (br s, 1H), 8.06 (t, J = 5.6 Hz, 1H), 8.01 (ddd, J = 7.7, 1.7, 1.2 Hz, 1H), 7.81 (br s, 1H), 7.98- 7.44 (m, 1H), 4.91-4.88 (m, 1H), 4.86-4.83 (m, 1H), 4.82-4.77 (m, 2H), 3.28 (td, J = 6.9, 5.6 Hz, 2H), 1.53-1.46 (m, 2H), 1.40- 1.32 (m, 2H), 0.90 (t, J = 7.3 Hz, 3H). |
| 24.4 | 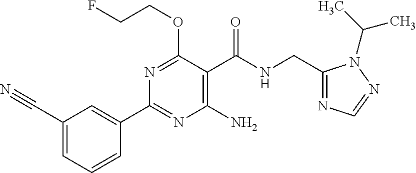 [SMILES: CC(C)n1ncnc1CNC(=O)c1c(N)nc(-c2cccc(C#N)c2)nc1OCCF] 4-Amino-2-(3-cyanophenyl)-6-(2-fluoro ethoxy)- N-[(2-isopropyl-1,2,4-triazol-3- yl)methyl]pyrimidine-5-carboxamide | LC-MS (Method 8B): Rt 4.52 mins; MS m/z 425.3 = [M + H]+ 1H NMR (500 MHz, DMSO-d6) δ 8.79 (t, J = 5.3 Hz, 1H), 8.66 (br apr t, J = 1.4 Hz, 2H), 8.60 (apr dt, J = 8.0, 1.4 Hz, 1H), 8.56 (br s, 1H), 8.02 (apr dt, J = 7.8, 1.4 Hz, 1H), 7.89 (s, 1H), 7.82 (br s, 1H), 7.74 (t, J = 7.8 Hz, 1H), 4.95- 4.91 (m, 1H), 4.89-4.86 (m, 1H), 4.85-4.81 (m, 2H), 4.77 (hept, J = 6.5 Hz, 1H), 4.68 (d, J = 5.3 Hz, 2H), 1.39 (d, J = 6.5 Hz, 6H). |
| 24.5 | 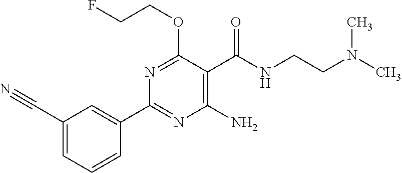 [SMILES: CN(C)CCNC(=O)c1c(N)nc(-c2cccc(C#N)c2)nc1OCCF] 4-Amino-2-(3-cyanophenyl)-N-[2-(dimethylamino) ethyl]-6-(2-fluoroethoxy) pyrimidine-5- | LC-MS (Method 8B): Rt 4.68 mins; MS m/z 373.2 = [M + H]+ 1H NMR (500 MHz, Chloroform- d) δ 9.37 (s, 1H), 8.67 (ddd, J = 1.7, 1.7, 0.4 Hz, 1H), 8.59 (ddd, J = 8.0, 1.7, 1.3 Hz, 1H), 8.42 (t, J = 4.1 Hz, 1H), 7.76 (ddd, J = 7.7, 1.7, 1.3 Hz, 1H), 7.57 (ddd, J = 8.0, 7.7, 0.4 Hz, 1H), 5.67 (s, 1H), 4.96-4.91 (m, 1H), 4.91-4.86 (m, 1H), 4.86-4.79 (m, 2H), 3.53 (dt, J = 5.8, 4.1 Hz, 2H), 2.59 (d, J = 5.8 Hz, 2H), 2.35 (s, 6H). |
| | carboxamide | |
| 24.6 | 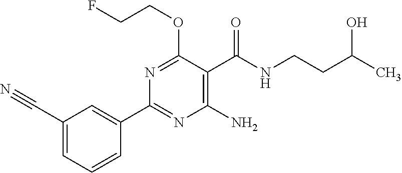 [SMILES: CC(O)CCNC(=O)c1c(N)nc(-c2cccc(C#N)c2)nc1OCCF] 4-Amino-2-(3-cyanophenyl)-6-(2-fluoro ethoxy)- N-(3-hydroxybutyl)pyrimidine-5-carboxamide | LC-MS (Method 8B): Rt 4.24 mins; MS m/z 374.2 = [M + H]+ 1H NMR (500 MHz, DMSO-d6) δ 8.69 (br s, 1H), 8.65 (t, J = 1.5 Hz, 1H), 8.59 (dt, J = 8.0, 1.5 Hz, 1H), 8.20 (t, J = 5.4 Hz, 1H), 8.01 (dt, J = 7.7, 1.5 Hz, 1H), 7.78 (br s, 1H), 7.74 (apr t, J = 7.8 Hz, 1H), 4.93-4.87 (m, 1H), 4.88-4.82 (m, 1H), 4.83-4.76 (m, 2H), 4.60 (d, J = 4.6 Hz, 1H), 3.78-3.67 (m, 1H), 3.42-3.33 (m, 2H), 1.65- 1.48 (m, 2H), 1.09 (d, J = 6.3 Hz, 3H). |
| 24.7 | 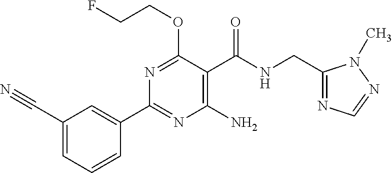 [SMILES: Cn1ncnc1CNC(=O)c1c(N)nc(-c2cccc(C#N)c2)nc1OCCF] 4-Amino-2-(3-cyanophenyl)-6-(2-fluoro ethoxy)- | LC-MS (Method 8B): Rt 3.99 mins; MS m/z 397.2 = [M + H]+ 1H NMR (500 MHz, DMSO-d6) δ 8.78 (t, J = 5.3 Hz, 1H), 8.66 (td, J = 1.8, 0.6 Hz, 1H), 8.60 (ddd, J = 8.0, 1.8, 1.2 Hz, 1H), 8.48 (br s, 1H), 8.02 (ddd, J = 7.7, 1.8, 1.2 Hz, 1H), 7.90 (br s, 1H), 7.86 (s, 1H), 7.76-7.72 (m, 1H), 4.95- 4.86 (m, 2H), 4.86-4.79 (m, 2H), 4.65 (d, J = 5.3 Hz, 2H), 3.87 (s, 3H). |
| | N-[(2-methyl-1,2,4-triazol-3-yl) | |
| | methyl]pyrimidine-5-carboxamide | |
| 24.8 | 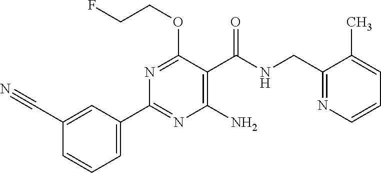 [SMILES: Cc1cccnc1CNC(=O)c1c(N)nc(-c2cccc(C#N)c2)nc1OCCF] 4-Amino-2-(3-cyanophenyl)-6-(2-fluoroethoxy)- | LC-MS (Method 7B): Rt 3.78 mins; MS m/z 407.3 = [M + H]+ 1H NMR (400 MHz, DMSO-d6) δ 9.53 (t, J = 3.9 Hz, 1H), 8.68 (t, 1H), 8.63 (d, J = 8.0 Hz, 1H), 8.42 (d, J = 4.2 Hz, 1H), 8.02 (d, J = 7.7 Hz, 1H), 7.75 (t, J = 7.8 Hz, 1H), 7.65 (d, J = 7.4 Hz, 1H), 7.29 (dd, J = 7.5, 4.9 Hz, 1H), 5.11- 4.82 (m, 4H), 4.59 (d, J = 3.9 Hz, 2H), 2.31 (s, 3H). |
| | N-[(3-methyl-2-pyridyl)methyl]pyrimidine-5- | |
| | carboxamide | |

### Example 25

### 4-Amino-6-(2-fluoroethoxy)-N-[(3-methyl-2-pyridyl)methyl]-2-oxazol-2-yl-pyrimidine-5-carboxamide

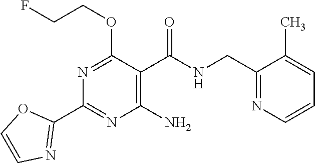 [SMILES: Cc1cccnc1CNC(=O)c1c(N)nc(-c2ncco2)nc1OCCF]

### Step 1: Ethyl 4-amino-6-chloro-2-oxazol-2-yl-pyrimidine-5-carboxylate

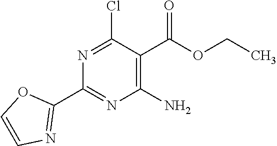 [SMILES: CCOC(=O)c1c(N)nc(-c2ncco2)nc1Cl]

Solution 1: A mixture comprising commercially available oxazole (398 μL, 6.06 mmol) in THF (15 mL) was cooled to −78° C. and treated dropwise with n-butyllithium (2.5 M in hexanes, 2.83 mL, 7.06 mmol). After 20 mins, zinc chloride (2162 mg, 16.15 mmol) was added portion wise and the mixture was stirred at −78° C. for 15 mins and, after warming to room temperature, stirred for a further 30 mins.

Solution 2: A solution comprising ethyl 4-amino-6-chloro-2-methylsulfanyl-pyrimidine-5-carboxylate (Example 13 step 2) (0.5 g, 2.02 mmol), Pd(PPh₃)₄ (117 mg, 0.1 mmol) and copper(I) 2-thiophenecarboxylate (1.15 g, 6.06 mmol) in THF (15 mL) under nitrogen was stirred at room temperature for 10 mins.

Solution 1 was added slowly to solution 2 and the resulting mixture was stirred at 80° C., under nitrogen for 4 hours. After cooling to room temperature, the reaction was quenched with dropwise addition of water and diluted with ethyl acetate (100 mL). The resulting mixture was washed with 10% NH₄OH solution (3×50 mL) and the combined aqueous portions were extracted with ethyl acetate (2×50 mL). The combined organic extracts were washed with brine (100 mL), dried over MgSO₄ and concentrated in vacuo. Purification of the crude material by column chromatography on silica eluting with 0 to 100% EtOAc in heptane afforded a solid that was triturated with EtOH to give the titled compound.

LC-MS (Method 2A): Rt 0.93 mins; MS m/z=268.9 [M+H]+

1H NMR (500 MHz, DMSO-d6) δ 8.34 (d, J=0.6 Hz, 1H), 7.51 (d, J=0.6 Hz, 1H), 4.35 (q, J=7.1 Hz, 2H), 1.31 (t, J=7.1 Hz, 3H).

### Step 2: 4-Amino-6-(2-fluoroethoxy)-2-oxazol-2-yl-pyrimidine-5-carboxylic Acid

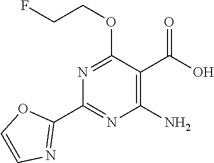 [SMILES: Nc1nc(-c2ncco2)nc(OCCF)c1C(=O)O]

To a stirred suspension of sodium hydride (60% in oil) (24 mg, 0.6 mmol) in DMF (1 mL) was added 2-fluoroethanol (27 μL, 0.45 mmol). After stirring for 10 minutes, ethyl 4-amino-6-chloro-2-oxazo-2-yl-pyrimidine-5-carboxylate (step 1) (85 mg, 0.3 mmol) and stirring continued for 3 hours. Further portions of sodium hydride (60% in oil) (24 mg, 0.6 mmol) and 2-fluoroethanol (27 μL, 0.45 mmol) were added and stirring continued for 5 hours. The reaction was quenched with water (3 mL) and the mixture was stirred overnight. The mixture was neutralised with 1M HCl and concentrated in vacuo to afford the titled compound as a brown gum which was used in the next step without further purification.

LC-MS (Method 2A): Rt 0.79 mins; MS m/z 269=[M+H]+

1H NMR (500 MHz, DMSO-d6) δ 8.23 (d, J=0.6 Hz, 1H), 7.40 (d, J=0.6 Hz, 1H), 4.78-4.73 (m, 1H), 4.72-4.64 (m, 1H), 4.62-4.57 (m, 1H), 4.55-4.49 (m, 1H).

### Step 3: 4-Amino-6-(2-fluoroethoxy)-N-[(3-methyl-2-pyridyl)methyl]-2-oxazol-2-yl-pyrimidine-5-carboxamide

The titled compound was prepared from 4-amino-6-(2-fluoroethoxy)-2-oxazol-2-yl-pyrimidine-5-carboxylic acid (step 2) and (3-methyl-2-pyridyl)methanamine analogously to Example 3 step 3.

LC-MS (Method 7B): Rt 2.62 mins; MS m/z 373.2=[M+H]+

1H NMR (500 MHz, DMSO-d6) δ 9.50 (t, J=4.0 Hz, 1H), 9.13 (br.s, 1H), 8.41 (d, J=3.9 Hz, 1H), 8.33 (d, J=0.6 Hz, 1H), 8.03 (br.s, 1H), 7.65 (d, J=6.8 Hz, 1H), 7.49 (d, J=0.6 Hz, 1H), 7.29 (dd, J=7.5, 4.9 Hz, 1H), 5.05-5.00 (m, 1H), 4.95-4.90 (m, 1H), 4.85-4.82 (m, 1H), 4.79-4.76 (m, 1H), 4.59 (d, J=4.1 Hz, 2H), 2.31 (s, 3H).

### BIOLOGICAL EXAMPLES

### Example 26—Adenosine Receptor Time-Resolved Fluorescence Resonance Energy Transfer (TRFRET) Binding Assay

All FRET binding experiments were conducted at room temperature in white 384-well plates, in assay binding buffer containing 1× LabMed (Cisbio, France), 100 μg/mL saponin, 1% DMSO and 0.02% pluronic acid. Binding of the fluorescently labelled Adenosine receptor antagonist XAC (CA200645, FRET acceptor) to terbium-labelled A1, A2a, A2b and A3 adenosine receptors (FRET donors) was detected by time-resolved FRET due to the close proximity of the donor and acceptor in a binding event. To investigate the ability of unlabelled test compounds to bind to Adenosine A1, A2a, A2b and A3 receptors, dose response curves were constructed that determined the ability of a range of concentrations to inhibit the binding of 30 nM CA200645 to the A2b receptor and 100 nM CA200645 to the A1, A2a, and A3 receptor.

Serial dilution (1:3 dilutions) of test compounds in neat DMSO and transfer of a 400 nL sample of test compound into the assay plate was carried out using the Mosquito (TTP Labtech, UK). The compound samples were incubated for 2 hours at room temperature with a fixed concentration of CA200645 defined for each receptor (see above) and CHO cell membranes containing the human Adenosine A1 (0.5 μg/well), A2a (0.3 μg/well), A2b (1 μg/well) or A3 (1 μg/well) receptor in 40 μL of assay buffer. Total and non-specific binding of CA200645 was determined in the absence and presence of 10 μM XAC, respectively.

Following 2 hours incubation, the level of CA200645 binding was detected on a Pherastar FSX (BMG Labtech, Germany) using standard TR-FRET settings. The terbium donor was excited with three laser flashes at a wavelength of 337 nm, and donor and acceptor emission was detected at 620 nm and 665 nm wavelengths, respectively. FRET ratios were obtained by multiplying the acceptor/donor ratio value by 10,000. Specific binding was determined by subtracting the non-specific binding FRET ratio from the total binding FRET ratio. Compound IC50 curves were analysed using GraphPad Prism 7.0 (GraphPad, USA) and Ki affinity values were determined from the obtained 10C50 values using the method of Cheng and Prusoff. The results are presented in Tables 8 and 9.

TABLE 8

| Compound | A2a |
|---|---|
| of Example | Ki (nM) |
| 1 | 60 |
| 2 | 0.9 |
| 3 | 1.7 |
| 4 | 74 |
| 4.1 | 141 |
| 4.2 | 112 |
| 4.3 | 12 |
| 5 | 329 |
| 5.1 | 90 |
| 5.2 | 20 |
| 5.3 | 20 |
| 5.4 | 50 |
| 5.5 | 278 |
| 5.6 | 29 |
| 5.7 | 505 |
| 5.8 | 21 |
| 5.9 | 76 |
| 5.10 | 150 |
| 5.11 | 495 |
| 5.12 | 4.1 |
| 5.13 | 20 |
| 5.14 | 33 |
| 5.15 | 5.1 |
| 5.16 | 0.93 |
| 5.17 | 24 |
| 5.18 | 3.8 |
| 5.19 | 15 |
| 5.20 | 1.1 |
| 5.21 | 1.5 |
| 5.22 | 871 |
| (Comparative) | |
| 5.23 | 10 |
| 5.24 | 2.4 |
| 5.25 | 1.2 |
| 5.26 | 24 |
| 5.27 | 17 |
| 5.28 | 298 |
| 5.29 | 52 |
| 6 | 556 |
| (Comparative) | |
| 7 | 11 |
| 8 | 51 |
| 9 | 312 |
| 10 | 796 |
| 10.1 | 13 |
| 10.2 | 194 |
| 10.3 | 180 |
| 10.4 | 959 |
| 10.5 | 137 |
| 10.6 | 32 |
| 10.7 | 104 |
| 10.8 | 27 |
| 10.9 | 661 |
| 10.10 | 96 |
| 10.11 | 976 |
| 10.12 | 36 |
| 10.13 | 4.7 |
| 10.14 | 42 |
| 10.15 | 34 |
| 11.1 | 31 |
| 11.2 | 1.2 |
| 12 | 27 |
| 13 | 20 |
| 14 | 744 |
| 15 | 265 |
| 16 | 34 |
| 17 | 3.8 |
| 18 | 1.1 |
| 19 | 105 |

With further testing, the following data was obtained:

TABLE 9

| Example | A2a Ki | A2b Ki | A1 Ki | A3 Ki |
|---|---|---|---|---|
| 1 | 61 | 382 | 314 | 101 |
| 2 | 0.79 | 95 | 318 | 50 |
| 3 | 3.3 | 440 | 1721 | 908 |
| 3.1 | 424 | 5540 | 995 | 818 |
| 4 | 75 | 1207 | | |
| 4.1 | 142 | 1727 | | |
| 4.2 | 112 | 8059 | | |
| 4.3 | 12 | 689 | 49 | 54 |
| 5 | 329 | | | |
| 5.1 | 90 | 172 | | |
| 5.10 | 150 | 10000 | | |
| 5.11 | 495 | | | |
| 5.12 | 3.9 | 1573 | 962 | 1161 |
| 5.13 | 20 | 3971 | 3555 | 2682 |
| 5.14 | 33 | 6629 | | 1823 |
| 5.15 | 5.1 | 1211 | 3986 | 2165 |
| 5.16 | 0.64 | 297 | 1353 | |
| 5.17 | 24 | | 1885 | 3051 |
| 5.18 | 3.3 | 215 | 1764 | 1936 |
| 5.19 | 15 | | | |
| 5.2 | 20 | 113 | 66 | |
| 5.20 | 0.95 | 1194 | 426 | 757 |
| 5.21 | 1.5 | 1161 | 1854 | 1185 |
| 5.22 | 871 | | 4241 | |
| 5.23 | 10 | | 4747 | |
| 5.24 | 2.6 | 1604 | 1269 | |
| 5.25 | 0.83 | 1474 | 706 | 506.3 |
| 5.26 | 24 | 4694 | 4857 | 6028 |
| 5.27 | 17 | | 2682 | |
| 5.28 | 298 | | 5656 | 5776 |
| 5.29 | 52 | 5470 | 1700 | 2455 |
| 5.3 | 20 | 341 | | |
| 5.30 | 2.7 | 2354 | 4752 | 2304 |
| 5.31 | 1.2 | 4222 | 1531 | |
| 5.32 | 3.3 | 437 | 1894 | 1412 |
| 5.33 | 9.4 | 6676 | | |
| 5.34 | 10 | 1415 | 1038 | 1911 |
| 5.35 | 12 | 1329 | 2623 | 2799 |
| 5.36 | 14 | 970 | 2662 | |
| 5.37 | 16 | 1597 | | |
| 5.38 | 20 | 2356 | | |
| 5.39 | 21 | 2131 | 520.7 | 753.2 |
| 5.4 | 50 | 138 | | |
| 5.4 | 48 | 1562 | 4224 | 3671 |
| 5.41 | 52 | 1086 | 4735 | |
| 5.42 | 53 | | | |
| 5.43 | 58 | 5141 | 4781 | 5925 |
| 5.44 | 78 | | 364 | 1087 |
| 5.45 | 123 | | | |
| 5.46 | 131 | 6209 | | |
| 5.47 | 230 | | 4774 | |
| 5.48 | 23 | 420 | 7958 | 3045 |
| 5.49 | 159 | | | |
| 5.5 | 278 | | | |
| 5.5 | 258 | | | |
| 5.51 | 41 | | 2846 | 148 |
| 5.52 | 11 | 5133 | 4795 | 3090 |
| 5.53 | 11 | 644 | 6020 | 4633 |
| 5.54 | 0.87 | 415 | 430.65 | 658 |
| 5.54 | 734 | | | |
| 5.55 | 21 | | | |
| 5.57 | 40 | | 4479 | 1232 |
| 5.58 | 105 | 2119 | | |
| 5.59 | 236 | | | 4606 |
| 5.6 | 29 | 622 | | |
| 5.6 | 412 | | | |
| 5.61 | 54 | | 4085 | 1469 |
| 5.7 | 505 | 3263 | | |
| 5.8 | 21 | 679 | | |
| 5.9 | 76 | 195 | | |
| 6 | 556 | | | |
| 7 | 12 | 383 | 42 | 13 |
| 8 | 51 | 1103 | 137 | 24 |
| 9 | 312 | | | |
| 10 | 796 | 8492 | 4439 | 1795 |
| 10.1 | 13 | 54 | 93 | 421 |
| 10.10 | 96 | 378 | | |
| 10.11 | 976 | 3194 | 632 | 1850 |
| 10.12 | 36 | 536 | 400 | 371 |
| 10.13 | 3.3 | 415 | 38 | 105 |
| 10.14 | 42 | 1136 | 159 | 609 |
| 10.15 | 34 | 1284 | 90 | 2506 |
| 10.2 | 194 | 1480 | 280 | 582 |
| 10.3 | 180 | 1357 | 598 | 504 |
| 10.4 | 959 | 6063 | 3800 | 1822 |
| 10.5 | 137 | 2973 | 658 | 300 |
| 10.6 | 32 | 1308 | 231 | 160 |
| 10.7 | 104 | 2416 | 238 | 778 |
| 10.8 | 27 | 1748 | 143 | 230 |
| 10.9 | 661 | 1173 | 811 | 1162 |
| 11.1 | 31 | | 2384 | |
| 11.2 | 0.71 | 50 | 787 | |
| 12 | 27 | 1564 | 17 | 1027 |
| 13 | 20 | 3895 | 314 | 1190 |
| 14 | 744 | | 740 | 202 |
| 15 | 265 | | | |
| 16 | 34 | | 5648 | |
| 16.1 | 40 | 7168 | | |
| 17 | 2.4 | 405 | 767 | 442 |
| 18 | 0.64 | 231 | 202 | 201 |
| 18.1 | 0.23 | 495 | 176 | 149 |
| 18.3 | 0.078 | 5.4 | 18 | 194 |
| 19 | 105 | | 871 | 675 |
| 20 | 1.1 | 508 | 109 | 830 |
| 20.1 | 0.13 | 107 | 5.4 | 86 |
| 20.10 | 1.8 | 1222 | 4.9 | 11 |
| 20.11 | 0.11 | 80 | 1.7 | 11 |
| 20.12 | 0.21 | 540 | 13 | 1.4 |
| 20.13 | 0.099 | 611 | 9 | 14 |
| 20.2 | 0.21 | 37 | 4 | 9.9 |
| 20.3 | 0.47 | 174 | 4.1 | 1.7 |
| 20.6 | 0.54 | 382 | 19 | 5.6 |
| 20.7 | 0.11 | 29 | 6.6 | 15 |
| 20.8 | 0.199 | 165 | 7.5 | 24 |
| 20.9 | 0.52 | 384 | 44 | 18 |
| 21 | 233 | | | |
| 22 | 6.8 | | 598 | |
| 23 | 864 | | 8526 | |
| 23.1 | 0.62 | 490 | 59 | 73 |
| 23.5 | 23 | 3060 | 1056 | 2148 |
| 24 | 3.3 | | | |
| 24.1 | 5.7 | | | |
| 24.2 | 2.0 | | | |
| 24.3 | 0.90 | | | |
| 24.4 | 1.08 | | 22 | 611 |
| 24.5 | 29 | | | |
| 24.6 | 7 | 1069 | 629 | 2225 |
| 24.7 | 8.1 | | 145 | 1606 |
| 25 | 1.3 | 77 | 260 | 62 |
| 23.2 | 287 | | 7546 | 6702 |
| 24.8 | 0.1 | | 3.8 | 28.5 |

### Example 27—CD3/CD28 Stimulated IL-2 Release NECA Reversal Assay in Human PBMCs

Blood is drawn from healthy volunteers using sodium citrate as the anticoagulant (0.3% final concentration). After centrifugation of the blood over Histopaque-1077, PBMCs are collected from the Histopaque/plasma interface and washed twice in PBS (300 g for 10 mins at room temp). Cells are plated at 50,000 cells/well in 150 μl RPMI/10% FCS in 96-well cell culture plates that have been precoated with 1 ug/ml CD3 antibody. 50 μl diluted compound mix is added to the cells, to obtain final concentrations of 1 ug/ml CD28 antibody, 1 uM NECA and 0.003-10 μM adenosine receptor antagonist. Assay plates are incubated for 24 hours at 37° C. in a humidified incubator. Culture supernatant is tested for IL-2 levels using the human IL-2 Tissue Culture Kit (Meso Scale Discovery). Data for dose-response curves is calculated as % inhibition with 100% inhibition defined from no agonist control wells (+CD3/28-NECA).

TABLE 10

| | IL-2 release |
|---|---|
| | NECA reversal |
| Example | IC₅₀ (nM) |
| 4.3 | 705 |
| 5.2 | 1918 |
| 5.3 | 1545 |
| 3 | 77 |
| 7 | 830 |
| 12 | 1198 |
| 2 | 408 |
| 10.1 | 216 |
| 10.6 | 685 |
| 10.8 | 2005 |
| 10.13 | 926 |
| 10.14 | 1237 |
| 10.15 | 1292 |
| 11.2 | 71 |
| 17 | 309 |
| 5.12 | 345 |
| 5.16 | 102 |
| 5.18 | 230 |
| 5.20 | 122 |
| 5.21 | 123 |
| 5.23 | 381 |
| 5.24 | 364 |
| 18 | 37 |
| 5.25 | 15 |
| 5.54 | 83 |
| 20.7 | 14 |
| 20.11 | 9 |
| 20.13 | 19 |
| 20.10 | 62 |
| 18.1 | 28 |
| 18.3 | 5 |
| 24.6 | 207 |
| 24.7 | 212 |
| 25 | 11 |

### Example 28—LPS Stimulated TNF-Alpha Release NECA Reversal Assay in Human Whole Blood

Blood is drawn from healthy volunteers using sodium citrate (0.3% final concentration) or heparin (19.5 U/ml) as the anticoagulant. A 15 minute incubation may be given with adenosine deaminase (2 u/ml) prior to plating the blood into assay plates, as indicated. 20 μl diluted compound mix containing LPS, NECA and adenosine receptor antagonist diluted in RPMI medium is added to U-bottom cell culture plates. 180 μl anticoagulated blood is added. Assay plates are incubated for 5 hours at 37° C. in a humidified incubator. Plasma obtained by centrifugation at 2000 rpm/10 mins is tested for TNF alpha levels using the human TNF alpha Tissue Culture Kit (Meso Scale Discovery). For bar charts, data is expressed as raw counts from a Mesoscale Sector Imager 6000. Dose-response curves are calculated as % inhibition with 100% inhibition defined from no agonist control wells (+LPS-NECA).

TABLE 11

| | TNF release |
|---|---|
| | NECA reversal |
| Example | IC₅₀ (nM) |
| 3 | 2291 |
| 20.11 | 221 |
| 18.3 | 476 |

### Example 29—Measurement of pCREB in CD8+T Cells in Human Whole Blood

Heparinised human whole blood was pre-incubated at 37° C. with serial dilutions of A2a antagonists for 20 min. and the phosphodiesterase inhibitor rolipram to amplify the pCREB response. The adenosine receptor agonist NECA is then added at a final concentration of 3 μM and following a 60 min incubation the blood is fixed and red blood cells lysed. White blood cells are isolated, permeabilized and stained with directly conjugated fluorescent antibodies to phospho-CREB (Alexa Fluor 488) and CD8 (Alexa Fluor 647) and the level of phospho-CREB in CD8+ T cells is measured by FACS using a BD Accuri C6 Flow Cytometer.

TABLE 12

| | pCREB |
|---|---|
| Example | IC50 (nM) |
| 3 | 1214 |
| 20.11 | 42 |
| 18.3 | 17 |
| 25 | 106 |

### REFERENCES

1. Sukari A Nagasaka M AI-Hadidi A and Lum L G (2016). Anticancer Res. 36(11):5593-5606.
2. Vijayan D, Young A, Teng M W L, and Smyth M J (2017), Nat Rev Cancer. 17(12):709-724.
3. Houthuys, E, Marillier R, Deregnaucourt, T, Brouwer, M, Pirson, R, Marchante, J, et al (2016). SITC 2017 Conference, Maryland.
4. Gao Z W, Dong K, Zhang H Z (2014). “The roles of CD73 in cancer”. Biomed Res Int: 2014:460654.
5. Loi S, Pommey S, Haibe-Kains B, Beavis P A, Darcy P K, Smyth M J, et al. (2013), “CD73 promotes anthracycline resistance and poor prognosis in triple negative breast cancer” Proc Nat Acad Sci USA.; 110(27):11091-6.
6. Deaglio S, Dwyer K M, Gao W, Friedman D, Usheva A, Erat A et al (2007). J. Exp Med. 204, No. 6, Jun. 11, 2007 1257-1265

The following numbered clauses 1-27 are not claims, but instead serve to define particular aspects and embodiments of the invention:

1. A compound of general formula (I) including all tautomeric forms, enantiomers, isotopic variants, salts and solvates thereof:

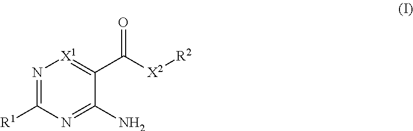

wherein
X¹ is CR³ or N
R³ is H or halo; or
C₁₋₆ alkyl, —O(C₁₋₆ alkyl), —NH(C₁₋₆ alkyl) or —N(C₁₋₆ alkyl)₂, any of which may optionally be substituted with one or more substituents selected from halo, OH, —O(C₁₋₆ alkyl), —NR⁹R¹⁰, —NRC(O)R¹⁰, NR⁹C(═NR⁴)NR¹⁰, NR⁹C(S)R¹⁰, carbocyclyl, heterocyclyl, aryl and heteroaryl;
   wherein R⁴ is H or methyl and each R⁹ and R¹⁰ is independently selected from H, C₁₋₆ alkyl and C₁₋₆ haloalkyl; or
carbocyclyl, heterocyclyl, aryl or heteroaryl;
wherein carbocyclyl, heterocyclyl, aryl and heteroaryl groups are optionally substituted with one or more substituents selected from halo, C₁₋₆ alkyl, C₁₋₆ haloalkyl and
—NR¹¹R¹²;
   wherein each R¹¹ and R¹² is independently selected from H, C₁₋₆ alkyl and C₁₋₆ haloalkyl;

### X² is O or NH

R¹ is aryl or heteroaryl optionally substituted with one or more substituents selected from halo, OH, CN, R⁵, OR⁵ and NR⁵R⁶,
each R⁵ and R⁶ is independently H, C₁₋₆ alkyl or C₃₋₇ cycloalkyl, either of which is optionally substituted with one or more substituents selected from halo, OH, aryl and heteroaryl, wherein aryl and heteroaryl groups are optionally substituted with one or more substituents selected from halo, OH, C₁₋₆ alkyl and C₁₋₆ haloalkyl

### R² is:

C₁₋₆ alkyl optionally substituted with one or more substituents selected from halo, OH, O—C₁₋₆ alkyl, NH(C₁₋₆ alkyl), N(C₁₋₆ alkyl)₂ and R⁸; or

### R⁸;

wherein each R⁸ is independently aryl or heteroaryl, either or which may optionally be substituted with one or more substituents selected from halo, OH, NH₂, CN, NO₂, R⁷, OR⁷ NHR⁷ or N(R⁷)₂;
   each R⁷ is independently C₁₋₆ alkyl optionally substituted with one or more substituents selected from halo, OH, —O(C₁₋₆ alkyl) and —O(C₁₋₆ haloalkyl).

2. A compound according to clause 1 wherein X¹ is N.

3. A compound according to clause 1 wherein X¹ is CR³, wherein R³ is as defined in clause 1.

4. A compound according to clause 3 wherein R³ is H; halo; or —O(C₁₋₆ alkyl) optionally substituted with one or more substituents selected from halo, OH and —O(C₁₋₆ alkyl); or

a 6-membered aryl or 5- or 6-membered heteroaryl, either of which is optionally substituted with one or more substituents selected from halo, OH, C₁₋₆ alkyl, —O(C₁₋₆ alkyl), C₁₋₆ haloalkyl and —O(C₁₋₆ haloalkyl).

5. A compound according to any one of clauses 1 to 4 wherein X² is NR⁴, where R⁴ is as defined in clause 1 and the compound is of general formula (Ia):

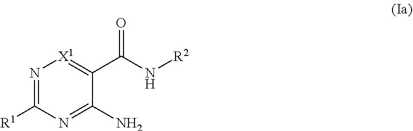

wherein X¹, R¹, R² and R⁴ are as defined in clause 1.

6. A compound according to any one of clauses 1 to 4 wherein X² is O and the compound is of general formula (Ib):

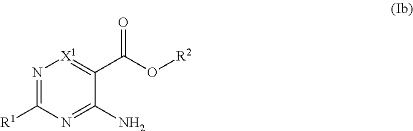

wherein X¹, R¹ and R² are as defined in clause 1.

7. A compound according to clause 6 wherein R² is a group R^{2b}, where R^{2b} is C₁₋₆ alkyl.

8. A compound according to any one of clauses 1 to 7 wherein R¹ is phenyl or 5- or 6-membered heteroaryl optionally substituted as defined in clause 1.

9. A compound according to clause 8 wherein R¹ is phenyl, furanyl, oxazolyl or pyrazolyl, any of which may be unsubstituted or substituted as defined in clause 1.

10. A compound according to clause 9, wherein R¹ is:

phenyl substituted with halo or cyano; or
unsubstituted furan-2-yl.

11. A compound according to any one of clauses 1 to 6 or 8 to 10 wherein R² is C₁₋₆ alkyl optionally substituted with one or more substituents selected from halo, OH, O—C₁₋₆ alkyl and R⁸, wherein R⁸ is as defined in clause 1.

12. A compound according to clause 11 wherein when:

### X¹ is CH;

### X² is NH; and

R² is C₁₋₆ alkyl substituted with R⁸;
R¹ and R⁸ are not both selected from unsubstituted phenyl, phenyl substituted with methyl, unsubstituted pyridyl, unsubstituted furyl and unsubstituted thienyl.

13. A compound according to clause 11 or clause 12 wherein R² is CH₂—R⁸, CH₂—CH₂—R⁸ or CH(CH₃)—R⁸, where R⁸ is as defined in clause 1.

14. A compound according to clause 13 wherein R⁸ is phenyl, indan-1-yl, indan-2-yl, pyridin-2-yl, imidazol-2-yl, quinolin-8-yl and triazol-3-yl, any of which is optionally substituted with one or more substituents selected from halo; OH; C₁₋₆ alkyl and —O(C₁₋₆ alkyl), either of which is optionally substituted with halo, OH or —O(C₁₋₆ alkyl).
15. A compound according to clause 14 wherein R³ is:
   pyridyl optionally substituted with Me, OH, OMe, OEt, CF₃, F; or
   quinolinyl optionally substituted with Me, OH, OMe, OEt, CF₃, F; or
   phenyl optionally substituted with Me, OH, OMe, OEt, CF₃, F.

16. A compound according to clause 1 wherein R¹ is furan-2-yl, R² is CH₂-(3-methylpyridin-2-yl); and X² is NH.

17. A compound according to clause 1 selected from:
ethyl 5-amino-3-(2-furyl)-1,2,4-triazine-6-carboxylate;
5-Amino-3-(2-furyl)-N-[(3-methyl-2-pyridyl)methyl]-1,2,4-triazine-6-carboxamide;
4-Amino-2-(2-furyl)-N-[(3-methyl-2-pyridyl)methyl]pyrimidine-5-carboxamide;
4-Amino-N-benzyl-2-(3-fluorophenyl)pyrimidine-5-carboxamide;
4-Amino-2-(3-fluorophenyl)-N-(2-phenylethyl)pyrimidine-5-carboxamide;
4-Amino-2-(3-fluorophenyl)-N-phenyl-pyrimidine-5-carboxamide;
4-amino-2-(3-fluorophenyl)-N-[(3-methyl-2-pyridyl)methyl]pyrimidine-5-carboxamide;
4-Amino-2-(3-fluorophenyl)-N-[(1R)-1-phenylethyl]pyrimidine-5-carboxamide;
4-Amino-2-(3-fluorophenyl)-N-[(2-fluorophenyl)methyl]pyrimidine-5-carboxamide;
4-Amino-N-[(2-fluoro-6-methoxy-phenyl)methyl]-2-(3-fluorophenyl)pyrimidine-5-carboxamide;
4-Amino-N-[(2-ethoxy-6-fluoro-phenyl)methyl]-2-(3-fluorophenyl)pyrimidine-5-carboxamide;
4-Amino-N-[(2,6-difluorophenyl)methyl]-2-(3-fluorophenyl)pyrimidine-5-carboxamide;
4-Amino-2-(3-fluorophenyl)-N-(pyrimidin-2-ylmethyl)pyrimidine-5-carboxamide;
Amino-2-(3-fluorophenyl)-N-[[3-(trifluoromethyl)-2-pyridyl]methyl]pyrimidine-5-carboxamide;
4-Amino-2-(3-fluorophenyl)-N-[2-(4-hydroxyphenyl)ethyl]pyrimidine-5-carboxamide;
4-Amino-N-[(3-ethoxy-2-pyridyl)methyl]-2-(3-fluorophenyl)pyrimidine-5-carboxamide;
4-Amino-2-(3-fluorophenyl)-N-(2-pyridylmethyl)pyrimidine-5-carboxamide;
4-Amino-2-(3-fluorophenyl)-N-(o-tolylmethyl)pyrimidine-5-carboxamide;
4-Amino-2-(2-furyl)-N-[(4-methoxyphenyl)methyl]pyrimidine-5-carboxamide;
4-Amino-N-[(3-chlorophenyl)methyl]-2-(2-furyl)pyrimidine-5-carboxamide;
4-Amino-N-[(3-fluorophenyl)methyl]-2-(2-furyl)pyrimidine-5-carboxamide;
4-Amino-N-[(4-fluorophenyl)methyl]-2-(2-furyl)pyrimidine-5-carboxamide;
4-Amino-N-[(2-chlorophenyl)methyl]-2-(2-furyl)pyrimidine-5-carboxamide;
4-Amino-N-[(2,6-dichlorophenyl)methyl]-2-(2-furyl)pyrimidine-5-carboxamide;
4-Amino-N-[(3,5-difluorophenyl)methyl]-2-(2-furyl)pyrimidine-5-carboxamide;
4-Amino-N-[(2-fluorophenyl)methyl]-2-(2-furyl)pyrimidine-5-carboxamide;
4-Amino-N-[(2,4-dichlorophenyl)methyl]-2-(2-furyl)pyrimidine-5-carboxamide;
4-Amino-2-(2-furyl)-N-[(2-methoxyphenyl)methyl]pyrimidine-5-carboxamide;
4-Amino-N-[(3-chloro-2-pyridyl)methyl]-2-(2-furyl)pyrimidine-5-carboxamide;
4-Amino-2-(2-furyl)-N-indan-1-yl-pyrimidine-5-carboxamide;
4-Amino-2-(2-furyl)-N-indan-2-yl-pyrimidine-5-carboxamide;
4-amino-2-(2-furyl)-N-(8-quinolylmethyl) pyrimidine-5-carboxamide;
4-Amino-N-[(1-ethylimidazol-2-yl)methyl]-2-(2-furyl)pyrimidine-5-carboxamide;
4-amino-2-(2-furyl)-N-[(2-isopropyl-1,2,4-triazol-3-yl)methyl]pyrimidine-5-carboxamide;
4-amino-2-(2-furyl)-N-[[4-(trifluoromethyl)phenyl]methyl]pyrimidine-5-carboxamide;
4-amino-2-(2-furyl)-N-[[3-(trifluoro methyl)phenyl]methyl]pyrimidine-5-carboxamide;
4-amino-2-(2-furyl)-N-[[3-(trifluoromethyl)-2-pyridyl]methyl]pyrimidine-5-carboxamide;
ethyl 5-amino-3-(3-fluorophenyl)-1,2,4-triazine-6-carboxylate;
5-Amino-3-(3-fluorophenyl)-N-[(3-methyl-2-pyridyl)methyl]-1,2,4-triazine-6-carboxamide;
5-Amino-3-(4-fluorophenyl)-N-[(3-methyl-2-pyridyl)methyl]-1,2,4-triazine-6-carboxamide;
4-Amino-2-(2-fluorophenyl)-N-[(3-methyl-2-pyridyl)methyl]pyrimidine-5-carboxamide;
4-Amino-2-(3-fluorophenyl)-N-(p-tolylmethyl)pyrimidine-5-carboxamide;
4-Amino-2-(3-fluorophenyl)-N-[(2-hydroxyphenyl)methyl]pyrimidine-5-carboxamide;
4-Amino-N-[(3-chlorophenyl)methyl]-2-(3-fluorophenyl)pyrimidine-5-carboxamide;
4-Amino-2-(3-fluorophenyl)-N-[(3-fluorophenyl)methyl]pyrimidine-5-carboxamide;
4-Amino-2-(3-fluorophenyl)-N-[(4-fluorophenyl) methyl]pyrimidine-5-carboxamide;
4-Amino-N-[(2-chlorophenyl)methyl]-2-(3-fluorophenyl)pyrimidine-5-carboxamide;
4-Amino-N-[(2,6-dichlorophenyl)methyl]-2-(3-fluorophenyl)pyrimidine-5-carboxamide;
4-Amino-N-[(3,5-difluorophenyl)methyl]-2-(3-fluorophenyl)pyrimidine-5-carboxamide;
4-Amino-2-(3-fluorophenyl)-N-[(2-methoxyphenyl)methyl]pyrimidine-5-carboxamide;
4-Amino-N-[(3,5-dimethoxyphenyl)methyl]-2-(3-fluorophenyl)pyrimidine-5-carboxamide;
4-Amino-N-[(2,3-dimethoxyphenyl)methyl]-2-(3-fluorophenyl)pyrimidine-5-carboxamide;
4-Amino-2-(3-fluorophenyl)-N-[[3-(trifluoromethyl)phenyl]methyl]pyrimidine-5-carboxamide;
4-Amino-N-[(3-chloro-2-pyridyl)methyl]-2-(3-fluorophenyl)pyrimidine-5-carboxamide;
4-Amino-2-(3-fluorophenyl)-N-(8-quinolylmethyl)pyrimidine-5-carboxamide;
4-Amino-N-[(1-ethylimidazol-2-yl)methyl]-2-(3-fluorophenyl)pyrimidine-5-carboxamide;
4-Amino-2-(3-fluorophenyl)-N-[(2-isopropyl-1,2,4-triazol-3-yl)methyl]pyrimidine-5-carboxamide;
4-amino-2-(2-furyl)-N-(p-tolylmethyl)pyrimidine-5-carboxamide;
4-amino-2-(2-furyl)-N-[(2-hydroxyphenyl) methyl]pyrimidine-5-carboxamide;
4-Amino-2-(3-cyanophenyl)-N-[(3-methyl-2-pyridyl)methyl]pyrimidine-5-carboxamide;
4-Amino-6-chloro-2-(2-furyl)-N-[(3-methyl-2-pyridyl)methyl]pyrimidine-5-carboxamide;
4-Amino-2-(4-fluorophenyl)-N-[(3-methyl-2-pyridyl)methyl]pyrimidine-5-carboxamide;
4-Amino-N-[(3-methyl-2-pyridyl)methyl]-2-pyrazol-1-yl-pyrimidine-5-carboxamide;
4-Amino-N-[(3-methyl-2-pyridyl)methyl]-2-oxazol-2-yl-pyrimidine-5-carboxamide;
5-Amino-3-(3-cyanophenyl)-N-[(3-methyl-2-pyridyl)methyl]-1,2,4-triazine-6-carboxamide;
4-Amino-2-(2-furyl)-6-methoxy-N-[(3-methyl-2-pyridyl)methyl]pyrimidine-5-carboxamide;
4-Amino-2-(2-furyl)-N-[(3-methyl-2-pyridyl)methyl]-6-pyrazol-1-yl-pyrimidine-5-carboxamide;
   and pharmaceutically acceptable salts and solvates thereof.

18. A process for the preparation of a compound according to clause 1 comprising

A. for a compound of general formula (Ia):
reacting a compound of general formula (II):

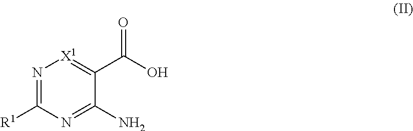

wherein X¹ and R¹ are as defined for general formula (I);
with a compound of general formula (III):

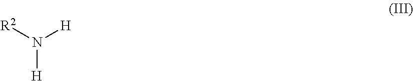

wherein R² is as defined for general formula (I);
B. for a compound of general formula (Ib) in which X¹ is N or C—R³:
reacting a compound of general formula (IV):

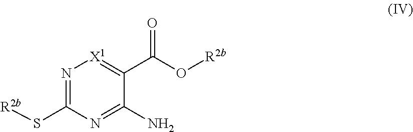

wherein each R^{2b} is independently C₁₋₆ alkyl;
X¹ is N or CR³, wherein R³ is as defined for general formula (I);
with a compound of general formula (V):

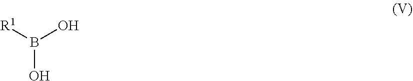

wherein R¹ is as defined for general formula (I);
C. for a compound of general formula (Ib) in which X¹ is CH:
reacting a compound of general formula (V) as defined above with a compound of general formula (XV):

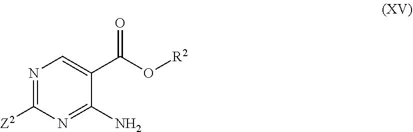

wherein R^{2b} is as defined for general formula (IV) and Z² is halo, suitably chloro;
D. for a compound of general formula (Ia) wherein X¹ is CH:
reacting a compound of general formula (XVI):

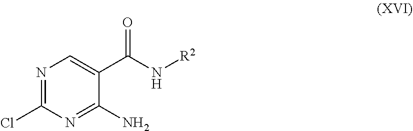

wherein R² is as defined for general formula (I);
with a compound of general formula (V) as defined above;
E. for a compound of general formula (Ib) in which R² is R^{2b} and X¹ is N:
reacting a compound of general formula (XX):

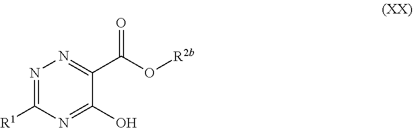

wherein R¹ is as defined for general formula (I) and R^{2b} is as defined for general formula (IV);
with ammonium hydroxide;
F. for a compound of general formula (Ia) in which X¹ is CH:
Reacting a compound of general formula (XXXI):

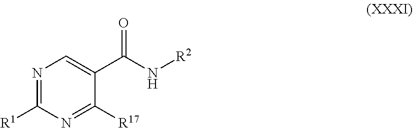

wherein R¹ and R² are as defined for general formula (I) and R¹⁷ is as defined for general formula (XXX);
with sodium azide followed by reduction with triphenylphosphine.

19. A compound according to any one of clauses 1 to 17 for use in medicine.

20. A compound according to any one of clauses 1 to 17 for use in the treatment of cancer, particularly solid tumours, for example non-small cell lung cancer, head and neck squamous cancer and urothelial cancer.

21. The use of a compound according to any one of clauses 1 to 17 in the manufacture of a medicament for the treatment of cancer, particularly solid tumours, for example non-small cell lung cancer, head and neck squamous cancer and urothelial cancer.

22. A method for the treatment of cancer, particularly solid tumours, for example non-small cell lung cancer, head and neck squamous cancer and urothelial cancer, the method comprising administering to a patient in need of such treatment an effective amount of a compound according to any one of clauses 1 to 17.

23. A pharmaceutical composition comprising a compound according to any one of clauses 1 to 17 and a pharmaceutically acceptable excipient.

24. A composition according to clause 23 further including one or more other active agents which are useful in the treatment or prophylaxis of cancer.

25. A product comprising a compound according to any one of clauses 1 to 17 and an additional agent useful in the treatment or prevention of cancer as a combined preparation for simultaneous, sequential or separate use in the treatment of cancer, in particular solid tumours for example non-small cell lung cancer, head and neck squamous cancer and urothelial cancer.

26. A compound according to any one of clauses 1 to 17 in combination with an additional agent useful in the treatment of cancer as a combined preparation for simultaneous, sequential or separate use in the treatment of treatment of cancer, in particular solid tumours for example non-small cell lung cancer, head and neck squamous cancer and urothelial cancer.

27. A composition according to clause 24, a product according to clause 25 or a compound in combination according to clause 26, wherein the additional agent useful in the treatment of cancer is selected from:
other forms of cancer immunotherapy and anti-cancer chemotherapeutic agents; A2b antagonists;
anti-PD-1 and PDL-1 antibodies including pembrolizumab, nivolumab, durvalumab, avelumab and atezolizumab;
anti-CTLA4 antibodies including ipilimumab; and
cell-based immunotherapy and cancer vaccines that include CAR T cell therapy.

1. A compound of general formula (I), including all tautomeric forms, enantiomers, isotopic variants, salts and solvates thereof: 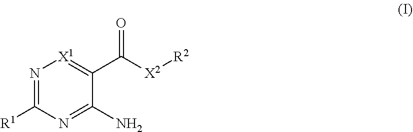
wherein
X¹ is CR³ or N
wherein R³ is:
(i) H;
(ii) halo; or
(iii) C₁₋₆ alkyl, —O(C₁₋₆ alkyl) or —NH(C₁₋₆ alkyl), any of which may optionally be substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl; wherein R⁴ is H or methyl and each R⁹ and R¹⁰ is independently selected from H, C₁₋₆ alkyl and C₁₋₆ haloalkyl;
wherein any carbocyclyl, heterocyclyl, aryl and heteroaryl groups are optionally substituted with one or more substituents selected from halo, C₁₋₆ alkyl, C₁₋₆ haloalkyl and —NR¹¹R¹²; wherein each R¹¹ and R¹² is independently selected from H, C₁₋₆ alkyl and C₁₋₆ haloalkyl;
X² is O or NH;
R¹ is aryl or heteroaryl optionally substituted with one or more substituents selected from halo, OH, CN, R⁵, OR⁵ and NR⁵R⁶,
wherein each R⁵ and R⁶ is independently H, C₁₋₆ alkyl or C₃₋₇ cycloalkyl, either of which is optionally substituted with one or more substituents selected from halo, OH, aryl and heteroaryl, wherein aryl and heteroaryl groups are optionally substituted with one or more substituents selected from halo, OH, C₁₋₆ alkyl and C₁₋₆ haloalkyl; and
R² is:
(i) C₁₋₆ alkyl optionally substituted with one or more substituents selected from halo, OH, O—C₁₋₆ alkyl, NH(C₁₋₆ alkyl), N(C₁₋₆ alkyl)₂ and R⁸; or
(ii) R⁸;
wherein each R⁸ is independently aryl or heteroaryl, either or which may optionally be substituted with one or more substituents selected from halo, OH, NH₂, CN, NO₂, R⁷, OR⁷ NHR⁷ or N(R⁷)₂;
each R⁷ is independently C₁₋₆ alkyl optionally substituted with one or more substituents selected from halo, OH, —O(C₁₋₆ alkyl) and —O(C₁₋₆ haloalkyl);
for use in therapy (e.g. the treatment of cancer).

2. A compound having the general formula (I) defined in claim 1, including all tautomeric forms, enantiomers, isotopic variants, salts and solvates thereof wherein the compound is not one of the following compounds:
ethyl 4-amino-2-(2-methoxyphenyl)pyrimidine-5-carboxylate;
ethyl 4-amino-2-(o-tolyl)pyrimidine-5-carboxylate;
ethyl 4-amino-2-(2-chlorophenyl)pyrimidine-5-carboxylate;
ethyl 4-amino-2-phenylpyrimidine-5-carboxylate;
ethyl 4-amino-2-(4-chlorophenyl)pyrimidine-5-carboxylate;
ethyl 4-amino-2-(2-hydroxyphenyl)pyrimidine-5-carboxylate;
2-(3,5-dimethyl-pyrazol-1-yl)-4-amino-5-carbethoxypyrimidine (ethyl 4-amino-2-(3,5-dimethyl-1H-pyrazol-1-yl)pyrimidine-5-carboxylate);
ethyl 4-amino-2-(2-isopropoxyphenyl)pyrimidine-5-carboxylate;
ethyl-4-amino-6-methyl-2-(p-chloro-phenyl)pyrimidin-5-carboxylate;
ethyl-5-amino-3-phenyl-1,2,4-triazine-6-carboxylate;
ethyl-5-amino-3-(pyridin-2-yl)-1,2,4-triazine-6-carboxylate;
ethyl-5-amino-3-(pyrimidin-2-yl)-1,2,4-triazine-6-carboxylate;
ethyl-5-amino-3-(pyrazin-2-yl)-1,2,4-triazine-6-carboxylate;
5-Pyrimidinecarboxamide, 4-amino-2-(2-furanyl)-N-(3-methylbutyl)-;
5-Pyrimidinecarboxamide, 4-amino-N-hexyl-2-(2-thienyl)-;
5-Pyrimidinecarboxamide, 4-amino-N-(1,1-dimethylethyl)-2-(2-furanyl)-;
5-Pyrimidinecarboxamide, 4-amino-N-(1-methylpropyl)-2-(2-thienyl)-;
5-Pyrimidinecarboxamide, 4-amino-N-propyl-2-(2-thienyl)-;
5-Pyrimidinecarboxamide, 4-amino-N-(4,6-dimethyl-2-pyrimidinyl)-2-(2-furanyl)-;
5-Pyrimidinecarboxamide, 4-amino-2-(2-furanyl)-N-(5-methyl-1,3,4-thiadiazol-2-yl)-;
5-Pyrimidinecarboxamide, 4-amino-N-2-pyrimidinyl-2-(2-thienyl)-;
5-Pyrimidinecarboxamide, 4-amino-N-[3-(dimethylamino)propyl]-2-(2-thienyl)-;
5-Pyrimidinecarboxamide, 4-amino-2-(2-furanyl)-N-(4-pyridinylmethyl)-;
5-Pyrimidinecarboxamide, 4-amino-N-(2,4-dimethylphenyl)-2-(2-furanyl)-;
5-Pyrimidinecarboxamide, 4-amino-2-(2-furanyl)-N-2-thiazolyl-;
5-Pyrimidinecarboxamide, 4-amino-2-(2-furanyl)-N-(6-methyl-2-pyridinyl)-;
5-Pyrimidinecarboxamide, 4-amino-N-[2-(dimethylamino)ethyl]-2-(2-furanyl)-;
5-Pyrimidinecarboxamide, 4-amino-2-(2-furanyl)-N-2H-tetrazol-5-yl-;
5-Pyrimidinecarboxamide, 4-amino-N-(3,5-dimethylphenyl)-2-(2-furanyl)-;
5-Pyrimidinecarboxamide, 4-amino-2-(2-furanyl)-N-4-pyridinyl-;
5-Pyrimidinecarboxamide, 4-amino-2-(2-furanyl)-N-(3-methylphenyl)-;
5-Pyrimidinecarboxamide, 4-amino-2-(2-furanyl)-N-(2-pyridinylmethyl)-;
5-Pyrimidinecarboxamide, 4-amino-2-(5-bromo-2-furanyl)-N-methyl-;
5-Pyrimidinecarboxamide, 4-amino-2-(2-furanyl)-N-(4-methoxyphenyl)-;
5-Pyrimidinecarboxamide, 4-amino-2-(2-furanyl)-N-3-pyridinyl-;
5-Pyrimidinecarboxamide, 4-amino-2-(2-furanyl)-N-methyl-;
5-Pyrimidinecarboxamide, 4-amino-N-[3-(dimethylamino)propyl]-2-(2-furanyl)-;
5-Pyrimidinecarboxamide, 4-amino-N-(5-methyl-3-isoxazolyl)-2-(2-thienyl)-;
5-Pyrimidinecarboxamide, 4-amino-N-2-thiazolyl-2-(2-thienyl)-;
5-Pyrimidinecarboxamide, 4-amino-N-(2-fluorophenyl)-2-(2-furanyl)-;
5-Pyrimidinecarboxamide, 4-amino-N-(2-furanylmethyl)-2-(2-thienyl)-;
5-Pyrimidinecarboxamide, 4-amino-N-2H-tetrazol-5-yl-2-(2-thienyl)-;
5-Pyrimidinecarboxamide, 4-amino-N-(4-methylphenyl)-2-(2-thienyl)-;
5-Pyrimidinecarboxamide, 4-amino-2-(2-furanyl)-N-pentyl-;
5-Pyrimidinecarboxamide, 4-amino-N-(3-methoxypropyl)-2-(2-thienyl)-;
5-Pyrimidinecarboxamide, 4-amino-N-butyl-2-(2-furanyl)-;
5-Pyrimidinecarboxamide, 4-amino-N-ethyl-2-(2-furanyl)-;
5-Pyrimidinecarboxamide, 4-amino-N-cyclohexyl-2-(2-thienyl)-;
5-Pyrimidinecarboxamide, 4-amino-2-(2-furanyl)-N-phenyl-;
5-Pyrimidinecarboxamide, 4-amino-N-2-pyridinyl-2-(2-thienyl)-;
5-Pyrimidinecarboxamide, 4-amino-N-ethyl-2-(2-thienyl)-;
5-Pyrimidinecarboxamide, 4-amino-N-3-pyridinyl-2-(2-thienyl)-;
5-Pyrimidinecarboxamide, 4-amino-2-(2-furanyl)-N-(1-phenylethyl)-;
5-Pyrimidinecarboxamide, 4-amino-N-phenyl-2-(2-thienyl)-;
5-Pyrimidinecarboxamide, 4-amino-2-(2-furanyl)-N-(3-pyridinylmethyl)-;
5-Pyrimidinecarboxamide, 4-amino-2-(2-furanyl)-N-(1-methylpropyl)-;
5-Pyrimidinecarboxamide, 4-amino-N-(2-ethylphenyl)-2-(2-furanyl)-;
5-Pyrimidinecarboxamide, 4-amino-2-(2-furanyl)-N-(2-methylphenyl)-;
5-Pyrimidinecarboxamide, 4-amino-N-(2,3-dimethylphenyl)-2-(2-furanyl)-;
5-Pyrimidinecarboxamide, 4-amino-2-(2-furanyl)-N-(5-methyl-2-pyridinyl)-;
5-Pyrimidinecarboxamide, 4-amino-2-(2-furanyl)-N-(phenylmethyl)-;
5-Pyrimidinecarboxamide, 4-amino-2-(2-furanyl)-N-(5-methyl-3-isoxazolyl)-;
5-Pyrimidinecarboxamide, 4-amino-2-(2-furanyl)-N-(2-furanylmethyl)-;
5-Pyrimidinecarboxamide, 4-amino-2-(2-furanyl)-N-(4-methylphenyl)-;
5-Pyrimidinecarboxamide, 4-amino-2-(2-furanyl)-N-hexyl-;
5-Pyrimidinecarboxamide, 4-amino-N-(3-ethoxypropyl)-2-(2-furanyl)-;
5-Pyrimidinecarboxamide, 4-amino-N-(phenylmethyl)-2-(2-thienyl)-;
5-Pyrimidinecarboxamide, 4-amino-N-(3-ethoxypropyl)-2-(2-thienyl)-;
5-Pyrimidinecarboxamide, 4-amino-N-(3,4-dimethylphenyl)-2-(2-furanyl)-;
5-Pyrimidinecarboxamide, 4-amino-N-(1,1-dimethylethyl)-2-(2-thienyl)-;
5-Pyrimidinecarboxamide, 4-amino-2-(2-furanyl)-N-(2-methylpropyl)-;
5-Pyrimidinecarboxamide, 4-amino-N-4-pyridinyl-2-(2-thienyl)-;
5-Pyrimidinecarboxamide, 4-amino-2-(2-furanyl)-N-(4-methyl-2-pyridinyl)-;
5-Pyrimidinecarboxamide, 4-amino-N-[2-(dimethylamino)ethyl]-2-(2-thienyl)-;
5-Pyrimidinecarboxamide, 4-amino-2-(2-furanyl)-N-2-pyridinyl-;
5-Pyrimidinecarboxamide, 4-amino-2-(2-furanyl)-N-propyl-;
5-Pyrimidinecarboxamide, 4-amino-2-(2-furanyl)-N-(1-methylethyl)-;
5-Pyrimidinecarboxamide, 4-amino-2-(2-furanyl)-N-(3-methyl-2-pyridinyl)-;
5-Pyrimidinecarboxamide, 4-amino-N-(3-methylbutyl)-2-(2-thienyl)-;
5-Pyrimidinecarboxamide, 4-amino-N-(4-ethylphenyl)-2-(2-furanyl)-;
5-Pyrimidinecarboxamide, 4-amino-N-(2-methylpropyl)-2-(2-thienyl)-;
5-Pyrimidinecarboxamide, 4-amino-2-(2-furanyl)-N-(2-methoxyphenyl)-;
5-Pyrimidinecarboxamide, 4-amino-N-butyl-2-(2-thienyl)-;
5-Pyrimidinecarboxamide, 4-amino-N-methyl-2-(2-thienyl)-;
5-Pyrimidinecarboxamide, 4-amino-2-(2-furanyl)-N-(3-methoxypropyl)-;
5-Pyrimidinecarboxamide, 4-amino-N-pentyl-2-(2-thienyl)-;
5-Pyrimidinecarboxamide, 4-amino-N-(2-methoxyethyl)-2-(2-thienyl)-;
5-Pyrimidinecarboxamide, 4-amino-2-(2-furanyl)-N-2-pyrimidinyl-;
5-Pyrimidinecarboxamide, 4-amino-N-(2-methylphenyl)-2-(2-thienyl)-;
5-Pyrimidinecarboxamide, 4-amino-N-[2-(diethylamino)ethyl]-2-(2-furanyl)-;
5-Pyrimidinecarboxamide, 4-amino-2-(2-furanyl)-N-(2-methoxyethyl)-;
5-Pyrimidinecarboxamide, 4-amino-N-(4-fluorophenyl)-2-(2-furanyl)-;
5-Pyrimidinecarboxamide, 4-amino-2-(2-furanyl)-N-(2-phenylethyl)-;
5-Pyrimidinecarboxamide, 4-amino-N-(2,6-dimethylphenyl)-2-(2-furanyl)-;
5-Pyrimidinecarboxamide, 4-amino-N-(3-fluorophenyl)-2-(2-furanyl)-;
5-Pyrimidinecarboxamide, 4-amino-2-(2-furanyl)-N-(4-methyl-2-thiazolyl)-;
5-Pyrimidinecarboxamide, 4-amino-N-(1-methylethyl)-2-(2-thienyl)-;
5-Pyrimidinecarboxamide, 4-amino-2-(2-furanyl)-N-[(4-methylphenyl)methyl]-;
5-Pyrimidinecarboxamide, 4-amino-N-(3-methylphenyl)-2-(2-thienyl)-;
5-Pyrimidinecarboxamide, 4-amino-N-(2,5-dimethylphenyl)-2-(2-furanyl)-;
5-Pyrimidinecarboxamide, 4-amino-2-(2-furanyl)-N-[3-(1-methylethoxy)propyl]-;
5-Pyrimidinecarboxamide, 4-amino-N-methyl-2-phenyl-;
5-Pyrimidinecarboxamide, 4-amino-N-methyl-2-(2-pyridinyl)-;
5-Pyrimidinecarboxamide, 4-amino-N-methyl-2-(4-pyridinyl)-;
5-Pyrimidinecarboxamide, 4-amino-N-methyl-2-(3-pyridinyl)-;
5-Pyrimidinecarboxamide, 4-amino-N-ethyl-2-phenyl-;
5-Pyrimidinecarboxamide, 4-amino-N-methyl-2-(2-methylphenyl)-;
5-Pyrimidinecarboxamide, 4-amino-N-methyl-2-(3-methylphenyl)-;
5-Pyrimidinecarboxamide, 4-amino-N-methyl-2-(4-methylphenyl)-;
5-Pyrimidinecarboxamide, 4-amino-N-ethyl-2-(3-pyridinyl)-;
5-Pyrimidinecarboxamide, 4-amino-N-ethyl-2-(4-pyridinyl)-;
5-Pyrimidinecarboxamide, 4-amino-N-ethyl-2-(2-pyridinyl)-;
5-Pyrimidinecarboxamide, 4-amino-2-(4-fluorophenyl)-N-methyl-;
5-Pyrimidinecarboxamide, 4-amino-2-(3-fluorophenyl)-N-methyl-;
5-Pyrimidinecarboxamide, 4-amino-2-(2-fluorophenyl)-N-methyl-;
5-Pyrimidinecarboxamide, 4-amino-N-(1-methylethyl)-2-phenyl-;
5-Pyrimidinecarboxamide, 4-amino-N-ethyl-2-(2-methylphenyl)-;
5-Pyrimidinecarboxamide, 4-amino-2-(4-ethylphenyl)-N-methyl-;
5-Pyrimidinecarboxamide, 4-amino-N-ethyl-2-(3-methylphenyl)-;
5-Pyrimidinecarboxamide, 4-amino-2-phenyl-N-propyl-;
5-Pyrimidinecarboxamide, 4-amino-N-ethyl-2-(4-methylphenyl)-;
5-Pyrimidinecarboxamide, 4-amino-N-(1-methylethyl)-2-(2-pyridinyl)-;
5-Pyrimidinecarboxamide, 4-amino-N-propyl-2-(3-pyridinyl)-;
5-Pyrimidinecarboxamide, 4-amino-N-(1-methylethyl)-2-(4-pyridinyl)-;
5-Pyrimidinecarboxamide, 4-amino-N-propyl-2-(2-pyridinyl)-;
5-Pyrimidinecarboxamide, 4-amino-N-propyl-2-(4-pyridinyl)-;
5-Pyrimidinecarboxamide, 4-amino-N-(1-methylethyl)-2-(3-pyridinyl)-;
5-Pyrimidinecarboxamide, 4-amino-2-(3-methoxyphenyl)-N-methyl-;
5-Pyrimidinecarboxamide, 4-amino-2-(4-methoxyphenyl)-N-methyl-;
5-Pyrimidinecarboxamide, 4-amino-2-(2-methoxyphenyl)-N-methyl-;
5-Pyrimidinecarboxamide, 4-amino-N-ethyl-2-(4-fluorophenyl)-;
5-Pyrimidinecarboxamide, 4-amino-N-ethyl-2-(2-fluorophenyl)-;
5-Pyrimidinecarboxamide, 4-amino-N-ethyl-2-(3-fluorophenyl)-;
5-Pyrimidinecarboxamide, 4-amino-2-(2-chlorophenyl)-N-methyl-;
5-Pyrimidinecarboxamide, 4-amino-2-(3-chlorophenyl)-N-methyl-;
5-Pyrimidinecarboxamide, 4-amino-2-(4-chlorophenyl)-N-methyl-;
5-Pyrimidinecarboxamide, 4-amino-N-ethyl-2-(4-ethylphenyl)-;
5-Pyrimidinecarboxamide, 4-amino-2-(3-methylphenyl)-N-propyl-;
5-Pyrimidinecarboxamide, 4-amino-N-(1-methylethyl)-2-(4-methylphenyl)-;
5-Pyrimidinecarboxamide, 4-amino-N-(1-methylethyl)-2-(2-methylphenyl)-;
5-Pyrimidinecarboxamide, 4-amino-2-(2-methylphenyl)-N-propyl-;
5-Pyrimidinecarboxamide, 4-amino-N-butyl-2-phenyl-;
5-Pyrimidinecarboxamide, 4-amino-N-(1-methylpropyl)-2-phenyl-;
5-Pyrimidinecarboxamide, 4-amino-2-(4-methylphenyl)-N-propyl-;
5-Pyrimidinecarboxamide, 4-amino-N-(1-methylethyl)-2-(3-methylphenyl)-;
5-Pyrimidinecarboxamide, 4-amino-N-methyl-2-[4-(1-methylethyl)phenyl]-;
5-Pyrimidinecarboxamide, 4-amino-N-(1,1-dimethylethyl)-2-phenyl-;
5-Pyrimidinecarboxamide, 4-amino-N-(2-methylpropyl)-2-phenyl-;
5-Pyrimidinecarboxamide, 4-amino-N-(2-methylpropyl)-2-(4-pyridinyl)-;
5-Pyrimidinecarboxamide, 4-amino-N-(1,1-dimethylethyl)-2-(3-pyridinyl)-;
5-Pyrimidinecarboxamide, 4-amino-N-(1-methylpropyl)-2-(3-pyridinyl)-;
5-Pyrimidinecarboxamide, 4-amino-N-(1,1-dimethylethyl)-2-(4-pyridinyl)-;
5-Pyrimidinecarboxamide, 4-amino-N-(1,1-dimethylethyl)-2-(2-pyridinyl)-;
5-Pyrimidinecarboxamide, 4-amino-N-(2-methylpropyl)-2-(3-pyridinyl)-;
5-Pyrimidinecarboxamide, 4-amino-N-(1-methylpropyl)-2-(2-pyridinyl)-;
5-Pyrimidinecarboxamide, 4-amino-N-(2-methylpropyl)-2-(2-pyridinyl)-;
5-Pyrimidinecarboxamide, 4-amino-N-butyl-2-(2-pyridinyl)-;
5-Pyrimidinecarboxamide, 4-amino-N-(1-methylpropyl)-2-(4-pyridinyl)-;
5-Pyrimidinecarboxamide, 4-amino-N-butyl-2-(4-pyridinyl)-;
5-Pyrimidinecarboxamide, 4-amino-N-butyl-2-(3-pyridinyl)-;
5-Pyrimidinecarboxamide, 4-amino-2-(3-ethoxyphenyl)-N-methyl-;
5-Pyrimidinecarboxamide, 4-amino-2-(4-ethoxyphenyl)-N-methyl-;
5-Pyrimidinecarboxamide, 4-amino-N-ethyl-2-(2-methoxyphenyl)-;
5-Pyrimidinecarboxamide, 4-amino-2-(2-ethoxyphenyl)-N-methyl-;
5-Pyrimidinecarboxamide, 4-amino-N-ethyl-2-(4-methoxyphenyl)-;
5-Pyrimidinecarboxamide, 4-amino-N-(2-methoxyethyl)-2-phenyl-;
5-Pyrimidinecarboxamide, 4-amino-N-ethyl-2-(3-methoxyphenyl)-;
5-Pyrimidinecarboxamide, 4-amino-N-methyl-2-(3-nitrophenyl)-;
5-Pyrimidinecarboxamide, 4-amino-N-methyl-2-(4-nitrophenyl)-;
5-Pyrimidinecarboxamide, 4-amino-N-(2-methoxyethyl)-2-(2-pyridinyl)-;
5-Pyrimidinecarboxamide, 4-amino-N-(2-methoxyethyl)-2-(3-pyridinyl)-;
5-Pyrimidinecarboxamide, 4-amino-N-(2-methoxyethyl)-2-(4-pyridinyl)-;
5-Pyrimidinecarboxamide, 4-amino-2-(3-fluorophenyl)-N-(1-methylethyl)-;
5-Pyrimidinecarboxamide, 4-amino-2-(4-fluorophenyl)-N-propyl-;
5-Pyrimidinecarboxamide, 4-amino-2-(2-fluorophenyl)-N-(1-methylethyl)-;
5-Pyrimidinecarboxamide, 4-amino-2-(4-fluorophenyl)-N-(1-methylethyl)-;
5-Pyrimidinecarboxamide, 4-amino-2-(3-fluorophenyl)-N-propyl-;
5-Pyrimidinecarboxamide, 4-amino-2-(2-fluorophenyl)-N-propyl-;
5-Pyrimidinecarboxamide, 4-amino-2-(2-chlorophenyl)-N-ethyl-;
5-Pyrimidinecarboxamide, 4-amino-2-(4-chlorophenyl)-N-ethyl-;
5-Pyrimidinecarboxamide, 4-amino-2-(3-chlorophenyl)-N-ethyl-;
5-Pyrimidinecarboxamide, 4-amino-2-(2-chloro-6-fluorophenyl)-N-methyl-;
5-Pyrimidinecarboxamide, 4-amino-2-phenyl-N-2H-tetrazol-5-yl-;
5-Pyrimidinecarboxamide, 4-amino-2-(3-pyridinyl)-N-2H-tetrazol-5-yl-;
5-Pyrimidinecarboxamide, 4-amino-2-(2-pyridinyl)-N-2H-tetrazol-5-yl-;
5-Pyrimidinecarboxamide, 4-amino-2-(4-pyridinyl)-N-2H-tetrazol-5-yl-;
5-Pyrimidinecarboxamide, 4-amino-2-(2-methylphenyl)-N-(2-methylpropyl)-;
5-Pyrimidinecarboxamide, 4-amino-N-butyl-2-(3-methylphenyl)-;
5-Pyrimidinecarboxamide, 4-amino-2-(4-methylphenyl)-N-(2-methylpropyl)-;
5-Pyrimidinecarboxamide, 4-amino-N-butyl-2-(4-methylphenyl)-;
5-Pyrimidinecarboxamide, 4-amino-N-butyl-2-(2-methylphenyl)-;
5-Pyrimidinecarboxamide, 4-amino-N-ethyl-2-[4-(1-methylethyl)phenyl]-;
5-Pyrimidinecarboxamide, 4-amino-2-[4-(1,1-dimethylethyl)phenyl]-N-methyl-;
5-Pyrimidinecarboxamide, 4-amino-2-(4-ethylphenyl)-N-propyl-;
5-Pyrimidinecarboxamide, 4-amino-N-(1,1-dimethylethyl)-2-(2-methylphenyl)-;
5-Pyrimidinecarboxamide, 4-amino-N-pentyl-2-phenyl-;
5-Pyrimidinecarboxamide, 4-amino-2-(3-methylphenyl)-N-(1-methylpropyl)-;
5-Pyrimidinecarboxamide, 4-amino-N-(3-methylbutyl)-2-phenyl-;
5-Pyrimidinecarboxamide, 4-amino-2-(4-ethylphenyl)-N-(1-methylethyl)-;
5-Pyrimidinecarboxamide, 4-amino-2-(2-methylphenyl)-N-(1-methylpropyl)-;
5-Pyrimidinecarboxamide, 4-amino-N-(1,1-dimethylethyl)-2-(4-methylphenyl)-;
5-Pyrimidinecarboxamide, 4-amino-N-(1,1-dimethylethyl)-2-(3-methylphenyl)-;
5-Pyrimidinecarboxamide, 4-amino-2-(3-methylphenyl)-N-(2-methylpropyl)-;
5-Pyrimidinecarboxamide, 4-amino-2-(4-methylphenyl)-N-(1-methylpropyl)-;
5-Pyrimidinecarboxamide, 4-amino-N-[2-(dimethylamino)ethyl]-2-phenyl-;
5-Pyrimidinecarboxamide, 4-amino-N-(3-methylbutyl)-2-(3-pyridinyl)-;
5-Pyrimidinecarboxamide, 4-amino-N-(3-methylbutyl)-2-(2-pyridinyl)-;
5-Pyrimidinecarboxamide, 4-amino-N-pentyl-2-(2-pyridinyl)-;
5-Pyrimidinecarboxamide, 4-amino-N-(3-methylbutyl)-2-(4-pyridinyl)-;
5-Pyrimidinecarboxamide, 4-amino-N-pentyl-2-(4-pyridinyl)-;
5-Pyrimidinecarboxamide, 4-amino-N-pentyl-2-(3-pyridinyl)-;
5-Pyrimidinecarboxamide, 4-amino-2-(2-methoxyphenyl)-N-propyl-;
5-Pyrimidinecarboxamide, 4-amino-N-(3-methoxypropyl)-2-phenyl-;
5-Pyrimidinecarboxamide, 4-amino-2-(2-methoxyphenyl)-N-(1-methylethyl)-;
5-Pyrimidinecarboxamide, 4-amino-2-(4-ethoxyphenyl)-N-ethyl-;
5-Pyrimidinecarboxamide, 4-amino-N-methyl-2-[2-(1-methylethoxy)phenyl]-;
5-Pyrimidinecarboxamide, 4-amino-2-(3-methoxyphenyl)-N-(1-methylethyl)-;
5-Pyrimidinecarboxamide, 4-amino-2-(4-methoxyphenyl)-N-(1-methylethyl)-;
5-Pyrimidinecarboxamide, 4-amino-N-methyl-2-(4-propoxyphenyl)-;
5-Pyrimidinecarboxamide, 4-amino-2-(3-methoxyphenyl)-N-propyl-;
5-Pyrimidinecarboxamide, 4-amino-2-(4-methoxyphenyl)-N-propyl-;
5-Pyrimidinecarboxamide, 4-amino-N-methyl-2-[4-(1-methylethoxy)phenyl]-;
5-Pyrimidinecarboxamide, 4-amino-N-methyl-2-[3-(1-methylethoxy)phenyl]-;
5-Pyrimidinecarboxamide, 4-amino-N-(2-methoxyethyl)-2-(4-methylphenyl)-;
5-Pyrimidinecarboxamide, 4-amino-N-(2-methoxyethyl)-2-(3-methylphenyl)-;
5-Pyrimidinecarboxamide, 4-amino-2-(3-ethoxyphenyl)-N-ethyl-;
5-Pyrimidinecarboxamide, 4-amino-N-methyl-2-(2-propoxyphenyl)-;
5-Pyrimidinecarboxamide, 4-amino-2-(2-ethoxyphenyl)-N-ethyl-;
5-Pyrimidinecarboxamide, 4-amino-N-(2-methoxyethyl)-2-(2-methylphenyl)-;
5-Pyrimidinecarboxamide, 4-amino-N-methyl-2-(3-propoxyphenyl)-;
5-Pyrimidinecarboxamide, 4-amino-N-[2-(dimethylamino)ethyl]-2-(3-pyridinyl)-;
5-Pyrimidinecarboxamide, 4-amino-N-[2-(dimethylamino)ethyl]-2-(2-pyridinyl)-;
5-Pyrimidinecarboxamide, 4-amino-N-[2-(dimethylamino)ethyl]-2-(4-pyridinyl)-;
5-Pyrimidinecarboxamide, 4-amino-N-(3-methoxypropyl)-2-(4-pyridinyl)-;
5-Pyrimidinecarboxamide, 4-amino-N-(3-methoxypropyl)-2-(2-pyridinyl)-;
5-Pyrimidinecarboxamide, 4-amino-N-(3-methoxypropyl)-2-(3-pyridinyl)-;
5-Pyrimidinecarboxamide, 4-amino-2-(2,3-dimethoxyphenyl)-N-methyl-;
5-Pyrimidinecarboxamide, 4-amino-2-(2,5-dimethoxyphenyl)-N-methyl-;
5-Pyrimidinecarboxamide, 4-amino-2-(3,4-dimethoxyphenyl)-N-methyl-;
5-Pyrimidinecarboxamide, 4-amino-2-(2,4-dimethoxyphenyl)-N-methyl-;
5-Pyrimidinecarboxamide, 4-amino-2-(2-fluorophenyl)-N-(2-methylpropyl)-;
5-Pyrimidinecarboxamide, 4-amino-N-(1,1-dimethylethyl)-2-(2-fluorophenyl)-;
5-Pyrimidinecarboxamide, 4-amino-N-butyl-2-(2-fluorophenyl)-;
5-Pyrimidinecarboxamide, 4-amino-N-butyl-2-(4-fluorophenyl)-;
5-Pyrimidinecarboxamide, 4-amino-N-(1,1-dimethylethyl)-2-(3-fluorophenyl)-;
5-Pyrimidinecarboxamide, 4-amino-2-(4-fluorophenyl)-N-(1-methylpropyl)-;
5-Pyrimidinecarboxamide, 4-amino-2-(3-fluorophenyl)-N-(2-methylpropyl)-;
5-Pyrimidinecarboxamide, 4-amino-2-(4-fluorophenyl)-N-(2-methylpropyl)-;
5-Pyrimidinecarboxamide, 4-amino-2-(3-fluorophenyl)-N-(1-methylpropyl)-;
5-Pyrimidinecarboxamide, 4-amino-N-(1,1-dimethylethyl)-2-(4-fluorophenyl)-;
5-Pyrimidinecarboxamide, 4-amino-N-butyl-2-(3-fluorophenyl)-;
5-Pyrimidinecarboxamide, 4-amino-2-(2-fluorophenyl)-N-(1-methylpropyl)-;
5-Pyrimidinecarboxamide, 4-amino-2-(4-fluorophenyl)-N-(2-methoxyethyl)-;
5-Pyrimidinecarboxamide, 4-amino-2-(3-fluorophenyl)-N-(2-methoxyethyl)-;
5-Pyrimidinecarboxamide, 4-amino-2-(2-fluorophenyl)-N-(2-methoxyethyl)-;
5-Pyrimidinecarboxamide, 4-amino-N,2-diphenyl-;
5-Pyrimidinecarboxamide, 4-amino-2-(2-chlorophenyl)-N-propyl-;
5-Pyrimidinecarboxamide, 4-amino-2-(2-chlorophenyl)-N-(1-methylethyl)-;
5-Pyrimidinecarboxamide, 4-amino-2-(3-chlorophenyl)-N-propyl-;
5-Pyrimidinecarboxamide, 4-amino-2-(3-chlorophenyl)-N-(1-methylethyl)-;
5-Pyrimidinecarboxamide, 4-amino-2-(4-chlorophenyl)-N-(1-methylethyl)-;
5-Pyrimidinecarboxamide, 4-amino-2-(4-chlorophenyl)-N-propyl-;
5-Pyrimidinecarboxamide, 4-amino-N-phenyl-2-(4-pyridinyl)-;
5-Pyrimidinecarboxamide, 4-amino-N-phenyl-2-(3-pyridinyl)-;
5-Pyrimidinecarboxamide, 4-amino-N-phenyl-2-(2-pyridinyl)-;
5-Pyrimidinecarboxamide, 4-amino-2-phenyl-N-2-pyridinyl-;
5-Pyrimidinecarboxamide, 4-amino-2-phenyl-N-3-pyridinyl-;
5-Pyrimidinecarboxamide, 4-amino-2-phenyl-N-4-pyridinyl-;
5-Pyrimidinecarboxamide, 4-amino-N-3-pyridinyl-2-(4-pyridinyl)-;
5-Pyrimidinecarboxamide, 4-amino-N,2-di-4-pyridinyl-;
5-Pyrimidinecarboxamide, 4-amino-2-(2-pyridinyl)-N-3-pyridinyl-;
5-Pyrimidinecarboxamide, 4-amino-2-(2-pyridinyl)-N-4-pyridinyl-;
5-Pyrimidinecarboxamide, 4-amino-N,2-di-3-pyridinyl-;
5-Pyrimidinecarboxamide, 4-amino-N-2-pyridinyl-2-(3-pyridinyl)-;
5-Pyrimidinecarboxamide, 4-amino-N-2-pyridinyl-2-(4-pyridinyl)-;
5-Pyrimidinecarboxamide, 4-amino-2-(3-pyridinyl)-N-4-pyridinyl-;
5-Pyrimidinecarboxamide, 4-amino-2-phenyl-N-2-pyrimidinyl-;
5-Pyrimidinecarboxamide, 4-amino-N,2-di-2-pyridinyl-;
5-Pyrimidinecarboxamide, 4-amino-2-(3-chloro-4-methoxyphenyl)-N-methyl-;
5-Pyrimidinecarboxamide, 4-amino-2-(5-chloro-2-methoxyphenyl)-N-methyl-;
5-Pyrimidinecarboxamide, 4-amino-2-(2-pyridinyl)-N-2-pyrimidinyl-;
5-Pyrimidinecarboxamide, 4-amino-2-(4-pyridinyl)-N-2-pyrimidinyl-;
5-Pyrimidinecarboxamide, 4-amino-2-(3-pyridinyl)-N-2-pyrimidinyl-;
5-Pyrimidinecarboxamide, 4-amino-N-(2-furanylmethyl)-2-phenyl-;
5-Pyrimidinecarboxamide, 4-amino-2-(2-chloro-6-fluorophenyl)-N-ethyl-;
5-Pyrimidinecarboxamide, 4-amino-N-(2-furanylmethyl)-2-(2-pyridinyl)-;
5-Pyrimidinecarboxamide, 4-amino-N-(2-furanylmethyl)-2-(3-pyridinyl)-;
5-Pyrimidinecarboxamide, 4-amino-N-(5-methyl-3-isoxazolyl)-2-phenyl-;
5-Pyrimidinecarboxamide, 4-amino-N-(2-furanylmethyl)-2-(4-pyridinyl)-;
5-Pyrimidinecarboxamide, 4-amino-N-(5-methyl-3-isoxazolyl)-2-(4-pyridinyl)-;
5-Pyrimidinecarboxamide, 4-amino-N-(5-methyl-3-isoxazolyl)-2-(2-pyridinyl)-;
5-Pyrimidinecarboxamide, 4-amino-N-(5-methyl-3-isoxazolyl)-2-(3-pyridinyl)-;
5-Pyrimidinecarboxamide, 4-amino-2-(2-methylphenyl)-N-2H-tetrazol-5-yl-;
5-Pyrimidinecarboxamide, 4-amino-2-(4-methylphenyl)-N-2H-tetrazol-5-yl-;
5-Pyrimidinecarboxamide, 4-amino-2-(3-methylphenyl)-N-2H-tetrazol-5-yl-;
5-Pyrimidinecarboxamide, 4-amino-2-(2,4-dichlorophenyl)-N-methyl-;
5-Pyrimidinecarboxamide, 4-amino-2-(2,6-dichlorophenyl)-N-methyl-;
5-Pyrimidinecarboxamide, 4-amino-2-(3,4-dichlorophenyl)-N-methyl-;
5-Pyrimidinecarboxamide, 4-amino-2-phenyl-N-2-thiazolyl-;
5-Pyrimidinecarboxamide, 4-amino-2-(2-pyridinyl)-N-2-thiazolyl-;
5-Pyrimidinecarboxamide, 4-amino-2-(4-pyridinyl)-N-2-thiazolyl-;
5-Pyrimidinecarboxamide, 4-amino-2-(3-pyridinyl)-N-2-thiazolyl-;
5-Pyrimidinecarboxamide, 4-amino-2-phenyl-N-[(tetrahydro-2-furanyl)methyl]-;
5-Pyrimidinecarboxamide, 4-amino-2-(4-ethylphenyl)-N-(2-methylpropyl)-;
5-Pyrimidinecarboxamide, 4-amino-N-(1,1-dimethylethyl)-2-(4-ethylphenyl)-;
5-Pyrimidinecarboxamide, 4-amino-2-(2-methylphenyl)-N-pentyl-;
5-Pyrimidinecarboxamide, 4-amino-2-[4-(1-methylethyl)phenyl]-N-propyl-;
5-Pyrimidinecarboxamide, 4-amino-N-(1-methylethyl)-2-[4-(1-methylethyl)phenyl]-;
5-Pyrimidinecarboxamide, 4-amino-2-(4-ethylphenyl)-N-(1-methylpropyl)-;
5-Pyrimidinecarboxamide, 4-amino-2-[4-(1,1-dimethylethyl)phenyl]-N-ethyl-;
5-Pyrimidinecarboxamide, 4-amino-N-hexyl-2-phenyl-;
5-Pyrimidinecarboxamide, 4-amino-N-butyl-2-(4-ethylphenyl)-;
5-Pyrimidinecarboxamide, 4-amino-N-(3-methylbutyl)-2-(2-methylphenyl)-;
5-Pyrimidinecarboxamide, 4-amino-N-(3-methylbutyl)-2-(3-methylphenyl)-;
5-Pyrimidinecarboxamide, 4-amino-N-(3-methylbutyl)-2-(4-methylphenyl)-;
5-Pyrimidinecarboxamide, 4-amino-2-(4-methylphenyl)-N-pentyl-;
5-Pyrimidinecarboxamide, 4-amino-2-(3-methylphenyl)-N-pentyl-;
5-Pyrimidinecarboxamide, 4-amino-2-(3-pyridinyl)-N-[(tetrahydro-2-furanyl)methyl]-;
5-Pyrimidinecarboxamide, 4-amino-2-(4-pyridinyl)-N-[(tetrahydro-2-furanyl)methyl]-;
5-Pyrimidinecarboxamide, 4-amino-2-(2-pyridinyl)-N-[(tetrahydro-2-furanyl)methyl]-;
5-Pyrimidinecarboxamide, 4-amino-N-[2-(dimethylamino)ethyl]-2-(4-methylphenyl)-;
5-Pyrimidinecarboxamide, 4-amino-N-hexyl-2-(4-pyridinyl)-;
5-Pyrimidinecarboxamide, 4-amino-N-[2-(dimethylamino)ethyl]-2-(3-methylphenyl)-;
5-Pyrimidinecarboxamide, 4-amino-N-[3-(dimethylamino)propyl]-2-phenyl-;
5-Pyrimidinecarboxamide, 4-amino-N-hexyl-2-(3-pyridinyl)-;
5-Pyrimidinecarboxamide, 4-amino-N-[2-(dimethylamino)ethyl]-2-(2-methylphenyl)-;
5-Pyrimidinecarboxamide, 4-amino-N-hexyl-2-(2-pyridinyl)-;
5-Pyrimidinecarboxamide, 4-amino-2-(4-fluorophenyl)-N-2H-tetrazol-5-yl-;
5-Pyrimidinecarboxamide, 4-amino-2-(3-fluorophenyl)-N-2H-tetrazol-5-yl-;
5-Pyrimidinecarboxamide, 4-amino-2-(2-fluorophenyl)-N-2H-tetrazol-5-yl-;
5-Pyrimidinecarboxamide, 4-amino-2-(2-ethoxyphenyl)-N-(1-methylethyl)-;
5-Pyrimidinecarboxamide, 4-amino-N-ethyl-2-[4-(1-methylethoxy)phenyl]-;
5-Pyrimidinecarboxamide, 4-amino-N-(1,1-dimethylethyl)-2-(2-methoxyphenyl)-;
5-Pyrimidinecarboxamide, 4-amino-N-(3-methoxypropyl)-2-(3-methylphenyl)-;
5-Pyrimidinecarboxamide, 4-amino-N-methyl-2-[3-(1-methylpropoxy)phenyl]-;
5-Pyrimidinecarboxamide, 4-amino-N-ethyl-2-[2-(1-methylethoxy)phenyl]-;
5-Pyrimidinecarboxamide, 4-amino-N-methyl-2-[4-(2-methylpropoxy)phenyl]-;
5-Pyrimidinecarboxamide, 4-amino-N-ethyl-2-(4-propoxyphenyl)-;
5-Pyrimidinecarboxamide, 4-amino-N-(1,1-dimethylethyl)-2-(4-methoxyphenyl)-;
5-Pyrimidinecarboxamide, 4-amino-2-(4-ethoxyphenyl)-N-propyl-;
5-Pyrimidinecarboxamide, 4-amino-N-(3-methoxypropyl)-2-(2-methylphenyl)-;
5-Pyrimidinecarboxamide, 4-amino-2-(4-ethoxyphenyl)-N-(1-methylethyl)-;
5-Pyrimidinecarboxamide, 4-amino-N-ethyl-2-(2-propoxyphenyl)-;
5-Pyrimidinecarboxamide, 4-amino-N-ethyl-2-(3-propoxyphenyl)-;
5-Pyrimidinecarboxamide, 4-amino-2-(4-butoxyphenyl)-N-methyl-;
5-Pyrimidinecarboxamide, 4-amino-2-(2-butoxyphenyl)-N-methyl-;
5-Pyrimidinecarboxamide, 4-amino-2-(4-methoxyphenyl)-N-(1-methylpropyl)-;
5-Pyrimidinecarboxamide, 4-amino-2-(2-methoxyphenyl)-N-(1-methylpropyl)-;
5-Pyrimidinecarboxamide, 4-amino-N-methyl-2-[2-(2-methylpropoxy)phenyl]-;
5-Pyrimidinecarboxamide, 4-amino-2-(2-ethoxyphenyl)-N-propyl-;
5-Pyrimidinecarboxamide, 4-amino-N-ethyl-2-[3-(1-methylethoxy)phenyl]-;
5-Pyrimidinecarboxamide, 4-amino-2-(4-ethylphenyl)-N-(2-methoxyethyl)-;
5-Pyrimidinecarboxamide, 4-amino-N-(3-methoxypropyl)-2-(4-methylphenyl)-;
5-Pyrimidinecarboxamide, 4-amino-N-butyl-2-(3-methoxyphenyl)-;
5-Pyrimidinecarboxamide, 4-amino-2-(3-ethoxyphenyl)-N-(1-methylethyl)-;
5-Pyrimidinecarboxamide, 4-amino-2-(3-butoxyphenyl)-N-methyl-;
5-Pyrimidinecarboxamide, 4-amino-N-butyl-2-(4-methoxyphenyl)-;
5-Pyrimidinecarboxamide, 4-amino-N-butyl-2-(2-methoxyphenyl)-;
5-Pyrimidinecarboxamide, 4-amino-2-(3-ethoxyphenyl)-N-propyl-;
5-Pyrimidinecarboxamide, 4-amino-N-(3-ethoxypropyl)-2-phenyl-;
5-Pyrimidinecarboxamide, 4-amino-2-(3-methoxyphenyl)-N-(2-methylpropyl)-;
5-Pyrimidinecarboxamide, 4-amino-2-(4-methoxyphenyl)-N-(2-methylpropyl)-;
5-Pyrimidinecarboxamide, 4-amino-N-methyl-2-[3-(2-methylpropoxy)phenyl]-;
5-Pyrimidinecarboxamide, 4-amino-2-(3-methoxyphenyl)-N-(1-methylpropyl)-;
5-Pyrimidinecarboxamide, 4-amino-N-(1,1-dimethylethyl)-2-(3-methoxyphenyl)-;
5-Pyrimidinecarboxamide, 4-amino-2-(2-methoxyphenyl)-N-(2-methylpropyl)-;
5-Pyrimidinecarboxamide, 4-amino-N-methyl-2-[4-(1-methylpropoxy)phenyl]-;
5-Pyrimidinecarboxamide, 4-amino-N-methyl-2-[2-(1-methylpropoxy)phenyl]-;
5-Pyrimidinecarboxamide, 4-amino-N-[3-(dimethylamino)propyl]-2-(2-pyridinyl)-;
5-Pyrimidinecarboxamide, 4-amino-N-[3-(dimethylamino)propyl]-2-(4-pyridinyl)-;
5-Pyrimidinecarboxamide, 4-amino-N-[3-(dimethylamino)propyl]-2-(3-pyridinyl)-;
5-Pyrimidinecarboxamide, 4-amino-N-(3-ethoxypropyl)-2-(3-pyridinyl)-;
5-Pyrimidinecarboxamide, 4-amino-N-(3-ethoxypropyl)-2-(4-pyridinyl)-;
5-Pyrimidinecarboxamide, 4-amino-N-(3-ethoxypropyl)-2-(2-pyridinyl)-;
5-Pyrimidinecarboxamide, 4-amino-2-(3-ethoxy-4-methoxyphenyl)-N-methyl-;
5-Pyrimidinecarboxamide, 4-amino-2-(2,4-dimethoxyphenyl)-N-ethyl-;
5-Pyrimidinecarboxamide, 4-amino-2-(4-ethoxy-3-methoxyphenyl)-N-methyl-;
5-Pyrimidinecarboxamide, 4-amino-N-(2-methoxyethyl)-2-(2-methoxyphenyl)-;
5-Pyrimidinecarboxamide, 4-amino-N-(2-methoxyethyl)-2-(3-methoxyphenyl)-;
5-Pyrimidinecarboxamide, 4-amino-2-(2,5-dimethoxyphenyl)-N-ethyl-;
5-Pyrimidinecarboxamide, 4-amino-2-(2-ethoxy-3-methoxyphenyl)-N-methyl-;
5-Pyrimidinecarboxamide, 4-amino-2-(2,3-dimethoxyphenyl)-N-ethyl-;
5-Pyrimidinecarboxamide, 4-amino-N-(2-methoxyethyl)-2-(4-methoxyphenyl)-;
5-Pyrimidinecarboxamide, 4-amino-2-(3,4-dimethoxyphenyl)-N-ethyl-;
5-Pyrimidinecarboxamide, 4-amino-2-(3-fluorophenyl)-N-pentyl-;
5-Pyrimidinecarboxamide, 4-amino-2-(3-fluorophenyl)-N-(3-methylbutyl)-;
5-Pyrimidinecarboxamide, 4-amino-2-(2-fluorophenyl)-N-pentyl-;
5-Pyrimidinecarboxamide, 4-amino-2-(2-fluorophenyl)-N-(3-methylbutyl)-;
5-Pyrimidinecarboxamide, 4-amino-2-(4-fluorophenyl)-N-pentyl-;
5-Pyrimidinecarboxamide, 4-amino-2-(4-fluorophenyl)-N-(3-methylbutyl)-;
5-Pyrimidinecarboxamide, 4-amino-2-(2-methoxy-5-nitrophenyl)-N-methyl-;
5-Pyrimidinecarboxamide, 4-amino-2-(4-methoxy-3-nitrophenyl)-N-methyl-;
5-Pyrimidinecarboxamide, 4-amino-N-[2-(dimethylamino)ethyl]-2-(4-fluorophenyl)-;
5-Pyrimidinecarboxamide, 4-amino-N-[2-(dimethylamino)ethyl]-2-(3-fluorophenyl)-;
5-Pyrimidinecarboxamide, 4-amino-N-[2-(dimethylamino)ethyl]-2-(2-fluorophenyl)-;
5-Pyrimidinecarboxamide, 4-amino-2-(2-fluorophenyl)-N-(3-methoxypropyl)-;
5-Pyrimidinecarboxamide, 4-amino-2-(4-fluorophenyl)-N-(3-methoxypropyl)-;
5-Pyrimidinecarboxamide, 4-amino-2-(3-fluorophenyl)-N-(3-methoxypropyl)-;
5-Pyrimidinecarboxamide, 4-amino-N-(3-methylphenyl)-2-phenyl-;
5-Pyrimidinecarboxamide, 4-amino-2-(2-methylphenyl)-N-phenyl-;
5-Pyrimidinecarboxamide, 4-amino-N-(4-methylphenyl)-2-phenyl-;
5-Pyrimidinecarboxamide, 4-amino-2-(4-methylphenyl)-N-phenyl-;
5-Pyrimidinecarboxamide, 4-amino-2-phenyl-N-(phenylmethyl)-;
5-Pyrimidinecarboxamide, 4-amino-2-(3-methylphenyl)-N-phenyl-;
5-Pyrimidinecarboxamide, 4-amino-N-(2-methylphenyl)-2-phenyl-;
5-Pyrimidinecarboxamide, 4-amino-2-(4-chlorophenyl)-N-(1-methylpropyl)-;
5-Pyrimidinecarboxamide, 4-amino-N-butyl-2-(3-chlorophenyl)-;
5-Pyrimidinecarboxamide, 4-amino-2-(2-chlorophenyl)-N-(1,1-dimethylethyl)-;
5-Pyrimidinecarboxamide, 4-amino-2-(3-chlorophenyl)-N-(1-methylpropyl)-;
5-Pyrimidinecarboxamide, 4-amino-N-butyl-2-(2-chlorophenyl)-;
5-Pyrimidinecarboxamide, 4-amino-N-butyl-2-(4-chlorophenyl)-;
5-Pyrimidinecarboxamide, 4-amino-2-(2-chlorophenyl)-N-(1-methylpropyl)-;
5-Pyrimidinecarboxamide, 4-amino-2-(4-chlorophenyl)-N-(1,1-dimethylethyl)-;
5-Pyrimidinecarboxamide, 4-amino-2-(2-chlorophenyl)-N-(2-methylpropyl)-;
5-Pyrimidinecarboxamide, 4-amino-2-(3-chlorophenyl)-N-(1,1-dimethylethyl)-;
5-Pyrimidinecarboxamide, 4-amino-2-(4-chlorophenyl)-N-(2-methylpropyl)-;
5-Pyrimidinecarboxamide, 4-amino-2-(3-chlorophenyl)-N-(2-methylpropyl)-;
5-Pyrimidinecarboxamide, 4-amino-N-(6-methyl-2-pyridinyl)-2-phenyl-;
5-Pyrimidinecarboxamide, 4-amino-2-(4-methylphenyl)-N-4-pyridinyl-;
5-Pyrimidinecarboxamide, 4-amino-2-(3-methylphenyl)-N-4-pyridinyl-;
5-Pyrimidinecarboxamide, 4-amino-N-(3-methylphenyl)-2-(3-pyridinyl)-;
5-Pyrimidinecarboxamide, 4-amino-N-(3-methyl-2-pyridinyl)-2-phenyl-;
5-Pyrimidinecarboxamide, 4-amino-N-(phenylmethyl)-2-(3-pyridinyl)-;
5-Pyrimidinecarboxamide, 4-amino-N-(3-methylphenyl)-2-(2-pyridinyl)-;
5-Pyrimidinecarboxamide, 4-amino-N-(phenylmethyl)-2-(4-pyridinyl)-;
5-Pyrimidinecarboxamide, 4-amino-N-(2-methylphenyl)-2-(3-pyridinyl)-;
5-Pyrimidinecarboxamide, 4-amino-N-(3-methylphenyl)-2-(4-pyridinyl)-;
5-Pyrimidinecarboxamide, 4-amino-2-phenyl-N-(3-pyridinylmethyl)-;
5-Pyrimidinecarboxamide, 4-amino-2-(2-methylphenyl)-N-4-pyridinyl-;
5-Pyrimidinecarboxamide, 4-amino-2-(3-methylphenyl)-N-3-pyridinyl-;
5-Pyrimidinecarboxamide, 4-amino-N-(4-methylphenyl)-2-(3-pyridinyl)-;
5-Pyrimidinecarboxamide, 4-amino-N-(4-methylphenyl)-2-(4-pyridinyl)-;
5-Pyrimidinecarboxamide, 4-amino-N-(4-methylphenyl)-2-(2-pyridinyl)-;
5-Pyrimidinecarboxamide, 4-amino-2-phenyl-N-(4-pyridinylmethyl)-;
5-Pyrimidinecarboxamide, 4-amino-2-(4-methylphenyl)-N-3-pyridinyl-;
5-Pyrimidinecarboxamide, 4-amino-N-(2-methylphenyl)-2-(2-pyridinyl)-;
5-Pyrimidinecarboxamide, 4-amino-2-phenyl-N-(2-pyridinylmethyl)-;
5-Pyrimidinecarboxamide, 4-amino-2-(2-methylphenyl)-N-3-pyridinyl-;
5-Pyrimidinecarboxamide, 4-amino-N-(phenylmethyl)-2-(2-pyridinyl)-;
5-Pyrimidinecarboxamide, 4-amino-N-(2-methylphenyl)-2-(4-pyridinyl)-;
5-Pyrimidinecarboxamide, 4-amino-N-(4-methyl-2-pyridinyl)-2-phenyl-;
5-Pyrimidinecarboxamide, 4-amino-2-(2-methylphenyl)-N-2-pyridinyl-;
5-Pyrimidinecarboxamide, 4-amino-2-(4-methylphenyl)-N-2-pyridinyl-;
5-Pyrimidinecarboxamide, 4-amino-2-(3-methylphenyl)-N-2-pyridinyl-;
5-Pyrimidinecarboxamide, 4-amino-N-(5-methyl-2-pyridinyl)-2-phenyl-;
5-Pyrimidinecarboxamide, 4-amino-N-(5-methyl-2-pyridinyl)-2-(2-pyridinyl)-;
5-Pyrimidinecarboxamide, 4-amino-N-(3-methyl-2-pyridinyl)-2-(4-pyridinyl)-;
5-Pyrimidinecarboxamide, 4-amino-2-(4-methylphenyl)-N-2-pyrimidinyl-;
5-Pyrimidinecarboxamide, 4-amino-2-(3-pyridinyl)-N-(4-pyridinylmethyl)-;
5-Pyrimidinecarboxamide, 4-amino-2-(2-pyridinyl)-N-(4-pyridinylmethyl)-;
5-Pyrimidinecarboxamide, 4-amino-2-(3-pyridinyl)-N-(3-pyridinylmethyl)-;
5-Pyrimidinecarboxamide, 4-amino-N-(6-methyl-2-pyridinyl)-2-(3-pyridinyl)-;
5-Pyrimidinecarboxamide, 4-amino-N-(4-methyl-2-pyridinyl)-2-(4-pyridinyl)-;
5-Pyrimidinecarboxamide, 4-amino-N-(3-methyl-2-pyridinyl)-2-(2-pyridinyl)-;
5-Pyrimidinecarboxamide, 4-amino-2-(4-pyridinyl)-N-(2-pyridinylmethyl)-;
5-Pyrimidinecarboxamide, 4-amino-2-(4-pyridinyl)-N-(4-pyridinylmethyl)-;
5-Pyrimidinecarboxamide, 4-amino-N-(4-methyl-2-pyridinyl)-2-(2-pyridinyl)-;
5-Pyrimidinecarboxamide, 4-amino-2-(2-methylphenyl)-N-2-pyrimidinyl-;
5-Pyrimidinecarboxamide, 4-amino-N-(6-methyl-2-pyridinyl)-2-(2-pyridinyl)-;
5-Pyrimidinecarboxamide, 4-amino-N-(3-methyl-2-pyridinyl)-2-(3-pyridinyl)-;
5-Pyrimidinecarboxamide, 4-amino-2-(2-pyridinyl)-N-(3-pyridinylmethyl)-;
5-Pyrimidinecarboxamide, 4-amino-2-(4-pyridinyl)-N-(3-pyridinylmethyl)-;
5-Pyrimidinecarboxamide, 4-amino-N-(4-methyl-2-pyridinyl)-2-(3-pyridinyl)-;
5-Pyrimidinecarboxamide, 4-amino-2-(3-pyridinyl)-N-(2-pyridinylmethyl)-;
5-Pyrimidinecarboxamide, 4-amino-N-(5-methyl-2-pyridinyl)-2-(4-pyridinyl)-;
5-Pyrimidinecarboxamide, 4-amino-N-(5-methyl-2-pyridinyl)-2-(3-pyridinyl)-;
5-Pyrimidinecarboxamide, 4-amino-2-(2-pyridinyl)-N-(2-pyridinylmethyl)-;
5-Pyrimidinecarboxamide, 4-amino-2-(3-methylphenyl)-N-2-pyrimidinyl-;
5-Pyrimidinecarboxamide, 4-amino-N-(6-methyl-2-pyridinyl)-2-(4-pyridinyl)-;
5-Pyrimidinecarboxamide, 4-amino-2-(2-chloro-6-fluorophenyl)-N-2-propen-1-yl-;
5-Pyrimidinecarboxamide, 4-amino-2-(2-chlorophenyl)-N-(2-methoxyethyl)-;
5-Pyrimidinecarboxamide, 4-amino-2-(5-chloro-2-ethoxyphenyl)-N-methyl-;
5-Pyrimidinecarboxamide, 4-amino-2-(3-chloro-4-methoxyphenyl)-N-ethyl-;
5-Pyrimidinecarboxamide, 4-amino-2-(4-chlorophenyl)-N-(2-methoxyethyl)-;
5-Pyrimidinecarboxamide, 4-amino-2-(3-chloro-4-ethoxyphenyl)-N-methyl-;
5-Pyrimidinecarboxamide, 4-amino-2-(5-chloro-2-methoxyphenyl)-N-ethyl-;
5-Pyrimidinecarboxamide, 4-amino-2-(3-chlorophenyl)-N-(2-methoxyethyl)-;
5-Pyrimidinecarboxamide, 4-amino-N-(3-fluorophenyl)-2-phenyl-;
5-Pyrimidinecarboxamide, 4-amino-N-(2-fluorophenyl)-2-phenyl-;
5-Pyrimidinecarboxamide, 4-amino-N-(4-fluorophenyl)-2-phenyl-;
5-Pyrimidinecarboxamide, 4-amino-2-(4-fluorophenyl)-N-phenyl-;
5-Pyrimidinecarboxamide, 4-amino-2-(2-fluorophenyl)-N-phenyl-;
5-Pyrimidinecarboxamide, 4-amino-2-(3-fluorophenyl)-N-phenyl-;
5-Pyrimidinecarboxamide, 4-amino-N-(2-furanylmethyl)-2-(3-methylphenyl)-;
5-Pyrimidinecarboxamide, 4-amino-N-(2-furanylmethyl)-2-(4-methylphenyl)-;
5-Pyrimidinecarboxamide, 4-amino-N-(2-furanylmethyl)-2-(2-methylphenyl)-;
5-Pyrimidinecarboxamide, 4-amino-2-(2-chloro-6-fluorophenyl)-N-propyl-;
5-Pyrimidinecarboxamide, 4-amino-2-(2-chloro-6-fluorophenyl)-N-(1-methylethyl)-;
5-Pyrimidinecarboxamide, 4-amino-N-(3-fluorophenyl)-2-(3-pyridinyl)-;
5-Pyrimidinecarboxamide, 4-amino-2-(4-fluorophenyl)-N-4-pyridinyl-;
5-Pyrimidinecarboxamide, 4-amino-N-(2-fluorophenyl)-2-(4-pyridinyl)-;
5-Pyrimidinecarboxamide, 4-amino-2-(2-fluorophenyl)-N-3-pyridinyl-;
5-Pyrimidinecarboxamide, 4-amino-2-(2-fluorophenyl)-N-4-pyridinyl-;
5-Pyrimidinecarboxamide, 4-amino-N-(3-fluorophenyl)-2-(4-pyridinyl)-;
5-Pyrimidinecarboxamide, 4-amino-2-(3-fluorophenyl)-N-2-pyridinyl-;
5-Pyrimidinecarboxamide, 4-amino-2-(2-fluorophenyl)-N-2-pyridinyl-;
5-Pyrimidinecarboxamide, 4-amino-N-(2-fluorophenyl)-2-(3-pyridinyl)-;
5-Pyrimidinecarboxamide, 4-amino-2-(4-fluorophenyl)-N-3-pyridinyl-;
5-Pyrimidinecarboxamide, 4-amino-2-(4-fluorophenyl)-N-2-pyridinyl-;
5-Pyrimidinecarboxamide, 4-amino-N-(2-fluorophenyl)-2-(2-pyridinyl)-;
5-Pyrimidinecarboxamide, 4-amino-2-(3-fluorophenyl)-N-3-pyridinyl-;
5-Pyrimidinecarboxamide, 4-amino-N-(4-fluorophenyl)-2-(2-pyridinyl)-;
5-Pyrimidinecarboxamide, 4-amino-N-(4-fluorophenyl)-2-(3-pyridinyl)-;
5-Pyrimidinecarboxamide, 4-amino-2-(3-fluorophenyl)-N-4-pyridinyl-;
5-Pyrimidinecarboxamide, 4-amino-N-(4-fluorophenyl)-2-(4-pyridinyl)-;
5-Pyrimidinecarboxamide, 4-amino-N-(3-fluorophenyl)-2-(2-pyridinyl)-;
5-Pyrimidinecarboxamide, 4-amino-N-(5-methyl-3-isoxazolyl)-2-(3-methylphenyl)-;
5-Pyrimidinecarboxamide, 4-amino-N-(5-methyl-3-isoxazolyl)-2-(2-methylphenyl)-;
5-Pyrimidinecarboxamide, 4-amino-N-(5-methyl-3-isoxazolyl)-2-(4-methylphenyl)-;
5-Pyrimidinecarboxamide, 4-amino-2-(3-fluorophenyl)-N-2-pyrimidinyl-;
5-Pyrimidinecarboxamide, 4-amino-2-(2-fluorophenyl)-N-2-pyrimidinyl-;
5-Pyrimidinecarboxamide, 4-amino-2-(4-fluorophenyl)-N-2-pyrimidinyl-;
5-Pyrimidinecarboxamide, 4-amino-2-(4-ethylphenyl)-N-2H-tetrazol-5-yl-; and
5-Pyrimidinecarboxamide, 4-amino-N-[(2-chlorophenyl)methyl]-2-(4-fluorophenyl)-.

3. A compound according to claim 1 wherein X¹ is N.

4. A compound according to claim 1, wherein X¹ is CR³, wherein R³ is as defined in claim 1.

5. A compound according to claim 4 wherein R³ is H; halo; or
—O(C₁₋₆ alkyl) optionally substituted with one or more substituents selected from halo, OH and —O(C₁₋₆ alkyl); or
a 6-membered aryl or 5- or 6-membered heteroaryl, either of which is optionally substituted with one or more substituents selected from halo, OH, C₁₋₆ alkyl, —O(C₁₋₆ alkyl), C₁₋₆ haloalkyl and —O(C₁₋₆ haloalkyl).

6. A compound according to claim 4, wherein R³ is —O(C₁₋₆ alkyl) optionally substituted with one or more substituents selected from heterocyclyl, NH₂, halo, OH and —O(C₁₋₆ alkyl).

7. A compound according to claim 1, wherein R³ is —O(C₁₋₄ alkyl) optionally substituted with one or more substituents selected from heterocyclyl, NH₂, halo, OH and —O(C₁₋₄ alkyl).

8. A compound according to claim 1 wherein X² is NR⁴, where R⁴ is as defined in claim 1 and the compound is of general formula (Ia): 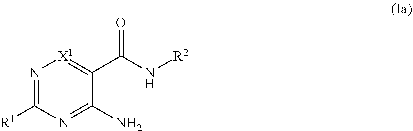
wherein X¹, R¹ and R² are as defined in claim 1.

9. A compound according to claim 1 wherein X² is O and the compound is of general formula (Ib): 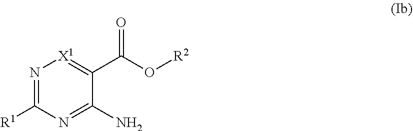
wherein X¹, R¹ and R² are as defined in claim 1.

10. A compound according to claim 1 wherein R¹ is phenyl or 5- or 6-membered heteroaryl optionally substituted as defined in claim 1.

11. A compound according to claim 1 wherein R¹ is phenyl, furanyl, oxazolyl or pyrazolyl, any of which may be unsubstituted or substituted as defined in claim 1.

12. A compound according to claim 1 wherein R¹ is unsubstituted furan-2-yl, 3-fluorophenyl and 3-cyanophenyl.

13. A compound according to claim 1, wherein R¹ is:
phenyl substituted with halo or cyano; or
unsubstituted furan-2-yl.

14. A compound according to claim 1 wherein R² is C₁₋₆ alkyl optionally substituted with one or more substituents selected from halo, OH, O—C₁₋₆ alkyl and R⁸, wherein R⁸ is as defined in claim 1.

15. A compound according to claim 1 wherein R² is CH₂—R⁸, CH₂—CH₂—R⁸ or CH(CH₃)—R⁸, where R⁸ is as defined in claim 1.

16. A compound according to claim 15 wherein R⁸ is phenyl, indan-1-yl, indan-2-yl, pyridin-2-yl, imidazol-2-yl, quinolin-8-yl and triazol-3-yl, any of which is optionally substituted with one or more substituents selected from halo; OH; C₁₋₆ alkyl and —O(C₁₋₆ alkyl), either of which is optionally substituted with halo, OH or —O(C₁₋₆ alkyl).

17. A compound according to claim 15 wherein R⁸ is:
pyridyl optionally substituted with Me, OH, OMe, OEt, CF₃, F; or
quinolinyl optionally substituted with Me, OH, OMe, OEt, CF₃, F; or
phenyl optionally substituted with Me, OH, OMe, OEt, CF₃, F.

18. A compound according to claim 1 wherein R¹ is furan-2-yl, R² is CH₂-(3-methylpyridin-2-yl); and X² is NH.

19. A compound according to claim 1 selected from any one of the following:
Ethyl 5-amino-3-(2-furyl)-1,2,4-triazine-6-carboxylate;
5-Amino-3-(2-furyl)-N-[(3-methyl-2-pyridyl)methyl]-1,2,4-triazine-6-carboxamide;
4-Amino-2-(2-furyl)-N-[(3-methyl-2-pyridyl)methyl]pyrimidine-5-carboxamide;
4-Amino-2-(5-methyl-2-furyl)-N-[(3-methyl-2-pyridyl)methyl]pyrimidine-5-carboxamide;
4-Amino-N-benzyl-2-(3-fluorophenyl)pyrimidine-5-carboxamide;
4-Amino-2-(3-fluorophenyl)-N-(2-phenylethyl)pyrimidine-5-carboxamide;
4-Amino-2-(3-fluorophenyl)-N-phenyl-pyrimidine-5-carboxamide;
4-Amino-2-(3-fluorophenyl)-N-[(3-methyl-2-pyridyl)methyl]pyrimidine-5-carboxamide;
4-Amino-2-(3-fluorophenyl)-N-[(1R)-1-phenylethyl]pyrimidine-5-carboxamide;
4-Amino-2-(3-fluorophenyl)-N-[(2-fluorophenyl) methyl]pyrimidine-5-carboxamide;
4-Amino-N-[(2-fluoro-6-methoxy-phenyl) methyl]-2-(3-fluorophenyl)pyrimidine-5-carboxamide;
4-Amino-N-[(2-ethoxy-6-fluoro-phenyl)methyl]-2-(3-fluorophenyl)pyrimidine-5-carboxamide;
4-Amino-N-[(2,6-difluorophenyl)methyl]-2-(3-fluorophenyl)pyrimidine-5-carboxamide;
4-Amino-2-(3-fluorophenyl)-N-(pyrimidin-2-ylmethyl)pyrimidine-5-carboxamide;
4-Amino-2-(3-fluorophenyl)-N-[[3-(trifluoromethyl)-2-pyridyl]methyl]pyrimidine-5-carboxamide;
4-Amino-2-(3-fluorophenyl)-N-[2-(4-hydroxyphenyl)ethyl]pyrimidine-5-carboxamide;
4-Amino-N-[(3-ethoxy-2-pyridyl)methyl]-2-(3-fluorophenyl)pyrimidine-5-carboxamide;
4-Amino-2-(3-fluorophenyl)-N-(2-pyridyl methyl)pyrimidine-5-carboxamide;
4-Amino-2-(3-fluorophenyl)-N-(o-tolylmethyl) pyrimidine-5-carboxamide;
4-Amino-2-(2-furyl)-N-[(4-methoxyphenyl) methyl]pyrimidine-5-carboxamide;
4-Amino-N-[(3-chlorophenyl)methyl]-2-(2-furyl)pyrimidine-5-carboxamide;
4-Amino-N-[(3-fluorophenyl)methyl]-2-(2-furyl)pyrimidine-5-carboxamide;
4-Amino-N-[(4-fluorophenyl)methyl]-2-(2-furyl)pyrimidine-5-carboxamide;
4-Amino-N-[(2-chlorophenyl)methyl]-2-(2-furyl)pyrimidine-5-carboxamide;
4-Amino-N-[(2,6-dichlorophenyl)methyl]-2-(2-furyl)pyrimidine-5-carboxamide;
4-Amino-N-[(3,5-difluorophenyl)methyl]-2-(2-furyl)pyrimidine-5-carboxamide;
4-Amino-N-[(2-fluorophenyl)methyl]-2-(2-furyl)pyrimidine-5-carboxamide;
4-Amino-N-[(2,4-dichlorophenyl)methyl]-2-(2-furyl)pyrimidine-5-carboxamide;
4-Amino-2-(2-furyl)-N-[(2-methoxyphenyl)methyl]pyrimidine-5-carboxamide;
4-Amino-N-[(3-chloro-2-pyridyl)methyl]-2-(2-furyl)pyrimidine-5-carboxamide;
4-Amino-2-(2-furyl)-N-indan-1-yl-pyrimidine-5-carboxamide;
4-Amino-2-(2-furyl)-N-indan-2-yl-pyrimidine-5-carboxamide;
4-Amino-2-(2-furyl)-N-(8-quinolylmethyl) pyrimidine-5-carboxamide;
4-Amino-N-[(1-ethylimidazol-2-yl)methyl]-2-(2-furyl)pyrimidine-5-carboxamide;
4-Amino-2-(2-furyl)-N-[(2-isopropyl-1,2,4-triazol-3-yl)methyl]pyrimidine-5-carboxamide;
4-Amino-2-(2-furyl)-N-[[4-(trifluoromethyl) phenyl]methyl]pyrimidine-5-carboxamide;
4-Amino-2-(2-furyl)-N-[[3-(trifluoro methyl)phenyl]methyl]pyrimidine-5-carboxamide;
4-Amino-2-(2-furyl)-N-[[3-(trifluoromethyl)-2-pyridyl]methyl]pyrimidine-5-carboxamide;
4-Amino-N-[(2,6-dimethoxyphenyl)methyl]-2-(2-furyl)pyrimidine-5-carboxamide;
4-Amino-2-(2-furyl)-N-[(6-methyl-2-pyridyl)methyl]pyrimidine-5-carboxamide;
4-Amino-2-(2-furyl)-N-[(1-isopropylimidazol-2-yl)methyl]pyrimidine-5-carboxamide;
4-Amino-2-(2-furyl)-N-(m-tolylmethyl) pyrimidine-5-carboxamide;
4-Amino-N-[[2-(difluoromethyl)phenyl]methyl]-2-(2-furyl)pyrimidine-5-carboxamide;
4-Amino-2-(2-furyl)-N-(3-isoquinolylmethyl) pyrimidine-5-carboxamide;
4-Amino-2-(2-furyl)-N-[(1-methylimidazol-2-yl)methyl]pyrimidine-5-carboxamide;
4-Amino-N-[(2,3-dimethoxyphenyl)methyl]-2-(2-furyl)pyrimidine-5-carboxamide;
4-Amino-N-[(3,5-dimethylphenyl)methyl]-2-(2-furyl)pyrimidine-5-carboxamide;
4-Amino-2-(2-furyl)-N-[[3-(trifluoromethoxy) phenyl]methyl]pyrimidine-5-carboxamide;
4-Amino-2-(2-furyl)-N-(1H-indol-5-ylmethyl) pyrimidine-5-carboxamide;
4-Amino-N-[(1S)-6-fluoroindan-1-yl]-2-(2-furyl)pyrimidine-5-carboxamide;
4-Amino-N-[(3,5-dimethoxyphenyl)methyl]-2-(2-furyl)pyrimidine-5-carboxamide;
4-Amino-N-[[3-fluoro-5-(trifluoromethyl) phenyl]methyl]-2-(2-furyl)pyrimidine-5-carboxamide;
4-Amino-2-(2-furyl)-N-(oxazol-2-ylmethyl)pyrimidine-5-carboxamide;
4-Amino-2-(2-furyl)-N-(6-isoquinolylmethyl) pyrimidine-5-carboxamide;
4-Amino-N-[(3-fluoro-2-pyridyl)methyl]-2-(2-furyl)pyrimidine-5-carboxamide;
4-Amino-2-(2-furyl)-N-(5,6,7,8-tetrahydro quinolin-8-yl)pyrimidine-5-carboxamide;
4-Amino-2-(2-furyl)-N-(isoxazol-5-ylmethyl) pyrimidine-5-carboxamide;
4-Amino-N-(1,3-benzodioxol-4-ylmethyl)-2-(2-furyl)pyrimidine-5-carboxamide;
4-Amino-N-[(3-amino-2-pyridyl)methyl]-2-(2-furyl)pyrimidine-5-carboxamide;
4-Amino-2-(2-furyl)-N-[[2-(trifluoromethyl) phenyl]methyl]pyrimidine-5-carboxamide;
4-Amino-N-[(2-fluoro-6-methoxy-phenyl) methyl]-2-(2-furyl)pyrimidine-5-carboxamide;
4-Amino-2-(2-furyl)-N-[1-(3-methyl-2-pyridyl)ethyl]pyrimidine-5-carboxamide;
4-Amino-2-(2-furyl)-N-(pyrimidin-2-ylmethyl) pyrimidine-5-carboxamide;
4-Amino-2-(2-furyl)-N-[[4-(trifluoromethyl) pyrimidin-2-yl]methyl]pyrimidine-5-carboxamide;
4-Amino-2-(2-furyl)-N-(3-hydroxypropyl) pyrimidine-5-carboxamide;
4-Amino-2-(2-furyl)-N-[(1S)-2-hydroxy-1-phenyl-ethyl]pyrimidine-5-carboxamide;
5-Amino-3-(3-fluorophenyl)-N-[(3-methyl-2-pyridyl)methyl]-1,2,4-triazine-6-carboxamide;
5-Amino-3-(4-fluorophenyl)-N-[(3-methyl-2-pyridyl)methyl]-1,2,4-triazine-6-carboxamide;
4-Amino-2-(2-fluorophenyl)-N-[(3-methyl-2-pyridyl)methyl]pyrimidine-5-carboxamide;
4-Amino-2-(3-fluorophenyl)-N-(p-tolylmethyl)pyrimidine-5-carboxamide;
4-Amino-2-(3-fluorophenyl)-N-[(2-hydroxyphenyl)methyl]pyrimidine-5-carboxamide;
4-Amino-N-[(3-chlorophenyl)methyl]-2-(3-fluorophenyl)pyrimidine-5-carboxamide;
4-Amino-2-(3-fluorophenyl)-N-[(3-fluorophenyl)methyl]pyrimidine-5-carboxamide;
4-Amino-2-(3-fluorophenyl)-N-[(4-fluorophenyl) methyl]pyrimidine-5-carboxamide;
4-Amino-N-[(2-chlorophenyl)methyl]-2-(3-fluorophenyl)pyrimidine-5-carboxamide;
4-Amino-N-[(2,6-dichlorophenyl)methyl]-2-(3-fluorophenyl)pyrimidine-5-carboxamide;
4-Amino-N-[(3,5-difluorophenyl)methyl]-2-(3-fluorophenyl)pyrimidine-5-carboxamide;
4-Amino-2-(3-fluorophenyl)-N-[(2-methoxyphenyl)methyl]pyrimidine-5-carboxamide;
4-Amino-N-[(3,5-dimethoxyphenyl)methyl]-2-(3-fluorophenyl)pyrimidine-5-carboxamide;
4-Amino-N-[(2,3-dimethoxyphenyl)methyl]-2-(3-fluorophenyl)pyrimidine-5-carboxamide;
4-Amino-2-(3-fluorophenyl)-N-[[3-(trifluoromethyl)phenyl]methyl]pyrimidine-5-carboxamide;
4-Amino-N-[(3-chloro-2-pyridyl)methyl]-2-(3-fluorophenyl)pyrimidine-5-carboxamide;
4-Amino-2-(3-fluorophenyl)-N-(8-quinolylmethyl)pyrimidine-5-carboxamide;
4-Amino-N-[(1-ethylimidazol-2-yl)methyl]-2-(3-fluorophenyl)pyrimidine-5-carboxamide;
4-Amino-2-(3-fluorophenyl)-N-[(2-isopropyl-1,2,4-triazol-3-yl)methyl]pyrimidine-5-carboxamide;
4-Amino-2-(2-furyl)-N-(p-tolylmethyl) pyrimidine-5-carboxamide;
4-amino-2-(2-furyl)-N-[(2-hydroxyphenyl) methyl]pyrimidine-5-carboxamide;
4-Amino-2-(3-cyanophenyl)-N-[(3-methyl-2-pyridyl)methyl]pyrimidine-5-carboxamide;
4-Amino-6-chloro-2-(2-furyl)-N-[(3-methyl-2-pyridyl)methyl]pyrimidine-5-carboxamide;
4-Amino-2-(4-fluorophenyl)-N-[(3-methyl-2-pyridyl)methyl]pyrimidine-5-carboxamide;
4-Amino-N-[(3-methyl-2-pyridyl)methyl]-2-pyrazol-1-yl-pyrimidine-5-carboxamide;
4-Amino-N-[(3-methyl-2-pyridyl)methyl]-2-oxazol-2-yl-pyrimidine-5-carboxamide;
4-Amino-N-[(3-methyl-2-pyridyl)methyl]-2-thiazol-2-yl-pyrimidine-5-carboxamide;
5-Amino-3-(3-cyanophenyl)-N-[(3-methyl-2-pyridyl)methyl]-1,2,4-triazine-6-carboxamide;
4-Amino-2-(2-furyl)-6-methoxy-N-[(3-methyl-2-pyridyl)methyl]pyrimidine-5-carboxamide;
4-Amino-2-(2-furyl)-6-methoxy-N-[[3-(trifluoromethyl)-2-pyridyl]methyl]pyrimidine-5-carboxamide;
4-Amino-N-[(2,6-dichlorophenyl)methyl]-2-(2-furyl)-6-methoxy-pyrimidine-5-carboxamide;
4-Amino-2-(2-furyl)-N-[(3-methyl-2-pyridyl)methyl]-6-pyrazol-1-yl-pyrimidine-5-carboxamide;
4-(2-Acetamidoethoxy)-6-amino-2-(2-furyl)-N-[(3-methyl-2-pyridyl)methyl]pyrimidine-5-carboxamide;
4-Amino-2-(2-furyl)-6-(2-hydroxyethoxy)-N-[(3-methyl-2-pyridyl)methyl]pyrimidine-5-carboxamide;
4-Amino-2-(2-furyl)-N-[(3-methyl-2-pyridyl)methyl]-6-(2,2,2-trifluoroethoxy) pyrimidine-5-carboxamide;
4-Amino-2-(2-furyl)-6-isobutoxy-N-[(3-methyl-2-pyridyl)methyl]pyrimidine-5-carboxamide;
4-Amino-2-(2-furyl)-6-(2-methoxyethoxy)-N-[(3-methyl-2-pyridyl)methyl]pyrimidine-5-carboxamide;
4-Amino-6-(2-fluoroethoxy)-2-(2-furyl)-N-[(3-methyl-2-pyridyl)methyl]pyrimidine-5-carboxamide;
4-Amino-2-(2-furyl)-6-(3-hydroxypropoxy)-N-[(3-methyl-2-pyridyl)methyl]pyrimidine-5-carboxamide;
4-Amino-2-(2-furyl)-N-[(3-methyl-2-pyridyl) methyl]-6-propoxy-pyrimidine-5-carboxamide;
4-Amino-6-(2,2-dimethylpropoxy)-2-(2-furyl)-N-[(3-methyl-2-pyridyl)methyl]pyrimidine-5-carboxamide;
4-Amino-2-(2-furyl)-6-[(2S)-2-hydroxy propoxy]-N-[(3-methyl-2-pyridyl)methyl]pyrimidine-5-carboxamide;
4-Amino-2-(2-furyl)-6-(2-methoxy-1-methyl-ethoxy)-N-[(3-methyl-2-pyridyl)methyl]pyrimidine-5-carboxamide;
4-Amino-2-(2-furyl)-6-[(2R)-2-hydroxy propoxy]-N-[(3-methyl-2-pyridyl)methyl]pyrimidine-5-carboxamide;
4-(2-Acetamidoethylamino)-6-amino-2-(2-furyl)-N-[(3-methyl-2-pyridyl)methyl]pyrimidine-5-carboxamide;
4-Amino-2-(2-furyl)-N-[(3-methyl-2-pyridyl)methyl]-6-[2-(2-piperidyl)ethoxy]pyrimidine-5-carboxamide;
4-Amino-6-(2-aminoethoxy)-2-(2-furyl)-N-[(3-methyl-2-pyridyl)methyl]pyrimidine-5-carboxamide;
4-Amino-2-(2-furyl)-N-[(3-methyl-2-pyridyl)methyl]-6-(2-piperazin-1-ylethoxy) pyrimidine-5-carboxamide;
4-Amino-6-(3-aminopropoxy)-2-(2-furyl)-N-[(3-methyl-2-pyridyl)methyl]pyrimidine-5-carboxamide;
4-Amino-2-(3-cyanophenyl)-6-(2-fluoroethoxy)-N-isopropyl-pyrimidine-5-carboxamide;
4-Amino-2-(3-cyanophenyl)-6-(2-fluoroethoxy)-N-propyl-pyrimidine-5-carboxamide;
4-Amino-2-(3-cyanophenyl)-6-(2-fluoroethoxy)-N-isobutyl-pyrimidine-5-carboxamide;
4-Amino-N-butyl-2-(3-cyanophenyl)-6-(2-fluoroethoxy)pyrimidine-5-carboxamide;
4-Amino-2-(3-cyanophenyl)-6-(2-fluoroethoxy)-N-[(2-isopropyl-1,2,4-triazol-3-yl)methyl]pyrimidine-5-carboxamide;
4-Amino-2-(3-cyanophenyl)-N-[2-(dimeth ylamino)ethyl]-6-(2-fluoroethoxy) pyrimidine-5-carboxamide;
4-Amino-2-(3-cyanophenyl)-6-(2-fluoroethoxy)-N-(3-hydroxybutyl)pyrimidine-5-carboxamide;
4-Amino-2-(3-cyanophenyl)-6-(2-fluoroethoxy)-N-[(2-methyl-1,2,4-triazol-3-yl) methyl]pyrimidine-5-carboxamide;
4-Amino-2-(3-cyanophenyl)-6-(2-fluoroethoxy)-N-[(3-methyl-2-pyridyl)methyl]pyrimidine-5-carboxamide;
4-Amino-6-(2-fluoroethoxy)-N-[(3-methyl-2-pyridyl)methyl]-2-oxazol-2-yl-pyrimidine-5-carboxamide;
and pharmaceutically acceptable salts and solvates thereof.

20. A process for the preparation of a compound according to claim 1 comprising
A. for a compound of general formula (Ia):
reacting a compound of general formula (II): 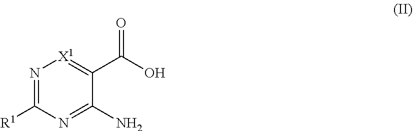
wherein X¹ and R¹ are as defined for general formula (I);
with a compound of general formula (III): 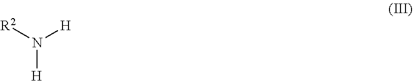
wherein R² is as defined for general formula (I);
B. for a compound of general formula (Ib) in which X¹ is N or C—R³:
reacting a compound of general formula (IV): 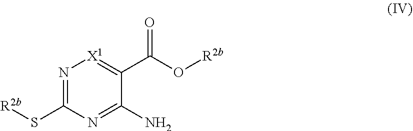
wherein each R^{2b} is independently C₁₋₆ alkyl;
X¹ is N or CR³, wherein R³ is as defined for general formula (I);
with a compound of general formula (V): 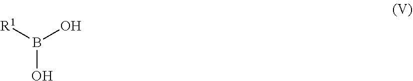
wherein R¹ is as defined for general formula (I);
C. for a compound of general formula (Ib) in which X¹ is CH:
reacting a compound of general formula (V) as defined above with a compound of general formula (XV): 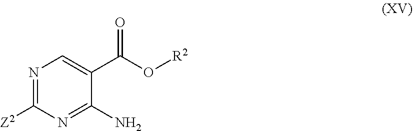
wherein R^{2b} is as defined for general formula (IV) and Z² is halo, suitably chloro;
D. for a compound of general formula (Ia) wherein X¹ is CH:
reacting a compound of general formula (XVI): 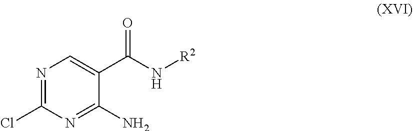
wherein R² is as defined for general formula (I);
with a compound of general formula (V) as defined above;
E. for a compound of general formula (Ib) in which R² is R^{2b} and X¹ is N:
reacting a compound of general formula (XX): 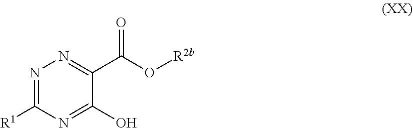
wherein R¹ is as defined for general formula (I) and R^{2b} is as defined for general formula (IV);
with ammonium hydroxide;
F. for a compound of general formula (Ia) in which X¹ is CH:
Reacting a compound of general formula (XXXI): 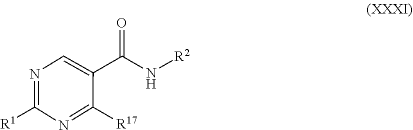
wherein R¹ and R² are as defined for general formula (I) and R¹⁷ is as defined for general formula (XXX);
with sodium azide followed by reduction with triphenylphosphine.

21. A process for the preparation of a compound having the general formula (I) defined in claim 1, in which X¹ is CR³ and R³ is O(C₁₋₆ alkyl), optionally substituted as defined in claim 1, wherein the process comprises reacting a compound of Formula (Ya): 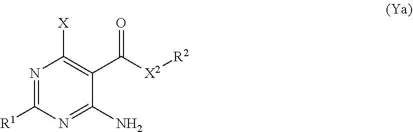
wherein R¹, R², and X² are as defined in claim 1, and X is a leaving group, for example halo;
with a compound of general formula (Za);
R₃ₐ—OH (Za)
wherein R₃ₐ is (C₁₋₆ alkyl), optionally substituted with one or more substituents selected from halo, OH, —O(C₁₋₆ alkyl), —NR⁹R¹⁰, —NR⁹C(O)R¹⁰, NR⁹C(═NR⁴)NR¹⁰, NR⁹C(S)R¹⁰, carbocyclyl, heterocyclyl, aryl and heteroaryl,
wherein each R⁹ and R¹⁰ is independently selected from H, C₁₋₆ alkyl and C₁₋₆ haloalkyl;
and any carbocyclyl, heterocyclyl, aryl and heteroaryl groups are optionally substituted with one or more substituents selected from halo, C₁₋₆ alkyl, C₁₋₆ haloalkyl and —NR¹¹R¹²; wherein each R¹¹ and R¹² is independently selected from H, C₁₋₆ alkyl and C₁₋₆ haloalkyl;

22. (canceled)

23. (canceled)

24. A method for the treatment of cancer, particularly solid tumours, for example non-small cell lung cancer, head and neck squamous cancer and urothelial cancer, the method comprising administering to a patient in need of such treatment an effective amount of a compound according to claim 1, including all tautomeric forms, enantiomers, isotopic variants, salts and solvates thereof.

25. A pharmaceutical composition comprising a compound according to claim 1, including all tautomeric forms, enantiomers, isotopic variants, salts and solvates thereof and a pharmaceutically acceptable excipient.

26. A composition according to claim 25 further including one or more other active agents which are useful in the treatment or prophylaxis of cancer.

27. A product comprising a compound according to claim 1, including all tautomeric forms, enantiomers, isotopic variants, salts and solvates thereof and an additional agent useful in the treatment or prevention of cancer as a combined preparation for simultaneous, sequential or separate use in the treatment of cancer, in particular solid tumours for example non-small cell lung cancer, head and neck squamous cancer and urothelial cancer.

28. A compound according to claim 1, including all tautomeric forms, enantiomers, isotopic variants, salts and solvates thereof in combination with an additional agent useful in the treatment of cancer as a combined preparation for simultaneous, sequential or separate use in the treatment of treatment of cancer, in particular solid tumours for example non-small cell lung cancer, head and neck squamous cancer and urothelial cancer.

29. A compound in combination according to claim 28, wherein the additional agent useful in the treatment of cancer is selected from:
other forms of cancer immunotherapy and anti-cancer chemotherapeutic agents;
A2b antagonists;
anti-PD-1 and PDL-1 antibodies including pembrolizumab, nivolumab, durvalumab, avelumab and atezolizumab;
anti-CTLA4 antibodies including ipilimumab; and
cell-based immunotherapy and cancer vaccines that include CAR T cell therapy.
